(19) United States
(12) Patent Application Publication
(10) Pub. No.: US 2026/0027145 A1
(43) Pub. Date: Jan. 29, 2026

(54) METHODS OF TRANSDUCTION USING A VIRAL VECTOR AND INHIBITORS OF ANTIVIRAL RESTRICTION FACTORS

(71) Applicant: Sana Biotechnology, Inc., Seattle, WA (US)

(72) Inventors: Mariliis OTT, Seattle, WA (US); Daryl HUMES, Seattle, WA (US); Neal VAN HOEVEN, Seattle, WA (US)

(73) Assignee: Sana Biotechnology, Inc., Seattle, WA (US)

(21) Appl. No.: 18/998,337

(22) PCT Filed: Jul. 26, 2023
(86) PCT No.: PCT/US2023/071070

§ 371 Date: Jan. 24, 2025

Related U.S. Application Data
(60) Provisional application No. 63/392,837, filed on Jul. 27, 2022.
(60) Provisional application No. 63/460,290, filed on Apr. 18, 2023.
(60) Provisional application No. 63/466,703, filed on May 15, 2023.

(51) Int. Cl. A61K 31/7088; A61K 31/436; A61K 31/7048; A61K 35/76; A61K 38/20; A61K 47/08; A61P 35/02; C12N 5/078; C12N 15/86

(52) U.S. Cl. CPC A61K 31/7088; A61K 31/436; A61K 31/7048; A61K 35/76; A61K 38/2046; A61K 38/2086; A61K 47/08; A61P 35/02; C12N 5/0634; C12N 15/86

(57) ABSTRACT

Provided herein are methods of transducing or delivering an exogenous agent to a cell using a viral vector pseudotyped with a paramyxovirus and an inhibitor of the mammalian target of rapamycin (mTOR). In some embodiments, the methods can further include the administration of an antiviral restriction factor inhibitor and/or a cytokine.

### CROSS-REFERENCE TO RELATED APPLICATIONS

This application claims priority to a U.S. Provisional Patent Application No. 63/392,837, filed Jul. 27, 2022, entitled “METHODS OF TRANSDUCTION USING A VIRAL VECTOR AND INHIBITORS OF ANTIVIRAL RESTRICTION FACTORS”, U.S. Provisional Patent Application No. 63/460,290, filed Apr. 18, 2023, entitled “METHODS OF TRANSDUCTION USING A VIRAL VECTOR AND INHIBITORS OF ANTIVIRAL RESTRICTION FACTORS”, and U.S. Provisional Patent Application No. 63/466,703, filed May 15, 2023, entitled “METHODS OF TRANSDUCTION USING A VIRAL VECTOR AND INHIBITORS OF ANTIVIRAL RESTRICTION FACTORS”, each of which is herein incorporated by reference in its entirety for all purposes.

### REFERENCE TO AN ELECTRONIC SEQUENCE LISTING

The present application is being filed along with a Sequence Listing in electronic format. The Sequence Listing is provided as a file entitled 18615_2007040_Seq.XML created Jul. 26, 2023 which is 309,878 bytes in size. The information in the electronic format of the Sequence Listing is incorporated by reference in its entirety.

### FIELD

The present disclosure related to methods of transducing or delivering an exogenous agent to a cell using a viral vector and an inhibitor of the mammalian target of rapamycin (mTOR). In some embodiments, the methods include an inhibitor of an antiviral restriction factor. In some embodiments, the methods are in-line methods of administration of a viral vector, such as for the delivery of an exogenous agent.

### SUMMARY

Provided herein is a method of transducing cells in subject, the method comprising: (a) administering to a subject an inhibitor of the mammalian target of rapamycin (mTOR), and (b) administering to the subject a viral vector comprising a viral fusogen embedded in the lipid bilayer. In some of any of the provided embodiments, the viral vector comprises an exogenous agent.

Provided herein is a method of delivering an exogenous agent to a subject, the method comprising: (a) administering to a subject an inhibitor of mTOR, and (b) administering to the subject a viral vector comprising an exogenous agent, wherein the viral vector comprises a fusogen embedded in the lipid bilayer.

Provided herein is a method of transducing cells in a subject, the method comprising (a) administering to a subject an inhibitor of the mammalian target of rapamycin (mTOR), and (b) administering to the subject a lentiviral vector comprising one or more Nipah envelope proteins or functional variants thereof wherein the vector comprises a polynucleotide encoding a chimeric antigen receptor (CAR), and (c) administering to the subject IL-7 or a functional variant thereof.

Provided herein is a method of transducing cells in a subject, the method comprising (a) administering to a subject an inhibitor of the mammalian target of rapamycin (mTOR), and (b) administering to the subject a lentiviral vector comprising one or more Nipah envelope proteins or functional variants thereof wherein the vector comprises (i) a viral VPX protein and (ii) a polynucleotide encoding a chimeric antigen receptor (CAR).

In some of any of the provided embodiments, the inhibitor of mTOR and the viral vector are administered separately or in the same composition. In some of any of the provided embodiments, the inhibitor of mTOR and the viral vector are administered separately. In some of any of the provided embodiments, the inhibitor of mTOR is administered prior to, consecutively, or after administering the viral vector. In some of any of the provided embodiments, the time period between the administration of the inhibitor of mTOR and viral vector is no more than three days. In some of any of the provided embodiments, the time period between the administration of the inhibitor of mTOR and viral vector is no more than one day. In some of any of the provided embodiments, the time period between the administration of the inhibitor of mTOR and viral vector is no more than 12, 6, or 3 hours. In some of any of the provided embodiments, the inhibitor of mTOR and the viral vector are administered on the same day.

In some of any of the provided embodiments, the method further comprises administering to the subject an inhibitor of an antiviral restriction factor.

Provided herein is a method of transducing cells in subject, the method comprising: (a) administering to a subject an inhibitor of an antiviral restriction factor, and (b) administering to the subject a viral vector comprising a viral fusogen embedded in the lipid bilayer.

In some of any of the provided embodiments, the inhibitor of an antiviral restriction factor is an inhibitor of SAMHD1, an inhibitor of IFITM1, and/or an inhibitor of IFITM3. In some of any of the provided embodiments, the inhibitor of an antiviral restriction factor is an inhibitor of SAMHD1, optionally wherein the inhibitor increases phosphorylation and/or degradation of SAMHD1. In some of any of the provided embodiments, the inhibitor of an antiviral restriction factor is an inhibitor of IFITM1 and/or an inhibitor of IFITM3, optionally wherein the inhibitor reduces expression of IFITM1. In some of any of the provided embodiments, the inhibitor of an antiviral restriction factor is an oligonucleotide, optionally wherein the inhibitor of an antiviral restriction factor is an anti-sense oligonucleotide complementary to an RNA encoding said cellular restriction factor. In some of any of the provided embodiments, the inhibitor of an antiviral restriction factor is a resveratrol cyclotrimer, optionally caraphenol A, a-viniferin or resveratrol, or an analog compound thereof.

In some of any of the provided embodiments, the inhibitor of an antiviral restrictions factor is an antifungal agent. In some of any of the provided embodiments, the inhibitor of an antiviral restriction factor is a polyene antifungal agent, optionally nystatin, pimaricin, or amphotericin B. In some of any of the provided embodiments, wherein the inhibitor of an antiviral restriction factor is amphotericin B.

In some of any of the provided embodiments, the time period between the administration of the inhibitor of the antiviral restriction factor and viral vector is no more than one day. In some of any of the provided embodiments, the time period between the administration of the inhibitor of the antiviral restriction factor and viral vector is no more than 12 hours. In some of any of the provided embodiments, the time period between the administration of the inhibitor of the antiviral restriction factor and viral vector is no more than 1, 2, 3, 4, or 5 hours. In some of any of the provided embodiments, the inhibitor of an antiviral restriction factor is administered at a dose of at or about 1-10 mg, 10-20 mg, 20-30 mg, 30-40 mg, or 40-50, or any value between the foregoing.

Provided herein is a method for administering a viral vector to a subject, the method comprising: (a) contacting peripheral blood mononuclear cells (PBMCs) or a subset thereof from a subject with a composition comprising a viral vector and an inhibitor of mTOR to create a transduction mixture; and (b) administering the transduction mixture to the subject, thereby administering the viral vector to the subject.

Provided here is a method for administering a viral vector to a subject, the method comprising: (a) contacting peripheral blood mononuclear cells (PBMCs) or a subset thereof from a subject with a composition comprising a viral vector and an inhibitor of mTOR that is temsirolimus to create a transduction mixture; and (b) administering the transduction mixture to the subject, thereby administering the viral vector to the subject.

Provided herein is a method for administering a viral vector to a subject, the method comprising: (a) contacting peripheral blood mononuclear cells (PBMCs) or a subset thereof from a subject with a composition comprising a viral vector and an inhibitor of an antiviral restriction factor to create a transduction mixture; and (b) administering the transduction mixture to the subject, thereby administering the viral vector to the subject.

Also provided herein is a method for administering a viral vector to a subject, the method comprising: (a) contacting peripheral blood mononuclear cells (PBMCs) or a subset thereof from a subject with a composition comprising a viral vector and a polyene antifungal agent to create a transduction mixture; and (b) administering the transduction mixture to the subject, thereby administering the viral vector to the subject.

In some of any of the provided embodiments, the contacting and administering is performed in a closed fluid circuit. In some of any of the provided embodiments, an inhibitor of mTOR is administered prior to contacting in step (A). In some of any of the provided embodiments, the inhibitor of an antiviral restriction factor is administered prior to contacting in step (A). In some of any of the provided embodiments, the polyene antifungal agent is administered prior to contacting in step (A). In some of any of the provided embodiments, the polyene antifungal agent is selected from the group comprising nystatin, pimaricin, or amphotericin B.

Provided herein is a method for administering a viral vector to a subject, the method comprising: (a) obtaining whole blood from a subject; (b) collecting from the whole blood a fraction of blood containing PBMCs or a subset thereof; (c) contacting the fraction of blood containing PBMCs or a subset thereof with a viral vector and an inhibitor of mTOR to create a transduction mixture; and (d) reinfusing the transduction mixture to the subject, thereby administering the viral vector to the subject, wherein steps (a)-(d) are performed in-line in a closed fluid circuit.

Provided herein is a method for administering a viral vector to a subject, the method comprising: (a) obtaining whole blood from a subject; (b) collecting from the whole blood a fraction of blood containing PBMCs or a subset thereof; (c) contacting the fraction of blood containing PBMCs or a subset thereof with a viral vector and an inhibitor of an antiviral restriction factor to create a transduction mixture; and (d) reinfusing the transduction mixture to the subject, thereby administering the viral vector to the subject, wherein steps (a)-(d) are performed in-line in a closed fluid circuit.

Provided herein is a method for administering a viral vector to a subject, the method comprising: (a) Administering to a subject an inhibitor of mTOR; (b) obtaining whole blood from a subject; (c) collecting from the whole blood a fraction of blood containing PBMCs or a subset thereof; (d) contacting the fraction of blood containing PBMCs or a subset thereof with a viral vector to create a transduction mixture; and (e) reinfusing the transduction mixture to the subject, thereby administering the viral vector to the subject, wherein steps (a)-(e) are performed in-line in a closed fluid circuit.

Provided herein is a method for administering a viral vector to a subject, the method comprising: (a) administering to a subject an inhibitor of an antiviral restriction factor; (b) obtaining whole blood from a subject; (c) collecting from the whole blood a fraction of blood containing PBMCs or a subset thereof; (d) contacting the fraction of blood containing PBMCs or a subset thereof with a viral vector to create a transduction mixture; and (e) reinfusing the transduction mixture to the subject, thereby administering the viral vector to the subject, wherein steps (a)-(e) are performed in-line in a closed fluid circuit.

In some of any of the provided embodiments, the method further comprises contacting the fraction of blood containing PBMCs or a subset thereof with a viral vector and an inhibitor of mTOR to create a transduction mixture in step (d). In some of any of the provided embodiments, the method further comprises contacting the fraction of blood containing PBMCs or a subset thereof with a viral vector and an inhibitor of an antiviral restriction factor to create a transduction mixture in step (d).

In some of any of the provided embodiments, the viral vector comprises an exogenous agent.

Provided herein is a method for delivering an exogenous agent to a subject, the method comprising: (a) contacting PBMCs or a subset thereof from a subject with a viral vector and an inhibitor of mTOR to create a transduction mixture, wherein the viral vector comprises an exogenous agent; and (b) administering the transduction mixture to the subject, thereby administering the exogenous agent to the subject.

Provided herein is a method for delivering an exogenous agent to a subject, the method comprising: (a) contacting PBMCs or a subset thereof from a subject with a viral vector and an inhibitor of mTOR that is temsirolimus to create a transduction mixture, wherein the viral vector comprises an exogenous agent; and (b) administering the transduction mixture to the subject, thereby administering the exogenous agent to the subject.

Provided herein is a method for delivering an exogenous agent to a subject, the method comprising: (a) contacting PBMCs or a subset thereof from a subject with a viral vector and an inhibitor of an antiviral restriction factor to create a transduction mixture, wherein the viral vector comprises an exogenous agent; and (b) administering the transduction mixture to the subject, thereby administering the exogenous agent to the subject.

Provided herein is a method for delivering an exogenous agent to a subject, the method comprising: (a) contacting PBMCs or a subset thereof from a subject with a viral vector and a polyene antifungal agent to create a transduction mixture, wherein the viral vector comprises an exogenous agent; and (b) administering the transduction mixture to the subject, thereby administering the exogenous agent to the subject.

In some of any of the provided embodiments, the contacting and administering is performed in a closed fluid circuit. In some of any of the provided embodiments, an inhibitor of mTOR is administered prior to contacting in step (A). In some of any of the provided embodiments, the inhibitor of an antiviral restriction factor is administered prior to contacting in step (A). In some of any of the provided embodiments, the polyene antifungal agent is administered prior to contacting in step (A). In some of any of the provided embodiments, the polyene antifungal agent is selected from the group comprising nystatin, pimaricin, or amphotericin B.

Provided herein is a method for delivering an exogenous agent to a subject, the method comprising: (a) obtaining whole blood from a subject; (b) collecting from the whole blood a fraction of blood containing peripheral blood mononuclear cells (PBMCs) or a subset thereof; (c) contacting the fraction of blood containing peripheral blood mononuclear cells (PBMCs) or a subset thereof with a viral vector and an inhibitor of mTOR to create a transduction mixture, wherein the viral vector comprises an exogenous agent; and (d) reinfusing the transduction mixture to the subject, thereby administering the exogenous agent to the subject, wherein steps (a)-(d) are performed in-line in a closed fluid circuit.

Provided herein is a method for delivering an exogenous agent to a subject, the method comprising: (a) obtaining whole blood from a subject; (b) collecting from the whole blood a fraction of blood containing peripheral blood mononuclear cells (PBMCs) or a subset thereof; (c) contacting the fraction of blood containing peripheral blood mononuclear cells (PBMCs) or a subset thereof with a viral vector and an inhibitor of an antiviral restriction factor to create a transduction mixture, wherein the viral vector comprises an exogenous agent; and (d) reinfusing the transduction mixture to the subject, thereby administering the exogenous agent to the subject wherein steps (a)-(d) are performed in-line in a closed fluid circuit.

Provided herein is a method for delivering an exogenous agent to a subject, the method comprising: (a) Administering to the subject inhibitor of mTOR; (b) obtaining whole blood from a subject; (c) collecting from the whole blood a fraction of blood containing PBMCs or a subset thereof; (d) contacting the fraction of blood containing PBMCs or a subset thereof with a viral vector to create a transduction mixture; and (e) reinfusing the transduction mixture to the subject, thereby administering the viral vector to the subject, wherein steps (a)-(e) are performed in-line in a closed fluid circuit.

Provided herein is a method for delivering an exogenous agent to a subject, the method comprising: (a) administering to the subject inhibitor of an antiviral restriction factor; (b) obtaining whole blood from a subject; (c) collecting from the whole blood a fraction of blood containing PBMCs or a subset thereof; (d) contacting the fraction of blood containing PBMCs or a subset thereof with a viral vector to create a transduction mixture; and (e) reinfusing the transduction mixture to the subject, thereby administering the viral vector to the subject, wherein steps (a)-(e) are performed in-line in a closed fluid circuit.

In some of any of the provided embodiments, the method further comprises contacting the fraction of blood containing PBMCs or a subset thereof with a viral vector and an inhibitor of mTOR to create a transduction mixture in step (d). In some of any of the provided embodiments, the method further comprises contacting the fraction of blood containing PBMCs or a subset thereof with a viral vector and an inhibitor of an antiviral restriction factor to create a transduction mixture in step (d).

Provided herein is a method for administering a viral vector to a subject, the method comprising: (a) contacting peripheral blood mononuclear cells (PBMCs) or a subset thereof from a subject with a composition comprising a lentiviral vector comprising one or more Nipah envelope proteins or functional variants thereof, an inhibitor of an antiviral restriction factor, and Il-7 or a functional variant thereof to create a transduction mixture; and (b) administering the transduction mixture to the subject, thereby administering the viral vector to the subject, wherein the vector comprises a chimeric antigen receptor (CAR).

Provided herein is a method for administering a viral vector to a subject, the method comprising: (a) contacting peripheral blood mononuclear cells (PBMCs) or a subset thereof from a subject with a composition comprising a lentiviral vector comprising one or more Nipah envelope proteins or functional variants thereof and an inhibitor of an antiviral restriction factor, to create a transduction mixture; and (b) administering the transduction mixture to the subject, thereby administering the viral vector to the subject, wherein the vector comprises a viral VPX protein and a chimeric antigen receptor (CAR).

Provided herein is a method for transducing cells in a subject, the method comprising: (a) contacting peripheral blood mononuclear cells (PBMCs) or a subset thereof from a subject with a composition comprising a lentiviral vector comprising one or more Nipah envelope proteins or functional variants thereof, and an inhibitor of an antiviral restriction factor to create a transduction mixture; and (b) administering the transduction mixture to the subject, thereby administering the viral vector to the subject, wherein the vector comprises a viral VPX protein and a chimeric antigen receptor (CAR).

Provided herein is a method for transducing cells in a subject, the method comprising:

(a) contacting peripheral blood mononuclear cells (PBMCs) or a subset thereof from a subject with a composition comprising a lentiviral vector comprising one or more Nipah envelope proteins or functional variants thereof, an inhibitor of an antiviral restriction factor, and Il-7 or a functional variant thereof to create a transduction mixture; and (b) administering the transduction mixture to the subject, thereby administering the viral vector to the subject, wherein the vector comprises a chimeric antigen receptor (CAR).

Provided herein is a method for administering a viral vector to a subject, the method comprising: (a) contacting peripheral blood mononuclear cells (PBMCs) or a subset thereof from a subject with a composition comprising a lentiviral vector comprising one or more Nipah envelope proteins or functional variants thereof, an inhibitor of mTOR, and Il-7 or a functional variant thereof to create a transduction mixture; and (b) administering the transduction mixture to the subject, thereby administering the viral vector to the subject, wherein the vector comprises a chimeric antigen receptor (CAR).

Provided herein is a method for administering a viral vector to a subject, the method comprising: (a) contacting peripheral blood mononuclear cells (PBMCs) or a subset thereof from a subject with a composition comprising a lentiviral vector comprising one or more Nipah envelope proteins or functional variants thereof and an inhibitor of mTOR, to create a transduction mixture; and (b) administering the transduction mixture to the subject, thereby administering the viral vector to the subject, wherein the vector comprises a viral VPX protein and a chimeric antigen receptor (CAR).

Provided herein is a method for transducing cells in a subject, the method comprising: (a) contacting peripheral blood mononuclear cells (PBMCs) or a subset thereof from a subject with a composition comprising a lentiviral vector comprising one or more Nipah envelope proteins or functional variants thereof, and an inhibitor of mTOR to create a transduction mixture; and (b) administering the transduction mixture to the subject, thereby administering the viral vector to the subject, wherein the vector comprises a viral VPX protein and a chimeric antigen receptor (CAR).

Provided herein is a method for transducing cells in a subject, the method comprising: (a) contacting peripheral blood mononuclear cells (PBMCs) or a subset thereof from a subject with a composition comprising a lentiviral vector comprising one or more Nipah envelope proteins or functional variants thereof, an inhibitor of mTOR, and Il-7 or a functional variant thereof to create a transduction mixture; and (b) administering the transduction mixture to the subject, thereby administering the viral vector to the subject, wherein the vector comprises a chimeric antigen receptor (CAR).

In some of any of the provided embodiments, the PBMCs or subset are further contacted with an inhibitor of an antiviral restriction factor. In some of any of the provided embodiments, the transduction mixture further comprises an inhibitor of an antiviral restriction factor. In some of any of the provided embodiments, the inhibitor of an antiviral restriction factor is an inhibitor of SAMHD1, an inhibitor of IFITM1, and/or an inhibitor of IFITM3. In some of any of the provided embodiments, the inhibitor of an antiviral restriction factor is an inhibitor of SAMHD1, optionally wherein the inhibitor increases phosphorylation and/or degradation of SAMHD1. In some of any of the provided embodiments, the inhibitor of an antiviral restriction factor is an inhibitor of IFITM1, optionally wherein the inhibitor reduces expression of IFITM1. In some of any of the provided embodiments, the inhibitor of an antiviral restriction factor is an oligonucleotide, optionally wherein the inhibitor of an antiviral restriction factor is an anti-sense oligonucleotide complementary to an RNA encoding said cellular restriction factor. In some of any of the provided embodiments, the inhibitor of an antiviral restriction factor is a resveratrol cyclotrimer, optionally caraphenol A, a-viniferin or resveratrol, or an analog compound thereof. In some of any of the provided embodiments, the inhibitor of an antiviral restriction factor is an antifungal agent, optionally a polyene antifungal agent, further optionally amphotericin B. In some of any of the provided embodiments, the inhibitor of an antiviral restriction factor is contacted with PBMCs at a dose of at or about 1-10 μM, 10-20 μM, 20-30 μM, 30-40 μM, 40-50 μM, 50-60 μM, 60-70 μM, 70-80 μM, 80-90 μM, or 90-100 μM, or any value between the foregoing.

In some of any of the provided embodiments, the method is carried out in a single in-line procedure to maintain a closed or functionally closed fluid circuit. In some of any of the provided embodiments, the method is characterized by the whole blood, PBMCs or subset thereof, and transduction mixture having not been subjected to cryopreservation or freezing. In some of any of the provided embodiments, the PBMCs or subset thereof, and transfection mixture are not formulated with a cryoprotectant (e.g., DMSO).

In some of any of the provided embodiments, the transduction mixture is directly reinfused to the subject, optionally without any further processing or washing steps. In some of any of the provided embodiments, the closed fluid circuit comprises one or more of a blood processing set for obtaining the whole blood from the subject, a separation chamber for the separating the PBMCS or subset from the blood to collect the PBMCs or subset, a contacting container for the contacting the collected PBMCs or subset thereof with the composition comprising lipid particles (e.g. lentiviral vector), and a transfer container containing the contacted PBMCs or subset thereof and/or the transfection mixture for reinfusion to the subject.

In some of any of the provided embodiments, the closed fluid circuit further comprises a collection container operably connected to the separation chamber to collect the PBMCs or subset, optionally wherein the collection container is a bag, more optionally a sterile bag. In some of any of the provided embodiments, during at least a portion of the contacting the method comprises mixing the transduction mixture comprising the PBMCs or subset and the composition comprising the viral vector. In some of any of the provided embodiments, the mixing is by physical manipulation and/or centrifugation.

In some of any of the provided embodiments, the collected fraction of blood contains PBMCs or subset thereof separated from other blood components. In some of any of the provided embodiments, collecting the fraction of blood is by apheresis. In some of any of the provided embodiments, the apheresis device comprises membrane apheresis or centrifugal apheresis. In some of any of the provided embodiments, the collected fraction comprises leukocytes or precursors thereof. In some of any of the provided embodiments, the precursors thereof comprise hematopoietic stem cells. In some of any of the provided embodiments, collecting the fraction of blood is by leukapheresis. In some of any of the provided embodiments, the collected fraction of blood contains leukocytes.

In some of any of the provided embodiments, the inhibitor of an antiviral restriction factor is a cytokine. In some of any of the provided embodiments, the cytokine comprises IL-7, IL-15, or both IL-7 and IL-15. In some of any of the provided embodiments, the inhibitor of an antiviral restriction factor is an antifungal agent, optionally a polyene antifungal agent, further optionally amphotericin B. In some of any of the provided embodiments, the inhibitor of mTOR is rapamycin or a rapamycin analogue. In some of any of the provided embodiments, the inhibitor of mTOR is selected from the group comprising rapamycin, everolimus, temsirolimus, or ridaforolimus. In some of any of the provided embodiments, the inhibitor of mTOR is rapamycin.

In some of any of the provided embodiments, the inhibitor of mTOR is administered at a dose of 1 mg to 1000 mg per day or 1 mg/m²/day to 500 mg/m²/day, or as a single dose of 1 mg to 1000 mg or 1 mg/m² to 500 mg/m²/dose. In some of any of the provided embodiments, the inhibitor of mTOR is administered as a single dose of 2 mg to 50 mg. In some of any of the provided embodiments, the inhibitor of mTOR is administered as a single dose of 25 mg. In some of any of the provided embodiments, the inhibitor of mTOR is administered as a single dose of 100 mg/m² to 300 mg/m²/dose. In some of any of the provided embodiments, the inhibitor of mTOR is administered at a dose of 220 mg/m²/dose.

In some of any of the provided embodiments, administration of the inhibitor of mTOR further comprising a loading dose. In some of any of the provided embodiments, the loading dose is administered at a dose of 1 mg to 1000 mg per day or 1 mg/m²/day to 500 mg/m²/day, or as a single dose of 1 mg to 1000 mg or 1 mg/m² to 500 mg/m²/dose. In some of any of the provided embodiments, the loading dose is administered at a dose of 25 mg per day, 50 mg per day, or 500 mg per day.

In some of any of the provided embodiments, the inhibitor of mTOR is administered orally or intravenously, optionally wherein the inhibitor of mTOR is administered intravenously.

In some of any of the provided embodiments, the inhibitor of mTOR is contacted with the PBMCs or the subset thereof in an amount from 1 μM to 50 μM. In some of any of the provided embodiments, the inhibitor of mTOR is contacted with the PBMCs or the subset thereof in an amount of at or about 5 μM, 10 μM, 15 μM, 20 μM, 25 μM, 30 μM, 35 μM, or 40 μM, or any value between any of the foregoing.

In some of any of the provided embodiments, the method further comprises administration of one or more recombinant cytokine to the subject. In some of any of the provided embodiments, the PBMCs or subset are further contacted with one or more recombinant cytokine. In some of any of the provided embodiments, the transduction mixture further comprises one or more recombinant cytokines. In some of any of the provided embodiments, the one or more recombinant cytokines comprise recombinant IL-7, recombinant IL-15, or both recombinant IL-7 and recombinant IL-15. In some of any of the provided embodiments, the one or more recombinant cytokine further comprises IL-2. In some of any of the provided embodiments, the time period between the administration of the recombinant cytokine and viral vector is no more than one day. In some of any of the provided embodiments, the time period between the administration of the cytokine and viral vector is no more than 12 hours. In some of any of the provided embodiments, the time period between the administration of the cytokine and viral vector is no more than 1, 2, 3, 4, or 5 hours. In some of any of the provided embodiments, the recombinant cytokine is administered at a dose of from at or about 0.001 mg/kg to at or about 0.1 mg/kg, at or about 0.001 mg/kg to at or about 0.05 mg/kg, at or about 0.001 mg/kg to at or about 0.01 mg/kg, at or about 0.01 mg/kg to at or about 0.1 mg/kg, at or about 0.01 mg/kg to at or about 0.05 mg/kg or at or about 0.05 mg/kg to at or about 0.1 mg/kg. In some of any of the provided embodiments, the recombinant cytokine is administered at a dose of from or from about 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.02 mg/kg, 0.03 mg/kg, 0.04 mg/kg, or 0.05 mg/kg, or any value between any of the foregoing.

In some of any of the provided embodiments, the viral vector further comprises a viral accessory protein, optionally wherein the viral accessory protein is a viral kinase. In some of any of the provided embodiments, the viral accessory protein is an inhibitor of an antiviral restriction factor, optionally wherein the viral accessory protein is an inhibitor of SAMHD1 activity. In some of any of the provided embodiments, the viral accessory protein directly or indirectly phosphorylates SAMHD1. In some of any of the provided embodiments, the viral accessory protein is selected from the group consisting of: BLG4, UL97, and U69. In some of any of the provided embodiments, the viral accessory protein degrades SAMHD1. In some of any of the provided embodiments, the viral accessory protein is a fusion protein, optionally a fusion protein with VPX and/or Vpr.

In some of any of the provided embodiments, the viral vector is a retroviral vector. In some of any of the provided embodiments, the viral vector is a lentiviral vector. In some of any of the provided embodiments, the viral vector is pseudotyped with the fusogen. In some of any of the provided embodiments, the viral fusogen is selected from a Class I viral membrane fusion protein, a Class II viral membrane protein, a Class II viral membrane fusion protein, a viral membrane glycoprotein, or a viral envelope protein. In some of any of the provided embodiments, the viral fusogen comprises a viral envelope protein or a functional variant thereof. In some of any of the provided embodiments, the viral fusogen is a vesicular stomatitis virus envelope glycoprotein (VSV-G). In some of any of the provided embodiments, the viral fusogen is a baboon endogenous virus (BaEV) envelope glycoprotein. In some of any of the provided embodiments, the viral fusogen is a Cocal virus envelope glycoprotein. In some of any of the provided embodiments, the viral fusogen is an Alphavirus class II fusion protein or a functional variant thereof, optionally wherein the Alphavirus is a Sindbis virus.

In some of any of the provided embodiments, the viral fusogen comprises a Paramyxovirus fusion (F) protein or a biologically active portion thereof, optionally wherein the Paramyxovirus is a Morbillivirus or a Henipavirus. In some of any of the provided embodiments, the viral fusogen comprises a Morbillivirus fusion (F) protein. In some of any of the provided embodiments, the Morbillivirus F proteins from a measles virus (MeV), canine distemper virus, Cetacean morbillivirus, Peste-des-petits-ruminants virus, Phocine distemper virus, Rinderpest virus or a biologically active portion or functional variant thereof of any of the foregoing.

In some of any of the provided embodiments, the viral fusogen comprises a Henipavirus F protein from a Nipah virus, Hendra virus, Cedar virus, Kumasi virus, Mojiang virus or a biologically active portion or functional variant thereof.

In some of any of the provided embodiments, the viral fusogen comprises a Nipah virus F protein or a biologically active portion or functional variant thereof. In some of any of the provided embodiments, the fusogen comprises a paramyxovirus G, paramyxovirus H, or paramyxovirus HN protein, or a biologically active portion or functional variant thereof. In some of any of the provided embodiments, the paramyxovirus G, paramyxovirus H, or paramyxovirus HN protein further comprises a targeting moiety that binds to a molecule on a target cell.

In some of any of the provided embodiments, the viral fusogen comprises an F protein molecule or a biologically active portion thereof from a Paramyxovirus and a glycoprotein G (G protein) or a biologically active portion thereof from a Paramyxovirus. In some of any of the provided embodiments, the Paramyxovirus is a henipavirus.

In some of any of the provided embodiments, the Paramyxovirus is Nipah virus. In some of any of the provided embodiments, the viral fusogen and/or Nipah envelope protein comprises a Nipah virus F glycoprotein (NiV-F) or a biologically active portion or functional variant thereof and a Nipah virus G glycoprotein (NiV-G) or a biologically active portion or functional variant thereof.

In some of any of the provided embodiments, the Paramyxovirus is Hendra virus.

In some of any of the provided embodiments, the G protein or the biologically active portion thereof is a mutant NiV-G protein or biologically active portion thereof that exhibits reduced binding to Ephrin B2 or Ephrin B3. In some of any of the provided embodiments, the mutant NiV-G protein comprises one or more amino acid substitutions corresponding to amino acid substitutions selected from the group consisting of E501A, W504A, Q530A and E533A with reference to numbering set forth in SEQ ID NO:14. In some of any of the provided embodiments, the G protein or biologically active portion is a biologically active portion of wild-type NiV-G that has a deletion of up to 40 amino acids at or near the N-terminus, optionally not including the initial methionine. In some of any of the provided embodiments, the G protein is a biologically active portion that is a truncated NiV-G that has a deletion of amino acids 2-34 at or near the N-terminus of wild-type NiV-G set forth in SEQ ID NO:14. In some of any of the provided embodiments, the G protein or the biologically active portion has the amino acid sequence set forth in SEQ ID NO: 19 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:19.

In some of any of the provided embodiments, the F protein or the biologically active portion thereof is a NiV-F protein or a biologically active portion thereof. In some of any of the provided embodiments, the F protein or the biologically active portion is a truncated NiV-F that is truncated by at least or at 22 amino acids or at least or at 20 amino acids at or near the C-terminus of wild-type NiV-F set forth in SEQ ID NO:2, optionally not including the initial methionine. In some of any of the provided embodiments, the F protein or the biologically active portion is a truncated NiV-F that lacks amino acids 525-546 of SEQ ID NO:2. In some of any of the provided embodiments, the F protein or the biologically active portion has the amino acid sequence set forth in SEQ ID NO: 12 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:12. In some of any of the provided embodiments, the NiV-G protein comprises the amino acid sequence set forth in SEQ ID NO: 19, and the NiV-F protein comprises the amino acid sequence set forth in SEQ ID NO:12.

In some of any of the provided embodiments, the targeted moiety is specific for a cell surface receptor on a target cell. In some of any of the provided embodiments, the targeting domain is a Design ankyrin repeat proteins (DARPin), a single domain antibody (sdAb), a VHH fragment, a single chain variable fragment (scFv), or an antigen-binding fibronectin type III (Fn3) scaffold. In some of any of the provided embodiments, the fusogen and the targeting moiety are directly linked. In some of any of the provided embodiments, the fusogen and targeting moiety are indirectly linked via a linker. In some of any of the provided embodiments, the linker is a peptide linker. In some of any of the provided embodiments, the peptide linker is (GmS)n (SEQ ID NO: 11), wherein each of m and n is an integer between 1 to 4, inclusive.

In some of any of the provided embodiments, the exogenous agent is a nucleic acid or a polypeptide. In some of any of the provided embodiments, the exogenous agent is a nucleic acid encoding a payload gene, optionally wherein the nucleic acid encodes a chimeric antigen receptor. In some of any of the provided embodiments, the target cell is one or more of a monocyte, macrophage, neutrophil, dendritic cell, eosinophil, mast cell, platelet, large granular lymphocyte, Langerhans' cell, natural killer (NK) cell, T lymphocyte (e.g., T cell), a Gamma delta T cell, B lymphocyte (e.g., B cell), CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, a CD30+ lung epithelial cell, a liver sinusoidal endothelial cell or myocyte. In some of any of the provided embodiments, the target cell is a T cell, optionally wherein the target cell is a resting T cell. In some of any of the provided embodiments, the target cell is a T cell that has not been activated. In some of any of the provided embodiments, the viral vector comprises (i) a re-targeted Nipah virus G glycoprotein (NiV-G) that is a truncated NiV-G set forth in SEQ ID NO:19 linked to a targeting moiety that binds to a T cell, and (ii) a truncated Nipah virus F glycoprotein (NiV-F) set forth in SEQ ID NO:12.

In some of any of the provided embodiments, the targeting moiety that binds to a T cell is a CD8 binding agent, CD4 binding agent or CD3 binding agent. In some of any of the provided embodiments, the targeting moiety is an sdAb or an ScFv.

Provided herein is a composition, comprising the transduction mixture of any of the provided methods. Also provided herein is a composition, comprising a leukapheresis composition for delivering a viral vector to a subject, wherein the leukapheresis composition comprises peripheral blood mononuclear cells (PBMCs) or a subset thereof from the subject, a viral vector, and an inhibitor of mTOR. Also provided herein is a composition comprising a leukapheresis composition for delivering a viral vector to a subject, wherein the leukapheresis composition comprises peripheral blood mononuclear cells (PBMCs) or a subset thereof from the subject, a viral vector, and an inhibitor of an antiviral restriction factor. In of any of the provided embodiments, the delivery to the subject is with an apheresis device.

Provided herein is a method of treating a disease or condition in a subject comprising administering a viral vector or exogenous agent by any of the provided methods to a subject in need thereof. Also provided herein is a method of treating a disease or condition comprising infusing any of the provided compositions into a subject in need thereof.

In of any of the provided embodiments, the disease or disorder is treatable by administration of the viral vector or the exogenous agent. In of any of the provided embodiments, the disease or condition is a cancer. In of any of the provided embodiments, the cancer is a solid tumor, a lymphoma or a leukemia.

### BRIEF DESCRIPTION OF THE DRAWINGS

FIG. 1A depicts transduction efficiency following knock-out of IFITM1 in a VSV-G pseudotyped lentiviral vectors. FIG. 1B depicts transduction efficiency following knock-out of IFITM1 in a lentiviral vector pseudotyped with the exemplary CD8-retargeted Nipah fusogen.

FIG. 2A depicts transduction of CD8+ cells with the exemplary CD8-retargeted fusogen pseudotyped lentiviral vector encoding a reporter gene GFP in thawed cells, FIG. 2B depicts transduction of CD4+ cells with the exemplary CD4-retargeted fusogen pseudotyped lentiviral vector encoding a reporter gene GFP in cells freshly isolated. FIG. 2C depicts verification of gene knockout (IFITM1 and SAMHD1) by western blot in cell lysates harvested on the day of transduction. CD8+ cells were assessed for expression of IFITM1 and phosphorylated SAMHD1 in FIG. 3A. FIG. 3B depicts expression of IFITM1 and phosphorylated SAMHD1 in CD4+ cells. Representative western blots are shown in FIG. 3C (CD8+) and FIG. 3D (CD4+).

FIG. 4A depicts transduction efficiency in T cells transduced with a CD4-retargeted Nipah fusogen pseudotyped lentiviral vector from donor 1, while FIG. 4B depicts the same from a second donor. Transduction efficiency of T cells with a CD8-retargeted Nipah fusogen pseudotyped lentiviral vector from two donors is shown in FIG. 4C or FIG. 4E for donor 1, and FIG. 4D or FIG. 4F for donor 2. Aggregate data across multiple donors are also shown for a CD4-retargeted Nipah fusogen pseudotyped lentiviral vector in FIG. 4G, and for a CD8-retargeted Nipah fusogen pseudotyped lentiviral vector in FIG. 4H.

Expression of restriction factors in the presence of cytokines with or without rapamycin following transduction is shown for a first exemplary donor in FIG. 5A, and a second donor in FIG. 5B.

FIG. 6 depicts an exemplary flow diagram of one embodiment of the provided method of administering a viral vector to a subject.

FIG. 7 depicts an exemplary flow diagram outlining an alternative embodiment of the method in FIG. 6 in which one or more various optional features can be additionally incorporated into the method.

FIG. 8 depicts transduction efficiency in resting pan-T cells transduced with CD8-retargeted Nipah fusogen pseudotyped lentiviral vector from a single donor. Cells were incubated with (+ TEM) or without (no TEM) and either IL-7 at increasing concentrations (0.6 ng/mL to 75 ng/mL), active IL-2, active IL-7 or IL2.

FIG. 9 depicts transduction efficiency in resting pan-T cells transduced with CD8-retargeted Nipah fusogen pseudotyped lentiviral vector from four donors. Cells were incubated with IL-2 or IL-7 in the presence of TEM (i.e., IL-2 TEM or IL-7 TEM) or the absence of TEM (i.e., IL-2 no drug or IL-7 no drug).

FIG. 10A depicts expression of IFITM1 and phosphorylated SAMHD1 in CD8+ cells after incubation with IL-2 or IL-7 in the presence or absence of TEM. Representative western blots are shown for three donors. FIG. 10B depicts the percentage of SAMHD1/IFITM1 knockout CD8+ cells after pretreatment with IL-2 or IL-7, in the presence or absence of RAP or TEM. FIG. 10C depicts the integrated viral copies per diploid genome of SAMHD1/IFITM1 knockout CD8+ cells after pretreatment with IL-2 or IL-7, in the presence or absence of RAP or TEM.

FIGS. 11A-11C depict the percentage of CAR expressed and the viral copy number (VCN) of pan-T cells isolated from three donors and incubated with a combination of: rapamycin (RAP), everolimus (EVO), or temsirolimus (TEM) and IL-2; or RAP, EVO, TEM and IL-7. FIG. 11A shows data from Donor 1, FIG. 11B shows data from Donor 2, and FIG. 11C shows data from Donor 3.

FIG. 12 depicts the percentage of CAR expressed in pan-T cells from six donors treated with a combination of rapamycin (RAP), everolimus (EVO), or temsirolimus (TEM) and IL-2, or RAP, EVO, TEM and IL-7.

FIG. 13A depicts the % of pan-T cells that are CD8+CAR+ after incubation with a combination of rapamycin (RAP), everolimus (EVO), or temsirolimus (TEM) and IL-2, or RAP, EVO, TEM and IL-7. FIG. 13B depicts TU/mL of the pan-T cells isolated from a donor and incubated with RAP, EVO, TEM and IL-2, or RAP, EVO, TEM and IL-7.

FIG. 14A depicts an experimental timeline in a Nalm6 killing assay. FIG. 14B depicts transduction efficiency via viral copy number per diploid genome in untreated CD8+ cells, TEM treated CD8+ cells, IL-7 treated CD8+ cells, and IL-7+ TEM treated CD8+ cells. FIG. 14C depicts cytotoxicity of Nalm6 cells across 10 days.

FIG. 15A depicts transduction efficiency (% GFP) and CD8 cell counts for Donor 1 in the presence of an exemplary antifungal agent, where these same data are shown for a second donor in FIG. 15B. Similarly, FIG. 15C depicts transduction efficiency (% GFP) and CD4 cell counts for Donor 1, where these same data are shown for a second donor in FIG. 15D.

FIG. 16A depicts transduction efficiency (% GFP) and CD8 cell counts for Donor 1 in the presence of an exemplary inhibitor of mTOR, where these same data are shown for a second donor in FIG. 16B. As above, FIG. 16C depicts transduction efficiency (% GFP) and CD4 cell counts for Donor 1, where these same data are shown for a second donor in FIG. 16D.

### DETAILED DESCRIPTION

Provided herein are methods for transducing cells in a subject involving administering to the subject an inhibitor of the mammalian target of rapamycin (mTOR) and a viral vector containing a fusogen. In some embodiments, the methods further include administering to the subject an inhibitor of an antiviral restriction factor. Also provided herein are methods for transducing cells in a subject involving administering to the subject an antiviral restriction factor inhibitor and a viral vector containing a fusogen. Also provided herein are methods for transducing cells in a subject involving administering to the subject an inhibitor of the mammalian target of rapamycin (mTOR), an antiviral restriction factor inhibitor and a viral vector containing a fusogen. In some embodiments, the fusogen is embedded in the lipid bilayer. In some embodiments, the fusogen is exposed on the outside surface of the viral vector. In some embodiments, the viral vector further contains an exogenous agent. In some embodiments, the exogenous agent is a nucleic acid or protein agent. In some embodiments, the methods further include administering to the subject a cytokine, such as IL-7. In some embodiments, the cytokine is a cytokine that is an inhibitor of an antiviral restriction factor.

In some embodiments, the provided methods provide for transducing cells in a subject and/or delivering an exogenous agent to a subject via viral vector. In some embodiments, the provided methods provide for extracorporeal or ex vivo administration of a viral vector including for delivery of an exogenous agent contained therein to a subject. In some embodiments, the viral vector may be a retroviral vector, such as a viral vector that is pseudotyped for targeting to a desired target cell (e.g., CD8- or CD4-targeted viral vector for delivery to a T cell). Thus, in some embodiments, the provided methods provide for transduction for delivery of a viral vector or exogenous agent to target cells of interest for therapy. In some embodiments, delivery of the exogenous agent to target cells may provide a therapeutic intervention or treatment for a disease or condition, such as cancer or a genetic deficiency.

Resting human T cells are difficult to transduce with lentiviral vectors (LVV) primarily due to blocks imposed by cellular restriction factors during viral replication. These blocks collectively limit the potency of LVV-based T cell gene therapies. While restriction factors have been extensively studied in CD4+ T cells infected with HIV-1, the impact of these proteins in CD8+ cells is not as well-described. IFITM1 and SAMHD1 impose potent blocks in primary CD8+ cells. Mechanistically, IFITM1 impedes LVV fusion at the cell membrane while SAMHD1 interferes with reverse transcription by cleaving cellular dNTPs and reducing available nucleotide pools. In some embodiments, the present disclosure provides pharmaceutical intervention(s) that inactivate these restriction factors to increase potency and reduce the minimal efficacious dose of the fusogen-containing viral vector (also called a “fusosome”).

The provided embodiments relate to methods for transducing cells with a viral vector in the presence of an inhibitor of the mammalian target of rapamycin (mTOR), and optionally also a recombinant cytokine such as IL-7. In some embodiments, results herein identify restriction factors that limit transduction by CD8-targeted viral vectors such as fusosome and show that potency can be significantly increased in resting T cells following treatment with rapamycin or a rapamycin analog (e.g., temsirolimus (TEM)) and IL-7 in a transgene-independent manner. Among provided embodiments, the present disclosure identifies key restriction factors that limit transduction of viral vectors, such as retargeted lentiviral vectors (e.g., CD8-targeted fusosome-LVVs), particularly in resting T cells. For example, results herein demonstrate that IFITM1 can be downregulated by pre-treating resting T cells with rapamycin, while IL-7 pre-treatment can lead to enhanced levels of the inactive form of SAMHD1 (pSAMHD1). The working examples further demonstrate that potency is significantly increased in resting T cells following incubation with a cytokine (e.g., IL-7) and a rapamycin analog (e.g., temsirolimus) independent of the transgene, which is consistent with the inactivation or loss of two critical restriction factors, SAMDH1 and IFITM1. Having identified both factors as being potent restriction factors in CD8+ T cells through restriction factor knockout experiments, in some embodiments the present disclosure provides a combination treatment including IL-7 and a rapamycin analog (rapalog), such as temsirolimus (TEM). The provided methods are based on observations that such as combination dramatically improves gene delivery efficiency and downstream efficacy of a CD8-targeted viral vector such as fusosomes in vitro, thus demonstrating that overcoming restriction factors in cell and gene therapy may enhance the efficacy of these viral vectors. Collectively, these results suggest a strategy toward a combination in vivo vector-based therapy that is supplemented with pharmaceutical agents to improve vector potency.

In some embodiments, the methods provide for a strategy for administration of viral vectors, as carriers for exogenous agent, in the presence of an inhibitor of mTOR. In some embodiments, the methods provide for a strategy for administration of viral vectors, as carriers for exogenous agent, in the presence of an inhibitor of an antiviral restriction factor, such as IFITM1 or SAMDH1. The provided methods can in some aspects increase efficiency of transduction and reduce total amount of viral vector needed for treatment. For instance, transduction in the presence of an inhibitor of mTOR and/or inhibitor of an antiviral restriction factor as provided allows for increased rate of transfection and/or transduction, and reduces the effective dose of the viral vector or exogenous agent required to treat a subject.

Accordingly, the provided administration of the viral vectors, such as to transduce a cell or deliver a payload gene, is such that the initial contact between the viral vector, such as containing an exogenous agent, and cells is in vivo or ex vivo.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described. Those skilled in the art will recognize that several embodiments are possible within the scope and spirit of the present disclosure. The following description illustrates the disclosure and, of course, should not be construed in any way as limiting the scope of the inventions described herein.

### I. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Unless defined otherwise, all technical and scientific terms, acronyms, and abbreviations used herein have the same meaning as commonly understood by one of ordinary skill in the art to which the invention pertains. Unless indicated otherwise, abbreviations and symbols for chemical and biochemical names is per IUPAC-IUB nomenclature. Unless indicated otherwise, all numerical ranges are inclusive of the values defining the range as well as all integer values in-between.

As used herein, the articles “a” and “an” refer to one or to more than one (i.e., to at least one) of the grammatical object of the article. By way of example, “an element” means one element or more than one element.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “CDR” denotes a complementarity determining region as defined by at least one manner of identification to one of skill in the art. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

In some embodiments, CDRs can be defined in accordance with any of the Chothia numbering schemes, the Kabat numbering scheme, a combination of Kabat and Chothia, the AbM definition, and/or the contact definition. A VHH comprises three CDRs, designated CDR1, CDR2, and CDR3. Table 1, below, lists exemplary position boundaries of CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-H1 located before CDR-H1, FR-H2 located between CDR-H1 and CDR-H2, FR-H3 located between CDR-H2 and CDR-H3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 1
Boundaries of CDRs according to various numbering schemes.

| CDR | Kabat | Chothia | AbM | Contact |
|---|---|---|---|---|
| CDR-H1 | H31--H35B | H26--H32 . . . 34 | H26--H35B | H30--H35B |
| (Kabat Numbering¹) | | | | |
| CDR-H1 | H31--H35 | H26--H32 | H26--H35 | H30--H35 |
| (Chothia Numbering²) | | | | |
| CDR-H2 | H50--H65 | H52--H56 | H50--H58 | H47--H58 |
| CDR-H3 | H95--H102 | H95--H102 | H95--H102 | H93--H101 |

¹Kabat et al. (1991), “Sequences of Proteins of Immunological Interest,” 5th Ed. Public Health Service, National Institutes of Health, Bethesda, MD
²Al-Lazikani et al., (1997) JMB 273, 927-948

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes. For example, where it is stated that a particular CDR (e.g., a CDR-H3) contains the amino acid sequence of a corresponding CDR in a given VHH amino acid sequence, it is understood that such a CDR has a sequence of the corresponding CDR (e.g., CDR-H3) within the VHH, as defined by any of the aforementioned schemes. In some embodiments, specific CDR sequences are specified. Exemplary CDR sequences of provided antibodies are described using various numbering schemes (see e.g., Table 1), although it is understood that a provided antibody can include CDRs as described according to any of the other aforementioned numbering schemes or other numbering schemes known to a skilled artisan.

The terms “viral vector particle” and “viral vector” are used interchangeably herein and refer to a vector for transfer of an exogenous agent (e.g. non-viral or exogenous nucleic acid) into a recipient or target cell and that contains one or more viral structural proteins in addition to at least one non-structural viral genomic component or functional fragment thereof (i.e., a polymerase, an integrase, a protease or other non-structural component). The viral vector thus contains the exogenous agent, such as heterologous nucleic acid that includes non-viral coding sequences, to be transferred into a cell. Examples of viral vectors are retroviral vectors, such as lentiviral vectors.

The term “retroviral vector” refers to a viral vector that contains retroviral nucleic acid or is derived from a retrovirus. A retroviral vector particle includes the following components: a vector genome (retrovirus nucleic acid), a nucleocapsid encapsidating the nucleic acid, and a membrane envelope surrounding the nucleocapsid. Typically, a retroviral vector contains sufficient retroviral genetic information to allow packaging of an RNA genome, in the presence of packaging components, into a viral particle capable of infecting a target cell. Infection of the target cell may include reverse transcription and integration into the target cell genome. A retroviral vector may be a recombinant retroviral vector that is replication defective and lacks genes essential for replication, such as a functional gag-pol and/or env gene and/or other genes essential for replication. A retroviral vector also may be a self-inactivating (SIN) vector.

As used herein, a “lentiviral vector” or LV refers to a viral vector that contains lentiviral nucleic acid or is derived from a lentivirus. A lentiviral vector particle includes the following components: a vector genome (lentivirus nucleic acid), a nucleocapsid encapsidating the nucleic acid, and a membrane surrounding the nucleocapsid. Typically, a lentiviral vector contains sufficient lentiviral genetic information to allow packaging of an RNA genome, in the presence of packaging components, into a viral particle capable of infecting a target cell. Infection of the target cell may include reverse transcription and integration into the target cell genome. A lentiviral vector may be a recombinant lentiviral vector that is replication defective and lacks genes essential for replication, such as a functional gag-pol and/or env gene and/or other genes essential for replication. A lentiviral vector also may be a self-inactivating (SIN) vector.

As used herein, a “retroviral nucleic acid,” refers to a nucleic acid containing at least the minimal sequence requirements for packaging into a retroviral vector, alone or in combination with a helper cell, helper virus, or helper plasmid. In the case of “lentiviral nucleic acid” the nucleic acid refers to at least the minimal sequence requirements for packaging into a lentiviral vector, alone or in combination with a helper cell, helper virus, or helper plasmid. In some embodiments, the viral nucleic acid comprises one or more of (e.g., all of) a 5′ LTR (e.g., to promote integration), U3 (e.g., to activate viral genomic RNA transcription), R (e.g., a Tat-binding region), U5, a 3′ LTR (e.g., to promote integration), a packaging site (e.g., psi (Ψ)), RRE (e.g., to bind to Rev and promote nuclear export). The viral nucleic acid can comprise RNA (e.g., when part of a virion) or DNA (e.g., when being introduced into a source cell or after reverse transcription in a recipient cell). In some embodiments, the viral nucleic acid is packaged using a helper cell, helper virus, or helper plasmid which comprises one or more of (e.g., all of) gag, pol, and env.

As used herein, “fusosome” refers to a lipid particle containing a bilayer of amphipathic lipids enclosing a lumen or cavity and a fusogen that interacts with the amphipathic lipid bilayer. In some embodiments, the fusosome is a membrane enclosed preparation. In some embodiments, the fusosome is derived from a source cell. A fusosome also may include an exogenous agent or a nucleic acid encoding an exogenous agent, which may be present in the lumen of the fusosome.

As used herein, “fusosome composition” refers to a composition comprising one or more fusosomes.

As used herein, “fusogen” refers to an agent or molecule that creates an interaction between two membrane enclosed lumens. In embodiments, the fusogen facilitates fusion of the membranes. In other embodiments, the fusogen creates a connection, e.g., a pore, between two lumens (e.g., a lumen of a retroviral vector and a cytoplasm of a target cell). In some embodiments, the fusogen comprises a complex of two or more proteins, e.g., wherein neither protein has fusogenic activity alone. In some embodiments, the fusogen comprises a targeting domain. Examples of fusogens include paramyxovirus F and G proteins such as those from Nipah Virus (NiV) and biologically active portions or variants thereof including any as described.

As used herein, a “re-targeted fusogen,” such as a re-targeted G protein, refers to a fusogen that comprises a targeting moiety having a sequence that is not part of the naturally occurring form of the fusogen in which the targeting moiety targets or binds a molecule on a desired cell type. In embodiments, the fusogen comprises a different targeting moiety relative to the targeting moiety in the naturally occurring form of the fusogen. In embodiments, the naturally occurring form of the fusogen lacks a targeting domain, and the re-targeted fusogen comprises a targeting moiety that is absent from the naturally occurring form of the fusogen. In embodiments, the fusogen is modified to comprise a targeting moiety. In some such embodiments, the attachment of the targeting moiety to a fusogen (e.g., G protein) may be directly or indirectly via a linker, such as a peptide linker. In embodiments, the fusogen comprises one or more sequence alterations outside of the targeting moiety relative to the naturally occurring form of the fusogen, e.g., in a transmembrane domain, fusogenically active domain, or cytoplasmic domain.

As used herein, a “target cell” refers to a cell of a type to which it is desired that a targeted lipid particle or viral vector delivers an exogenous agent. In embodiments, a target cell is a cell of a specific tissue type or class, e.g., an immune effector cell, e.g., a T cell. In some embodiments, a target cell is a diseased cell, e.g., a cancer cell. In some embodiments, the fusogen, e.g., re-targeted fusogen leads to preferential delivery of the exogenous agent to a target cell compared to a non-target cell.

As used herein a “non-target cell” refers to a cell of a type to which it is not desired that a targeted lipid particle or viral vector delivers an exogenous agent. In some embodiments, a non-target cell is a cell of a specific tissue type or class. In some embodiments, a non-target cell is a non-diseased cell, e.g., a non-cancerous cell. In some embodiments, the fusogen, e.g., re-targeted fusogen leads to lower delivery of the exogenous agent to a non-target cell compared to a target cell.

As used herein a “biologically active portion,” such as with reference to a protein such as a G protein or an F protein, refers to a portion of the protein that exhibits or retains an activity or property of the full-length of the protein. For example, a biologically active portion of an F protein retains fusogenic activity in conjunction with the G protein when each are embedded in a lipid bilayer. A biologically active portion of the G protein retains fusogenic activity in conjunction with an F protein when each is embedded in a lipid bilayer. The retained activity can include 10%-150% or more of the activity of a full-length or wild-type F protein or G protein. Examples of biologically active portions of F and G proteins include proteins with truncations of the cytoplasmic domain, such as any of the described NiV-F with a truncated cytoplasmic tail.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 2. Amino acid substitutions may be introduced into an antibody of interest and the products screened for a desired activity, for example, retained/improved binding.

TABLE 2

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Len, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term, “corresponding to” with reference to positions of a protein, such as recitation that nucleotides or amino acid positions “correspond to” nucleotides or amino acid positions in a disclosed sequence, such as set forth in the Sequence listing, refers to nucleotides or amino acid positions identified upon alignment with the disclosed sequence based on structural sequence alignment or using a standard alignment algorithm, such as the GAP algorithm. For example, corresponding residues of a similar sequence (e.g. fragment or species variant) can be determined by alignment to a reference sequence by structural alignment methods. By aligning the sequences, one skilled in the art can identify corresponding residues, for example, using conserved and identical amino acid residues as guides.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The term “effective amount” as used herein means an amount of a pharmaceutical composition which is sufficient to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

An “exogenous agent” as used herein with reference to a lipid particle or viral vector refers to an agent that is neither comprised by nor encoded in the corresponding wild-type virus or fusosome made from a corresponding wild-type source cell. In some embodiments, the exogenous agent does not naturally exist, such as a protein or nucleic acid that has a sequence that is altered (e.g., by insertion, deletion, or substitution) relative to a naturally occurring protein. In some embodiments, the exogenous agent does not naturally exist in the source cell. In some embodiments, the exogenous agent exists naturally in the source cell but is exogenous to the virus. In some embodiments, the exogenous agent does not naturally exist in the recipient cell. In some embodiments, the exogenous agent exists naturally in the recipient cell, but is not present at a desired level or at a desired time. In some embodiments, the exogenous agent comprises RNA or protein.

As used herein, a “promoter” refers to a cis-regulatory DNA sequence that, when operably linked to a gene coding sequence, drives transcription of the gene. The promoter may comprise a transcription factor binding sites. In some embodiments, a promoter works in concert with one or more enhancers which are distal to the gene.

As used herein, a composition refers to any mixture of two or more products, substances, or compounds, including cells. It may be a solution, a suspension, liquid, powder, a paste, aqueous, non-aqueous or any combination thereof.

As used herein, the term “pharmaceutically acceptable” refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

A “disease” or “disorder” as used herein refers to a condition where treatment is needed and/or desired.

As used herein, the terms “treat,” “treating,” or “treatment” refer to ameliorating a disease or disorder, e.g., slowing or arresting or reducing the development of the disease or disorder or reducing at least one of the clinical symptoms thereof. For purposes of this disclosure, ameliorating a disease or disorder can include obtaining a beneficial or desired clinical result that includes, but is not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total).

The terms “individual” and “subject” are used interchangeably herein to refer to an animal; for example a mammal. The term patient includes human and veterinary subjects. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. The subject can be male or female and can be any suitable age, including infant, juvenile, adolescent, adult, and geriatric subjects. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder. In particular embodiments, the subject is a human, such as a human patient.

### II. METHODS OF TRANSDUCTION AND ADMINISTRATION

Provided herein are methods of transducing cells in which the methods involve contacting a cell with a viral vector, such as a lentiviral vector, and contacting the cell with an inhibitor of mTOR. The contacting may be performed in vitro (e.g., with cells derived from a healthy donor or a donor in need of cellular therapy) or in vivo by administration of the viral vector to a subject. In some embodiments, the contacting is carried out in vivo in a subject by administering to a subject a viral vector and an inhibitor of mTOR. In some embodiments, the methods may further include contacting a cell with an antiviral restriction factor inhibitor. In some embodiments, the methods may further include contacting a cell with cytokine, such as IL-7.

Also provided herein are methods of transducing cells in which the methods involve contacting a cell with a viral vector, such as a lentiviral vector, and contacting the cell with an antiviral restriction factor inhibitor. The contacting may be performed in vitro (e.g., with cells derived from a healthy donor or a donor in need of cellular therapy) or in vivo by administration of the viral vector to a subject. In some embodiments, the contacting is carried out in vivo in a subject by administering to a subject a viral vector and an antiviral restriction factor inhibitor. In some embodiments, the methods may further include contacting a cell with cytokine, such as IL-7.

In some embodiments, the viral vector is administered by ex vivo administration of the lentiviral vector to the subject. In some embodiments, the provided method include a) administering a viral vector (e.g. a lentiviral vector) to a subject; and b) administering to the subject an inhibitor of mTOR. In some embodiments, the viral vector is administered directly to the subject. In some embodiments, the viral vector is administered by extracorporeal delivery methods. In some embodiments, the viral vector is administered in a single in-line procedure to maintain a closed or functionally closed fluid circuit.

In some embodiments, the viral vector is a cell-targeted viral vector, such as a cell-targeted lentiviral vector. In some aspects, the viral vector contains an extracellular targeting moiety that is linked to an envelope protein or embedded in the lipid bilayer for specific targeted recognition of a molecule on the surface of a target cell. In some embodiments the cell is a T cell and, in some aspects, the targeting moiety is a T-cell targeting moiety (also called T cell binding agent) such as for targeting a CD3, CD4 or CD8 molecule on the T cell.

In some embodiments, the viral vector, such as a lentiviral vector, includes a nucleic acid encoding an exogenous agent and thus, in some aspects, can be used to deliver an exogenous agent to a T cell. In some embodiments, the provided methods include administering a viral vector (e.g. lentiviral vector) to a subject, and b) administering to the subject an inhibitor of mTOR. In some embodiments, the transgene encodes an engineered receptor that binds to or recognizes a protein or antigen expressed by or on cells associated with a disease or condition. In some embodiments, the engineered receptor is a chimeric antigen receptor (CAR). In some embodiments, the engineered receptor is a T cell receptor (TCR).

In some embodiments, the provided methods include administering a viral vector (e.g. lentiviral vector) comprising a T cell binding agent to a subject, wherein the lentiviral vector comprising a nucleic acid encoding a transgene and the T cell binding agent binds a surface molecule on a T cell to target the viral vector to the T cell; and b) administering to the subject an inhibitor of mTOR. In some embodiments, the lentiviral vector comprises an exogenous agent that is a nucleic acid which encodes an engineered receptor that binds to or recognizes a protein or antigen expressed by or on cells associated with a disease or condition. In some embodiments, the engineered receptor is a chimeric antigen receptor (CAR). In some embodiments, the engineered receptor is a T cell receptor (TCR).

In some embodiments, the provided methods can be used to treat a disease or condition in a subject. In some embodiments, the transgene is a gene or encodes a protein that is a therapeutic agent or provides a therapeutic effect or activity for treating a disease or condition in a subject. For example, in some embodiments, the engineered receptor, such as a CAR or a TCR binds to or recognizes a protein or antigen expressed by cells associated with the disease or condition. In some embodiments, the provided methods include administering a viral vector (e.g. lentiviral vector) comprising a T cell binding agent to a subject, wherein the viral vector comprises a nucleic acid that encodes an engineered receptor that binds to or recognizes a protein or antigen expressed by or on cells associated with a disease or condition, and wherein the T cell binding agent binds a surface molecule on a T cell to target the lentiviral vector to the T cell; and b) administering to the subject an inhibitor of mTOR. In some embodiments, the engineered receptor is a chimeric antigen receptor (CAR). In some embodiments the disease or condition is a cancer and the engineered receptor, such as a CAR, binds to or recognizes a protein or antigen expressed by tumor cells. In some embodiments, the antigen expressed by tumor cells is CD19, CD22, CD20 or BCMA. In some embodiments, the CAR is an anti-CD19 CAR. In some embodiments, the CAR is an anti-CD22 CAR. In some embodiments, the CAR is an anti-CD20 CAR. In some embodiments, the CAR is an anti-BCMA CAR.

### A. System and Methods for In Vivo Transduction Via Viral Vector

Provided herein are methods of transducing cells by in vivo by administration of the viral vector to a subject in combination with an mTOR inhibitor. In some embodiments, the subject is administered or has been administered an inhibitor of mTOR in combination with the viral vector (e.g. lentiviral vector) in accord with the provided methods. In some embodiments, the provided methods involve administering to a subject a viral vector, such as lentiviral vector, and an inhibitor of mTOR. In some embodiments, the inhibitor of mTOR and viral vector (e.g. lentiviral vector) are administered simultaneously. In some embodiments, the subject is administered or has been administered an inhibitor of mTOR and a viral vector consecutively. In some embodiments, the inhibitor of mTOR and the viral vector are administered on the same day. In some embodiments, the inhibitor of mTOR is administered intermittently such as in a particular dosing regimen with a defined frequency or schedule. In some embodiments, the methods may further include administering an antiviral restriction factor inhibitor. In some embodiments, the methods may further include administering a cytokine, such as IL-7.

Provided herein are methods of transducing cells by in vivo by administration of the viral vector to a subject in combination with an antiviral restriction factor inhibitor. In some embodiments, the subject is administered or has been administered an antiviral restriction factor inhibitor in combination with the viral vector (e.g. lentiviral vector) in accord with the provided methods. In some embodiments, the provided methods involve administering to a subject a viral vector, such as lentiviral vector, and an antiviral restriction factor inhibitor. In some embodiments, the antiviral restriction factor inhibitor and viral vector (e.g. lentiviral vector) are administered simultaneously. In some embodiments, the subject is administered or has been administered an antiviral restriction factor inhibitor and a viral vector consecutively. In some embodiments, the antiviral restriction factor inhibitor and the viral vector are administered on the same day. In some embodiments, the antiviral restriction factor inhibitor is administered intermittently such as in a particular dosing regimen with a defined frequency or schedule.

In certain embodiments, the subject is administered or has been administered an inhibitor of mTOR 1 month before or after administration of the viral vector or a first dose of the viral vector. In some embodiments, the subject is administered or has been administered an inhibitor of mTOR within 1 month before administration of the viral vector or a first dose of the viral vector, such as within or at or about 4 weeks, 3 weeks, 2 weeks or 1 weeks, such as at or about 1 day, 2 days, 3 days, 4 days, 5 days, 6 days or 7 days before administration of the viral vector or a first dose of the viral vector. In some embodiments, the subject is administered or has been administered an inhibitor of mTOR within 3 days before administration of the viral vector.

In certain embodiments, the subject is administered or has been administered an inhibitor of mTOR 1 day before or after administration of the viral vector or a first dose of the viral vector. In some embodiments, the subject is administered or has been administered an inhibitor of mTOR within 1 day before administration of the viral vector or a first dose of the viral vector, such as within or at or about 24, 23, 22, 21, 20, 19, 18, 17, 16, 15, 14, 13, or 12 hours, or such as 12, 11, 10, 9, 8, 7, 6, 5, 4, 3, 2, or 1 hour before administration of the viral vector or a first dose of the viral vector. In some embodiments, the subject is administered or has been administered an inhibitor of mTOR within 3 hours before administration of the viral vector.

In some embodiments, the inhibitor of mTOR is administered at a dose of from at or about 1-10 mg, 10-100 mg, or 100-1000 mg, or any value between the foregoing. In some embodiments, the inhibitor of mTOR is administered at a dose from at or about 100-200 mg, 200-300 mg, 300-400 mg, 400-500 mg, 500-600 mg, 600-700 mg, 700-800 mg, 800-900 mg, or 900-1000 mg, or any value between the foregoing. In some embodiments, the inhibitor of mTOR is administered at a dose of 100 mg, 200 mg, 300 mg, 400 mg, 500 mg, 600 mg, 700 mg, 800 mg, 900 mg, or 1000 mg. In some embodiments, the inhibitor of mTOR is administered as a dose of 500 mg. In some embodiments, the inhibitor of mTOR is administered as a dose of 800 mg. In some embodiments, the inhibitor of mTOR is administered at a dose of from at or about 1-10 mg, 10-20 mg, 20-30 mg, 30-40 mg, or 40-50 mg, or any value between the foregoing. In some embodiments, the inhibitor of mTOR is administered at a dose of 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33, mg 34 mg, or 35 mg. In some embodiments, the inhibitor of mTOR is administered as a dose of 50, 25, or 10 mg. In some embodiments, the inhibitor of mTOR is administered as a dose of 40 mg. In some embodiments, the inhibitor of mTOR is administered as a dose of 25 mg.

In some embodiments, the inhibitor of mTOR is administered at a dose of from at or about 1-10 mg/m², 10-100 mg/m², or 100-1000 mg/m², or any value between the foregoing. In some embodiments, the inhibitor of mTOR is administered at a dose from at or about 100-200 mg/m², 200-300 mg/m², 300-400 mg/m², 400-500 mg/m², 500-600 mg/m², 600-700 mg/m², 700-800 mg/m², 800-900 mg/m², or 900-1000 mg/m², or any value between the foregoing. In some embodiments, the inhibitor of mTOR is administered at a dose of 100 mg/m², 200 mg/m², 300 mg/m², 400 mg/m², 500 mg/m², 600 mg/m², 700 mg/m², 800 mg/m², 900 mg/m², or 1000 mg/m². In some embodiments, the inhibitor of mTOR is administered at a dose of 50 mg/m², 100 mg/m², 150 mg/m², 200 mg/m², 250 mg/m², 300 mg/m², 350 mg/m², 400 mg/m², 450 mg/m², or 500 mg/m². In some embodiments, the inhibitor of mTOR is administered as a dose of 200 mg/m². In some embodiments, the inhibitor of mTOR is administered as a dose of 220 mg/m². In some embodiments, the inhibitor of mTOR is administered at a dose of from at or about 1-10 mg/m², 10-20 mg/m², 20-30 mg/m², 30-40 mg/m², or 40-50 mg/m², or any value between the foregoing. In some embodiments, the inhibitor of mTOR is administered at a dose of 1 mg/m², 2 mg/m², 3 mg/m², 4 mg/m², 5 mg/m², 6 mg/m², 7 mg/m², 8 mg/m², 9 mg/m², 10 mg/m², 11 mg/m², 12 mg/m², 13 mg/m², 14 mg/m², 15 mg/m², 16 mg/m², 17 mg/m², 18 mg/m², 19 mg/m², 20 mg/m², 21 mg/m², 22 mg/m², 23 mg/m², 24 mg/m², 25 mg/m², 26 mg/m², 27 mg/m², 28 mg/m², 29 mg/m², 30 mg/m², 31 mg/m², 32 mg/m², 33, mg/m², 34 mg/m², or 35 mg/m². In some embodiments, the inhibitor of mTOR is administered as a dose of 1, 5, or 10 mg/m². In some embodiments, the inhibitor of mTOR is administered as a dose of 10 mg/m². In some embodiments, the inhibitor of mTOR is administered as a dose of 25 mg/m².

In some embodiments, the inhibitor of mTOR is administered once. In some embodiments, the inhibitor of mTOR is administered at least twice. In some embodiments, the administration of mTOR further includes administering a loading dose.

In some embodiments, the inhibitor of mTOR is administered as a loading dose at a dose of from at or about 1-10 mg, 10-100 mg, or 100-1000 mg, or any value between the foregoing. In some embodiments, the inhibitor of mTOR is administered as a loading dose at a dose from at or about 100-200 mg, 200-300 mg, 300-400 mg, 400-500 mg, 500-600 mg, 600-700 mg, 700-800 mg, 800-900 mg, or 900-1000 mg, or any value between the foregoing. In some embodiments, the inhibitor of mTOR is administered as a loading dose at a dose of 100 mg, 200 mg, 300 mg, 400 mg, 500 mg, 600 mg, 700 mg, 800 mg, 900 mg, or 1000 mg. In some embodiments, the inhibitor of mTOR is administered as a loading dose as a dose of 500 mg. In some embodiments, the inhibitor of mTOR is administered as a loading dose as a dose of 800 mg. In some embodiments, the inhibitor of mTOR is administered at a dose of from at or about 1-10 mg, 10-20 mg, 20-30 mg, 30-40 mg, or 40-50, or any value between the foregoing. In some embodiments, the inhibitor of mTOR is administered as a loading dose at a dose of 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33, mg 34 mg, or 35 mg. In some embodiments, the inhibitor of mTOR is administered as a loading dose as a dose of 50, 25, or 10 mg. In some embodiments, the inhibitor of mTOR is administered as a loading dose as a dose of 40 mg. In some embodiments, the inhibitor of mTOR is administered as a loading dose as a dose of 25 mg. In some embodiments, the inhibitor of mTOR is administered as a loading dose at a dose of from at or about 1-10 mg/m², 10-100 mg/m², or 100-1000 mg/m², or any value between the foregoing. In some embodiments, the inhibitor of mTOR is administered as a loading dose at a dose from at or about 100-200 mg/m², 200-300 mg/m², 300-400 mg/m², 400-500 mg/m², 500-600 mg/m², 600-700 mg/m², 700-800 mg/m², 800-900 mg/m², or 900-1000 mg/m², or any value between the foregoing. In some embodiments, the inhibitor of mTOR is administered as a loading dose at a dose of 100 mg/m², 200 mg/m², 300 mg/m², 400 mg/m², 500 mg/m², 600 mg/m², 700 mg/m², 800 mg/m², 900 mg/m², or 1000 mg/m². In some embodiments, the inhibitor of mTOR is administered as a loading dose at a dose of 50 mg/m², 100 mg/m², 150 mg/m², 200 mg/m², 250 mg/m², 300 mg/m², 350 mg/m², 400 mg/m², 450 mg/m², or 500 mg/m². In some embodiments, the inhibitor of mTOR is administered as a loading dose as a dose of 200 mg/m². In some embodiments, the inhibitor of mTOR is administered as a loading dose as a dose of 220 mg/m². In some embodiments, the inhibitor of mTOR is administered as a loading dose at a dose of from at or about 1-10 mg/m², 10-20 mg/m², 20-30 mg/m², 30-40 mg/m², or 40-50 mg/m², or any value between the foregoing. In some embodiments, the inhibitor of mTOR is administered as a loading dose at a dose of 1 mg/m², 2 mg/m², 3 mg/m², 4 mg/m², 5 mg/m², 6 mg/m², 7 mg/m², 8 mg/m², 9 mg/m², 10 mg/m², 11 mg/m², 12 mg/m², 13 mg/m², 14 mg/m², 15 mg/m², 16 mg/m², 17 mg/m², 18 mg/m², 19 mg/m², 20 mg/m², 21 mg/m², 22 mg/m², 23 mg/m², 24 mg/m², 25 mg/m², 26 mg/m², 27 mg/m², 28 mg/m², 29 mg/m², 30 mg/m², 31 mg/m², 32 mg/m², 33, mg/m², 34 mg/m², or 35 mg/m². In some embodiments, the inhibitor of mTOR is administered as a loading dose as a dose of 1, 5, or 10 mg/m². In some embodiments, the inhibitor of mTOR is administered as a loading dose as a dose of 10 mg/m². In some embodiments, the inhibitor of mTOR is administered as a loading dose as a dose of 25 mg/m².

In some embodiments, each dose of the inhibitor of mTOR is administered daily.

In some embodiments, the inhibitor of mTOR is administered subcutaneously, intravenously, and/or intramuscularly.

In some embodiments, the subject is administered or has been administered an inhibitor of a cellular restriction factor that is an antiviral restriction factor in combination with the viral vector (e.g. lentiviral vector) and inhibitor of mTOR in accord with the provided methods. In some embodiments, the antiviral restriction factor is an antiviral restriction factor as described in Section III, such as a SAMHD1 inhibitory viral proteins. In some embodiments, the SAMHD1 inhibitory viral protein is linked to the viral vector.

In some embodiments, the subject is administered or has been administered a recombinant cytokine in combination with the viral vector (e.g. lentiviral vector) and inhibitor of mTOR in accord with the provided methods. Exemplary recombinant cytokines that can be administered in combination with provided methods are described in Section III.C. In some embodiments, the recombinant cytokine is IL-7 or IL-15 or a combination thereof. In some embodiments, the cytokine, such as IL-7 or IL-15, has activity as an antiviral restriction factor.

In some embodiments, the subject is administered or has been administered an inhibitor of an antiviral restriction factor in combination with the viral vector (e.g. lentiviral vector) and an inhibitor of mTOR in accord with the provided methods. In some embodiments, the inhibitor of an antiviral restriction factor and viral vector (e.g. lentiviral vector) are administered simultaneously. In some embodiments, the subject is administered or has been administered an inhibitor of an antiviral restriction factor and a viral vector consecutively. In some embodiments, the inhibitor of an antiviral restriction factor and the viral vector are administered on the same day. In some embodiments, the inhibitor of an antiviral restriction factor is administered intermittently such as in a particular dosing regimen with a defined frequency or schedule.

In certain embodiments, the subject is administered or has been administered an inhibitor of an antiviral restriction factor 1 month before or after administration of the viral vector or a first dose of the viral vector. In some embodiments, the subject is administered or has been administered an inhibitor of an antiviral restriction factor within 1 month before administration of the viral vector or a first dose of the viral vector, such as within or at or about 4 weeks, 3 weeks, 2 weeks or 1 weeks, such as at or about 1 day, 2 days, 3 days, 4 days, 5 days, 6 days or 7 days before administration of the viral vector or a first dose of the viral vector. In some embodiments, the subject is administered or has been administered an inhibitor of an antiviral restriction factor within 3 days before administration of the viral vector.

In certain embodiments, the subject is administered or has been administered inhibitor of an antiviral restriction factor 1 day before or after administration of the viral vector or a first dose of the viral vector. In some embodiments, the subject is administered or has been administered inhibitor of an antiviral restriction factor within 1 day before administration of the viral vector or a first dose of the viral vector, such as within or at or about 24, 23, 22, 21, 20, 19, 18, 17, 16, 15, 14, 13, or 12 hours, or such as 12, 11, 10, 9, 8, 7, 6, 5, 4, 3, 2, or 1 hour before administration of the viral vector or a first dose of the viral vector. In some embodiments, the subject is administered or has been administered inhibitor of an antiviral restriction factor within 3 hours before administration of the viral vector.

In some embodiments, the inhibitor of an antiviral restriction factor is administered at a dose of from at or about 1-10 mg, 10-20 mg, 20-30 mg, 30-40 mg, or 40-50, or any value between the foregoing. In some embodiments, the inhibitor of an antiviral restriction factor is administered at a dose of 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33, mg 34 mg, or 35 mg. In some embodiments, the inhibitor of an antiviral restriction factor is administered as a dose of 50, 25, or 10 mg. In some embodiments, the inhibitor of an antiviral restriction factor is administered as a dose of 40 mg. In some embodiments, the inhibitor of an antiviral restriction factor is administered as a dose of 25 mg.

In some embodiments, the inhibitor of an antiviral restriction factor is administered once. In some embodiments, the inhibitor of an antiviral restriction factor is administered at least twice.

In some embodiments, each dose of the inhibitor of an antiviral restriction factor is administered daily. In some embodiments, the inhibitor of an antiviral restriction factor is administered subcutaneously, intravenously, and/or intramuscularly.

In some embodiments, the viral vector is administered at a dose of from about 10⁹ to about 10⁵ genome copies (GC) units. In some embodiments, the viral vector is administered at a dose of 10⁵, 10⁶, 10⁷, 10⁸, 10⁹, 10¹⁰, 10¹¹, 10¹², 10¹³, 10¹⁴ or 10¹⁵ GC units, or any value between any of the foregoing. In some embodiments, the viral vector is administered at a dose of from about from or from about 10⁸ GC/kg to at or about 10¹⁴ GC/kg of the subject's body weight. In some embodiments, the viral vector is administered at a dose of 10⁵ GC/kg, 10⁶ GC/kg, 10⁷ GC/kg, 10⁸ GC/kg, 10⁹ GC/kg, 10¹⁰ GC/kg, 10¹¹ GC/kg, 10¹² GC/kg, 10¹³ GC/kg, 10¹⁴ GC/kg, 10¹⁵ GC/kg, 10¹⁶ GC/kg, 10¹⁷ GC/kg, 10¹⁸ GC/kg, 10¹⁹ GC/kg, or 10²⁰ GC/kg of the subject's body weight, or any value between any of the foregoing.

In some embodiments, the viral vector is administered at a dose of from about 10⁹ to about 10¹⁵ infectious units (IU). In some embodiments, the viral vector is administered at a dose of 10⁵, 10⁶, 10⁷, 10⁸, 10⁹, 10¹⁰, 10¹¹, 10¹², 10¹³, 10¹⁴ or 10¹⁵ IU, or any value between any of the foregoing. In some embodiments, the viral vector is administered at a dose of from about from or from about 10⁸ IU/kg to at or about 10¹⁴ IU/kg of the subject's body weight. In some embodiments, the viral vector is administered at a dose of 10⁵ IU/kg, 10⁶ IU/kg, 10⁷ IU/kg, 10⁸ IU/kg, 10⁹ IU/kg, 10¹⁰IU/kg, 10¹¹ U/kg, 10¹² U/kg, 10¹³ IU/kg, 10¹⁴IU/kg, 10¹⁵IU/kg, 10¹⁶IU/kg, 10¹⁷ IU/kg, 10¹⁸ IU/kg, 10¹⁹ IU/kg, or 10²⁰IU/kg of the subject's body weight, or any value between any of the foregoing.

In some embodiments, the viral vector is administered at a dose of from about 10⁹ to about 10⁵ transduction units (TU). In some embodiments, the viral vector is administered at a dose of 10⁵, 10⁶, 10⁷, 10⁸, 10⁹, 10¹⁰, 10¹¹, 10¹², 10¹³, 10¹⁴ or 10¹⁵ TU, or any value between any of the foregoing. In some embodiments, the viral vector is administered at a dose of from about from or from about 10⁸ TU/kg to at or about 10¹⁴ TU/kg of the subject's body weight. In some embodiments, the viral vector is administered at a dose of 10⁵ TU/kg, 10⁶ TU/kg, 10 TU/kg, 10⁸ TU/kg, 10 TU/kg, 10¹⁰ TU/kg, 10¹¹ TU/kg, 10¹² TU/kg, 10¹³ TU/kg, 10¹⁴ TU/kg, 10¹⁵ TU/kg, 10¹⁶ TU/kg, 10¹⁷ TU/kg, 10¹⁸ TU/kg, 10¹⁹ TU/kg, or 10²⁰ TU/kg of the subject's body weight, or any value between any of the foregoing.

In some embodiments, the viral vector is administered as one dose. In some embodiments, the viral vector is administered in two doses. In some of the provided embodiments, administration of a total dose of the viral vector includes administration of a total desired dose that includes at least two repeated doses that are each separately administered resulting in multiple administrations over a specified time period. In some embodiments, each repeated dose may be administered from a separate composition containing the viral vector so that the total dose is provided as a plurality of compositions that are administered separately over a specified time period. In some embodiments, the plurality of compositions (e.g. providing a first dose and a second dose, and optionally one or more successive doses) are administered over a time period that is no more than one month. In some embodiments, a first dose and second dose, and in some cases one or more additional doses, are administered over more than one day. In some embodiments, the plurality of compositions (e.g. providing a first dose and a second dose, and optionally one or more successive doses) are administered over a time period that is no more than one week. In some embodiments, the repeated doses are administered over a period of no more than three days, such as once a day for two days (e.g. a first dose and a second dose) or once a day for three days (e.g. a first dose, a second dose, and a third dose).

In some embodiments, the methods are for transducing a target cell in a subject, such as a cell expressing a cell surface receptor to which a targeting moiety binds (e.g., See Section IV.D.2). In some embodiments, the lentiviral vector is a target cell targeting lentiviral vector and comprises a binding agent on its surface for targeted recognition of a molecule on a target cell. In some embodiments, the target cell binding agent is an antibody.

In some embodiments, the methods are for transducing a target cell that is one or more of a monocyte, macrophage, neutrophil, dendritic cell, eosinophil, mast cell, platelet, large granular lymphocyte, Langerhans' cell, natural killer (NK) cell, T lymphocyte (e.g., T cell), a Gamma delta T cell, B lymphocyte (e.g., B cell), CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, a CD30+ lung epithelial cell, a liver sinusoidal endothelial cell or myocyte. In some of any embodiments, the target cell is selected from the group consisting of tumor-infiltrating lymphocytes, T cells, neoplastic or tumor cells, virus-infected cells, stem cells, central nervous system (CNS) cells, hematopoietic stem cells (HSCs), liver cells or fully differentiated cells. In some embodiments, the target cell is selected from the group consisting of a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell. In some of any embodiments, the target cell is a hepatocyte.

In some embodiments, the methods are for transducing T cells in the subject. In some embodiments, the lentiviral vector is a T-cell targeting lentiviral vector and comprises a T cell binding agent on its surface for targeted recognition of a molecule on a T cell, such as CD3, CD4 or CD8. In some embodiments, the T cell binding agent is an antibody. In some embodiments, the T cell binding agent is an anti-CD3 antibody. In some embodiments, the T cell binding agent is an anti-CD4 antibody. In some embodiments, the T cell binding agent is an anti-CD8 antibody. Exemplary T cell binding agents are described in Section IV.

In some embodiments, prior to carrying out the provided methods the T cells are resting or non-activated. In some embodiments, prior to carrying out the provided methods, the resting or non-activated T cells are not treated with one or more T cell stimulatory molecules (e.g., an anti CD3 antibody), one or more T cell costimulatory molecules, and/or one or more T cell activating cytokines. In some embodiments, prior to carrying out the provided methods, the resting or non-activated T cells are not treated with any of one or more T cell stimulatory molecules (e.g., an anti CD3 antibody), one or more T cell costimulatory molecules, and/or one or more T cell activating cytokines.

In some embodiments, prior to carrying out the provided methods, the T cells are activated. In some embodiments, prior to carrying out the provided methods, the activated T cells are treated with one or more T cell stimulatory molecules (e.g., an anti CD3 antibody), one or more T cell costimulatory molecules, and/or one or more T cell activating cytokines.

In some embodiments, prior to carrying out the provided methods, the subject is not administered or has not been administered a T cell activating treatment. In some embodiments, the subject is not administered or has not been administered any of one or more T cell stimulatory molecules (e.g., an anti-CD-3 antibody), one or more T cell costimulatory molecules, and/or one or more T cell activating cytokines. In some embodiments, the T cell activating treatment is lymphodepletion. In some embodiments, the subject is not administered or has not been administered a lymphodepleting therapy.

In some embodiments, prior to carrying out the provided methods, the subject is administered or has been administered a T cell activating treatment. In some of any of the above embodiments, the T cell activating treatment includes one or more T cell stimulatory molecules (e.g., an anti-CD-3 antibody), one or more T cell costimulatory molecules, and/or one or more T cell activating cytokines. In certain embodiments, the subject is not administered or has not been administered the T cell activating treatment (other than an inhibitor of mTOR in accord with the provided methods) within 1 month before or after administration of the viral vector. In some embodiments, the subject is not administered or has not been administered the T cell activating treatment (other than an inhibitor of mTOR in accord with the provided methods) within 1 month before administration of the viral vector, such as within or at or about 4 weeks, 3 weeks, 2 weeks or 1 weeks, such as at or about 1 day, 2 days, 3 days, 4 days, 5 days, 6 days or 7 days before administration of the viral vector. In some embodiments, the subject is not administered the T cell activating treatment (other than an inhibitor of mTOR in accord with the provided methods) within 1 month after administration of the viral vector, such as within or at or about 4 weeks, 3 weeks, 2 weeks or 1 weeks, such as at or about 1 day, 2 days, 3 days, 4 days, 5 days, 6 days or 7 days after administration of the viral vector.

In certain embodiments, the subject is administered or has been administered a T cell activating treatment (other than an inhibitor of mTOR in accord with the provided methods) within 1 month before or after administration of the viral vector. In some embodiments, the subject is administered or has been administered the T cell activating treatment (other than an inhibitor of mTOR in accord with the provided methods) within 1 month before administration of the viral vector, such as within or at or about 4 weeks, 3 weeks, 2 weeks or 1 weeks, such as at or about 1 day, 2 days, 3 days, 4 days, 5 days, 6 days or 7 days before administration of the viral vector. In some embodiments, the subject is administered the T cell activating treatment (other than an inhibitor of mTOR in accord with the provided methods) within 1 month after administration of the viral vector, such as within or at or about 4 weeks, 3 weeks, 2 weeks or 1 weeks, such as at or about 1 day, 2 days, 3 days, 4 days, 5 days, 6 days or 7 days after administration of the viral vector.

In provided aspects, reference to a T cell activating agent (other than an inhibitor of mTOR in accord with the provided methods) may include an agent that binds to a receptor on a T cell or part of a TCR complex and stimulates a primary signal in a T cell and/or an agent that binds to a T cell costimulatory molecule and stimulates a costimulatory signal on a T cell. An example of an agent binds to a primary receptor on a T cell to stimulate a primary signal is an antibody directed against CD3. The use of anti-CD3 antibodies is well-known for activation of T cells. The anti-CD3 antibodies can be of any species, e.g., mouse, rabbit, human, humanized, or camelid. Exemplary antibodies include OKT3, CRIS-7, I2C the anti-CD3 antibody included in DYNABEADS Human T-Activator CD3/CD28 (Thermo Fisher), and the anti-CD3 domains of approved and clinically studied molecules such as blinatumomab, catumaxomab, fotetuzumab, teclistamab, ertumaxomab, epcoritamab, talquetamab, odronextamab, cibistamab, obrindatamab, tidutamab, duvortuxizumab, solitomab, eluvixtamab, pavurutamab, tepoditamab, vibecotamab, plamotamab, glofitamab, etevritamab, and tarlatamab. In some embodiments, the one or more T cell costimulatory molecules include CD28 ligands (e.g., CD80 and CD86); antibodies that bind to CD28 such as CD28.2, the anti-CD28 antibody included in DYNABEADS Human T-Activator CD3/CD28 (Thermo Fisher) and anti-CD28 domains disclosed in US2020/0199234, US2020/0223925, US2020/0181260, US2020/0239576, US2020/0199233, US2019/0389951, US2020/0299388, US2020/0399369, and US2020/0140552; CD137 ligand (CD137L); anti-CD137 antibodies such as urelumab and utomilumab; ICOS ligand (ICOS-L); and anti-ICOS antibodies such as feladilimab, vopratelimab, and the anti-ICOS domain of izuralimab. In some embodiments, the stimulating or activating agent comprises an anti-CD3 antibody or antigen-binding fragment thereof and an anti-CD28 antibody or antigen-binding fragment thereof. Thus, in some embodiments, incubating the T cells with a stimulating or activating agent comprises incubating the T cells in the presence of an anti-CD3 antibody or antigen-binding fragment thereof and an anti-CD28 antibody or antigen-binding fragment thereof. In some embodiments, the stimulating or activating agent that binds to a TCR/CD3 complex is an MHC molecule loaded with peptide, which leads to mediation of a T cell response, including, but not limited to, proliferation, activation, and/or differentiation.

In some aspects, the viral vector includes or encodes a T cell activating agent. In some embodiments, the viral vector includes or encodes a membrane-bound T cell activating agent. In some embodiments, the viral vector includes or encodes a T cell activating agent that is displayed on the surface. In some embodiments, the viral vector includes or encodes a T cell activating agent selected from a polypeptide capable of binding CD3 and activating T cells, a polypeptide capable of binding to CD28, or both. In some embodiments, the viral vector includes or encodes a T cell activating agent selected from an activating anti-CD3 antibody (e.g. an activating anti-CD3 scFv), a T cell activating cytokine (e.g. IL-2, IL-7, IL-15 or IL-21) or a T cell costimulatory molecule (e.g. anti-CD28 antibody, CD80, CD86, CD137L or ICOS-L. In some aspects, the viral vector includes one or more T cell stimulatory molecules (e.g., an activating anti CD3 antibody), one or more T cell costimulatory molecules, and/or one or more T cell activating cytokines.

In some embodiments, the vector does not include or encode an inhibitory RNA molecule. In some embodiments, the inhibitory RNA molecule targets an mRNA transcribed from a gene expressed by T cells, a gene encoding a component of a T cell receptor (TCR), or both. In some embodiments, the gene is PD-1, CTLA4, TCR□, TCR□, CD3□, SOCS1, SMAD2, a miR-155 target, IFN□, TRAIL2, and/or ABCG1.

In some embodiments, the vector includes or encodes an inhibitory RNA molecule. In some embodiments, the inhibitory RNA molecule targets an mRNA transcribed from a gene expressed by T cells, a gene encoding a component of a T cell receptor (TCR), or both. In some embodiments, the gene is PD-1, CTLA4, TCR□, TCR□, CD3□, SOCS1, SMAD2, a miR-155 target, IFN□, TRAIL2, and/or ABCG1.

In some embodiments, the methods further include administering a lymphodepleting therapy to a subject. Lymphodepletion may be induced by various treatments that destroy lymphocytes and T cells in the subject. For example, the lymphodepletion may include myeloablative chemotherapies, such as fludarabine, cyclophosphamide, bendamustine, and combinations thereof. Lymphodepletion may also be induced by irradiation (e.g., full-body irradiation) of the subject. In some embodiments, a lymphodepleting therapy comprises cyclophosphamide and/or fludarabine. In some embodiments, the methods further comprise administering cyclophosphamide and/or fludarabine.

### B. Systems and Methods for Extra Corporeal Transduction Via Viral Vector

Provided herein are methods for administration of a viral vector or an exogenous agent to a subject in combination with an mTOR inhibitor. In some embodiments, cells from a subject are contacted with a viral vector and mTOR inhibitor ex vivo and administered back to the subject. In some embodiments, the subject has been or is to be administered the mTOR inhibitor and cells from the subject are contacted with a viral vector ex vivo and administered back to a subject that has been or is to be administered mTOR inhibitor.

In some embodiments the method comprises a) obtaining whole blood from the subject; b) collecting the fraction of blood containing PBMC or a subset (e.g. containing leukocyte components); c) contacting the collected PBMC or subset (e.g. leukocyte components) with a composition comprising a viral vector an inhibitor of mTOR to create a transduction mixture; and d) reinfusing the contacted PBMC or subset (e.g. leukocyte components) and/or transduction mixture to the subject, thereby administering the viral vector and/or exogenous agent to the subject. In some embodiments, the method is performed ex vivo to the subject. In some embodiments, the method is performed extracorporeal or ex vivo to the subject. In some embodiments, a suitable device or devices to complete the provided method are comprised within a fluid circuit (e.g., in-line). In some embodiments, the in-line system is a closed system.

In some embodiments, the subject is administered or has been administered an inhibitor of mTOR in combination with the viral vector (e.g. lentiviral vector) in accord with the provided methods. In some embodiments the method comprises a) obtaining whole blood from the subject; b) collecting the fraction of blood containing PBMC or a subset (e.g. containing leukocyte components); c) contacting the collected PBMC or subset (e.g. leukocyte components) with a composition comprising a viral vector to create a transduction mixture; and d) reinfusing the contacted PBMC or subset (e.g. leukocyte components) and/or transduction mixture to the subject, thereby administering the viral vector and/or exogenous agent to the subject. In some embodiments, the method is performed ex vivo to the subject. In some embodiments, the method is performed extracorporeal or ex vivo to the subject. In some embodiments, a suitable device or devices to complete the provided method are comprised within a fluid circuit (e.g., in-line). In some embodiments, the in-line system is a closed system.

In some embodiments, the inhibitor of mTOR is administered to the subject prior to collecting the fraction of blood containing PBMC or subset thereof from the subject. In some embodiments, the inhibitor of mTOR is administered to the subject concurrently with or near concurrently with (e.g. within 12 hours, such as within 1, 2, 3, 4, 5, 6, 7, 8, 9, 10, 11 or 12 hours) administering to the subject the contacted PBMC or subset (e.g. leukocyte components) and/or transduction mixture. In some embodiments, the inhibitor of mTOR is administered intermittently such as in a particular dosing regimen with a defined frequency or schedule. Methods and dosing for administering the mTOR inhibitor to the subject can be any as described in Section III.A.

In some embodiments, the antiviral restriction factor inhibitor is administered to the subject prior to collecting the fraction of blood containing PBMC or subset thereof from the subject. In some embodiments, the antiviral restriction factor inhibitor is administered to the subject concurrently with or near concurrently with (e.g. within 12 hours, such as within 1, 2, 3, 4, 5, 6, 7, 8, 9, 10, 11 or 12 hours) administering to the subject the contacted PBMC or subset (e.g. leukocyte components) and/or transduction mixture. In some embodiments, the antiviral restriction factor inhibitor is administered intermittently such as in a particular dosing regimen with a defined frequency or schedule. Methods and dosing for administering the antiviral restriction factor inhibitor to the subject can be any as described in Section III.B.

The method according to the present disclosure is capable of delivering a viral vector and/or exogenous agent to a system for administration, such as an extracorporeal system. The extracorporeal system for use in the provided method may include a combination of various machine hardware components (i.e., apheresis and blood processing machines), a software control module, and/or a sensor module in-line to ensure monitor the process such as to assess efficiency of transduction, cell health and other aspects related to accuracy and safety of the dosing, and the use of replacement fluids designed to fully exploit the design of the system according to the present methods. It is understood that components described for one system according to the present invention can be implemented within other systems according to the present invention as well. In some embodiments, the method is performed in-line. In some embodiments, the method is performed in a closed fluid circuit, or functionally closed fluid circuit. In some embodiments, various components of the system of administration for use in the provided embodiments are operably connected to the subject, and/or to each other.

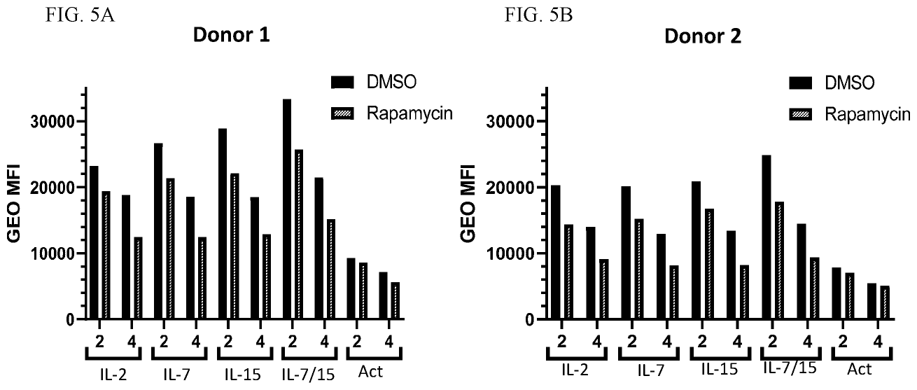

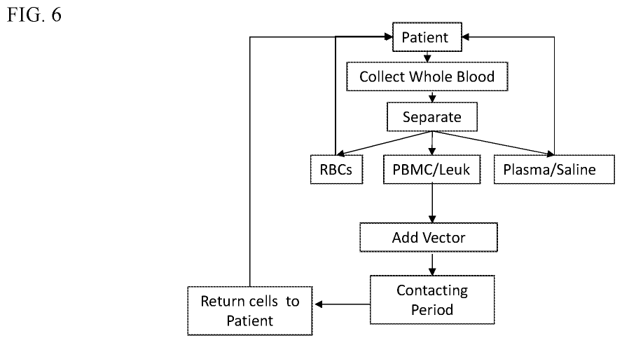

In some embodiments, the method for administration comprises the use of a blood processing set for obtaining the whole blood from the subject, a separation chamber for the separating the blood, a contacting container for the contacting the separated blood component with the composition comprising viral vectors, and a further fluid circuit for re-infusion of the contacted blood component to the patient. (see e.g. FIG. 6). In some embodiments, the contacting chamber is for contacting the separated blood component with a composition of viral vectors comprising nucleic acids encoding an exogenous agent. In some embodiments, the method further comprises the use of any of i) a washing component for concentrating cells of the separated blood component (i.e., leukocytes), and/or a ii) a sensor and/or module for monitoring cell density and/or concentration, In some embodiments, the methods allow processing of blood directly from the patient, transfection with viral vector (e.g. transduction with a viral vector), and reinfusion directly to the patient without any steps of selecting for the target cells to be transduced. For instance, if T cells are a desired target cell, the method does not include any step for selecting for T cells or for CD8+ T cells. Further the methods also can be carried out without cryopreserving or freezing any cells before or between any one or more of the steps, such that there is no step of formulating cells with a cryoprotectant, e.g. DMSO. In some embodiments, the provided methods also do not include a lymphodepletion regimen. In some embodiments, the method including steps (a)-(d) can be carried out for a time of no more than 24 hours, such as between 2 hours and 12 hours, for example 3 hours to 6 hours.

In some embodiments, the method is performed in-line. In some embodiments, the method is performed in a closed fluid circuit, or a functionally closed fluid circuit. In some embodiments, each of steps (a)-(d) are performed in-line in a closed fluid circuit in which all parts of the system are operably connected, such as via at least one tubing line. In some embodiments, the system is sterile. In some embodiments, the closed fluid circuit is sterile.

In some embodiments, operable connection of the system, for example such as via the blood processing unit, separation chamber, contacting chamber and reinfusion processing unit, is achieved by a connector set containing a least one tubing line and one or more optional connectors. The connector set may include at least one tubing line, such as a plurality of tubing lines, that provide for an operable connection of all containers or components of the system to provide for the closed fluid path. Thus, in some embodiments, the components of the provided system typically include at least one tubing line, and generally a set or system of tubing lines, and at least one connector. Exemplary connectors include valves, ports, spikes, welds, seals, and hose clamps. The connectors and/or other components may be aseptic, for example, to permit the entire process to be carried out in a closed, sterile system, which can eliminate or reduce the need for clean rooms, sterile cabinets, and/or laminar flow systems.

In some embodiments, the at least one tubing line includes a series of tubing lines. Tubing can be made of a plastic, such as polycarbonate, and may be of various sizes and/or volumes, generally designed to permit flow of the desired liquid compositions at the appropriate rate, and connection with the chamber and/or other components. The series of tubing lines generally allows for the flow of liquids between the chamber and/or one or more components of the system, such as the other containers, facilitated in some aspects by connectors. In some embodiments, the system includes tubing lines connecting each of the various components to at least one other of the components, where liquid is permitted to flow between each, and which may be permitted or stopped by the configuration of various connectors, such as valves, and/or clamps.

In some aspects, the connectors are such that they may be placed in or directed to alternative configurations, respectively blocking, allowing, and/or directing the flow of fluids through various components, such as between various containers and through certain tubing lines connecting various components, such as rotational and gate valves. In other embodiments, certain connectors and/or other components have a single configuration which permits, directs, or blocks passage of liquid or gas, such as seals, caps, and/or open ports or channels. Various components in the system may include valves, ports, seals, and clamps. Valves can include rotational valves, such as stopcocks, rotary valves, and gate valves. Valves can be arranged in a manifold array or as a single multiport rotational valve. Ports may include Luer ports or spike ports. Seals may include O-rings, gaskets, adhesive seals, and couplings. Clamps may include pinch clamps.

In some embodiments, the connector set (e.g. containing one or more tubing lines and/or connectors) is sterile. In some embodiments, the connection set is a disposable processing set that provides a sterile closed pathway between the blood processing unit (e.g. apheresis device) and the return processing unit. In some embodiments, the cells from the subject, including the separated leukocyte components, never leave the disposable set (except for closed system monitoring via the one or more monitoring modules) which, in some aspects, remains connected to the donor subject during the entire dosing administration procedure. Thus, the provided embodiments allow for an efficient process for harvesting leukocytes from whole blood, transfecting the leukocytes (or a subset or cell type therein) with a viral vector and reinfusing the transduction mixture (i.e. the leukocyte components contacted with the viral vectors) directly back to the subject, in which the connector set (e.g. disposable connector set) can provide for a sterile and closed fluid pathway between the blood processing unit (e.g. apheresis device) and the return processing unit so that the entire process occurs while the system is connected to the subject or patient.

Other components of a system include containers capable of holding or storing liquids. The containers can include bags, vials, boxes, syringes, bulbs, tanks, bottles, beakers, buckets, flasks, and tubing lines. Such components can hold compositions used in and produced by the methods, including byproducts and interim products and waste. Such compositions may include liquid, including buffers, growth media, transduction media, water, diluents, washes, and/or saline, and may also include the cells, viral vectors, and/or other agents for use in the processing steps, such as transfection (e.g. transduction).

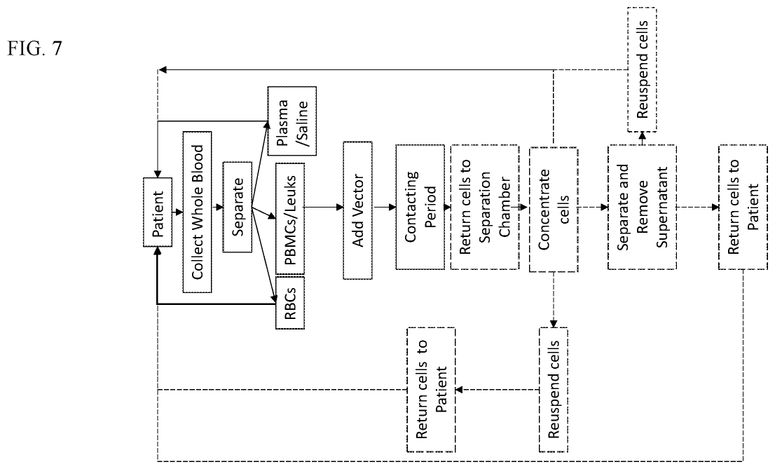

Also provided herein are systems for administration of viral vector comprising a exogenous agent for targeted delivery to a subject or a cell in the subject. In some embodiments, the methods and systems are for autologous administration to the subject. Exemplary systems for administration are shown in FIG. 6 and FIG. 7.

In some embodiments, the provided methods can be used to process about 3-8 liters (L) of blood by apheresis, such as leukapheresis, to separate leukocyte components or precursors from a whole blood sample. The leukocyte components or precursors thereof include peripheral blood mononuclear cells (PBMCs). In some embodiments, the collected (e.g. separated) leukocyte components or precursors thereof, such as PBMCs, are contacted with a viral vector to create a transduction mixture. In some embodiments, the amount to leukocyte components or precursors thereof, such as PBMCs, during the contacting is at or about 2×10⁶ to 6×10⁹ nucleated cells in which the cells are at a concentration of 5×10⁶ cells/mL to 1×10⁸ cells/mL, such as at or about 1×10⁷ cells/mL and/or are provided in a volume of 100-400 mL.

In some embodiments, the viral vector composition is a viral vector composition (e.g. lentiviral vector) containing at or about 1×10⁸ to 1×10¹¹ infectious units (IU). In some embodiments, the viral vector composition is a viral vector composition containing at or about 1×10⁹ to about 1×10¹⁵ infectious units (IU). In some embodiments, the viral vector composition is a viral vector composition containing at or about 10⁵, 10⁵, 10⁷, 10⁸, 10⁹, 10¹⁰, 10¹¹, 10¹², 10¹³, 10¹⁴ or 10¹⁵ IU, or any value between any of the foregoing.

In some embodiments, the transduction mixture is reinfused to the subject. In some embodiments, the transduction mixture is reinfused to the subject without additional processing steps.

### 1. Obtaining Whole Blood PMBCs

In some of any of the provided embodiments, the method comprises obtaining whole blood from a subject. In some embodiments, a method of collecting blood components is used. In some embodiments, the method includes inserting a venous-access device into a subject, and withdrawing whole blood from the subject. In some embodiments, the method withdraws the blood from the subject through a draw line, which is optionally operably connected to a blood processing set described below. In some embodiments, a draw line pump controls the flow through the draw line. In some embodiments, an anticoagulant is introduced into the withdrawn blood through an anticoagulant line. In some embodiments, the anticoagulant line pump controls the flow through the anticoagulant line.

In some of any of the provided embodiments, the collection of whole blood is performed in a blood processing set. A suitable blood processing set in some embodiments has at least one blood treatment device, such as a hemofilter or dialyzer. In some embodiments, the blood processing set has a blood chamber and a dialysate chamber separated from the blood chamber by a membrane.

In some embodiments, the method comprises obtaining whole blood from a patient using a blood processing set that contains a priming solution. In some embodiments, the priming solution comprises citrate, and/or citrate with another suitable buffer. In some embodiments, the citrate is concentrated. In some embodiments, the priming solution is a composition of citrate and another suitable buffer (i.e., a dialysis or replacement solution). In some embodiments, the priming fluid is present in the tubes and/or connectors of the blood processing set at the time of obtaining the whole blood.

In some embodiments, the method comprises filling the blood processing set with whole blood, or a fluid comprising whole blood, from the patient. In some embodiments, the blood processing set is filled prior to priming. In some embodiments, the blood processing set is filled following priming. In some embodiments, the filling of the blood processing set may be done within a closed fluid circuit (e.g., in-line). In some embodiments, the blood processing set may be isolated from the fluid circuit before or after collection and filling of the set. In some embodiments, the blood processing set may be connected to a fluid circuit following the filling of said blood processing set.

In some embodiments, the blood processing set comprises a dialysate compartment. In some of any embodiments, the method for collecting whole blood comprises filling the dialysate compartment of the blood processing set bypassing or passing over a membrane. Thus, in some embodiments, a portion of a priming solution described herein (which, as noted above, may include citrate) travels from the blood chamber of the blood processing set (e.g., hemofilter) to the side of the dialysate chamber. In some embodiments, the filling of the dialysate compartment can be through the fresh dialysate side with a solution having the same properties as the priming solution. In some embodiments, the dialysate solution comprises at least the same calcium concentration and/or citrate concentration as the priming solution.

In some embodiments, the blood processing set has at least one blood treatment device. In some embodiments, the blood treatment apparatus is a hemodialysis apparatus, a hemofiltration apparatus or a hemodiafiltration apparatus. In some embodiments, the venous line of the extracorporeal blood circuit is the section from which the blood of the ex vivo treatment patient flows to the body of the patient or from which it flows back after being treated in a blood treatment device (e.g. a dialyzer).

In some embodiments, the blood processing set has at least one sensor, module, control or regulating unit. In some embodiments, the at least one sensor, module, control or regulating unity is operably connected to one or more components disclosed herein with a fluid and/or signal connection.

In some of any of the provided embodiments, the at least one sensor, module, control or regulating unit is programmed to interact with a blood treatment device, such as a hemofilter or dialyzer as described herein, to perform a blood treatment or to control or regulate the blood processing set after priming according to one of the above-described embodiments. In some embodiments, no heparin or other anticoagulant and/or calcium is added to the ex vivo blood circuit and/or the patient.

In some exemplary embodiments according to the invention, in the method of blood treatment after priming, the blood pump is initially set slower than later, and later set faster than earlier.

In some embodiments, a blood pump (e.g., such as a peristaltic pump) is positioned on the blood extraction tube to pump of the whole blood from the subject to a next chamber for use in the method, e.g., a separation chamber as described in Section II.B.2. In some embodiments, the blood extraction pump is positioned midway between the point at which blood is withdrawn from the subject (e.g., the venipuncture site) and the point at which the blood enters the blood processing set and/or separation chamber (e.g., the inlet). In some embodiments, a “distal segment” of the blood extraction tube carries the withdrawn blood from the subject to the blood pump. In some embodiments, a “proximal segment” of the blood extraction tube carries the blood from the blood pump to a next apparatus for use in the method, e.g., a separation chamber.

In some aspects, it is common in the art to add a flow of anticoagulant solution (e.g. heparin-saline or warfarin-saline) into the “distal segment” of the blood extraction tube at a location close to the vascular access point. Such addition of anticoagulant solution near the vascular access point serves to prevent clotting or coagulation of the blood as it subsequently passes through the apheresis system. This addition of anticoagulant solution is typically accomplished by providing a bag or container of anticoagulant solution connected to the “distal segment” of the blood extraction tube by way of an anticoagulant solution delivery tube. An anticoagulant pump, such as a peristaltic pump, may be positioned on the anticoagulant delivery tube to pump a metered amount of anticoagulant solution through said anticoagulant delivery tube and into the distal end of the “distal segment” of the blood extraction tube to accomplish the desired anticoagulation effect.

The blood processing set may also have a plurality of lines including, but not limited to, a blood draw line, an anticoagulant line, and a return line. In some embodiments, a line specific pump controls the flow through each of these lines. In some embodiments, the blood draw line may be connected (e.g., via a fluid connection that may be closed) to the venous-access device and configured to transport the drawn whole blood to a separation chamber as described below. In some embodiments, a blood draw pump controls the flow through the blood draw line. An anticoagulant line may be connected to an anticoagulant source, and may introduce anticoagulant into the drawn whole blood, i.e., near the venous access device. In some embodiments, an anticoagulant pump controls the flow through the anticoagulant line. The return line may fluidly connect the venous-access device and the separation device, and may be used to return the first or second blood component or compensation fluid to the subject. A return pump may control the flow through the return line. In some embodiments, the return line fluidly connects to the venous-access device at a point between the blood draw pump and the venous-access device.

In some embodiments, the blood processing set is comprised in fluid circuit, optionally a closed in-line circuit. In some embodiments, the blood processing set can be operably connected in a fluid and/or signal connection with any of the disclosed units and/or devices, or in a fluid and/or signal connection with such units and/or devices. In some embodiments, the operable connection via at least one connector selected from the group consisting of valves, luer ports and spikes. In some embodiments, one or more of these connectors are disposable. In some embodiments, one or more components of the blood processing set is disposable. In some embodiments, the blood processing set is disposable.

2. Collecting Cells by Separation from the Blood Fraction

In some of any of the provided embodiments, the method further comprises the collection of one or more components from whole blood. In some embodiments, the method further comprises the collection of peripheral blood mononuclear cells (PBMCs) or precursors thereof from whole blood. In some embodiments, the method further comprises the collection of mononuclear cells or precursors thereof from whole blood. In some embodiments, the mononuclear cells are collected via apheresis from whole blood. In some embodiments, the PBMCs are collected via apheresis from whole blood. In some embodiments, the method further comprises the collection of leukocytes or precursors thereof from whole blood. In some embodiments, cells are collected via apheresis from whole blood. In some embodiments, leukocytes or precursors thereof are collected via apheresis from whole blood. In some embodiments, the leukocytes or precursors thereof are collected via leukapheresis. In some embodiments, the mononuclear cells or precursors thereof are collected via mononuclear collection (MNC) or continuous MNC (CMNC). In some embodiments, the leukocytes (white blood cells) include lymphocytes (e.g. T cells, NK cells and B cells), monocytes, macrophages and granulocytes (e.g. neutrophils, eosinophils and basophils). In some embodiments, the collected cells may also include red blood cells, such a hematocrit.

In some embodiments, the method comprises the collection of peripheral blood mononuclear cells (PBMCs). In some embodiments, the method comprises the collection of mononuclear cells. In some embodiments, PBMC's include peripheral blood cells having a round nucleus. In some embodiments, mononuclear cells include blood cells having a single spherical or near-spherical nucleus. In some embodiments, the collected cells are mononuclear cells and/or PBMC's that are lymphocytes (e.g. T cells, NK cells and B cells). In some embodiments, the collected cells are PBMC's that are monocytes. In some embodiments, the PBMC's include leukocyte precursors and/or hemapoietic stem cells. In some embodiments, the leukocyte precursors, such as hemapoietic stem cells, may be collected from the blood (i.e., wherein PBMC are collected). In some embodiments, leukocytes and precursors thereof (e.g. hematopoietic stem cells) are collected and separated from the blood fraction. In some embodiments, leukocyte components that are mature white blood cells are collected and separated from the blood fraction. In some embodiments, leukocyte precursor cells (e.g. hematopoietic stem cells) are collected and separated from the blood fraction.

In some aspects, apheresis is a process wherein whole blood is: (a) withdrawn (e.g., as described above); (b) separated into two or more fractions (i.e., components); and (c) at least one of the separated blood components is retransfused (reinfused) into the subject. In some aspects, the most common type of apheresis procedure is known as “plasmapheresis”. In plasmapheresis a quantity of liquid plasma is separated from a “cell concentrate” comprising the remaining liquid and cellular constituents of the blood and such cell concentrate is, thereafter, retransfused into the subject. Other types of apheresis procedures include “leukapheresis” (wherein leukocytes are separated from the whole blood) and “thrombocytapheresis” (wherein platelets are separated from the whole blood). In some embodiments, the method comprises a step of leukapheresis. In some aspects, apheresis procedures are performed through the use of automated and/or electronically-controlled apheresis instruments.

Examples of commercially available automated apheresis instruments include the Autopheresis-C® system (Baxter Healthcare Corporation, Fenwal Division, 1425 Lake Cook Road, Deerfield, Ill. 60015), and the (Haemonetics Corporation, City, State). Other commercially available apheresis machines for use in collection of mononuclear cells and/or PBMCs include Spectra Optia® and COBE Spectra®. In some embodiments, the apheresis is a two-step Sepctra Optia® mononuclear cell (MNC apheresis) system. In some embodiments, the apheresis is a Spectra® Optia continuous mononuclear cell (CMNC apheresis) system. In some embodiments, the apheresis device (e.g. Spectra® Optia) includes three major sub-systems, 1) the apheresis machine itself (centrifuge, centrifuge filler, pumps, valves, computerized safety and control systems, etc.), 2) a sterile, single-use, disposable blood tubing set, and 3) embedded software. In some embodiments, such a system can be used to collect mononuclear cells (MNC) from the peripheral blood.

In some embodiments, apheresis uses one or more blood separation apparatus such as a rotation, membrane or centrifugal separator (i.e., a separation chamber as described further below). In some embodiments, the collection of a fraction of blood is via extracorporeal apheresis.

In some of any of the provided embodiments, the collecting of the fraction of blood is via separation into one or more blood components in a separation chamber. In some of any of the provided embodiments, the fraction of blood containing leukocyte components or precursors thereof is collected via a separation chamber. In some embodiments, the separation chamber is configured to separate the PBMCs from whole blood by filtration, such as by membrane filtration. In some embodiments, the separation chamber is configured to separate the PBMCs from whole blood by centrifugation. In some embodiments, the remaining blood components (e.g. plasma, red blood cells and/or platelets) may be returned into the blood stream of the subject.

In some embodiments, the separation chamber includes a centrifuge in which PBMCs are separated by centrifugation. With centrifugation, blood components are separated in order of increasing density as follows: plasma, platelets, lymphocytes and monocytes, granulocytes, and red blood cells. Once blood components are separated, outlet tubes placed within the separation chamber (e.g. apheresis system) allow specific components (e.g. PBMCs) to be selectively removed from the subject based on the density variation into a container. The other components can be returned to the subject and, optionally, are mixed with replacement fluids, such as colloids and crystalloids, during return. In some embodiments, a packing factor (PF) for centrifugation is chosen to achieve the desired separation of cells. The packing factor is characterized by the g-force associated with the centrifugations, the sedimentation velocity at 1 g, the residence time in the separation chamber, and the distance over which sedimentation occurs. The packing factor provides a measure of the radial migration compared to the width of the centrifuge chamber, with adequate cell separation obtained when P>1. In some embodiments, the rotational speed of the centrifuge is from 800 rpm to 2400 rpm, such as 1000 rpm to 2000 rpm, for example at or about 1500 rpm (about 100 g). It is within the level of a skilled artisan to determine the appropriate packing factor for separating cells. For instance, the packing factor can depend on factors such as the particular apheresis device being used, the centrifugal speed, the residence time of cells in the chamber and other factors. In some embodiments, the packing factor is between 2 and 20, such as between 2 and 16, between 2 and 12, between 2 and 8, between 2 and 4, between 4 and 20, between 4 and 16, between 4 and 12, between 4 and 8, between 8 and 20, between 8 and 16, between 8 and 12, between 12 and 20, between 12 and 16 or between 16 and 20. In some embodiments, the packing factor is between 4 and 5, such as at or about 4.5.

In some embodiments, the separation chamber separates the drawn blood into at least a first blood component, and a second blood component. In some embodiments, the separation chamber separates the drawn blood into at least a first blood component containing leukocytes or precursors thereof, and a second blood component (e.g. red blood cells and/or plasma). In some embodiments, the separation chamber may be configured such that the blood components are sent to a first and second blood bag, respectively. In some embodiments, the blood component separation device also has an outlet and may optionally alternate between discharging the first blood component (i.e., leukocytes or precursors thereof) and the second blood component (i.e. red blood cells and/or plasma) through the outlet.

In some embodiments, the separation chamber is a centrifuge, optionally a centrifuge bowl. In some embodiments, the centrifuge may separate the drawn blood into a third blood component in addition to the first blood component and the second blood component blood component. In some embodiments, the second and/or third blood component may be returned to the subject in addition to the first blood component via the return line. In some embodiments, The first blood component can be leukocytes or precursors thereof and/or the second blood component can be red blood cells, and/or the third blood component can be plasma and/or platelets. In some embodiments, the separation chamber separates the whole blood into a first blood component (e.g., containing leukocytes or precursors thereof) and a second blood component, optionally wherein the whole blood is separated into a first, second, and third blood component. In some embodiments, the separation chamber extracts the first blood component from the separation chamber. In some embodiments, the separation chamber extracts leukocytes or precursors thereof from the separation chamber. In some embodiments, the second blood (e.g. red blood cells) and/or third blood component (e.g. plasma and/or platelets) is returned to the subject through the return line. In some embodiments, the return line operably connects to the venous-access device at a point between the draw line pump and the venous-access device.

In some embodiments, the separation chamber is an apheresis device. In some embodiments, the separation chamber is an apheresis device which separates cells based on their respective density. For example, a device which uses differential centrifugation to separate the most dense red blood cells, from the less dense cell components of (i) plasma and (ii) the “buffy coat”. In some embodiments, the collecting cells by separation of the blood is collecting cells of the “buffy coat”. In some embodiments, the “buffy coat” layer comprises lymphocytes (e.g., T, B, and NK cells) as well as monocytes and granulocytes. In some embodiments, the “buffy coat” layer comprises and/or further comprises PBMCs. In some embodiments, the “buffy coat” layer comprises HSCs.

In some embodiments, the separation chamber is an apheresis device. In some embodiments, the separation chamber is an apheresis device that separates cells based on their respective density with the use of a density gradient reagent. For example, a device which uses differential centrifugation to separate the most dense red blood cells and gradient reagent, from the less dense cell components of (i) plasma, and (ii) PBMCs. In some embodiments, the collecting cells by separation of the blood is collecting the PBMC. In some embodiments, the cells of the PBMC layer comprises lymphocytes (e.g., T, B, and NK cells), optionally wherein the cells of the PBMCs layer further comprise monocytes. In some embodiments, the cells of the PBMC comprises HSCs. Any density reagent known in the art is suitable for use in the method, for example sucrose, Percoll, and/or Ficoll can be used to perform density based differential centrifugation in a separation chamber (i.e., apheresis device).

In some embodiments, the separation chamber is a leukapheresis device. In some embodiments, the separation chamber is an leukapheresis device that separates cells based on their respective density with the use of a density gradient reagent. For example, a device which uses differential centrifugation to separate the most dense red blood cells and gradient reagent, from the less dense cell components of (i) plasma, and (ii) leukocytes and/or precursors thereof. In some embodiments, the collecting cells by separation of the blood is collecting the leukocytes. In some embodiments, the cells of the leukocyte layer comprises lymphocytes (e.g., T, B, and NK cells), optionally wherein the cells of the leukocyte layer further comprise monocytes.

In some embodiments, the collected cells contain 20-60% T cells, 5-40% monocytes, 2.5-30% B cells, 2.5-30% NK cells, 0.5-10% granulocytes and 0.5-10% hematocrit. For instance, in some embodiments, the collected cells contain up to 50% T cells, 10-30% monocytes, 5-20% B cells, 5-20% NK cells, 2-5% granulocytes and 2-5% hematocrit. In some embodiments, the collected cells contain on average up to 50% T cells, 20% monocytes, 10% B cells, and 10% NK cells, 3% granulocytes, and 3% hematocrit.

In any of the provided embodiments, the separated cells are collected into a container (also called a “collection container”). The container may be of different forms, including a flexible bag, similar to an IV bag, or a rigid container similar to a cell culture vessel. In particular embodiments, the container is a collection bag. Generally, the composition of the container will be any suitable, biologically inert material, such as glass or plastic, including polypropylene, polyethylene, etc. In particular embodiments, the container is sterile, such as a sterile bag. In some embodiments, the container includes one or more ports such that the cells or reagents can be introduced into or transferred out of the container. For instance, the container may include one or more ports so that reagents for transfection of cells (e.g. transduction with a composition containing viral particles) can be introduced to cells within the container. In some cases more than one port may be present for the introduction of one or more reagents, media, etc. and/or for transferring out the cells.

In some of any of the provided embodiments, the separation of cells is via apheresis, such as by leukapheresis. In some of any of the provided embodiments, the apheresis (e.g., leukapheresis) is for a set number of minutes. In some of any of the provided embodiments, the apheresis (e.g., leukapheresis) is for at most 100, at most 120, at most 140, at most 160, at most 180, at most 200, at most 220, at most 240, at most 260, at most 280, at most 300, at most 320, at most 340, at most 360, at most 380, or at most 400 minutes. In some of any of the provided embodiments, the apheresis (e.g., leukapheresis) is for 100-120, 120-140, 140-160, 160-180, 180-200, 200-220, 220-240, 240-260, 260-280, 280-300, 300-320, 320-340, 340-360, 360-380, or 380-400 minutes, each range inclusive. In some of any of the provided embodiments, the apheresis (e.g., leukapheresis) is for at most 200, 220, 240, 260, 280, or 300 minutes. In some of any of the provided embodiments, the apheresis (e.g., leukapheresis) is for 100, 120, 140, 160, 180, 200, 220, 240, 260, 280, 300, 320, 340, 360, 380, or 400 minutes.

In some embodiments, the collection device, such as the apheresis device (e.g. leukapheresis device) processes blood from a subject for separating the desired blood components (e.g. PBMCs). In some of any of the provided embodiments, the processed blood volume (i.e., the volume of blood obtained from whole blood as described above) is at least 5, 6, 7, 8, 9, 10, 11, 12, 13, 14, 15, 16, 17, 18, 19, or 20 liters. In some of any of the provided embodiments, the processed blood volume is 5-20, 5-18, 5-16, 5-14, 5-12, 5-10, 10-20, 10-18, 10-16, 10-14, 10-12, 12-20, 12-18, 12-16, 2-14, 14-20, 14-18, 14-16, 16-20, 16-18 or 18-20 liters, each range inclusive. In some of any of the provided embodiments, the processed blood volume is 5, 6, 7, 8, 9, 10, 11, 12, 13, 14, 15, 16, 17, 18, 19, or 20 liters, or any value between any of the foregoing. In some of any of the provided embodiments, the processed blood volume is 5, 6, 7, 8, 9, 10, 11, 12, or 13 liters. In some of any of the provided embodiments, the processed blood volume is at most 10, 11, 12, 13, 14, or 15 liters.

In some of any of the provided embodiments, the processed blood volume is at least the total blood volume of the patient and/or subject. For example and in some embodiments, any of the below formulas may be used for calculating the total blood volume of a patient and/or subject.

Formulas for Calculating Total Blood Volume (TBV) of Men and Women:

$$Female : 183 + ( 356 \times {height}^{3} ( meters ) ] + [ 33.1 \times weight ( kg ) ]$$

$$Male : 604 + ( 367 \times {height}^{3} ( meters ) ] + [ 32.2 \times weight ( kg ) ]$$

In some of any of the provided embodiments, the processed blood volume is at least 1, at least 2, at least 3, or at least 4 times the total blood volume of the patient and/or subject. In some of any of the provided embodiments, the processed blood volume is between 1 and 2 times the total blood volume, range inclusive. In some of any of the provided embodiments, the processed blood volume is between 2 and 3 times the total blood volume, range inclusive. In some of any of the provided embodiments, the processed blood volume is between 3 and 4 times the total blood volume, range inclusive. In some of any of the provided embodiments, the processed blood volume is or is about 1.5, 1.6, 1.7, 1.8, 1.9, 2.0, 2.1, 2.2, 2.3, 2.4, or 2.5 times the total blood volume.

In some embodiments, the separation chamber or device containing the same (e.g. apheresis device) is comprised in a fluid circuit, optionally a closed in-line circuit. In some embodiments, the separation chamber can be operably connected in a fluid and/or signal connection with any of the disclosed units and/or devices, or in a fluid and/or signal connection with such units and/or devices. In some embodiments, the operable connection via at least one connector selected from the group consisting of valves, luer ports and spikes. In some embodiments, one or more of these connectors are disposable. In some embodiments, one or more components of the separation chamber set is disposable. In some embodiments, the separation chamber is disposable.

In some of any of the provided embodiments, the cells of the whole blood are separated. In some of any of the provided embodiments, PBMCs or subsets thereof are separated from the whole blood. In some embodiments, the separated cells is or comprise PBMCs. In some embodiments, the separated cells include or are enriched leukocytes. In some of any of the provided embodiments, the leukocyte components or precursors thereof are separated from the whole blood. In some embodiments, the separated cells is or comprise leukocytes. In some embodiments, the separated cells are not leukocytes. In some embodiments, the separated cells are leukocyte precursors, such as hematopoietic stem cells. In some embodiments, the separated cells are stem cells. In some embodiments, the separated cells are hematopoietic stem cells (HSCs).

In some embodiments, the separated cells are or include T cells, such as CD4+ or CD8+ T cells. In some embodiments, the separated cells are or include Natural Killer cells (NK cells). In some embodiments, the separated cells are or include B cells. In some embodiments, the separated cells are or include macrophages. In some embodiments, the separated cells are myeloid derived suppressor cells. In some embodiments, the separated cells are a leukocyte belonging to the group selected from monocytes, lymphocytes, neutrophils, eosinophils, basophils, and macrophages.

In some of any of the provided embodiments, the method does not comprise selection of cells. In some embodiments, the method comprises collecting a cell component from the whole blood without selecting for cell surface expression of any protein. In some embodiments, the method does not comprise selecting T cells positive for a T cell marker (e.g. CD3, CD4 or CD8)). In some embodiments, the method does not comprise selecting cells position for the CD34. In some embodiment, the provided methods do not include a step of immunoaffinity-based selection.

In some embodiments, the separated cells are nucleated. In some embodiments, the separated cells are or comprise peripheral blood mononuclear cells (PBMCs). In some embodiments, the number of nucleated cells (e.g. PBMCs) is 5-10×10⁸, 10-20×108, 20-30×10⁸, 30-40×10⁸, 40-50×10⁸, 50-60×10⁸, 60-70×10⁸, 70-80×10⁸, 80-90×10⁸, 100-150×10⁸, 150-200×10⁸, 200-300×10⁸, or 300-400×10⁸ cells, each range inclusive. In some embodiments, the number of nucleated cells (e.g. PBMCs) is at least 5×10⁸, 10×10⁸, 20×10⁸, 30×10⁸, 40×10⁸, 50×10⁸, 60×10⁸, 70×10⁸, 80×10⁸, 90×10⁸, 100×10⁸, 150×10⁸, 200×10⁸, or 300×10⁸ cells. In some embodiments, the number of nucleated cells (e.g. PBMCs) is 5×10⁸, 10×10⁸, 20×10⁸, 30×10⁸, 40×10⁸, 50×10⁸, 60×10⁸, 70×10⁸, 80×10⁸, 90×10⁸, 100×10⁸, 150×10⁸, 200×10⁸, or 300×10⁸ cells. In some embodiments, the number of nucleated cells (e.g. PBMCs) is 1-5%, 5-10%, 10-20%, 20-30%, 30-40%, 40-50%, or 50-60% of the total number of separated cells, each range inclusive. In some embodiments, the number of nucleated cells (e.g. PBMCs) is at least 5%, 10%, 20%, 30%, 40%, 50%, 60%, 70%, 80%, or 90% of the total cell number of separated cells. In some embodiments, the number of nucleated cells (e.g. PBMCs) is 5%, 10%, 20%, 30%, 40%, 50%, 60%, 70%, 80%, 90%, or 100% of the total cell number of separated cells.

In some embodiments, the separated cells comprise CD3+ cells. In some embodiments, the total number of CD3+ cells is 5-10×10⁸, 10-20×10⁸, 20-30×10⁸, 30-40×10⁸, 40-50×10⁸, 50-60×10⁸, 60-70×10⁸, 70-80×10⁸, 80-90×10⁸, 100-125×10⁸, 125-150×10⁸, 150-175×10⁸, 175-200×10⁸ cells, or 200-300×10⁸ each range inclusive. In some embodiments, the number of CD3+ cells is at least 5×10⁸, 10×10⁸, 20×10⁸, 30×10⁸, 40×10⁸, 50×10⁸, 60×10⁸, 70×10⁸, 80×10⁸, 90×10⁸, 100×10⁸, 150×10⁸, 200×10⁸, or 300×10⁸ cells. In some embodiments, the number of CD3+ cells is 5×10⁸, 10×10⁸, 20×10⁸, 30×10⁸, 40×10⁸, 50×10⁸, 60×10⁸, 70×10⁸, 80×10⁸, 90×10⁸, 100×10⁸, 150×10⁸, 200×10⁸, or 300×10⁸ cells. In some embodiments, the number of CD3+ cells is 1-5%, 5-10%, 10-20%, 20-30%, 30-40%, 40-50%, or 50-60% of the total number of separated cells, each range inclusive. In some embodiments, the number of CD3+ cells is at least 5%, 10%, 20%, 30%, 40%, 50%, 60%, 70%, 80%, or 90% of the total cell number of separated cells. In some embodiments, the number of CD3+ cells is 5%, 10%, 20%, 30%, 40%, 50%, 60%, 70%, 80%, 90%, or 100% of the total cell number of separated cells.

In some embodiments, the separated cells comprise monocytes. In some embodiments, the total number of monocytes is 5-10×10⁸, 10-20×10⁸, 20-30×10⁸, 30-40×10⁸, 40-50×10⁸, 50-60×10⁸, 60-70×10⁸, 70-80×10⁸, 80-90×10⁸, 100-125×10⁸, 125-150×10⁸, 150-175×10⁸, 175-200×10⁸ cells, or 200-300×10⁸ each range inclusive. In some embodiments, the number of monocytes is at least 5×10⁸, 10×10⁸, 20×10⁸, 30×10⁸, 40×10⁸, 50×10⁸, 60×10⁸, 70×10⁸, 80×10⁸, 90×10⁸, 100×10⁸, 150×10⁸, 200×10⁸, or 300×10⁸ cells. In some embodiments, the number of monocytes is 5×10⁸, 10×10⁸, 20×10⁸, 30×10⁸, 40×10⁸, 50×10⁸, 60×10⁸, 70×10⁸, 80×10⁸, 90×10⁸, 100×10⁸, 150×10⁸, 200×10⁸, or 300×10⁸ cells. In some embodiments, the number of monocytes is 1-5%, 5-10%, 10-20%, 20-30%, 30-40%, 40-50%, or 50-60% of the total number of separated cells, each range inclusive. In some embodiments, the number of monocytes is at least 5%, 10%, 20%, 30%, 40%, 50%, 60%, 70%, 80%, or 90% of the total cell number of separated cells. In some embodiments, the number of monocytes is 5%, 10%, 20%, 30%, 40%, 50%, 60%, 70%, 80%, 90%, or 100% of the total cell number of separated cells.

In some embodiments, the separated cells include certain PBMC subsets, such as hematopoietic stem cells. In some embodiments, the separated cells include or are enriched in stem cells. In some embodiments, the separated cells include or are enriched hematopoietic stem cells (HSCs).

In some embodiments, the separated cells comprise stem cells, optionally wherein the separated cells comprise HSCs. In some embodiments, the total number of stem cells is 5-10×10⁸, 10-20×10⁸, 20-30×10⁸, 30-40×10⁸, 40-50×10⁸, 50-60×10⁸, 60-70×10⁸, 70-80×10⁸, 80-90×10⁸, 100-125×10⁸, 125-150×10⁸, 150-175×10⁸, 175-200×10⁸ cells, or 200-300×10⁸ each range inclusive. In some embodiments, the number of stem cells is at least 5×10⁸, 10×10⁸, 20×10⁸, 30×10⁸, 40×10⁸, 50×10⁸, 60×10⁸, 70×10⁸, 80×10⁸, 90×10⁸, 100×10⁸, 150×10⁸, 200×10⁸, or 300×10⁸ cells. In some embodiments, the number of stem cells is 5×10⁸, 10×10⁸, 20×10⁸, 30×10⁸, 40×10⁸, 50×10⁸, 60×10⁸, 70×10⁸, 80×10⁸, 90×10⁸, 100×10⁸, 150×10⁸, 200×10⁸, or 300×10⁸ cells. In some embodiments, the number of stem cells is 1-5%, 5-10%, 10-20%, 20-30%, 30-40%, 40-50%, or 50-60% of the total number of separated cells, each range inclusive. In some embodiments, the number of stem cells is at least 5%, 10%, 20%, 30%, 40%, 50%, 60%, 70%, 80%, or 90% of the total cell number of separated cells. In some embodiments, the number of stem cells is 5%, 10%, 20%, 30%, 40%, 50%, 60%, 70%, 80%, 90%, or 100% of the total cell number of separated cells.

In some embodiments, the separated cells comprise platelets. In some embodiments, the total number of platelets is 50-100×10⁸, 100-200×10⁸, 200-300×10⁸, 300-400×10⁸, 400-500×10⁸, 500-600×10⁸, 600-700×10⁸, 700-800×10⁸, 800-900×10⁸, 1000-1250×10⁸, 1250-1500×10⁸, 1500-1750×10⁸, 1750-2000×10⁸ cells, or 2000-3000×10⁸ each range inclusive. In some embodiments, the number of platelets is at least 50×10⁸, 100×10⁸, 200×10⁸, 300×10⁸, 400×10⁸, 500×10⁸, 600×10⁸, 700×10⁸, 800×10⁸, 900×10⁸, 1000×10⁸, 1500×10⁸, 2000×10⁸, or 3000×10⁸ cells. In some embodiments, the number of platelets is 50×10⁸, 100×10⁸, 200×10⁸, 300×10⁸, 400×10⁸, 500×10⁸, 600×10⁸, 700×10⁸, 800×10⁸, 900×10⁸, 1000×10⁸, 1500×10⁸, 2000×10⁸, or 3000×10⁸ cells.

In some embodiments, the separated cells have a hematocrit reading of 1-5%, range inclusive. In some embodiments, the hematocrit reading is at least 1%, 2%, 3%, 4%, or 5%. In some embodiments, the hematocrit reading is 1%, 2%, 3%, 4%, or 5%. In some embodiments, the hematocrit reading is at most 1%, 2%, 3%, 4%, or 5%.

In some embodiments, the separated cells are viable. In some embodiments, the percentage of viable cells within the separated cell component is 1-5%, 5-10%, 10-20%, 20-30%, 30-40%, 40-50%, 50-60%, 60-70%, 70-80%, 80-90%, 90-95%, or 95-100% of the total cell number each range inclusive. In some embodiments, the number of viable cells is at least 5%, 10%, 20%, 30%, 40%, 50%, 60%, 70%, 80%, or 90% of the total cell number of separated cells. In some embodiments, the number of viable cells is 5%, 10%, 20%, 30%, 40%, 50%, 60%, 70%, 80%, 90%, or 100% of the total cell number of separated cells.

In some embodiments, the separated cells are comprised within the separation chamber in a volume (i.e., within the lumen of a separation chamber). In some embodiments, the separated cells are transferred to a collection container. In some embodiments, the volume of separated cells is between 120-140 mL, 140-160 mL, 160-180 mL, 180-200 mL, 200-220 mL, 220-240 mL, 240-260 mL, 260-280 mL, or 280-300 mL, each range inclusive. In some embodiments, the volume of separated cells is at least 120 mL, 140 mL, 160 mL, 180 mL, 200 mL, 220 mL, 240 mL, 260 mL, 280 mL or 300 mL. In some embodiments, the volume of separated cells is 120 mL, 140 mL, 160 mL, 180 mL, 200 mL, 220 mL, 240 mL, 260 mL, 280 mL or 300 mL. In some embodiments, the volume of the separated cells is no more than 1000, 2000, 3000, 4000, or 5000 mL. In some embodiments, the volume of the separated cells is no more than 1000 mL.

In some embodiments, the concentration of separated cells is between 1×10⁷-2×10⁷, 2×10⁷-3×10⁷, 3×10⁷-4×10⁷, 4×10⁷-5×10⁷, 5×10⁷-6×10⁷, 6×10⁷-7×10⁷, 7×10⁷-8×10⁷, 8×10⁷-9×10⁷,or 9×10⁷-10×10⁷ cells/mL, each range inclusive. In some embodiments, the concentration of separated cells is at least 1×10⁷, 2×10⁷, 3×10⁷, 4×10⁷, 5×10⁷, 6×10⁷, 8×10⁷, 9×10⁷ or 10×10⁷ cells/mL. In some embodiments, the concentration of separated cells is 1×10⁷, 2×10⁷, 3×10⁷, 4×10⁷, 5×10⁷, 6×10⁷, 8×10⁷, 9×10⁷ or 10×10⁷ cells/mL. In some embodiments, the concentration of separated cells is between 1×10⁷ and 2×10⁷.

In some embodiments, an apheresis device (e.g. Spectra Optia or COBE Spectra) processes 10-12 L of blood and separates PBMCs containing leukocytes (while blood cells) by collection to a collection bag. In some embodiments, the volume of separated cells in the container is between 100 mL and 400 mL, such as between 200 mL and 250 mL, e.g. at or about 240 mL. In some embodiments, the number of collected nucleated cells is about 1×10⁸ to 30×10⁹. In some embodiments, the collected cells contain at or about 4×10⁸ to 20×10⁹ CD3+ T cells. In some embodiments, the T cells include CD8+T cells. The exact number of nucleated cells, CD3+ T cells or CD8+ T cells will vary depending on the subject, which can be impacted or different depending on the particular disease or condition of the subject. For instance, an apheresis yield is generally lower in subjects with ALL/CLL compared to lymphoma. In some embodiments, the remaining blood components (e.g. plasma, red blood cells and/or platelets) may be returned into the blood stream of the subject.

In some embodiments, the method does not comprise cryopreservation of the separated cells. Therefore in some embodiments, the separated cells are not subject to cryopreservation. In some embodiments, the separated cells are not subject to cryopreservation further in the method. In some embodiments, the separated cells are not treated with any cryopreservation media, optionally wherein the separated cells are not treated with DMSO.

In some embodiments, the separated cells are not expanded. In some embodiments, the separated cells are not cultured for growth or expansion. In some embodiments, the separated cells are not treated with compositions for expansion, such as adjuvants of cell growth or activation.

In some embodiments, the container (e.g. bag) may contain an anti-coagulant to prevent clotting while the cells and sample are processed ex vivo such as in an extracorporeal in-line device. In some embodiments, the anti-coagulant is citrate or heparin.

In some embodiments, the collection container containing separated cells is a Leukopak. A Leukopak is a sterile bag containing a highly-enriched leukapheresis-derived product. In some embodiments, Leukopaks contain high concentrations of mononuclear cells, B cells, T cells, stem/progenitor cells, dendritic cells, and other cell types.

In some embodiments, the container containing the separated cells (e.g. sterile bag such as a blood bag) may be transferred to a contacting chamber for contacting the cells with a viral vector as described below.

In other embodiments, the container containing the separated cells (e.g. sterile bag such as a blood bag) is used as the contacting chamber and the composition containing viral vector particles is introduced directly into the container (e.g. sterile bag such as a blood bag) containing the separated cells.

3. Contacting Separated Cells with a Viral Vector

In some of any of the provided embodiments, the method comprises contacting the separated cells (e.g. leukocyte components or precursors thereof) with a viral vector, such as a viral vector comprised within a composition, and an mTOR inhibitor. In particular embodiments, the viral vector is a lentiviral vector. In some embodiments, the contacting of the leukocyte components or precursors thereof with the viral vector and mTOR inhibitor creates a transduction mixture.

In some of any of the provided embodiments, the method comprises contacting the separated cells (e.g. leukocyte components or precursors thereof) with a viral vector, such as a viral vector comprised within a composition, and an antiviral restriction factor inhibitor. In particular embodiments, the viral vector is a lentiviral vector. In some embodiments, the contacting of the leukocyte components or precursors thereof with the viral vector and antiviral restriction factor inhibitor creates a transduction mixture.

In some of any of the provided embodiments, the contacting of the separated cells (e.g. leukocyte) is within a contacting chamber. In some embodiments, the contacting chamber is in-line with a blood processing set and/or separation chamber as described above. In some embodiments, the contacting chamber is operably connected to any of the blood processing set and/or separation chamber. In some embodiments, the contacting occurs in the collection container (e.g. bag) into which the separated cells have been collected as described above. Hence, in some cases the contacting chamber and the collection container are the same unit. In some embodiments, the separation chamber and contacting chamber are connected by a fluid circuit, optionally a closed fluid circuit. In some embodiments, the separation chamber and contacting chamber are connected via a fluid circuit that is a closed pathway between the separation and contacting chamber, optionally wherein the circuit is sterile.

In some embodiments, contacting the separated cells with a viral vector or a composition comprising viral vectors results in the transfection (e.g., transduction) of at least a portion of the separated cells. In some embodiments, the number of transfected (e.g., transduced) cells is 1-5%, 5-10%, 10-20%, 20-30%, 30-40%, 40-50%, or 50-60% of the total number of contacted cells, each range inclusive. In some embodiments, the number of transfected (e.g., transduced) cells is at least 5%, 10%, 20%, 30%, 40%, 50%, 60%, 70%, 80%, or 90% of the total cell number of contacted cells. In some embodiments, the number of transfected (e.g., transduced) cells is 5%, 10%, 20%, 30%, 40%, 50%, 60%, 70%, 80%, 90%, or 100% of the total cell number of contacted cells. In some embodiments, the total number of transfected (e.g., transduced) cells is 5-10×10⁸, 10-20×10⁸, 20-30×10⁸, 30-40×10⁸, 40-50×10⁸, 50-60×10⁸, 60-70×10⁸, 70-80×10⁸, 80-90×10⁸, 100-125×10⁸, 125-150×10⁸, 150-175×10⁸, 175-200×10⁸ cells, or 200-300×10⁸, each range inclusive. In some embodiments, the number of transfected (e.g., transduced) cells is at least 5×10⁸, 10×10⁸, 20×10⁸, 30×10⁸, 40×10⁸, 50×10⁸, 60×10⁸, 70×10⁸, 80×10⁸, 90×10⁸, 100×10⁸, 150×10⁸, 200×10⁸, or 300×10⁸ cells. In some embodiments, the number of transfected (e.g., transduced) cell is 5×10⁸, 10×10⁸, 20×10⁸, 30×10⁸, 40×10⁸, 50×10⁸, 60×10⁸, 70×10⁸, 80×10⁸, 90×10⁸, 100×10⁸, 150×10⁸, 200×10⁸, or 300×10⁸ cells. In some embodiments, the number of transfected (e.g., transduced) cells is or is about 1×10⁸, 5×10⁸, 10×10⁸, 20×10⁸, 30×10⁸, 40×10⁸, or 50×10⁸ cells.

In some embodiments, the contacting of separated cells is initiated within 0.5-1 hours, 1-2 hours, 2-4 hours, 4-6 hours, 6-8 hours, 8-10 hours, 10-12 hours, 12-14 hours, 14-16 hours, 16-18 hours, 18-20 hours, 20-22 hours, 22-24 hours after collection of the blood fraction comprising the separated cells (e.g., after apheresis for a first blood component as described in Section II. B.). In some embodiments, the contacting of separated cells is initiated no more than 12 hours after collection of the blood fraction comprising the separated cells. In some embodiments, the contacting of separated cells is initiated at most 12 hours after collection of the blood fraction comprising the separated cells. In some embodiments, the contacting of separated cells is initiated within at least 30 minutes, 1 hour, 2 hours, 2 hours, 4 hours, 5 hours, 6 hours, 7 hours, 8 hours, 9 hours, 10 hours, 11 hours, or 12 hours after collection of the blood fraction comprising the separated cells. In some embodiments, the contacting of separated cells is initiated within 30 minutes, 1 hour, 2 hours, 2 hours, 4 hours, 5 hours, 6 hours, 7 hours, 8 hours, 9 hours, 10 hours, 11 hours, or 12 hours after collection of the blood fraction comprising the separated cells. In some embodiments, the contacting of separated cells is initiated at least 12 hours after collection of the blood fraction comprising the separated cells. In some embodiments, the contacting of separated cells is initiated within 12 hours, 13 hours, 14 hours, 15 hours, 16 hours, 17 hours, 18 hours, 19 hours, 20 hours, 21 hours, 22 hours, 23 hours or 24 hours after collection of the blood fraction comprising the separated cells. In some embodiments, the contacting of separated cells is initiated no more than 1 hour after collection of the blood fraction comprising the separated cells. In some embodiments, the contacting of separated cells is initiated within 0-5 minutes, 5-10 minutes, 10-15 minutes, 15-30 minutes, 30-45 minutes, 45-60 minutes after collection of the blood fraction comprising the separated cells. In some embodiments, the contacting of separated cells is initiated at least 0-5 minutes, 5-10 minutes, 10-15 minutes, 15-30 minutes. 30-45 minutes, 45-60 minutes after collection of the blood fraction comprising the separated cells.

In some embodiments, separated cells (e.g. leukocyte components) are contacted with a composition in a contacting chamber. In some embodiments, the method comprises contacting the separated cells (e.g. leukocyte components) with a composition comprising viral vector. In some embodiments, the viral vectors carry an exogenous agent so that the method can be used to deliver the exogenous agent to a subject via vector based methods. In particular embodiments, the separated cells (e.g. leukocyte components) are contacted with a composition of a viral vector. In some embodiments, the method comprises contacting the separated cells (e.g. leukocyte components) with a composition comprising nucleic acids (e.g., such as nucleic acids encoding a exogenous agent). In some embodiments, the separated cells (e.g. leukocyte components) are contacted with a composition comprising viral vector within a contacting chamber.

In some embodiments, the separated cells (e.g. leukocyte components) that have been separated from whole blood as described above are pumped (e.g., via an in-line pump) into the inner cavity (i.e., lumen) of a contacting chamber (e.g. which in some cases can be the collection container). In some aspects, any suitable contacting chamber known in art may be used in the provided methods. In some embodiments, the contacting chamber is made from hard plastic and comprises a lumen with a set volume. In some embodiments, the contacting chamber is not made from hard plastic and comprises a lumen with a variable volume. In some embodiments, the contacting chamber is made from a flexible plastic such as polyvinyl chloride. In some embodiments, the contacting chamber is a blood bag.

In some embodiments, the contacting chamber is open along at least one wall. In some embodiments, the contacting chamber comprises at least one opening (e.g. inlet) capable of permitting the aspiration of liquid in and out of the internal cavity. In some embodiments, the contacting chamber is closed. In some embodiments, the contacting chamber is sterile.

In some embodiments, the contacting of the separated cells (e.g. leukocyte components) and the viral vector or nucleic acid can generate a transduction mixture. In some embodiments, the transduction mixture includes all of the separated cells (e.g. leukocyte components) collected from the whole blood of the subject and a fixed amount or concentration of the viral vector or nucleic acid. In some aspects, transfection is a process by which a non-endogenous nucleic acid is inserted into eukaryotic cells, such as by viral or plasmid vector (e.g., transduction). In some embodiments, transduction of the separated cells is via contacting the separated cells with a composition comprising viral vector or nucleic acid (e.g., contacting such as in the contacting chamber).

In some embodiments, the composition comprising viral vector or the composition comprising nucleic acids is present within the lumen of the contacting chamber. In some embodiments, the contacting chamber is pre-filled with the composition prior to the introduction of the separated cells (e.g. leukocyte components). In some embodiments, the composition comprising viral vector or nucleic acids is introduced into the contacting chamber simultaneously as the separated cells (e.g. leukocyte components). In some embodiments, the composition comprising viral vector or nucleic acids is introduced into the contacting chamber subsequent to the separated cells (e.g. leukocyte components). In some embodiments, the composition comprising viral vectors or nucleic acids is connected to the contacting chamber via an operable connection, optionally with a tube, line, valve, luer port, or spike. In some embodiments, the composition comprising viral vectors or nucleic acids is introduced (i.e., via an in-line pump as described above) directly into the lumen of the contacting chamber.

In some embodiments, the concentration of cells (e.g. leukocyte components, such as PBMCs) in the contacting chamber is between 1×10⁶ cells/mL and 1×10⁹ cells/mL, between 1×10⁶ cells/mL and 1×10⁸ cells/mL, between 1×10⁶ cells/mL and 1×10⁷ cells/mL, between 1×10⁷ cells/mL and 1×10⁹ cells/mL, between 1×10⁷ cells/mL and 1×10⁸ cells/mL or between 1×10⁸ cells/mL and 1×10⁹ cells/mL. In some embodiments, the concentration of cells (e.g. leukocyte components, such as PBMCs) in the contacting chamber is at or about 1×10⁶ cells/mL, 5×10⁶ cells/mL, 1×10⁷ cells/mL, 5×10⁷ cells/mL, 1×10⁸ cells/mL, 5×10⁸ cells/mL or 1×10⁹ cells/mL, or is any value between any of the foregoing. In some embodiments, the concentration of cells (e.g. leukocyte components, such as PBMCs) in the contacting chamber is at or about 1×10⁷ cells/mL.

In some embodiments, there is a fixed concentration of viral vectors within the lumen of the contacting chamber. In some embodiments, the fixed concentration viral vectors is 1-5×10⁹, 5-10×10⁹, 10-20×10⁹, 20-30×10⁹, 30-40×10⁹, 40-50×10⁹, 50-60×10⁹, 60-70×10⁹, 70-80×10⁹, 80-90×10⁹, 1-5×10⁹, 5-10×10⁹, 10-20×10⁹, 20-30×10⁹, 30-40×10⁹, or 40-50×10⁹ particles, each range inclusive. In some embodiments, the fixed concentration of viral vectors is 1-5×10¹⁰, 5-10×10¹⁰, 10-20×10¹⁰, 20-30×10¹⁰, 30-40×10¹⁰, 40-50×10¹⁰, 50-60×10¹⁰, 60-70×10¹⁰, 70-80×10¹⁰, 80-90×10¹⁰, 1-5×10¹⁰, 5-10×10¹⁰, 10-20×10¹⁰, 20-30×10¹⁰, 30-40×10¹⁰, or 40-50×10¹⁰ particles, each range inclusive. In some embodiments, the fixed concentration of or viral vectors is at least 5×10⁹, 10×10⁹, 20×10⁹, 30×10⁹, 40×10⁹, 50×10⁹, 60×10⁹, 70×10⁹, 80×10⁹, 90×10⁹, 1×10¹⁰, 5×10¹⁰, 10×10¹⁰, 20×10¹⁰, 30×10¹⁰, 40×10¹⁰ or 50×10¹⁰ particles. In some embodiments, the fixed concentration of viral vectors is at or about 5×10⁹, 10×10⁹, 20×10⁹, 30×10⁹, 40×10⁹, 50×10⁹, 60×10⁹, 70×10⁹, 80×10⁹, 90×10⁹, 1×10¹⁰, 5×10¹⁰, 10×10¹⁰, 20×10¹⁰, 30×10¹⁰, 40×10¹⁰ or 50×10¹⁰ particles, or any value between any of the foregoing. In some embodiments, the fixed concentration of viral vectors is or is at or about 1×10⁹, 10×10⁹, 20×10⁹, 30×10⁹, 40×10⁹, or 50×10⁹ particles, or any value between any of the foregoing. In some embodiments, the fixed concentration of viral vectors is or is at or about 1×10¹⁰, 10×10¹⁰, 20×10¹⁰, 30×10¹⁰, 40×10¹⁰, or 50×10¹⁰ particles, or any value between any of the foregoing.

In some embodiments, the fixed concentration of viral vectors is 1-5×10⁹, 5-10×10⁹, 10-20×10⁹, 20-30×10⁹, 30-40×10⁹, 40-50×10⁹, 50-60×10⁹, 60-70×10⁹, 70-80×10⁹, 80-90×10⁹, 1-5×10⁹, 5-10×10⁹, 10-20×10⁹, 20-30×10⁹, 30-40×10⁹, or 40-50×10⁹ infectious units (IU), each range inclusive. In some embodiments, the fixed concentration of viral vectors is 1-5×10¹⁰, 5-10×10¹⁰, 10-20×10¹⁰, 20-30×10¹⁰, 30-40×10¹⁰, 40-50×10¹⁰, 50-60×10¹⁰, 60-70×10¹⁰, 70-80×10¹⁰, 80-90×10¹⁰, 1-5×10¹⁰, 5-10×10¹′, 10-20×10¹⁰, 20-30×10¹⁰, 30-40×10¹⁰, or 40-50×10¹⁰ infectious units (IU), each range inclusive. In some embodiments, the fixed concentration of viral vectors is at least 5×10⁹, 10×10⁹, 20×10⁹, 30×10⁹, 40×10⁹, 50×10⁹, 60×10⁹, 70×10⁹, 80×10⁹, 90×10⁹, 1×10¹⁰, 5×10¹⁰, 10×10¹⁰, 20×10¹⁰, 30×10¹⁰, 40×10¹⁰ or 50×10¹⁰ IU. In some embodiments, the fixed concentration of viral vectors is at or about 5×10⁹, 10×10⁹, 20×10⁹, 30×10⁹, 40×10⁹, 50×10⁹, 60×10⁹, 70×10⁹, 80×10⁹, 90×10⁹, 1×10¹⁰, 5×10¹⁰, 10×10¹⁰, 20×10¹⁰, 30×10¹′, 40×10¹⁰ or 50×10¹⁰ IU, or any value between any of the foregoing. In some embodiments, the fixed concentration of viral vectors is or is at or about 1×10¹⁰, 10×10¹⁰, 20×10¹⁰, 30×10¹⁰, 40×10¹⁰, or 50×10¹⁰ IU, or any value between any of the foregoing.

In some embodiments, the fixed concentration of viral vectors is 1-5×10³, 5-10×10³, 10-20×10³, 20-30×10³, 30-40×10³, 40-50×10³, 50-60×10³, 60-70×10³, 70-80×10³, 80-90×10³, 1-5×10⁴, 5-10×10⁴, 10-20×10⁴, 20-30×10⁴, 30-40×10⁴, or 40-50×10⁴ viral genomic (Vg)/cell, each range inclusive. In some embodiments, the fixed concentration of viral vectors is at least 5×10³, 10×10³, 20×10³, 30×10³, 40×10³, 50×10³, 60×10³, 70×10³, 80×10³, 90×10³, 1×10⁴, 5×10⁴, 10×10⁴, 20×10⁴ cells, 30×10⁴, 40×10⁴ or 50×10⁴ Vg/cell. In some embodiments, the fixed concentration of viral vectors is at or about 5×10³, 10×10³, 20×10³, 30×10³, 40×10³, 50×10³, 60×10³, 70×10³, 80×10³, 90×10³, 1×10⁴, 5×10⁴, 10×10⁴, 20×10⁴ cells, 30×10⁴, 40×10⁴ or 50×10⁴ Vg/cell, or any value between any of the foregoing. In some embodiments, the fixed concentration of viral vectors is or is about 1×10³, 5×10³, 10×10³, 20×10³, 30×10³, 40×10³, or 50×10³ Vg/cell.

In some embodiments, there is a fixed amount of viral vectors within the lumen of the contacting chamber. In some embodiments the viral vector or is a retroviral vector, such as a lentiviral vector. In some embodiments, the fixed amount of the viral vector is from about 10⁴ to about 10¹⁰ plaque forming units (pfu), inclusive. In some embodiments, the fixed amount of a viral vector is from about 10⁹ to about 10⁵ pfu, inclusive In some embodiments, the fixed amount of a viral vector is from about 10⁵ to about 10⁹ pfu. In some embodiments, the fixed amount of a viral vector is from about 10⁶ to about 10⁹ pfu. In some embodiments, the fixed amount of a viral vector is from about 10¹² to about 10¹⁴ pfu, inclusive. In some embodiments, the fixed amount is 1.0×10⁹ pfu, 5.0×10⁹ pfu, 1.0×10¹⁰ pfu, 5.0×10¹⁰ pfu, 1.0×10¹¹ pfu, 5.0×10¹¹ pfu, 1.0×10¹² pfu, 5.0×10¹² pfu, or 1.0×10¹¹ pfu, 5.0×10¹¹ pfu, 1.0×10¹⁴ pfu, 5.0×10¹⁴ pfu, or 1.0×10¹⁵ pfu.

In some embodiments, the viral vector that is an adenovirus vector. In some aspects, the fixed amount of adenovirus to humans can range from about 10⁷ to 10′, inclusive, plaque forming units (pfu).

In some embodiments, there is a variable concentration of viral vectors within the lumen of the contacting chamber. In some embodiments, there is a variable concentration of nucleic acid within the lumen of the contacting chamber. In some embodiments, the concentration of viral vectors within the contacting chamber is variable over time, and/or variable with cell density. In some embodiments, the concentration of viral vectors is maintained over cell density such that more or less of the composition containing the viral vectors is introduced into the contacting chamber in accordance with the total number of cells (i.e., the concentration of viral vectors per cell is maintained over the contacting period).

In some embodiments, the composition comprising viral vectors or the composition comprising the nucleic acid is present within the lumen of the contacting chamber. In some embodiments, the composition comprising viral vectors or nucleic acids has a volume of 100, 200, 300, 400, or 500 milliliters. In some embodiments, the composition comprising viral vectors is present within the lumen of the contacting chamber and has a volume of at most 1 liter. In some embodiments, the composition comprising viral vectors is present within the lumen of the contacting chamber and has a volume of at most 500 milliliters.

In some embodiments, the contacting of separated cells (e.g. fraction of blood containing leukocyte components) with the composition comprising viral vector within the contacting chamber is for a set limit of time. In some embodiments, the contacting of separated cells within the contacting chamber is for 15 minutes to 12 hours, such as 15 minutes to 6 hours, 15 minutes to 4 hours, 15 minutes to 2 hours, 15 minutes to 1 hour, 1 hour to 12 hours, 1 hour to 6 hours, 1 hour to 4 hours, 1 hour to 2 hours, 2 hours to 12 hours, 2 hours to 6 hours, 2 hours to 4 hours, 4 hours to 12 hours, 4 hours to 6 hours or 6 hours to 12 hours.

In some embodiments, the contacting of separated cells within the contacting chamber is for 1-2 hours, 2-4 hours, 4-6 hours, 6-8 hours, 8-10 hours, 10-12 hours, 12-14 hours, 14-16 hours, 16-18 hours, 18-20 hours, 20-22 hours, 22-24 hours, each range inclusive. In some embodiments, the contacting of separated cells is for at most 12 hours. In some embodiments, the contacting of separated cells is for at most 1 hour, 2 hours, 2 hours, 4 hours, 5 hours, 6 hours, 7 hours, 8 hours, 9 hours, 10 hours, 11 hours, or 12 hours. In some embodiments, the contacting of separated cells is for 1 hour, 2 hours, 2 hours, 4 hours, 5 hours, 6 hours, 7 hours, 8 hours, 9 hours, 10 hours, 11 hours, or 12 hours. In some embodiments, the contacting of separated cells is for at least 12 hours. In some embodiments, the contacting of separated cells is for 12 hours, 13 hours, 14 hours, 15 hours, 16 hours, 17 hours, 18 hours, 19 hours, 20 hours, 21 hours, 22 hours, 23 hours or 24 hours.

In some embodiments, the contacting of separated cells with the composition comprising the viral vectors or nucleic acids is for no more than 1 hour. In some embodiments, the contacting of separated cells is for 0-5 minutes, 5-10 minutes, 10-15 minutes, 15-30 minutes. 30-45 minutes, 45-60 minutes, each range inclusive. In some embodiments, the contacting of separated cells within the contacting chamber is for 30-60 minutes. In some embodiments, the contacting of separated cells is for at or about 60 minutes. In some embodiments, the contacting of separated cells is for at or about 30 minutes. In some embodiments, the contacting of separated cells is for at or about 15 minutes.

In some embodiments, the transduction mixture comprises an inhibitor of mTOR in combination with the viral vector (e.g. lentiviral vector) in accord with the provided methods. In some embodiments, the inhibitor of mTOR and viral vector (e.g. lentiviral vector) are contacted with the cells simultaneously. In some embodiments, the transduction mixture comprises an inhibitor of an antiviral restriction factor in combination with the viral vector (e.g. lentiviral vector) in accord with the provided methods. In some embodiments, the inhibitor of an antiviral restriction factor and viral vector (e.g. lentiviral vector) are contacted with the cells simultaneously.

In some embodiments, the subject is administered or has been administered an inhibitor of mTOR in combination with the viral vector (e.g. lentiviral vector) in accord with the provided methods. In some embodiments, the inhibitor of mTOR and viral vector (e.g. lentiviral vector) are administered simultaneously. In some embodiments, the subject is administered or has been administered an inhibitor of mTOR and a viral vector consecutively. In some embodiments, the inhibitor of mTOR and the viral vector are administered on the same day. In some embodiments, the inhibitor of mTOR is administered intermittently such as in a particular dosing regimen with a defined frequency or schedule.

In certain embodiments, the subject is administered or has been administered an inhibitor of mTOR 1 month before or after administration of the viral vector or a first dose of the viral vector. In some embodiments, the subject is administered or has been administered an inhibitor of mTOR within 1 month before administration of the viral vector or a first dose of the viral vector, such as within or at or about 4 weeks, 3 weeks, 2 weeks or 1 weeks, such as at or about 1 day, 2 days, 3 days, 4 days, 5 days, 6 days or 7 days before administration of the viral vector or a first dose of the viral vector. In some embodiments, the subject is administered or has been administered an inhibitor of mTOR within 3 days before administration of the viral vector.

In certain embodiments, the subject is administered or has been administered an inhibitor of mTOR 1 day before or after administration of the viral vector or a first dose of the viral vector. In some embodiments, the subject is administered or has been administered an inhibitor of mTOR within 1 day before administration of the viral vector or a first dose of the viral vector, such as within or at or about 24, 23, 22, 21, 20, 19, 18, 17, 16, 15, 14, 13, or 12 hours, or such as 12, 11, 10, 9, 8, 7, 6, 5, 4, 3, 2, or 1 hour before administration of the viral vector or a first dose of the viral vector. In some embodiments, the subject is administered or has been administered an inhibitor of mTOR within 3 hours before administration of the viral vector.

In some embodiments, the inhibitor of mTOR is contacted with PBMCs or a subset thereof in an amount of from at or about 1-10 μM, 1-100 μM, 10-100 μM, or 100-1000 μM, or any value between the foregoing. In some embodiments, the inhibitor of mTOR is contacted with PBMCs or a subset thereof in an amount of from at or about 1-10 μM, 10-20 μM, 20-30 μM, 30-40 μM, or 40-50 μM, or any value between the foregoing. In some embodiments, the inhibitor of mTOR is contacted with PBMCs or a subset thereof in an amount of 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 m μM g, 28 m μM g, 29 μM, 30 μM, 31 μM, 32 μM, 33, μM 34 μM, or 35 μM. In some embodiments, the inhibitor of mTOR is contacted with PBMCs or a subset thereof in an amount of or about 50, 25, or 10 μM. In some embodiments, the inhibitor of mTOR is contacted with PBMCs or a subset thereof in an amount of or about 30 μM. In some embodiments, the inhibitor of mTOR is contacted with PBMCs or a subset thereof in an amount of or about 28 μM. In some embodiments, the inhibitor of mTOR is contacted with PBMCs or a subset thereof in an amount of or about 25 μM.

In some embodiments, the transduction mixture further comprises an inhibitor of a cellular restriction factor that is an antiviral restriction factor in combination with the viral vector (e.g. lentiviral vector) and, in some cases also the inhibitor of mTOR, in accord with the provided methods. In some embodiments, the antiviral restriction factor is an antiviral restriction factor as described in Section III, such as a SAMHD1 inhibitory viral protein. In some embodiments, the SAMHD1 inhibitory viral protein is linked to the viral vector.

In some embodiments, the transduction mixture further comprises a recombinant cytokine in combination with the viral vector (e.g. lentiviral vector) and, in some cases also the inhibitor of mTOR, in accord with the provided methods. Exemplary recombinant cytokines that can be present in the transduction mixture in accord with provided methods are described in Section III.C. In some embodiments, the recombinant cytokine is IL-7 or IL-15 or a combination thereof. In some embodiments, the cytokine, such as IL-7 or IL-15, has activity as an antiviral restriction factor.

In some embodiments, the transduction mixture further comprises an inhibitor of an antiviral restriction factor in combination with the viral vector (e.g. lentiviral vector) and an inhibitor of mTOR in accord with the provided methods. In some embodiments, the inhibitor of an antiviral restriction factor and viral vector (e.g. lentiviral vector) are contacted with the PBMCs or subset thereof simultaneously. In some embodiments, the PBMCs or subset thereof are contacted with an inhibitor of an antiviral restriction factor and a viral vector consecutively. In some embodiments, the inhibitor of an antiviral restriction factor and the viral vector are contacted with PBMCs on the same day. In some embodiments, the inhibitor of an antiviral restriction factor is administered intermittently such as in a particular dosing regimen with a defined frequency or schedule.

In certain embodiments, the subject is administered or has been administered an inhibitor of an antiviral restriction factor within 1 month before or after administration of the viral vector or a first dose of the viral vector. In some embodiments, the subject is administered or has been administered an inhibitor of an antiviral restriction factor within 1 month before administration of the viral vector or a first dose of the viral vector, such as within or at or about 4 weeks, 3 weeks, 2 weeks or 1 weeks, such as at or about 1 day, 2 days, 3 days, 4 days, 5 days, 6 days or 7 days before administration of the viral vector or a first dose of the viral vector. In some embodiments, the subject is administered or has been administered an inhibitor of an antiviral restriction factor within 3 days before administration of the viral vector.

In certain embodiments, the subject is administered or has been administered inhibitor of an antiviral restriction factor 1 day before or after administration of the viral vector or a first dose of the viral vector. In some embodiments, the subject is administered or has been administered inhibitor of an antiviral restriction factor within 1 day before administration of the viral vector or a first dose of the viral vector, such as within or at or about 24, 23, 22, 21, 20, 19, 18, 17, 16, 15, 14, 13, or 12 hours, or such as 12, 11, 10, 9, 8, 7, 6, 5, 4, 3, 2, or 1 hour before administration of the viral vector or a first dose of the viral vector. In some embodiments, the subject is administered or has been administered inhibitor of an antiviral restriction factor within 3 hours before administration of the viral vector.

In some embodiments, the inhibitor of an antiviral restriction factor is contacted with the PBMCs or a subset thereof at a dose from at or about 1-10 μM, 10-20 μM, 20-30 μM, 30-40 μM, 40-50 μM, 50-60 μM, 60-70 μM, 70-80 μM, 80-90 μM, or 90-100 μM, or any value between the foregoing. In some embodiments, the inhibitor of an antiviral restriction factor is contacted with the PBMCs or a subset thereof at a dose of 10 μM, 20 μM, 30 μM, 40 μM, 50 μM, 60 μM, 70 μM, 80 μM, 90 μM, or 100 μM. In some embodiments, the inhibitor of an antiviral restriction factor is contacted with the PBMCs or a subset thereof at a dose of 1 μM, 2 μM, 3 μM, 4 μM, 5 μM, 6 μM, 7 μM, 8 μM, 9 μM, or 10 μM. In some embodiments, the inhibitor of an antiviral restriction factor is contacted with the PBMCs or a subset thereof as a dose of 30 M. In some embodiments, the inhibitor of an antiviral restriction factor is contacted with the PBMCs or a subset thereof as a dose of 10 M. In some embodiments, the inhibitor of an antiviral restriction factor is contacted with the PBMCs or a subset thereof at a dose of from at or about 1-10 mg, 10-20 mg, 20-30 mg, 30-40 mg, or 40-50, or any value between the foregoing. In some embodiments, the inhibitor of an antiviral restriction factor is contacted with the PBMCs or a subset thereof at a dose of 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33, mg 34 mg, or 35 mg. In some embodiments, the inhibitor of an antiviral restriction factor is contacted with the PBMCs or a subset thereof as a dose of 50, 25, or 10 mg. In some embodiments, the inhibitor of an antiviral restriction factor is contacted with the PBMCs or a subset thereof as a dose of 40 mg. In some embodiments, the inhibitor of an antiviral restriction factor is contacted with the PBMCs or a subset thereof as a dose of 25 mg.

In some embodiments, the transduction mixture is mixed manually or by automatic methods during at least a portion of the contacting. In some embodiments, mixing is by physical manipulation of the contacting chamber (e.g. bag). In some embodiments, the mixing is carried out without disconnecting or disengaging the contacting chamber (e.g. bag) from the in-line system. In some embodiments, the mixing is carried out under sterile conditions.

In some embodiments, the contacting chamber is centrifugal. In some embodiments, the contacting chamber is rotatable about a rotation axis. In some embodiments, the contacting chamber is rotating for at least a portion of the contacting period. In some embodiments, the contacting chamber is rotating for 0-10%, 10-20%, 20-30%, 30-40%, 40-50%, 50-60%, 60-70%, 70-80%, 80-90%, or 90-100% of the total contacting period, each range inclusive. In some embodiments, the contacting chamber is rotating for the entire contacting period. In some embodiments, the contacting chamber is rotating for at least 5 minutes, at least 10 minutes, or at least 15 minutes, or at least 20 minutes, or at least 30 minutes, 45 minutes or more, or 60 minutes or more, or 90 minutes or more, or 120 minutes or more; or 5 minutes to 60 minutes, 10 minutes to 60 minutes, 15 minutes to 60 minutes, 15 minutes to 45 minutes, 30 minutes to 60 minutes, or 45 minutes to 60 minutes.

In some embodiments, centrifugation at high speeds, for example, at a force (relative centrifugal force (RCF)) of between 200 g and 3000 g, such as between 500 g and 2500 g, between 500 g and 2000 g, between 500 g and 1500 g, between 500 g and 1000 g, between 1000 g and 3000 g, between 1000 g and 2500 g, between 1000 g and 2000 g, between 1000 g and 1500 g, between 1500 g and 3000 g, between 1500 g and 2500 g, between 1500 g and 2000 g, between 2000 g and 3000 g, between 2000 g and 2500 g or between 2500 g and 3000 g. The term “relative centrifugal force” or RCF is generally understood to be the effective force imparted on an object or substance (such as a cell, sample, or pellet and/or a point in the chamber or other container being rotated), relative to the earth's gravitational force, at a particular point in space as compared to the axis of rotation. The value may be determined using well-known formulas, taking into account the gravitational force, rotation speed and the radius of rotation (distance from the axis of rotation and the object, substance, or particle at which RCF is being measured).

In some embodiments, the contacting chamber includes one or more opening(s), such as one or more inlet, one or more outlet, and/or one or more inlet/outlet, which can permit intake and output of liquid fluid to and from the cavity. In some embodiments, liquid (e.g. containing a composition of viral vectors) may be taken into the cavity through a tubing line or other channel that is or is placed in connection with the opening (e.g. inlet), for example, by placing the line or channel in connection with and control of a pump, syringe, or other machinery, which may be controlled in an automated fashion. In some embodiments, liquid (e.g. containing a composition of contacted leukocytes containing the separated leukocytes and viral vectors) may be expelled or outputted through the cavity through a tubing line or other channel that is or is placed in connection with the opening (e.g. outlet), for example, by placing the line or channel in connection with and control of a pump, syringe, or other machinery, which may be controlled in an automated fashion. In some embodiments, the chamber is pre-connected to one or more of the additional components, directly and/or indirectly. Such a chamber may be provided as part of a pre-assembled kit, e.g., a kit packaged for single, sterile, use in connection with the provided methods. In some embodiments, various components are packaged separately, for example, to allow for custom configurations in which a user connects and arranges the components for a particular embodiment of the processing methods.

The components typically include at least one tubing line, and generally a set or system of tubing lines, and at least one connector. Exemplary connectors include valves, ports, spikes, welds, seals, and hose clamps. The connectors and/or other components may be aseptic, for example, to permit the entire process to be carried out in a closed, sterile system, which can eliminate or reduce the need for clean rooms, sterile cabinets, and/or laminar flow systems.

In some embodiments, the contacting chamber is comprised in a fluid circuit, optionally a closed in-line circuit. In some embodiments, the contacting chamber can be operably connected in a fluid and/or signal connection with any of the disclosed units and/or devices, or in a fluid and/or signal connection with such units and/or devices. In some embodiments, the operable connection via at least one connector selected from the group consisting of valves, luer ports and spikes. In some embodiments, one or more of these connectors are disposable. In some embodiments, one or more components of the contacting chamber is disposable. In some embodiments, the contacting chamber is disposable. Thus, in some embodiments, the contacting chamber is part of a closed system, such as a sterile system, having various additional components such as tubing lines and connectors and caps, within which processing steps occur. Thus, in some embodiments, the provided methods and/or steps thereof are carried out in a completely closed or semi-closed environment, such as a closed or semi-closed sterile system, facilitating the processing of the viral vector for therapeutic administration to subjects without the need for a separate sterile environment, such as a biosafety cabinet or room. The methods in some embodiments are carried out in an automated or partially automated fashion.

In some embodiments, the composition comprising viral vector or nucleic acids as present in the contacting chamber is supplemented with at least one agent to enhance transfection and/or transduction (i.e., an adjuvant of transfection and/or transduction). In some embodiments, one or more transfection reagents are used. Any suitable transfection reagent known in the art may be used in the provided method, for example some commercially available transfection reagents such as Effectene and TransIT-X2 (e.g., Effectene and FuGENE 6) are specially dedicated for use with plasmid DNA, while some transfection reagents such as Lipofectamine RNAiMAX are more suited for use with small oligonucleotides. Other agents to enhance transfection may include members of the Lipofectamine and DharmaFECT families, which in some aspects are associated with higher transfection efficiencies in transfecting primary human cells (Hunt et al., 2010). In some embodiments, composition comprising viral vector or nucleic acids as present in the contacting chamber is supplemented with at least one agent chosen from the group comprising Lipofectamine, Lipofectamine 3000, Lipofectamine 2000, PEI-based reagents, Transporter™ 5 and PEI25, PEG, Xfect, Nanofectamin, TransIT-X2, TransIT-2020, FuGENE 6, Effectene, HiperFect, and ExGen 500.

In some embodiments, the methods are for transducing T cells in the subject. In some embodiments, the lentiviral vector is a T-cell targeting lentiviral vector and comprises a T cell binding agent on its surface for targeted recognition of a molecule on a T cell, such as CD3, CD4 or CD8. In some embodiments, the T cell binding agent is an antibody. In some embodiments, the T cell binding agent is an anti-CD3 antibody. In some embodiments, the T cell binding agent is an anti-CD4 antibody. In some embodiments, the T cell binding agent is an anti-CD8 antibody. Exemplary T cell binding agents are described in Section IV.

In some embodiments, the composition comprising viral vector or nucleic acids as present in the contacting chamber is supplemented with a T cell activation element. In some embodiments, the T cell activation element may be either in solution or on the surface of the viral vector (e.g. lentiviral vector particles) to facilitate genetic modification (e.g. transduction) of T cells in the transduction mixture. In some embodiments, the T cell activation element activates a T cell through T cell receptor associated complex. Such an activation element can be an anti-CD3 antibody, for example an anti-CD3 scFv or an anti-CD3 scFvFc. In some embodiments, the T cell activation agent includes anti-CD3 and another polypeptide that binds to a costimulatory receptor such as CD28. In some embodiments, the T cell activation element may include anti-CD3.anti-CD28 antibodies or T cell stimulatory cytokines such as IL-2, IL15 or IL-7. In some embodiments, the T cell activation element is a reagent that is soluble. In some embodiments, the T cell activation element is membrane bound of the surface of a viral vector. In some embodiments, the T cell activation element is part of a pseudotyping element on the surface of a viral vector, in which the T cell activation element is not encoded by a polynucleotide of in the viral vector.

In some embodiments, the T cell activation element can be an anti-CD3 antibody, such as an anti-CD3 scFv or anti-CD3 scFvFc. In some embodiments, the T cell activation element may include a polypeptide capable of binding to CD28. In some embodiments, the polypeptide capable of binding to CD28 is an anti-CD28 antibody, or a fragment thereof that retains the ability to bind to CD28. In other embodiments, the polypeptide capable of binding to CD28 is CD80, CD86, or a functional fragment thereof that is capable of binding CD28 and inducing CD28-mediated activation of Akt, such as an extracellular domain portion of CD80. In some embodiments, the anti-CD28 antibody or fragment thereof is a single chain anti-CD28 antibody, such as, but not limited to, an anti-CD28 scFv. In some embodiments, an activation element is fused to a heterologous signal sequence and/or a heterologous membrane attachment sequence, both of which help direct the activation element to the membrane. In some embodiments, the membrane attachment sequence is a GPI anchor. In some embodiments, the T cell activation element can be included on the surface of a viral vector, such as by pseudotyping as part of a fusogen (e.g. described in Section IV).

In some embodiments, the T cell activation element also may include a membrane bound cytokine, such as IL-2, IL-17, IL-15 or an active fragment thereof. In some embodiments, the cytokine a heterologous signal sequence and/or a heterologous membrane attachment sequence, both of which help direct the activation element to the membrane. In some embodiments, the membrane attachment sequence is a GPI anchor. In some embodiments, the T cell activation element can be included on the surface of a viral vector, such as by pseudotyping as part of a fusogen (e.g. described in Section IV).

Exemplary T cell activation elements and agents are described in WO20190559546 or WO2021042072.

In some embodiments, the composition comprising viral vector or nucleic acids as present in the contacting chamber are not supplemented with a T cell activation element. In some embodiments, the T cells of the leukocyte component are non-activated T cells.

In some embodiments, the contacting step is performed at a temperature between at or about 18° C. and 42° C. In some embodiments, the temperature is between 20° C. and 25° C., such as at or about 22° C. In some embodiments, the contacting step is performed at temperatures between 32° C. and 42° C., such as at or about 37° C. In some embodiments, the contacting step is performed at or about 5% CO2.

In some embodiments, the transduction mixture containing all separated cells collected from the whole blood fraction and the fixed amount or concentration of viral vector or nucleic acid(s) is not washed or subjected to further processing after the contacting. In some embodiments, the entire composition of the transduction mixture is used for reinfusion to the subject. In some embodiments, the entire composition of the transduction mixture is used for reinfusion to the subject without any additional processing steps.

### 4. Reinfusion of Viral Vectors to Subject

In some embodiments, the method further provides reinfusing the contacted cell component or the transduction mixture containing the viral vector (e.g. encoding an exogenous agent) to a subject. In some embodiments, the reinfusion thus administers the viral vector and/or exogenous agent to the subject. In some embodiments, the transduction mixture is directly administered to the subject. In some embodiments the transduction mixture is not further washed or processed after the contacting with the viral vector prior to reinfusion to the subject.

In some embodiments, the contacted cell component or the transduction mixture are contained in a transfer container for infusion to a subject. In some embodiments, the composition containing the contacted leukocyte components, such as the transduction mixture, are moved from the contacting chamber to the transfer chamber, such as via one or more operably connected tubing lines. In some embodiments, the transfer container is a bag. In some embodiments, the transfer container is a rigid container. In some embodiments, the transfer container is opaque or partially opaque.

In some embodiments, the transferred contacted leukocyte components, such as the transduction mixture, contained in the transfer container are severed or otherwise separated from the tubing sets used during the process, in which the reinfusion to the subject is offline. In some embodiments, offline reinfusion is a manual reinfusion. Thus, in some embodiments, the transfer container containing the contacted leukocyte components or precursors thereof are detached from the donor subject prior to their reinfusion to the donor subject.

In some embodiments, the transfer container remains in-line with the processing system for reinfusion of the contacted leukocyte components or precursors thereof, such as the transduction mixture, directly to the subject without detachment from the donor subject or separation from the tubing sets used during the process. The provided methods that improve efficiency of the process avoids any additional product labeling and/or traceable handling requirements because the transduction mixture for reinfusion never leaves the disposable set which remains connected to the donor subject during the entire treatment procedure.

In some embodiments, the time to reinfusion to the subject following the contacting is no more than 24 hours after obtaining the whole blood from the subject (e.g., as described in Section II. A.) In some embodiments, the time to reinfusion to the subject following the contacting of the separated cell is for a time of from 1 to 24 hours, 1 to 12 hours, 1 to 6 hours, 1 to 4 hours, 1 to 2 hours, 2 hours to 24 hours, 2 hours to 12 hours, 2 hours to 6 hours, 2 hours to 4 hours, 4 hours to 24 hours, 4 hours to 12 hours, 4 hours to 6 hours, 6 hours to 24 hours, 6 hours to 12 hours or 12 hours to 24 hours, after obtaining whole blood from the subject (e.g., as described in above.) In some embodiments, the time to reinfusion to the subject following the contacting of separated cells is for a time of 1-2 hours, 2-4 hours, 4-6 hours, 6-8 hours, 8-10 hours, 10-12 hours, 12-14 hours, 14-16 hours, 16-18 hours, 18-20 hours, 20-22 hours, 22-24 hours after obtaining whole blood from the subject (e.g., as described above.). In some embodiments, the time to reinfusion to the subject following the contacting of separated cells is no more than 1, 2, 3, 4, 5, or 6 hours. In some embodiments, the time to reinfusion to the subject following the contacting of the separated cells is at most 1, 2, 3, 4, 5, or 6 hours. In some embodiments, the time to reinfusion to the subject following the contacting of the separated cells is at or about 1, 2, 3, 4, 5, or 6 hours, or any value between any of the foregoing. In some embodiments, the time to reinfusion to the subject following the contacting of separated cells is no more than 1, 2, or 3 days after obtaining whole blood (e.g., as described above).

In some embodiments, the composition comprising contacted cells is connected to the return processing unit via an operable connection, optionally with a tube, line, valve, luer port, or spike. In some embodiments, the composition comprising contacted cells is pumped (i.e., via an in-line pump as described above) directly into the lumen of the return processing unit.

In some of any of the provided embodiments, the reinfusion of the contacted cells for administration of the viral vector or exogenous agent is via a return processing unit. In some embodiments, the return processing unit returns the separated cells, the first blood component, the second blood component, and/or the third blood component to the subject. In some embodiments, the return processing unit device has an inlet. In some embodiments, the return processing unit device also has an outlet and may optionally alternate between discharging the first blood component (e.g., leukocytes) and the second blood component (i.e. red blood cells and/or plasma) through the outlet. In some embodiments, the second and/or third blood component may be returned to the subject in addition to the first blood component via the return line, optionally wherein the return line is operably connected to the return processing unit. In some embodiments, The first blood component is leukocytes and/or the second blood component is red blood cells, and/or the third blood component is plasma and/or platelets. In some embodiments, the return line operably connects to the venous-access device at a point between the draw line pump and the venous-access device. In some embodiments, the venous-access device is operably connected to the return processing unit.

In some embodiments, the return processing unit is comprised in a fluid circuit, optionally a closed in-line circuit. In some embodiments, the return processing unit can be operably connected in a fluid and/or signal connection with any of the disclosed units and/or devices, or in a fluid and/or signal connection with such units and/or devices. In some embodiments, the operable connection via at least one connector selected from the group consisting of valves, luer ports and spikes. In some embodiments, one or more of these connectors are disposable. In some embodiments, one or more components of the return processing unit set is disposable. In some embodiments, the return processing unit is disposable. In some embodiments, the return processing unit is sterile.

In some embodiments, the composition comprising contacted cells present within the lumen of the return processing unit has a volume of 100-200 milliliters, 200-300 milliliters, 300-400 milliliters, or 400-500 milliliters, each range inclusive. In some embodiments, the composition comprising contacted cells present within the lumen of the contacting chamber has a volume of no more than 500 milliliters. In some embodiments, the composition comprising contacted cells present within the lumen of the contacting chamber has a volume of at least 100, 200, 300, 400, or 500 milliliters. In some embodiments, the composition comprising contacted cells present within the lumen of the contacting chamber has a volume of 100, 200, 300, 400, or 500 milliliters. In some embodiments, the composition comprising contacted cells present within the lumen of the return processing unit has a volume of no more than 1 liter.

In some embodiments, the return processing unit comprises an in-line pump for reinfusion of separated cells to the subject. In some embodiments, the total number of reinfused cells is 5-10×10⁸, 10-20×10⁸, 20-30×10⁸, 30-40×10⁸, 40-50×10⁸, 50-60×10⁸, 60-70×10⁸, 70-80×10⁸, 80-90×10⁸, 100-125×10⁸, 125-150×10⁸, 150-175×10⁸, 175-200×10⁸ cells, or 200-300×10⁸ each range inclusive. In some embodiments, the total number of reinfused cells is at least 5×10⁸, 10×10⁸, 20×10⁸, 30×10⁸, 40×10⁸, 50×10⁸, 60×10⁸, 70×10⁸, 80×10⁸, 90×10⁸, 100×10⁸, 150×10⁸, 200×10⁸, or 300×10⁸ cells. In some embodiments, the total number of reinfused cells is 5×10⁸, 10×10⁸, 20×10⁸, 30×10⁸, 40×10⁸, 50×10⁸, 60×10⁸, 70×10⁸, 80×10⁸, 90×10⁸, 100×10⁸, 150×10⁸, 200×10⁸, or 300×10⁸ cells. In some embodiments, the total number of reinfused cells is 1-5%, 5-10%, 10-20%, 20-30%, 30-40%, 40-50%, or 50-60% of the total number of separated cells, each range inclusive. In some embodiments, the total number of reinfused cells is at least 5%, 10%, 20%, 30%, 40%, 50%, 60%, 70%, 80%, or 90% of the total cell number of separated cells. In some embodiments, the total number of reinfused cells is 5%, 10%, 20%, 30%, 40%, 50%, 60%, 70%, 80%, 90%, or 100% of the total cell number of separated cells.

### 5. Modules for Monitoring and Adjusting Administration

In some embodiments, the system for administration comprises at least one module for monitoring and/or adjusting administration of the viral vectors or exogenous agent. In some embodiments, the system for administration in in-line, optionally wherein the system is a closed system. In some embodiments, the module for monitoring and/or adjusting administration is comprised in a fluid circuit, optionally a closed in-line circuit. In some embodiments, the module can be operably connected in a fluid and/or signal connection with any of the disclosed units and/or devices, or in a fluid and/or signal connection with such units and/or devices. In some embodiments, the operable connection via at least one connector selected from the group consisting of valves, luer ports and spikes. In some embodiments, one or more of these connectors are disposable.

In some embodiments, the module is operably connected to the return processing unit, optionally wherein the module is connected via a fluid and/or signal connection with the return processing unit. In some embodiments, the module is operably connected to the return processing unit and/or to an in-line pump, optionally wherein the module is connected via a fluid and/or signal connection with the return processing unit and/or to an in-line pump. In some embodiments, the module can adjust the speed and/or duration of reinfusion of the contacted cells according to the provided methods.

### III. INHIBITORS OF MTOR AND ANTIVIRAL RESTRICTION FACTORS

In provided embodiments, the methods, such as any as described above, include combination treatment of cells with viral vector and an mTOR inhibitor, such as in methods of transducing cells. In some embodiments, the provided methods can further include combination treatment with an antiviral restriction factor inhibitor or recombinant cytokine. Non-limiting examples of such agents are provided in the following subsections.

A. mTOR Inhibition

mTOR, or the “mammalian target of rapamycin,” is a protein that in humans is encoded by the FRAP1 gene. mTOR is a serine/threonine protein kinase that regulates cell growth, cell proliferation, cell motility, cell survival, protein synthesis, and transcription. mTOR, which belongs to the phosphatidylinositol 3-kinase-related kinase protein family, is the catalytic subunit of two molecular complexes: mTORC1 and mTORC2.

mTOR Complex 1 (mTORC1) is composed of mTOR, regulatory-associated protein of mTOR (Raptor), mammalian lethal with SEC13 protein 8 (MLST8) and partners PRAS40 and DEPTOR. This complex is characterized by the classic features of mTOR by functioning as a nutrient/energy/redox sensor and controlling protein synthesis. The activity of this complex is stimulated by insulin, growth factors, serum, phosphatidic acid, amino acids (particularly leucine), and oxidative stress. mTOR Complex 2 (mTORC2) is composed of mTOR, rapamycin-insensitive companion of mTOR (RICTOR), GOL, and mammalian stress-activated protein kinase interacting protein 1 (mSIN1). mTORC2 has been shown to function as an important regulator of the cytoskeleton through its stimulation of F-actin stress fibers, paxillin, RhoA, Rac1, Cdc42, and protein kinase Ca (PKCa). mTORC2 also appears to possess the activity of a previously elusive protein known as “PDK2”. mTORC2 phosphorylates the serine/threonine protein kinase Akt/PKB at a serine residue S473.

In some aspects, it is considered that inhibition of signaling of host cell mTOR allows for more efficient viral transduction into the host cell. “Inhibitors of mTOR” suitable for the invention are any compounds known in the art that inhibit or antagonize one or both of the mTOR complexes, mTORC1 and/or mTORC2. These include compounds that inhibit the mTOR kinase, as well as compounds that otherwise suppress or antagonize signaling activities of the mTOR complexes or negatively affect their biological properties (e.g., destabilizing or disrupting the protein complexes). For example, in some embodiments the inhibitor of mTOR is any compound that does not directly impact the mTOR kinase, but through other components of the mTOR protein complexes (e.g., Raptor or RICTOR) can disrupt, or inhibit the formation of, the mTORC1 complex and/or the mTORC2 complex or inhibit interaction of the complexes with downstream signaling molecules.

In some embodiments, the inhibitor of mTOR is a compound that antagonizes the mTOR kinase (mTOR inhibitors). Various mTOR inhibitors known in the art can be employed in the practice of the present invention. As used herein, the term “mTOR inhibitor” or “mTOR inhibitor compound” broadly encompasses any compounds that directly or indirectly inhibit or antagonize mTOR biological activities (e.g., kinase activity) or mTOR mediated signaling activities. Thus, the mTOR inhibitor can be a compound that suppresses mTOR expression or affects its cellular stability, a compound that inhibits or prevents formation of mTOR complexes, a compound that inhibits mTOR binding to its intracellular receptor FKBP12, a compound that inhibits or antagonizes enzymatic activities of mTOR, or a compound that otherwise inhibits mTOR interaction with downstream molecules.

In some embodiments, the inhibitor of mTOR is rapamycin, or an analogue thereof. Rapamycin (Vezina et al., J. Antibiot. 1975; 28: 721\u20136), also known as Sirolimus, is an immunosuppressant drug used to prevent rejection in organ transplantation. It prevents activation of T cells and B-cells by inhibiting their response to interleukin-2 (IL-2). It was approved by the FDA in September 1999 and is marketed under the trade name Rapamune by Pfizer. Rapamycin is an allosteric mTOR inhibitor. In some embodiments, the inhibitor of mTOR is any compound that specifically mimics or enhances the biological activity of rapamycin (e.g., binding to the FKBP12-rapamycin-binding domain of mTOR and/or inhibiting mTOR kinase activity). In some aspects, mTOR is the principal cellular target of rapamycin. Thus, an inhibitor of mTOR as disclosed herein may be rapamycin analogs or functional derivatives with similar or improved inhibitory activity on mTOR. These include rapamycin analog compounds known in the art. Examples include compounds described in, e.g., Ritacco et al., Appl Environ Microbiol. 2005; 71: 1971-1976; Bayle et al., Chemistry & Biology 2006; 13: 99-107; Wagner et al., Bioorg Med Chem Lett. 2005; 15:5340-3; Graziani et al., Org Lett. 2003; 5:2385-8; Ruan et al., Proc. Natl. Acad. Sci. USA 2008; 105:33-8; U.S. Pat. No. 5,138,051; WO 2014/10972 and WO/2009/131631. Several semi-synthetic rapamycin analogs (also known as rapalogues) have been evaluated by pharmaceutical companies for clinical development and/or been approved by the US FDA for various indications, e.g., temsirolimus (CCI-779, Torisel, Wyeth Pharmaceuticals), everolimus (RAD001, Afinitor, Novartis Pharmaceuticals), and ridaforolimus (AP23573; formerly deforolimus, ARIAD Pharmaceuticals).

In some embodiments, the inhibitor of mTOR is an ATP-competitive mTOR inhibitor. ATP-competitive mTOR inhibitors are ATP analogues that inhibit mTOR kinase activity by competing with ATP for binding to the kinase domain in mTOR. Unlike rapamycin, which primarily inhibits only mTORC1, the ATP analogues inhibit both mTORC1 and mTORC2. Because of the similarity between the kinase domains of mTOR and the PI3Ks, mTOR inhibition by some of these compounds overlaps with PI3K inhibition. Some of the ATP-competitive inhibitors are dual mTOR/PI3K inhibitors (which inhibit both kinases at similar effective concentrations). Examples of such inhibitors include P1103, PI540, PI620, NVP-BEZ235, GSK2126458, and XL765. These compounds are all well known in the art. See, e.g., Fan et al., Cancer Cell 9:341-349, 2006; Raynaud et al., Mol. Cancer Ther. 8:1725-1738, 2009; Maira et al., Mol. Cancer Ther. 7: 1851-63, 2008; Knight et al., ACS Med. Chem. Lett., 1: 39-43, 2010; and Prasad et al., Neuro. Oncol. 13: 384-92, 2011. Some other ATP-competitive mTOR inhibitors are more selective for mTOR (pan-mTOR inhibitors) which have an IC50 for mTOR inhibition that is significantly lower than that for PI3K. These include, e.g., PP242, INK128, AZD8055, AZD2014, OSI027, TORKi CC223; and Palomid 529. These compounds have also been structurally and functionally characterized in the art. See, e.g., Apsel et al., Nature Chem. Biol. 4: 691-9, 2008; Jessen et al., Mol. Cancer Ther. 8 (Suppl. 12), Abstr. B148, 2009; Pike et al., Bioorg. Med. Chem. Lett. 23:1212-6, 2013; Bhagwat et al., Mol. Cancer Ther. 10:1394-406, 2011; and Xue et al., Cancer Res. 68: 9551-7, 2008.

Additional ATP-competitive mTOR inhibitors that can be of use with respect to the present disclosure include, e.g., WAY600, WYE354, WYE687, and WYE125132. See, e.g., Yu et al., Cancer Res. 69: 6232-40, 2009; and Yu et al., Cancer Res. 70: 621-31, 2010. These compounds all have greater selectivity for mTORC1 and mTORC2 over PI3K. They are derived from WAY001, which is a lead compound identified from a high-throughput screen directed against recombinant mTOR and which is more potent against PI3K than against mTOR. Various other mTOR inhibitors known in the art can also be used in the practice of the methods disclosed herein. These include, e.g., Torin 1 (Thoreen et al., J. Biol. Chem. 284: 8023-32, 2009), Torin2 (Liu et al., J. Med. Chem. 54:1473-80, 2011), Ku0063794 (Garcia-Martinez et al., Biochem. J. 421: 29-42, 2009), WJD008 (Li et al., J. Pharmacol. Exp. Ther. 334: 830-8, 2010), PKI402 (Mallon et al., Mol. Cancer Ther. 9: 976-84, 2010), NVP-BBD130 (Marone et al., Mol. Cancer Res. 7: 601-13, 2009), NVP-BAG956 (Marone et al., Mol. Cancer Res. 7: 601-13, 2009), and OXA-01 (Falcon et al., Cancer Res. 71: 1573-83, 2011).

Other than mTOR inhibitors that bind to and directly inhibit mTORC1 and/or mTORC2 complexes, compounds which antagonize mTOR activities in other manners may also be employed in the practice of the methods disclosed herein. These include, e.g., Metformin which indirectly inhibits mTORC1 through activation of AMPK; compounds which are capable of targeted disruption of the multiprotein TOR complexes formed from mTORC1 and mTORC1, e.g., nutlin 3 and ABT-263 (Secchiero et al., Curr. Pharm. Des. 17, 569-77, 2011; and Tse et al., Cancer Res. 68: 3421-8, 2008); compounds which antagonize or inhibit phosphatidic acid mediated activation of mTORs, e.g., HTS-1 (Veverka et al., Oncogene 27: 585-95, 2008); and compounds which block the activity of mTORC1 activator RHEB, e.g., farnesylthiosalicylic acid (McMahon et al., Mol. Endocrinol. 19:175-83, 2005).

In some embodiments, the inhibitor of mTOR is a novel inhibitor of mTOR (e.g., other rapamycin analogs) that can be identified in accordance with screening assays routinely practiced in the art. For example, a library of candidate compounds can be screened in vitro for mTOR inhibitors or rapamycin analogs that inhibit mTOR. This can be performed using methods as described in, e.g., Yu et al., Cancer Res. 69: 6232-40, 2009; Livingstone et al., Chem Biol. 2009, 16:1240-9; Chen et al., ACS Chem Biol. 2012, 7:715-22; and Bhagwat et al., Assay Drug Dev Technol. 2009, 7:471-8. The candidate compounds can be randomly synthesized chemical compounds, peptide compounds or compounds of other chemical nature. The candidate compounds can also comprise molecules that are derived structurally from known mTOR inhibitors described herein (e.g., rapamycin or analogs).

The various inhibitors of mTOR described herein can be readily obtained from commercial sources. For example, rapamycin, some rapalogues described herein, and various ATP-competitive mTOR inhibitors (e.g., Torin 1) can be purchased from a number of commercial suppliers. These include, e.g., EMD Chemicals, R&D Systems, Sigma-Aldrich, MP Biomedicals, Enzo Life Sciences, Santa Cruz Biotech, and Invitrogen. Alternatively, the inhibitors of mTOR complexes can be generated by de novo synthesis based on teachings in the art via routinely practiced protocols of organic chemistry and biochemistry. For example, methods for synthesizing rapamycin are described in the art, e.g., Ley et al., Chemistry. 2009; 15:2874-914; Nicolaou et al., J. Am. Chem. Soc. 1993, 115: 4419; Hayward et al., J. Am. Chem. Soc. 1993, 115: 9345; Romo et al., J. Am. Chem. Soc. 1993, 115: 7906; Smith et al., J. Am. Chem. Soc. 1995, 117: 5407-5408; and Maddess et al., Angew. Chem. Int. Ed. 2007, 46, 591. Structures and chemical synthesis of various other mTOR inhibitors suitable for the invention are also well characterized in the art.

In some embodiments, the provided methods include administering one or more doses of the inhibitor of mTOR to the subject. In some embodiments, each of the one or more doses of the inhibitor of mTOR is from at or about 1 mg/m² to at or about 1000 mg/m². In some embodiments, each of the one or more doses of the inhibitor of mTOR is from at or about 1 mg/m2 to at or about 100 mg/m2. In some embodiments, each of the one or more doses of the inhibitor of mTOR is from at or about 1 mg/m2 to at or about 500 mg/m2, at or about 10 mg/m2 to at or about 1000 mg/m2, at or about 1 mg/m2 to at or about 10 mg/m2, at or about 10 mg/m2 to at or about 500 mg/m2 or at or about 500 mg/m2 to at or about 1000 mg/m2. In some embodiments, each of the one or more doses of the inhibitor of mTOR is from or from about 100 mg/m2, 200 mg/m2, 300 mg/m2, 400 mg/m2, 500 mg/m2, 600 mg/m2, 700 mg/m2, 800 mg/m2, 900 mg/m2, 1000 mg/m2, or any value between any of the foregoing. In some embodiments, each of the one or more doses of the inhibitor of mTOR is from at or about 1 mg/m2 to at or about 5 mg/m2, at or about 1 mg/m2 to at or about 10 mg/m2, at or about 1 mg/m2 to at or about 100 mg/m2, at or about 1 mg/m2 to at or about 500 mg/m2 or at or about 500 mg/m2 to at or about 1000 mg/m2. In some embodiments, each of the one or more doses of the inhibitor of mTOR is from or from about 10 mg/m2, 20 mg/m2, 30 mg/m2, 40 mg/m2, 50 mg/m2, 60 mg/m2, 70 mg/m2, 80 mg/m2, 90 mg/m2, 100 mg/m2, 200 mg/m2, 300 mg/m2, 400 mg/m2, or 500 mg/m2, or any value between any of the foregoing. In some embodiments, each of the one or more doses of the inhibitor of mTOR is from or from about 25 mg/m2, 50 mg/m2, 75 mg/m2, 100 mg/m2, 125 mg/m2, 150 mg/m2, 175 mg/m2, 200 mg/m2, 220 mg/m2, 225 mg/m2, 250 mg/m2, 275 mg/m2, 300 mg/m2, or 500 mg/m2, or any value between any of the foregoing.

In certain embodiments, the subject is administered or has been administered the inhibitor of mTOR 1 month before or after administration of the viral vector or a first dose of the viral vector. In some embodiments, the subject is administered or has been administered the inhibitor of mTOR within 1 month before administration of the viral vector or a first dose of the viral vector, such as within or at or about 4 weeks, 3 weeks, 2 weeks or 1 weeks, such as at or about 1 day, 2 days, 3 days, 4 days, 5 days, 6 days or 7 days before administration of the viral vector or a first dose of the viral vector. In some embodiments, the subject is administered or has been administered the inhibitor of mTOR within 3 days before administration of the viral vector.

In certain embodiments, the subject is administered or has been administered the inhibitor of mTOR 1 day before or after administration of the viral vector or a first dose of the viral vector. In some embodiments, the subject is administered or has been administered the inhibitor of mTOR within 1 day before administration of the viral vector or a first dose of the viral vector, such as within or at or about 24, 23, 22, 21, 20, 19, 18, 17, 16, 15, 14, 13, or 12 hours, or such as 12, 11, 10, 9, 8, 7, 6, 5, 4, 3, 2, or 1 hour before administration of the viral vector or a first dose of the viral vector. In some embodiments, the subject is administered or has been administered the inhibitor of mTOR within 3 hours before administration of the viral vector.

In some embodiments, the inhibitor of mTOR is administered daily. In some embodiments, the inhibitor of mTOR is administered once a week (Q1W). In some embodiments, the inhibitor of mTOR is administered once every two weeks (Q2W). In some embodiments, the inhibitor of mTOR is administered once every three weeks (Q3W). In some embodiments, the inhibitor of mTOR is administered once every four weeks (Q4W). In some embodiments, the inhibitor of mTOR is administered one time.

In some embodiments, the inhibitor of mTOR is administered for one week, two weeks, three weeks, four weeks, five weeks, six weeks, seven weeks or eight weeks. In some embodiments, the inhibitor of mTOR is administered for four weeks. In some embodiments, the inhibitor of mTOR is administered for five weeks. In some embodiments, the inhibitor of mTOR is administered for six weeks. In some embodiments, the inhibitor of mTOR is administered for seven weeks.

In some embodiments, the inhibitor of mTOR may be provided as a pharmaceutical composition. In some embodiments, the pharmaceutical composition contains the inhibitor of mTOR and a pharmaceutically acceptable carrier.

In some embodiments, the transduction mixture further comprises one or more inhibitor of mTORs. In some embodiments, the transduction mixture comprises one or more doses of the inhibitor of mTOR from at or about 1 μM to at or about 100 μM. In some embodiments, the transduction mixture comprises one or more doses of the inhibitor of mTOR from at or about 1 μM to at or about 10 μM. In some embodiments, the transduction mixture comprises one or more doses of the inhibitor of mTOR from at or about 1 μM to at or about 5 μM, at or about 1 μM to at or about 10 μM, at or about 1 μM to at or about 20 μM, at or about 1 μM to at or about 30 μM, at or about 1 μM to at or about 40 μM, or at or about 1 μM to at or about 50 μM. In some embodiments, the transduction mixture comprises one or more doses of the inhibitor of mTOR from at or about 1 μM to at or about 50 μM, at or about 1 μM to at or about 100 μM, at or about 1 μM to at or about 10 μM, at or about 1 μM to at or about 5 μM or at or about 5 μM to at or about 10 μM. In some embodiments, the transduction mixture comprises one or more doses of the inhibitor of mTOR from or from about 1 μM, 2 μM, 3 μM, 4 μM, 5 μM, 6 μM, 7 μM, 8 μM, 9 μM, M, 20 μM, 30 μM, 40 μM, or 50 μM, or any value between any of the foregoing. In some embodiments, the transduction mixture comprises one or more doses of the inhibitor of mTOR from or from about 10 μM, 20 μM, 30 μM, 40 μM, 50 μM, 60 μM, 70 μM, 70 μM, 70 μM, 100 μM, 200 μM, 300 μM, 400 μM, or 500 μM, or any value between any of the foregoing.

In some embodiments, the pharmaceutical compositions containing an inhibitor of mTOR may be suitably developed for intravenous, intratumoral oral, rectal, vaginal, parenteral, topical, pulmonary, intranasal, buccal, ophthalmic, or another route of administration. In some embodiments, inhibitor of mTOR is administered subcutaneously. In some embodiments, the inhibitor of mTOR is administered intravenously. In some embodiments, the inhibitor of mTOR is administered intramuscularly.

In some embodiments, administering the inhibitor of mTOR in combination with the lentiviral vector in accord with the provided methods increases the percentage of T cells in the subject transduced with the lentiviral vector compared to a similar method but in which the subject is not administered an inhibitor of mTOR. In some embodiments, the percentage of T cells in the subject comprising the exogenous agent, such as the payload gene (e.g., CAR), is increased compared to a similar method but in which the subject is not administered an inhibitor of mTOR. In some embodiments of the above improvements, the increase is by greater than at or about 1.5-fold, greater than at or about 2-fold, greater than at or about 3-fold, greater than at or about 5-fold, or greater than at or about 10-fold or more.

In some embodiments, a skilled artisan is familiar with methods to assess the exposure, number, concentration, and proliferation of the T cells in the subject or T cells expressing an exogenous agent, such as payload gene (e.g., CAR). In some embodiments, the concentration or number of T cells, e.g., CAR⁺ T cells, in the plasma following administration can be measured using any method known in the art suitable for assessing concentrations of cells or particular cells expressing a transgene, e.g., CAR⁺ T cells, in samples of blood, or any methods described herein. For example, nucleic acid-based methods, such as quantitative PCR (qPCR) or flow cytometry-based methods, or other assays, such as an immunoassay, ELISA, or chromatography/mass spectrometry-based assays can be used. In some embodiments, the presence and/or amount of cells expressing the engineered receptor (e.g., CAR-expressing cells administered for T cell based therapy) in the subject following the administration by the provided methods is detected. In some aspects, nucleic acid-based methods, such as quantitative PCR (qPCR), are used to assess the quantity of cells expressing the engineered receptor (e.g., CAR-expressing cells administered for T cell based therapy) in the blood or serum or organ or tissue sample (e.g., disease site, e.g., tumor sample) of the subject. In some aspects, persistence is quantified as copies of DNA or plasmid expressing the transgene, such as encoding the engineered receptor, e.g., CAR, per microgram of DNA, or as the number of transgene-expressing, e.g., CAR-expressing, cells per microliter of the sample, e.g., of blood or serum, or per total number of peripheral blood mononuclear cells (PBMCs) or white blood cells or T cells per microliter of the sample. In some embodiments, the primers or probe used for qPCR or other nucleic acid-based methods are specific for binding, recognizing and/or amplifying the exogenous agent, such as nucleic acids encoding the engineered receptor (e.g. CAR), and/or other components or elements of the vector, such as lentiviral vector, including regulatory elements, e.g., promoters, transcriptional and/or post-transcriptional regulatory elements or response elements, or markers, e.g., surrogate markers. In some embodiments, the primers can be specific for regulatory elements, such as the woodchuck hepatitis virus post-transcriptional regulatory element (WPRE). In some examples, the presence and/or amount of cells expressing the transgene, such as engineered receptor (e.g. CAR) is expressed as copies of the nucleic acid sequence (e.g., transgene sequence) per mass of DNA (e.g., copies/μg of DNA); AUC of the curve of copies/pg of DNA over time, maximum or peak copies/pg of DNA following treatment, or copies/pg of DNA. In some embodiments, the presence and/or amount of cells can be determined at any time after the administration or infusion by the provided methods, such as at day 1, 2, 3, 4, 5, 6, 7, 8, 9, 10, 11, 12, 13, 14, 15, 16, 17, 18, 19, 20 or 21, or week 1, 2, 3, 4, 5, 6, 7, 8, 9, 10, 11, or 12 or more post-treatment or initiation thereof.

### B. Antiviral Restriction Factor Inhibitor

In some embodiments, the methods provided herein include administering to a subject an inhibitor of an antiviral restriction factor. In some embodiments the inhibitor of an antiviral restriction factor is an agent which inhibits a protein capable of reducing viral infectivity or fitness.

Antiviral restriction factors in some aspects includes effectors that are part of the cell-autonomous innate immune system, whereby cells detect the presence of pathogens (e.g., virus) and respond by deploying both local and systemic defense measures (reviewed in Towers and Noursadeghi (2014), Cell Host Microbe. 16(1):10-18). During viral infections, antiviral restriction factors tend to be induced by interferon and contribute to the so-called ‘antiviral state’ in neighboring cells. For example, the prototype human restriction factors that act against HIV-1 are TRIM5a, APOBEC3G (A3G) and BST-2/tetherin, SAMHD1 and Mx2. These intracellular proteins directly inhibit various stages of the viral life-cycle and, in the examples of TRIM5a and BST-2, they also play a role in sensing HIV and signaling, so that the consequences of expression of these factors is expected to go beyond just protecting an individual cell.

In some embodiment, the inhibitor of an antiviral restriction factor is an inhibitor of mTOR, such as a resveratrol cyclotrimer compound, such as caraphenol A, a-viniferin or resveratrol, or an analog compound thereof. Any suitable inhibitor of mTOR known in the art can be used in the methods as disclosed herein.

In some embodiments, the inhibitor of an antiviral restriction factor is Amphotericin B, or an analog compound thereof. In some embodiments, the inhibitor of an antiviral restriction factor is a polyene antifungal agent. In some embodiments, the antifungal agent is any polyene antifungal agent known in the art. In some embodiments, the antifungal agent is selected from the group comprising nystatin, amphotericin B, and pimaricin. For example, in some embodiments, the inhibitor of an antiviral restriction factor is a polyene antifungal agent that is Amphotericin B.

In some embodiments, the inhibitor of an antiviral restriction factor inhibits one or more steps of a viral life cycle, such as a lentiviral life cycle. Restriction factors expressed by a host cell may, in some aspects, act on any of the following steps: fusion and/or binding, uncoating, transcription or reverse-transcription, nuclear import, genomic integration, nuclear export, translation, packaging, and/or budding. In some embodiments, the inhibitor of an antiviral restriction factor is an inhibitor of a cellular restriction factor.

In some embodiments, the inhibitor of an antiviral restriction factor is an inhibitor of SAMHD1. SAM domain and HD domain-containing protein 1 (SAMHD1) is a cellular enzyme, implicated in blocking replication of lentivirus, such a HIV or a lentiviral vector, in dendritic cells, macrophages, monocytes, and resting CD4+T lymphocytes. SAMHD1 is capable of converting deoxynucleoside triphosphates (dNTPs) to inorganic phosphate (iPPP) and a 2′-deoxynucleoside (i.e. deoxynucleosides without a phosphate group). In doing so, SAMHD1 depletes the pool of dNTPs available to a reverse transcriptase for viral cDNA synthesis and thus prevents viral replication. In some aspects, SAMHD1 may also have nuclease activity. In some embodiments, SAMHD1 can be modulated via phosphorylation at Thr residue 592. In some aspects, phosphorylated SAMHD1 is inactive. In some embodiments, the inhibitor of an antiviral restriction factor phosphorylates SAMHD1.

In some embodiments, the inhibitor of an antiviral restriction factor is an inhibitor of the IFITM (interferon inducible transmembrane protein) gene family. In some aspects, IFITM proteins inhibit virus entry at different stages of cell trafficking. Typically, viruses, including vector viruses, enter cells by fusing with a limiting cellular membrane. For most enveloped viruses fusion occurs either at the cell surface or, following uptake by endocytosis, from within endosomes. Acid-dependent viruses require acidification of the endosomal lumen by the membrane-associate vacuolar proton ATPase for fusion. Trafficking through the endocytic system, from early to late endosomes, exposes virions to increasingly acidic environments. IFITM proteins can inhibit entry and infection by a number of viruses that fuse at the cell surface or from within endosomes. In some aspects, IFITM1 is expressed primarily at the cell surface, while IFITM2 and 3 are primarily intracellular. In some aspects, IFITM3 has been localized to endosomal compartments. In some aspects, it is considered that the primary mechanism of antiviral IFITMs is the alteration of composition and mechanical properties of cell membrane. In some embodiments, the inhibitor of an antiviral restriction factor is an inhibitor of IFITMs 1-3. In some embodiments, the inhibitor of an antiviral restriction factor is an inhibitor of IFITM1, IFITM2, and/or IFITM3. In some embodiments, the inhibitor of an antiviral restriction factor is an inhibitor of IFITM1. In some embodiments, the inhibitor of an antiviral restriction factor is an inhibitor of IFITM3.

Any suitable inhibitor of IFITM proteins known in the art can be used in the methods as disclosed herein. For example, resveratrol or a resveratrol cyclotrimer compounds have been shown to inhibit the function of IFITM proteins such as is disclosed in WO2020198320, which is hereby incorporated in its entirety. In some embodiments, the inhibitor of an antiviral restriction factor is a resveratrol cyclotrimer compound, such as caraphenol A, a-viniferin or resveratrol, or an analog compound thereof.

In some embodiments, the inhibitor of an antiviral restriction factor is an inhibitor of IFITM1. In some embodiment, the inhibitor of IFITM1 is an inhibitor of mTOR, such as a resveratrol cyclotrimer compound, such as caraphenol A, a-viniferin or resveratrol, or an analog compound thereof. Any suitable inhibitor of mTOR known in the art can be used in the methods as disclosed herein.

In some embodiments, the inhibitor of an antiviral restriction factor is a recombinant protein, a chemically synthesized protein, or a conjugate. In some embodiments, the inhibitor of an antiviral restriction factor is a nucleotide or oligonucleotide sequence. In some embodiments, the inhibitor of an antiviral restriction factor is an oligonucleotide sequence that is complementary to the coding sequence or mRNA sequence encoding the antiviral restriction factor, such as an interfering RNA.

In some embodiments, administering the inhibitor of an antiviral restriction factor in combination with the lentiviral vector in accord with the provided methods increases the percentage of T cells in the subject transduced with the lentiviral vector compared to a similar method but in which the subject is not administered an inhibitor of an antiviral restriction factor. In some embodiments, the percentage of T cells in the subject comprising the exogenous agent, such as the payload gene (e.g. CAR), is increased compared to a similar method but in which the subject is not administered an inhibitor of an antiviral restriction factor. In some embodiments of the above improvements, the increase is by greater than at or about 1.5-fold, greater than at or about 2-fold, greater than at or about 3-fold, greater than at or about 5-fold, or greater than at or about 10-fold or more.

In some embodiments, a skilled artisan is familiar with methods to assess the exposure, number, concentration, and proliferation of the T cells in the subject or T cells expressing an exogenous agent, such as payload gene (e.g. CAR). In some embodiments, the concentration or number of T cells, e.g. CAR⁺ T cells, in the plasma following administration can be measured using any method known in the art suitable for assessing concentrations of cells or particular cells expressing a transgene, e.g., CAR⁺ T cells, in samples of blood, or any methods described herein. For example, nucleic acid-based methods, such as quantitative PCR (qPCR) or flow cytometry-based methods, or other assays, such as an immunoassay, ELISA, or chromatography/mass spectrometry-based assays can be used. In some embodiments, the presence and/or amount of cells expressing the engineered receptor (e.g., CAR-expressing cells administered for T cell based therapy) in the subject following the administration by the provided methods is detected. In some aspects, nucleic acid-based methods, such as quantitative PCR (qPCR), are used to assess the quantity of cells expressing the engineered receptor (e.g., CAR-expressing cells administered for T cell based therapy) in the blood or serum or organ or tissue sample (e.g., disease site, e.g., tumor sample) of the subject. In some aspects, persistence is quantified as copies of DNA or plasmid expressing the transgene, such as encoding the engineered receptor, e.g., CAR, per microgram of DNA, or as the number of transgene-expressing, e.g., CAR-expressing, cells per microliter of the sample, e.g., of blood or serum, or per total number of peripheral blood mononuclear cells (PBMCs) or white blood cells or T cells per microliter of the sample. In some embodiments, the primers or probe used for qPCR or other nucleic acid-based methods are specific for binding, recognizing and/or amplifying the transgene, such as nucleic acids encoding the engineered receptor (e.g. CAR), and/or other components or elements of the vector, such as lentiviral vector, including regulatory elements, e.g., promoters, transcriptional and/or post-transcriptional regulatory elements or response elements, or markers, e.g., surrogate markers. In some embodiments, the primers can be specific for regulatory elements, such as the woodchuck hepatitis virus post-transcriptional regulatory element (WPRE). In some examples, the presence and/or amount of cells expressing the transgene, such as engineered receptor (e.g. CAR) is expressed as copies of the nucleic acid sequence (e.g., transgene sequence) per mass of DNA (e.g., copies/pg of DNA); AUC of the curve of copies/pg of DNA over time, maximum or peak copies/pg of DNA following treatment, or copies/pg of DNA. In some embodiments, the presence and/or amount of cells can be determined at any time after the administration or infusion by the provided methods, such as at day 1, 2, 3, 4, 5, 6, 7, 8, 9, 10, 11, 12, 13, 14, 15, 16, 17, 18, 19, 20 or 21, or week 1, 2, 3, 4, 5, 6, 7, 8, 9, 10, 11, or 12 or more post-treatment or initiation thereof.

In some embodiments, formulations described above may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. In some embodiments, the pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. In some embodiments, pharmaceutical preparations may also be combined where desired with other active agents, e.g., other analgesic agents. In some embodiments, the pharmaceutical compositions may include an additional ingredient that include but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. In some embodiments, “additional ingredients” that may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

### 1) Viral Accessory Proteins

In some embodiments, vectors that package a viral accessory protein may be used to deliver an exogenous agent according to the provided methods.

Incorporation of foreign proteins (e.g., an exogenous agent) into retrovirus particles has previously been reported by fusion with gag. Unlike some retroviruses, human and simian immunodeficiency viruses (HIV/SIV) encode proteins in addition to Gag, Pol, and Env that are packaged into virus particles. These include the Vpr protein, present in all primate lentiviruses, and the Vpx protein, which is unique to the HIV-2/SIVSM/SIVMAC group of viruses. Vpr and Vpx packaging is mediated by the Gag precursor.

In some embodiments, the packaging vector is an expression vector or viral vector that lacks a packaging signal and comprises a polynucleotide encoding one, two, three, four or more viral structural and/or accessory genes. In some embodiments, the packaging vector is an expression vector or viral vector that comprises a polynucleotide encoding one, two, three, four or more viral structural and/or accessory fusion proteins. Typically, the packaging vectors are included in a packaging cell, and are introduced into the cell via transduction, transduction or infection. A retroviral, e.g., lentiviral, transfer vector can be introduced into a packaging cell line, via transduction, transduction or infection, to generate a source cell or cell line.

In some embodiments, the packaging plasmid or vector comprises or encodes a viral accessory protein to be packaged in a vector per the methods disclosed herein. In some embodiments, the packaging plasmid or vector comprises or encodes a viral accessory fusion protein to be packaged in a vector per the methods disclosed herein. In some aspects, a viral accessory proteins are those proteins encoded by the retroviral genome in addition to those encoded by the usual replicative genes gag, pro, pol, and env. In some embodiments, a viral accessory protein can be a kinase, or any other protein that is encoding by a viral genome that is not gag, pol, or rev.

In some embodiments, the viral accessory protein is an inhibitor of an antiviral restriction factor. In some embodiments, the viral accessory protein is an inhibitor of antiviral proteins. In some embodiments, the viral accessory protein inhibits an antiviral restriction factor via degradation of said antiviral restriction factor. In some embodiments, the viral accessory protein inhibits an antiviral restriction factor via phosphorylation of said antiviral restriction factor. In some embodiments, the viral accessory protein is an inhibitor of an antiviral restriction factor that is SAMHD1, and/or a protein member of the IFITM gene family.

In some embodiments, the inhibitor of an antiviral restriction factor is a fusion protein. In some embodiments, the viral accessory protein is a fusion protein with Vpr. In some embodiments, the viral accessory protein is a fusion protein with Vpx.

In some embodiments, the inhibitor of an antiviral restriction factor is a fusion protein comprising an inhibitor of an antiviral restriction factor as disclosed herein and a viral accessory protein, optionally Vpr or Vpx. In some embodiments, a fusion protein comprising an inhibitor of an antiviral restriction factor as disclosed herein and Vpr or Vpx are packaged into vector through virus type-specific interactions with the gag polyprotein precursor.

In some embodiments, the viral accessory protein is VPX, or a fusion protein thereof. In some embodiments, the viral accessory protein is a Herpes virus kinase, such as BGLF4, UL97, U69, or ORF36, or a fusion protein thereof. In some embodiments, the viral accessory protein is an inhibitor of an antiviral restriction factor that is SAMHD1 that degrades SAMHD1 In some embodiments, the viral accessory protein is an inhibitor of an antiviral restriction factor that is SAMHD1 that phosphorylates SAMHD1.

In some embodiments, the inhibitor of an antiviral restriction factor is a fusion protein comprising a linker. In some embodiments, the linker is a protease cleavage site. Any proteolytially cleavable linker known in the art can be contemplated in the present disclosure. In some embodiments, the linker has a protease cleavage site encoded by the sequences SQNY/PIV or ARVL/AEA, such as disclosed in Gene Therapy (1999) 6, 1590-1599.

In some embodiments, a source cell line includes a cell line which is capable of producing recombinant retroviral particles, comprising a producer cell line and a transfer vector construct comprising a packaging signal. Methods of preparing viral stock solutions are illustrated by, e.g., Y. Soneoka et al. (1995) Nucl. Acids Res. 23:628-633, and N. R. Landau et al. (1992) J. Virol. 66:5110-5113, which are incorporated herein by reference. Infectious virus particles may be collected from the producer cells, e.g., by cell lysis, or collection of the supernatant of the cell culture. The collected virus particles may be enriched or purified.

In some embodiments, the source cell comprises one or more plasmids coding for viral structural proteins and replication enzymes (e.g., gag, pol and env) which can package viral particles. In some embodiments, the sequences coding for at least two of the gag, pol, and env precursors are on the same plasmid. In some embodiments, the sequences coding for the gag, pol, and env precursors are on different plasmids. In some embodiments, the sequences coding for the gag, pol, and env precursors have the same expression signal, e.g., promoter. In some embodiments, the sequences coding for the gag, pol, and env precursors have a different expression signal, e.g., different promoters. In some embodiments, expression of the gag, pol, and env precursors is inducible. In some embodiments, the plasmids coding for viral structural proteins and replication enzymes are transfected (e.g., transduced) at the same time or at different times. In some embodiments, the plasmids coding for viral structural proteins and replication enzymes are transfected (e.g., transduced) at the same time or at a different time from the packaging vector.

In some embodiments, the source cell comprises one or more plasmids for the packaging of a viral accessory protein in a vector per the methods disclosed herein. In some aspects, a viral accessory proteins are those proteins encoded by the retroviral genome in addition to those encoded by the usual replicative genes gag, pro, pol, and env. A subset of these proteins, best studied in the lentiviral genus, have effects late in the viral life cycle or early in infection. In some embodiments, a viral accessory protein can be a kinase, or any other protein that is encoding by a viral genome that is not gag, pol, or rev. In some embodiments, the viral accessory protein is an inhibitor of an antiviral restriction factor. In some embodiments, the viral accessory protein is an inhibitor of antiviral proteins. In some embodiments, the viral accessory protein inhibits an antiviral restriction factor via degradation of said antiviral restriction factor. In some embodiments, the viral accessory protein inhibits an antiviral restriction factor via phosphorylation of said antiviral restriction factor. In some embodiments, the viral accessory protein is an inhibitor of an antiviral restriction factor that is SAMHD1, and/or a protein member of the IFITM gene family. In some embodiments, the viral accessory protein is VPX. In some embodiments, the viral accessory protein is a Herpes virus kinase, such as BGLF4, UL97, U69, or ORF36. In some embodiments, the viral accessory protein is an inhibitor of an antiviral restriction factor that is SAMHD1 that degrades SAMHD1 In some embodiments, the viral accessory protein is an inhibitor of an antiviral restriction factor that is SAMHD1 that phosphorylates SAMHD1.

### 2) Polyene Antifungal Agents

In some embodiments, the methods provided herein include administering to a subject an inhibitor of an antiviral restriction factor that is an antifungal agent. In some embodiments the inhibitor of an antiviral restriction factor is an antifungal agent which inhibits a protein capable of reducing viral infectivity or fitness.

In some embodiments, the antifungal agent is an inhibitor of an antiviral restriction factor. In some embodiments, the antifungal agent is an inhibitor of antiviral proteins. In some embodiments, the antifungal agent inhibits an antiviral restriction factor via degradation of said antiviral restriction factor. In some embodiments, the antifungal agent inhibits an antiviral restriction factor via phosphorylation of said antiviral restriction factor. In some embodiments, the antifungal agent is an inhibitor of an antiviral restriction factor that is SAMHD1, and/or a protein member of the IFITM gene family. In some embodiments, the antifungal agent is an inhibitor of an antiviral restriction factor that is IFITM1.

In some embodiments, the inhibitor of an antiviral restriction factor is a polyene antifungal agent. In some embodiments, the inhibitor of an antiviral restriction factor is a polyene antifungal agent which can bind membrane sterols, including the fungus-specific molecule ergosterol, and form ion leak channels. In some embodiments, the antifungal agent is any polyene antifungal agent known in the art. In some embodiments, the antifungal agent is selected from the group comprising nystatin, amphotericin B, and pimaricin. In some embodiments, the antifungal agent is amphotericin B.

In some embodiments, the antifungal agent is an inhibitor of an antiviral restriction factor that is IFITM1 that degrades IFITM1. In some embodiments, the antifungal agent is an inhibitor of an antiviral restriction factor that is IFITM1 that phosphorylates IFITM1. In some embodiments, the antifungal agent is an inhibitor of an antiviral restriction factor that is IFITM1 that prevents expression and/or prevents translation of IFITM1.

In some embodiments, the provided methods include administering one or more doses of the antifungal agent to the subject. In some embodiments, each of the one or more doses of the antifungal agent is from at or about 0.001 mg/kg to at or about 0.1 mg/kg. In some embodiments, each of the one or more doses of the antifungal agent is from at or about 0.1 mg/kg to at or about 10 mg/kg. In some embodiments, each of the one or more doses of the antifungal agent is from at or about 0.001 mg/kg to at or about 0.05 mg/kg, at or about 0.001 mg/kg to at or about 0.01 mg/kg, at or about 0.01 mg/kg to at or about 0.1 mg/kg, at or about 0.01 mg/kg to at or about 0.05 mg/kg or at or about 0.05 mg/kg to at or about 0.1 mg/kg. In some embodiments, each of the one or more doses of the recombinant cytokine is from or from about 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.02 mg/kg, 0.03 mg/kg, 0.04 mg/kg, or 0.05 mg/kg, or any value between any of the foregoing. In some embodiments, each of the one or more doses of the antifungal agent is from at or about 0.1 mg/kg to at or about 5.0 mg/kg, at or about 0.1 mg/kg to at or about 10 mg/kg, at or about 1 mg/kg to at or about 10 mg/kg, at or about 1 mg/kg to at or about 5 mg/kg or at or about 5 mg/kg to at or about 10 mg/kg. In some embodiments, each of the one or more doses of the recombinant cytokine is from or from about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, or 5 mg/kg, or any value between any of the foregoing.

In certain embodiments, the subject is administered or has been administered the antifungal agent 1 month before or after administration of the viral vector or a first dose of the viral vector. In some embodiments, the subject is administered or has been administered the antifungal agent within 1 month before administration of the viral vector or a first dose of the viral vector, such as within or at or about 4 weeks, 3 weeks, 2 weeks or 1 weeks, such as at or about 1 day, 2 days, 3 days, 4 days, 5 days, 6 days or 7 days before administration of the viral vector or a first dose of the viral vector. In some embodiments, the subject is administered or has been administered the antifungal agent within 3 days before administration of the viral vector.

In certain embodiments, the subject is administered or has been administered the antifungal agent 1 day before or after administration of the viral vector or a first dose of the viral vector. In some embodiments, the subject is administered or has been administered the antifungal agent within 1 day before administration of the viral vector or a first dose of the viral vector, such as within or at or about 24, 23, 22, 21, 20, 19, 18, 17, 16, 15, 14, 13, or 12 hours, or such as 12, 11, 10, 9, 8, 7, 6, 5, 4, 3, 2, or 1 hour before administration of the viral vector or a first dose of the viral vector. In some embodiments, the subject is administered or has been administered the antifungal agent within 3 hours before administration of the viral vector.

In some embodiments, the antifungal agent is administered daily. In some embodiments, the antifungal agent is administered once a week (Q1W). In some embodiments, the antifungal agent is administered once every two weeks (Q2W). In some embodiments, the antifungal agent is administered once every three weeks (Q3W). In some embodiments, the antifungal agent is administered once every four weeks (Q4W). In some embodiments, the antifungal agent is administered one time.

In some embodiments, the antifungal agent is administered for one week, two weeks, three weeks, four weeks, five weeks, six weeks, seven weeks or eight weeks. In some embodiments, the antifungal agent is administered for four weeks. In some embodiments, the antifungal agent is administered for five weeks. In some embodiments, the antifungal agent is administered for six weeks. In some embodiments, the antifungal agent is administered for seven weeks.

In some embodiments, the antifungal agent may be provided as a pharmaceutical composition. In some embodiments, the pharmaceutical composition contains the antifungal agent and a pharmaceutically acceptable carrier.

In some embodiments, the transduction mixture further comprises one or more antifungal agents. In some embodiments, the transduction mixture comprises one or more doses of the antifungal agent from at or about 0.001 μM to at or about 0.1 μM. In some embodiments, the transduction mixture comprises one or more doses of the antifungal agent from at or about 0.1 μM to at or about 10 μM. In some embodiments, the transduction mixture comprises one or more doses of the antifungal agent from at or about 0.001 μM to at or about 0.05 μM, at or about 0.001 μM to at or about 0.01 μM, at or about 0.01 μM to at or about 0.1 μM, at or about 0.01 μM to at or about 0.05 μM or at or about 0.05 μM to at or about 0.1 μM. In some embodiments, the transduction mixture comprises one or more doses of the antifungal agent from at or about 0.1 μM to at or about 5 μM, at or about 0.1 μM to at or about 1 μM, at or about 1 μM to at or about 10 μM, at or about 1 μM to at or about 5 μM or at or about 5 μM to at or about 10 μM. In some embodiments, the transduction mixture comprises one or more doses of the antifungal agent from or from about 0.001 μM, 0.002 μM, 0.003 μM, 0.004 μM, 0.005 μM, 0.006 μM, 0.007 μM, 0.008 μM, 0.009 μM, 0.01 μM, 0.02 μM, 0.03 μM, 0.04 μM, or 0.05 μM, or any value between any of the foregoing. In some embodiments, the transduction mixture comprises one or more doses of the antifungal agent from or from about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, or 5 μM, or any value between any of the foregoing.

In some embodiments, the pharmaceutical compositions containing an antifungal agent may be suitably developed for intravenous, intratumoral oral, rectal, vaginal, parenteral, topical, pulmonary, intranasal, buccal, ophthalmic, or another route of administration. In some embodiments, antifungal agent is administered subcutaneously. In some embodiments, the antifungal agent is administered intravenously. In some embodiments, the antifungal agent is administered intramuscularly.

In some embodiments, administering the antifungal agent in combination with the lentiviral vector in accord with the provided methods increases the percentage of T cells in the subject transduced with the lentiviral vector compared to a similar method but in which the subject is not administered an antifungal agent. In some embodiments, the percentage of T cells in the subject comprising the exogenous agent, such as the payload gene (e.g., CAR), is increased compared to a similar method but in which the subject is not administered an antifungal agent. In some embodiments of the above improvements, the increase is by greater than at or about 1.5-fold, greater than at or about 2-fold, greater than at or about 3-fold, greater than at or about 5-fold, or greater than at or about 10-fold or more.

In some embodiments, a skilled artisan is familiar with methods to assess the exposure, number, concentration, and proliferation of the T cells in the subject or T cells expressing an exogenous agent, such as payload gene (e.g., CAR). In some embodiments, the concentration or number of T cells, e.g., CAR⁺ T cells, in the plasma following administration can be measured using any method known in the art suitable for assessing concentrations of cells or particular cells expressing a transgene, e.g., CAR⁺ T cells, in samples of blood, or any methods described herein. For example, nucleic acid-based methods, such as quantitative PCR (qPCR) or flow cytometry-based methods, or other assays, such as an immunoassay, ELISA, or chromatography/mass spectrometry-based assays can be used. In some embodiments, the presence and/or amount of cells expressing the engineered receptor (e.g., CAR-expressing cells administered for T cell based therapy) in the subject following the administration by the provided methods is detected. In some aspects, nucleic acid-based methods, such as quantitative PCR (qPCR), are used to assess the quantity of cells expressing the engineered receptor (e.g., CAR-expressing cells administered for T cell based therapy) in the blood or serum or organ or tissue sample (e.g., disease site, e.g., tumor sample) of the subject. In some aspects, persistence is quantified as copies of DNA or plasmid expressing the transgene, such as encoding the engineered receptor, e.g., CAR, per microgram of DNA, or as the number of transgene-expressing, e.g., CAR-expressing, cells per microliter of the sample, e.g., of blood or serum, or per total number of peripheral blood mononuclear cells (PBMCs) or white blood cells or T cells per microliter of the sample. In some embodiments, the primers or probe used for qPCR or other nucleic acid-based methods are specific for binding, recognizing and/or amplifying the exogenous agent, such as nucleic acids encoding the engineered receptor (e.g. CAR), and/or other components or elements of the vector, such as lentiviral vector, including regulatory elements, e.g., promoters, transcriptional and/or post-transcriptional regulatory elements or response elements, or markers, e.g., surrogate markers. In some embodiments, the primers can be specific for regulatory elements, such as the woodchuck hepatitis virus post-transcriptional regulatory element (WPRE). In some examples, the presence and/or amount of cells expressing the transgene, such as engineered receptor (e.g. CAR) is expressed as copies of the nucleic acid sequence (e.g., transgene sequence) per mass of DNA (e.g., copies/pg of DNA); AUC of the curve of copies/pg of DNA over time, maximum or peak copies/pg of DNA following treatment, or copies/pg of DNA. In some embodiments, the presence and/or amount of cells can be determined at any time after the administration or infusion by the provided methods, such as at day 1, 2, 3, 4, 5, 6, 7, 8, 9, 10, 11, 12, 13, 14, 15, 16, 17, 18, 19, 20 or 21, or week 1, 2, 3, 4, 5, 6, 7, 8, 9, 10, 11, or 12 or more post-treatment or initiation thereof.

### C. Recombinant Cytokines

In some embodiments, the methods provided herein further include administering to a subject a recombinant cytokine. In some embodiments the recombinant cytokine is an agent which binds to a cytokine receptor on a T cell, such as any agent which interacts with a cytokine receptor and/or a cytokine that interacts with T cells. In some embodiments, the cytokine receptor is an IL-2 receptor, such as an intermediate affinity IL-2 receptor (IL-2Ry). In some embodiments, the cytokine receptor is an IL-7 receptor. In some embodiments, the cytokine receptor is an IL-15 receptor.

In some embodiments, the recombinant cytokine is a recombinant protein, a chemically synthesized protein, or a conjugate. In some embodiments, the recombinant cytokine is a cytokine or cytokine mutein, such as IL-2, IL-15, IL-7, and/or a combination of any of the foregoing.

In some embodiments, the provided methods include administering one or more doses of the recombinant cytokine to the subject. In some embodiments, each of the one or more doses of the recombinant cytokine is from at or about 0.001 mg/kg to at or about 0.1 mg/kg. In some embodiments, each of the one or more doses of the recombinant cytokine is from at or about 0.001 mg/kg to at or about 0.05 mg/kg, at or about 0.001 mg/kg to at or about 0.01 mg/kg, at or about 0.01 mg/kg to at or about 0.1 mg/kg, at or about 0.01 mg/kg to at or about 0.05 mg/kg or at or about 0.05 mg/kg to at or about 0.1 mg/kg. In some embodiments, each of the one or more doses of the recombinant cytokine is from or from about 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.02 mg/kg, 0.03 mg/kg, 0.04 mg/kg, or 0.05 mg/kg, or any value between any of the foregoing.

In certain embodiments, the subject is administered or has been administered the recombinant cytokine 1 month before or after administration of the viral vector or a first dose of the viral vector. In some embodiments, the subject is administered or has been administered the recombinant cytokine within 1 month before administration of the viral vector or a first dose of the viral vector, such as within or at or about 4 weeks, 3 weeks, 2 weeks or 1 weeks, such as at or about 1 day, 2 days, 3 days, 4 days, 5 days, 6 days or 7 days before administration of the viral vector or a first dose of the viral vector. In some embodiments, the subject is administered or has been administered the recombinant cytokine within 3 days before administration of the viral vector.

In certain embodiments, the subject is administered or has been administered the recombinant cytokine 1 day before or after administration of the viral vector or a first dose of the viral vector. In some embodiments, the subject is administered or has been administered the recombinant cytokine within 1 day before administration of the viral vector or a first dose of the viral vector, such as within or at or about 24, 23, 22, 21, 20, 19, 18, 17, 16, 15, 14, 13, or 12 hours, or such as 12, 11, 10, 9, 8, 7, 6, 5, 4, 3, 2, or 1 hour before administration of the viral vector or a first dose of the viral vector. In some embodiments, the subject is administered or has been administered the recombinant cytokine within 3 hours before administration of the viral vector.

In some embodiments, the recombinant cytokine is administered daily. In some embodiments, the recombinant cytokine is administered once a week (Q1W). In some embodiments, the recombinant cytokine is administered once every two weeks (Q2W). In some embodiments, the recombinant cytokine is administered once every three weeks (Q3W). In some embodiments, the recombinant cytokine is administered once every four weeks (Q4W). In some embodiments, the recombinant cytokine is administered one time.

In some embodiments, the recombinant cytokine is administered for one week, two weeks, three weeks, four weeks, five weeks, six weeks, seven weeks or eight weeks. In some embodiments, the recombinant cytokine is administered for four weeks. In some embodiments, the recombinant cytokine is administered for five weeks. In some embodiments, the recombinant cytokine is administered for six weeks. In some embodiments, the recombinant cytokine is administered for seven weeks.

In some embodiments, the recombinant cytokine may be provided as a pharmaceutical composition. In some embodiments, the pharmaceutical composition contains the recombinant cytokine and a pharmaceutically acceptable carrier.

In some embodiments, the transduction mixture further comprises one or more recombinant cytokine. In some embodiments, the transduction mixture comprises one or more doses of the recombinant cytokine from at or about 0.001 μM to at or about 0.1 μM. In some embodiments, the transduction mixture comprises one or more doses of the recombinant cytokine from at or about 0.001 μM to at or about 0.05 μM, at or about 0.001 μM to at or about 0.01 μM, at or about 0.01 μM to at or about 0.1 μM, at or about 0.01 μM to at or about 0.05 μM or at or about 0.05 μM to at or about 0.1 μM. In some embodiments, the transduction mixture comprises one or more doses of the recombinant cytokine from or from about 0.001 μM, 0.002 μM, 0.003 μM, 0.004 μM, 0.005 μM, 0.006 μM, 0.007 μM, 0.008 μM, 0.009 μM, 0.01 μM, 0.02 μM, 0.03 μM, 0.04 μM, or 0.05 μM, or any value between any of the foregoing. In some embodiments, the transduction mixture comprises one or more doses of the recombinant cytokine from at or about 1 μM to at or about 10 μM. In some embodiments, the transduction mixture comprises one or more doses of the recombinant cytokine from at or about 1 μM to at or about 5 μM, at or about 1 μM to at or about 10 μM, at or about 1 μM to at or about 100 μM, at or about 10 μM to at or about 50 μM or at or about 50 μM to at or about 100 M. In some embodiments, the transduction mixture comprises one or more doses of the recombinant cytokine from or from about 1 μM, 2 μM, 3 μM, 4 μM, 5 μM, 6 μM, 7 μM, 8 μM, 9 μM, 10 μM, 20 μM, 30 μM, 40 μM, or 50 μM, or any value between any of the foregoing.

In some embodiments, the pharmaceutical compositions containing a recombinant cytokine may be suitably developed for intravenous, intratumoral oral, rectal, vaginal, parenteral, topical, pulmonary, intranasal, buccal, ophthalmic, or another route of administration. In some embodiments, the recombinant cytokine is administered subcutaneously. In some embodiments, the recombinant cytokine is administered intravenously. In some embodiments, the recombinant cytokine is administered intramuscularly.

In some embodiments, administering the recombinant cytokine in combination with the lentiviral vector in accord with the provided methods increases the percentage of T cells in the subject transduced with the lentiviral vector compared to a similar method but in which the subject is not administered a recombinant cytokine. In some embodiments, the percentage of T cells in the subject comprising the exogenous agent, such as the payload gene (e.g. CAR), is increased compared to a similar method but in which the subject is not administered a recombinant cytokine. In some embodiments of the above improvements, the increase is by greater than at or about 1.5-fold, greater than at or about 2-fold, greater than at or about 3-fold, greater than at or about 5-fold, or greater than at or about 10-fold or more.

In some embodiments, a skilled artisan is familiar with methods to assess the exposure, number, concentration, and proliferation of the T cells in the subject or T cells expressing an exogenous agent, such as payload gene (e.g. CAR). In some embodiments, the concentration or number of T cells, e.g. CAR⁺ T cells, in the plasma following administration can be measured using any method known in the art suitable for assessing concentrations of cells or particular cells expressing a transgene, e.g., CAR⁺ T cells, in samples of blood, or any methods described herein. For example, nucleic acid-based methods, such as quantitative PCR (qPCR) or flow cytometry-based methods, or other assays, such as an immunoassay, ELISA, or chromatography/mass spectrometry-based assays can be used. In some embodiments, the presence and/or amount of cells expressing the engineered receptor (e.g., CAR-expressing cells administered for T cell based therapy) in the subject following the administration by the provided methods is detected. In some aspects, nucleic acid-based methods, such as quantitative PCR (qPCR), are used to assess the quantity of cells expressing the engineered receptor (e.g., CAR-expressing cells administered for T cell based therapy) in the blood or serum or organ or tissue sample (e.g., disease site, e.g., tumor sample) of the subject. In some aspects, persistence is quantified as copies of DNA or plasmid expressing the transgene, such as encoding the engineered receptor, e.g., CAR, per microgram of DNA, or as the number of transgene-expressing, e.g., CAR-expressing, cells per microliter of the sample, e.g., of blood or serum, or per total number of peripheral blood mononuclear cells (PBMCs) or white blood cells or T cells per microliter of the sample. In some embodiments, the primers or probe used for qPCR or other nucleic acid-based methods are specific for binding, recognizing and/or amplifying the exogenous agent, such as nucleic acids encoding the engineered receptor (e.g. CAR), and/or other components or elements of the vector, such as lentiviral vector, including regulatory elements, e.g., promoters, transcriptional and/or post-transcriptional regulatory elements or response elements, or markers, e.g., surrogate markers. In some embodiments, the primers can be specific for regulatory elements, such as the woodchuck hepatitis virus post-transcriptional regulatory element (WPRE). In some examples, the presence and/or amount of cells expressing the transgene, such as engineered receptor (e.g. CAR) is expressed as copies of the nucleic acid sequence (e.g., transgene sequence) per mass of DNA (e.g., copies/pg of DNA); AUC of the curve of copies/pg of DNA over time, maximum or peak copies/pg of DNA following treatment, or copies/pg of DNA. In some embodiments, the presence and/or amount of cells can be determined at any time after the administration or infusion by the provided methods, such as at day 1, 2, 3, 4, 5, 6, 7, 8, 9, 10, 11, 12, 13, 14, 15, 16, 17, 18, 19, 20 or 21, or week 1, 2, 3, 4, 5, 6, 7, 8, 9, 10, 11, or 12 or more post-treatment or initiation thereof.

In some embodiments, formulations described above may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. In some embodiments, the pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. In some embodiments, pharmaceutical preparations may also be combined where desired with other active agents, e.g., other analgesic agents. In some embodiments, the pharmaceutical compositions may include an additional ingredient that include but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. In some embodiments, “additional ingredients” that may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

### IV. VIRAL VECTORS FOR ADMINISTRATION

The provided methods and embodiments can be used to delivery of viral vectors or nucleic acids for administration to a subject. In some embodiments, the nucleic acid (e.g. polynucleotides) can be a naked nucleic acid (e.g. mRNA or DNA) or can be delivered in a carrier or vehicle for delivery. In some embodiments, a nucleic acid is contained in a vehicle, such as viral-particles. In some embodiments, the nucleic acid is delivered as a naked nucleic acid. In some embodiments, the nucleic acid is an mRNA. In some embodiments, the nucleic acid is a DNA, e.g., a plasmid.

In some embodiments, vectors that package a polynucleotide encoding a exogenous agent may be used to deliver the payload agent according to the provided methods. These vectors may be of any kind, including DNA vectors, RNA vectors, plasmids, viral vectors and particles. Viral vector technology is well known and described in Sambrook et al. (2001, Molecular Cloning: A Laboratory Manual, Cold Spring Harbor Laboratory, New York). Viruses, which are useful as vectors include, but are not limited to lentiviral vectors, adenoviral vectors, adeno-associated viral (AAV) vectors, herpes simplex viral vectors, retroviral vectors, oncolytic viruses, and the like.

In some embodiments, the vector may be a viral vector such as a lentiviral vector, a gamma-retroviral vector, a recombinant AAV, an adenoviral vector or an oncolytic viral vector. In other aspects, non-viral vectors for example, nanoparticles and liposomes may also be used for introducing and delivery of a polynucleotide encoding the exogenous agent.

In some embodiments, the viral vector is derived from a viral vector. In any of the provided embodiments, the viral vector or nucleic acid is or encodes a exogenous agent for delivery to a cell or a cell in a subject.

In particular embodiments, the nucleic acid encoding the exogenous agent is encapsulated within the lumen of a viral vector in which the viral vector contains a lipid bilayer, a lumen surrounded by the lipid bilayer.

In some embodiments, the lipid bilayer includes membrane components of the host cell from which the lipid bilayer is derived, e.g., phospholipids, membrane proteins, etc. In some embodiments, the lipid bilayer includes a cytosol that includes components found in the cell from which the vehicle is derived, e.g., solutes, proteins, nucleic acids, etc., but not all of the components of a cell, e.g., lacking a nucleus. In some embodiments, the lipid bilayer is considered to be exosome-like. The lipid bilayer may vary in size, and in some instances have a diameter ranging from 30 and 300 nm, such as from 30 and 150 nm, and including from 40 to 100 nm.

In some embodiments, the lipid bilayer is a viral envelope. In some embodiments, the viral envelope is obtained from a host cell. In some embodiments, the viral envelope is obtained by the viral capsid from the source cell plasma membrane. In some embodiments, the lipid bilayer is obtained from a membrane other than the plasma membrane of a host cell. In some embodiments, the viral envelope lipid bilayer is embedded with viral proteins, including viral glycoproteins.

In other aspects, the lipid bilayer includes synthetic lipid complex. In some embodiments, the synthetic lipid complex is a liposome. In some embodiments, the lipid bilayer is a vesicular structure characterized by a phospholipid bilayer membrane and an inner aqueous medium. In some embodiments, the lipid bilayer has multiple lipid layers separated by aqueous medium. In some embodiments, the lipid bilayer forms spontaneously when phospholipids are suspended in an excess of aqueous solution. In some examples, the lipid components undergo self-rearrangement before the formation of closed structures and entrap water and dissolved solutes between the lipid bilayers.

In some embodiments, the lipid particle comprises several different types of lipids. In some embodiments, the lipids are amphipathic lipids. In some embodiments, the amphipathic lipids are phospholipids. In some embodiments, the phospholipids comprise phosphatidylcholine, phosphatidylethanolamine, phosphatidylinositol, and phosphatidylserine. In some embodiments, the lipids comprise phospholipids such as phosphocholines and phosphoinositols. In some embodiments, the lipids comprise DMPC, DOPC, and DSPC.

### A. Viral Vectors

In some embodiment the viral particles include those derived from retroviruses or lentiviruses. In some embodiments, the viral particle's bilayer of amphipathic lipids is or comprises the viral envelope. In some embodiments, the viral particle's bilayer of amphipathic lipids is or comprises lipids derived from an infected host cell.

Biological methods for introducing an exogenous agent to a host cell include the use of DNA and RNA vectors. DNA and RNA vectors can also be used to house and deliver polynucleotides and polypeptides. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362. Methods for producing cells comprising vectors and/or exogenous acids are well-known in the art. See, for example, Sambrook et al., 2001, Molecular Cloning: A Laboratory Manual, Cold Spring Harbor Laboratory, New York.

In some embodiments, the polynucleotides (e.g. encoding a exogenous agent) are comprised within a viral vector. In some embodiments, the polynucleotides (e.g. encoding exogenous agent) are comprised within a recombinant virus particles.

In some embodiments, the viral vector is a vectors derived from adenoviruses and adeno-associated virus (AAV). Such vectors or viral particles may be designed to utilize any of the known serotype capsids or combinations of serotype capsids. The serotype capsids may include capsids from any identified AAV serotypes and variants thereof, for example, AAV1, AAV2, AAV2G9, AAV3, AAV4, AAV4-4, AAV5, AAV6, AAV7, AAV8, AAV9, AAV10, AAV11, AAV12 and AAVrh10. In some embodiments, the AAV serotype may be or have a sequence as described in United States Publication No. US20030138772; Pulicherla et al. Molecular Therapy, 2011, 19(6): 1070-1078; U.S. Pat. Nos. 6,156,303; 7,198,951; U.S. Patent Publication Nos. US2015/0159173 and US2014/0359799; and International Patent Publication NOs. WO1998/011244, WO2005/033321 and WO2014/14422.

In some embodiments, the AAV vector is of serotype 1, 2, 6, 8 or 9. In some embodiments, the AAV vector is of serotype 6.2. In some embodiments, the AAV vector includes a capsid that is a chimera between AAV2 (aa 1-128) and AAV5 (aa 129-725) with one point mutation (A581T) (AAV2.5T, Excoffon et al. Proc Natl Acad Sci. 106(10):3875-70, 2009). In some embodiments, the AAV is a single-stranded DNA parvovirus which is capable of host genome integration during the latent phase of infectivity. For example, AAV of serotype 2 is largely endemic to the human and primate populations and frequently integrates site-specifically into human chromosome 19 q13.3. In some aspects, AAV is considered a dependent virus because it requires helper functions from either adenovirus or herpes-virus in order to replicate. In the absence of either of these helper viruses, AAV has been observed to integrate its genome into the host cell chromosome. However, these virions are not capable of propagating infection to new cells.

AAV vectors include not only single stranded vectors but self-complementary AAV vectors (scAAVs). scAAV vectors contain DNA which anneals together to form double stranded vector genome. By skipping second strand synthesis, scAAVs allow for rapid expression in the cell. The rAAV vectors may be manufactured by standard methods in the art such as by triple transfection, in sf9 insect cells or in suspension cell cultures of human cells such as HEK293 cells.

In some embodiment, suitable host cells for producing AAV derived vehicles include microorganisms, yeast cells, insect cells, and mammalian cells. In some embodiments, the term host cell includes the progeny of the original cell which has been transfected (e.g., transduced). Thus, as indicated above, a “host cell,” or “producer cell,” as used herein, generally refers to a cell which has been transduced with a vector vehicle as described herein. For example, cells from the stable human cell line, 293 (ATCC Accession No. CRL1573) are familiar to those in the art as a producer cell for AAV vectors. The 293 cell line is a human embryonic kidney cell line that has been transformed with adenovirus type-5 DNA fragments (Graham et al., J. Gen. Virol., 36:59 (1977)), and expresses the adenoviral E1a and E1b genes (Aiello et al., Virol., 94:460 (1979)). The 293 cell line is readily transfected, and thus provides a particularly useful system in which to produce AAV virions.

Producer cells as described above containing the AAV vehicles provided herein must be rendered capable of providing AAV helper functions. In some embodiments, producer cells allow AAV vectors to replicate and encapsulate polynucleotide sequences. In some embodiments, producer cells yield AAV virions. AAV helper functions are generally AAV-derived coding sequences that may be expressed to provide AAV gene products that, in turn, function for productive AAV replication. In some embodiments, AAV helper functions are used to complement necessary AAV functions that are missing from the AAV vectors. In some embodiments, AAV helper functions include at least one of the major AAV ORFs. In some embodiments, the helper functions include at least the rep coding region, or a functional homolog thereof. In some embodiments, the helper function includes at least the cap coding region, or a functional homolog thereof.

In some embodiments, the AAV helper functions are introduced into the host cell by transfecting (e.g., transducing) the host cell with a mixture of AAV helper constructs either prior to, or concurrently with, the transduction of the AAV vector. In some embodiments, the AAV helper constructs are used to provide transient expression of AAV rep and/or cap genes. In some embodiments, the AAV helper constructs lack AAV packaging sequences and can neither replicate nor package themselves.

In some embodiments, an AAV genome can be cross-packaged with a heterologous virus. Cross-genera packing of the rAAV2 genome into the human bocavirus type 1 (HBoV1) capsid (rAAV2/HBoV1 hybrid vector), for example, results in a hybrid vector that is highly tropic for airway epithelium (Yan et al., 2013, Mol. Ther., 21:2181-94).

In some embodiments, the virus particles are retroviral or lentiviral particles. In some embodiments, the lentiviral vector particle is Human Immunodeficiency Virus-1 (HIV-1).

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740).

In some aspects, such viral vector particles contain viral nucleic acid, such as retroviral nucleic acid, for example lentiviral nucleic acid. In particular embodiments, the viral vector particle is replication defective. In some embodiments, the viral vector particle is a lentiviral vector.

Methods of lentiviral transduction are known. Exemplary methods are described in, e.g., Wang et al., J. Immunother. 35(9): 689-701, 2012; Cooper et al., Blood. 101:1637-1644, 2003; Verhoeyen et al., Methods Mol Biol. 506: 97-114, 2009; and Cavalieri et al., Blood. 102(2): 497-505, 2003. Exemplary methods for generating viral vectors including lentiviral vectors are described further below.

In some embodiments, the viral vector is a lentiviral vector. Lentiviral vectors are particularly useful means for successful viral transduction as they permit stable expression of the gene contained within the delivered nucleic acid transcript. Lentiviral vectors express reverse transcriptase and integrase, two enzymes required for stable expression of the gene contained within the delivered nucleic acid transcript. Reverse transcriptase converts an RNA transcript into DNA, while integrase inserts and integrates the DNA into the genome of the target cell. Once the DNA has been integrated stably into the genome, it divides along with the host. The gene of interest contained within the integrated DNA may be expressed constitutively or it may be inducible. As part of the host cell genome, it may be subject to cellular regulation, including activation or repression, depending on a host of factors in the target cell.

Lentiviruses are subgroup of the Retroviridae family of viruses, named because reverse transcription of viral RNA genomes to DNA is required before integration into the host genome. As such, the most important features of lentiviral vehicles/particles are the integration of their genetic material into the genome of a target/host cell. Some examples of lentivirus include the Human Immunodeficiency Viruses: HIV-1 and HIV-2, the Simian Immunodeficiency Virus (SIV), feline immunodeficiency virus (FIV), bovine immunodeficiency virus (BIV), Jembrana Disease Virus (JDV), equine infectious anemia virus (EIAV), equine infectious anemia, virus, visna-maedi and caprine arthritis encephalitis virus (CAEV).

Typically, lentiviral particles making up the gene delivery vehicle are replication defective on their own (also referred to as “self-inactivating”). Lentiviruses are able to infect both dividing and non-dividing cells by virtue of the entry mechanism through the intact host nuclear envelope (Naldini L et al., Curr. Opin. Bioiecknol, 1998, 9: 457-463). Recombinant lentiviral vehicles/particles have been generated by multiply attenuating the HIV virulence genes, for example, the genes Env, Vif, Vpr, Vpu, Nef and Tat are deleted making the vector biologically safe. Correspondingly, lentiviral vehicles, for example, derived from HIV-1/HIV-2 can mediate the efficient delivery, integration and long-term expression of transgenes into non-dividing cells.

Lentiviral particles may be generated by co-expressing the virus packaging elements and the vector genome itself in a producer cell such as human HEK293T cells. These elements are usually provided in three (in second generation lentiviral systems) or four separate plasmids (in third generation lentiviral systems). The producer cells are co-transfected with plasmids that encode lentiviral components including the core (i.e. structural proteins) and enzymatic components of the virus, and the envelope protein(s) (referred to as the packaging systems), and a plasmid that encodes the genome including a foreign transgene, to be transferred to the target cell, the vehicle itself (also referred to as the transfer vector). In general, the plasmids or vectors are included in a producer cell line. The plasmids/vectors are introduced via transfection, transduction or infection into the producer cell line. Methods for transfection, transduction or infection are well known by those of skill in the art. As non-limiting example, the packaging and transfer constructs can be introduced into producer cell lines by calcium phosphate transfection, lipofection or electroporation, generally together with a dominant selectable marker, such as neomycin (neo), dihydrofolate reductase (DHFR), glutamine synthetase or adenosine deaminase (ADA), followed by selection in the presence of the appropriate drug and isolation of clones.

The producer cell produces recombinant viral particles that contain the foreign gene, for example, the exogenous agent that is a payload gene. The recombinant viral particles are recovered from the culture media and titrated by standard methods used by those of skill in the art. The recombinant lentiviral vehicles can be used to infect target cells.

Cells that can be used to produce high-titer lentiviral particles may include, but are not limited to, HEK293T cells, 293G cells, STAR cells (Relander et al., Mol Ther. 2005, 11: 452-459), FreeStyle™ 293 Expression System (ThermoFisher, Waltham, MA), and other HEK293T-based producer cell lines (e.g., Stewart et al., Hum Gene Ther. _2011, 2,2.(3):357-369; Lee et al, Biotechnol Bioeng, 2012, 10996): 1551-1560; Throm et al., Blood. 2009, 113(21): 5104-5110).

In some aspects, the envelope proteins may be heterologous envelope protein from other viruses, such as the G protein of vesicular stomatitis virus (VSV G) or baculoviral gp64 envelop proteins. The VSV-G glycoprotein may especially be chosen among species classified in the vesiculovirus genus: Carajas virus (CJSV), Chandipura virus (CHPV), Cocal virus (COCV), Isfahan virus (ISFV), Maraba virus (MARAV), Piry virus (PIRYV), Vesicular stomatitis Aiagoas virus (VSAV), Vesicular stomatitis Indiana virus (VSTV) and Vesicular stomatitis New Jersey virus (VSNJV) and/or stains provisionally classified in the vesiculovims genus as Grass carp rhabdovirus, BeAn 157575 virus (BeAn 157575), Boteke virus (BTKV), Calchaqui virus (CQFV), Eel virus American (EVA), Gray Lodge virus (GLOV), Jurona virus (JURY), Klamath virus (KLAVj. Kwatta virus (KWAV), La Joya virus (LJV), Malpais Spring virus (MSPV), Mount Elgon bat virus (MEB V), Ferine t virus (PERV), Pike fry rhabdovirus (PFRV), Porton virus (PORV), Radi virus (RADIV), Spring viremia of carp virus (SVCV), Tupaia virus (TUPV), Ulcerative disease rhabdovirus (UDRV) and Yug Bogdanovac virus (YBV). The gp64 or other baculoviral env protein can be derived from Autographa californica nucleopolyhedroviras (AcMNPV), Anagrapha falcifera nuclear polyhedrosis virus, Bombyx mori nuclear polyhedrosis virus, Choristoneura fiimiferana nucleopolyhedroviras, Orgyia pseudotsugata single capsid nuclear polyhedrosis virus, Epiphyas postvittana nucleopolyhedroviras, Hypharitria cunea nucleopolyhedroviras, Galleria mellonella nuclear polyhedrosis virus, Dhori virus, Thogoto virus, Antheraea pemyi nucleopolyhedroviras or Batken virus.

In some embodiments, the envelope protein may be a fusogen. Exemplary fusogens include paramyxovirus fusogens such as described below.

Additional elements provided in lentiviral particles may comprise retroviral LTR (long-terminal repeat) at either 5′ or 3′ terminus, a retroviral export element, optionally a lentiviral reverse response element (RRE), a promoter or active portion thereof, and a locus control region (LCR) or active portion thereof. Other elements include central polypurine tract (cPPT) sequence to improve transduction efficiency in non-dividing cells, Woodchuck Hepatitis Virus (WHP) Posttranscriptional Regulatory Element (WPRE) which enhances the expression of the transgene, and increases titer.

Methods for generating recombinant lentiviral particles are known to a skilled artisan, for example, U.S. Pat. Nos. 8,846,385; 7,745,179; 7,629,153; 7,575,924; 7,179,903; and 6,808,905. Lentivirus vectors used may be selected from, but are not limited to pLVX, pLenti, pLenti6, pLJMl, FUGW, pWPXL, pWPI, pLenti CMV puro DEST, pLJMl-EGFP, pULTRA, pInducer2Q, pHIV-EGFP, pCW57.1, pTRPE, pELPS, pRRL, and pLionII, Any known lentiviral vehicles may also be used (See, U.S. Pat. Nos. 9,260,725: 9,068,199: 9,023,646: 8,900,858: 8,748,169; 8,709,799; 8,420,104; 8,329,462; 8,076,106; 6,013,516; and 5,994, 136; International Patent Publication NO.: WO2012079000).

Other retroviral vectors also may be used to package a payload agent (e.g., exogenous agent) for delivery to a target cell. Retroviral vectors (RVs) allow the permanent integration of a transgene in target cells. In addition to lentiviral vectors based on complex HIV-1/2, retroviral vectors based on simple gamma-retroviruses have been widely used to deliver therapeutic genes and demonstrated clinically as one of the most efficient and powerful gene delivery systems capable of transducing a broad range of cell types. Example species of Gamma retroviruses include the murine leukemia viruses (MLVs) and the feline leukemia viruses (FeLV).

In some embodiments, gamma-retro viral vectors derived from a mammalian gamma-retrovirus such as murine leukemia viruses (MLVs), are recombinant. The MLV families of gamma retroviruses include the ecotropic, amphotropic, xenotropic and polytropic subfamilies. Ecotropic viruses are able to infect only murine cells using mCAT-1 receptor. Examples of ecotropic viruses are Moloney MLV and AKV. Amphotropic viruses infect murine, human and other species through the Pit-2 receptor. One example of an amphotropic virus is the 4070A virus. Xenotropic and polytropic viruses utilize the same (Xprl) receptor, but differ in their species tropism. Xenotropic viruses such as NZB-9-1 infect human and other species but not murine species, whereas polytropic viruses such as focus-forming viruses (MCF) infect murine, human and other species.

Gamma-retroviral vectors may be produced in packaging cells by co-transfecting the cells with several plasmids including one encoding the retroviral structural and enzymatic (gag-pol) polyprotein, one encoding the envelope (env) protein, and one encoding the vector mRNA comprising polynucleotide encoding the exogenous agent that is to be packaged in newly formed viral particles.

In some aspects, the recombinant gamma-retroviral vectors are pseudotyped with envelope proteins from other viruses. Envelope glycoproteins are incorporated in the outer lipid layer of the viral particles which can increase/alter the cell tropism. Exemplary envelope proteins include the gibbon ape leukemia vims envelope protein (GALV) or vesicular stomatitis virus G protein (VSV-G), or Simian endogenous retrovirus envelope protein, or Measles Virus H and F proteins, or Human immunodeficiency virus gp120 envelope protein, or cocal vesiculovirus envelope protein (See, e.g., U.S. application publication NO.: 2012/164118). In other aspects, envelope glycoproteins may be genetically modified to incorporate targeting/binding ligands into gamma-retroviral vectors, binding ligands including, but not limited to, peptide ligands, single chain antibodies and growth factors (Waehier et aL, Nat. Rev. Genet. 2007, 8(8):573-587). These engineered glycoproteins can retarget vectors to cells expressing their corresponding target moieties. In other aspects, a “molecular bridge” may be introduced to direct vectors to specific cells. The molecular bridge has dual specificities: one end can recognize viral glycoproteins, and the other end can bind to the molecular determinant on the target cell. Such molecular bridges, for example ligand-receptor, avidin-biotin, and chemical conjugations, monoclonal antibodies and engineered fusogenic proteins, can direct the attachment of viral vectors to target cells for transduction (Yang et al, Biotechnol Bioeng., 2008, 101(2): 357-368; and Maetzig et al, Viruses, 2011, 3, 677-713).

Exemplary envelope proteins including fusogens retargeted with a target moiety for binding to a target cell are described below.

In some embodiments, the recombinant gamma-retroviral vectors are self-inactivating (SIN) gammaretroviral vectors. The vectors may be replication incompetent. SIN vectors may harbor a deletion within the 3′ U3 region initially comprising enhancer/promoter activity. Furthermore, the 5′ U3 region may be replaced with strong promoters (needed in the packaging cell line) derived from Cytomegalovirus or RSV, or an internal promoter of choice, and/or an enhancer element. The choice of the internal promoters may be made according to specific requirements of gene expression needed for a particular purpose.

In some embodiments, polynucleotides encoding the exogenous agent are inserted within the recombinant viral genome. The other components of the viral mRNA of a recombinant gamma-retroviral vector may be modified by insertion or removal of naturally occurring sequences (e.g., insertion of an IRES, insertion of a heterologous polynucleotide encoding a polypeptide or inhibitory nucleic acid of interest, shuffling of a more effective promoter from a different retrovirus or virus in place of the wild-type promoter and the like). In some examples, the recombinant gamma-retroviral vectors may comprise modified packaging signal, and/or primer binding site (PBS), and/or 5′-enhancer/promoter elements in the U3-region of the 5′-long terminal repeat (LTR), and/or 3′-SIN elements modified in the US-region of the 3-LTR. These modifications may increase the titers and the ability of infection. Gamma retroviral vectors suitable for delivering the heterologous agent(s) (e.g. CAR and/or immunomodulatory, such as a cytokine) may be selected from those disclosed in U.S. Pat. Nos. 8,828,718; 7,585,676; 7,351,585; U.S. application publication NO.: US2007/048285; PCT application publication NOs.: WO2010/113037; WO2014/121005; WO2015/056014; and EP Pat, NOs.: EP1757702; EP1757703).

### B. Methods of Generating Viral Vectors

The provided viral-based particles include particles derived from a virus, such as viral particles, including those derived from retroviruses or lentiviruses.

In some embodiments, the assembly of a viral particle or virus-like particle is initiated by binding of the core protein to a unique encapsidation sequence within the viral genome (e.g. UTR with stem-loop structure). In some embodiments, the interaction of the core with the encapsidation sequence facilitates oligomerization.

Any of a variety of known methods can be used to produce retroviral particles whose genome contains an RNA copy of the viral vector genome. In some embodiments, at least two components are involved in making a virus-based gene delivery system: first, packaging plasmids, encompassing the structural proteins as well as the enzymes necessary to generate a viral vector particle, and second, the viral vector itself, i.e., the genetic material to be transferred. Biosafety safeguards can be introduced in the design of one or both of these components.

In some embodiments, the packaging plasmid can contain all retroviral, such as HIV-1, proteins other than envelope proteins (Naldini et al., 1998). In other embodiments, viral vectors can lack additional viral genes, such as those that are associated with virulence, e.g. vpr, vif, vpu and nef, and/or Tat, a primary transactivator of HIV. In some embodiments, lentiviral vectors, such as HIV-based lentiviral vectors, comprise only three genes of the parental virus: gag, pol and rev, which reduces or eliminates the possibility of reconstitution of a wild-type virus through recombination.

In some embodiments, a vector herein is a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses.

In some embodiments, a viral vector comprises a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). In some embodiments, a viral vector comprises e.g., a virus or viral particle capable of transferring a nucleic acid into a cell, or to the transferred nucleic acid (e.g., as naked DNA). In some embodiments, a viral vectors and transfer plasmids comprise structural and/or functional genetic elements that are primarily derived from a virus. A retroviral vector can comprise a viral vector or plasmid containing structural and functional genetic elements, or portions thereof, that are primarily derived from a retrovirus. A lentiviral vector can comprise a viral vector or plasmid containing structural and functional genetic elements, or portions thereof, including LTRs that are primarily derived from a lentivirus.

In embodiments, a lentiviral vector (e.g., lentiviral expression vector) may comprise a lentiviral transfer plasmid (e.g., as naked DNA) or an infectious lentiviral particle. With respect to elements such as cloning sites, promoters, regulatory elements, heterologous nucleic acids, etc., it is to be understood that the sequences of these elements can be present in RNA form in lentiviral particles and can be present in DNA form in DNA plasmids.

In some embodiments, in the vectors described herein at least part of one or more protein coding regions that contribute to or are essential for replication may be absent compared to the corresponding wild-type virus. In some embodiments, the viral vector replication-defective. In some embodiments, the vector is capable of transducing a target non-dividing host cell and/or integrating its genome into a host genome.

In some embodiments, the structure of a wild-type retrovirus genome often comprises a 5′ long terminal repeat (LTR) and a 3′ LTR, between or within which are located a packaging signal to enable the genome to be packaged, a primer binding site, integration sites to enable integration into a host cell genome and gag, pol and env genes encoding the packaging components which promote the assembly of viral particles. More complex retroviruses have additional features, such as rev and RRE sequences in HIV, which enable the efficient export of RNA transcripts of the integrated provirus from the nucleus to the cytoplasm of an infected target cell. In the provirus, the viral genes are flanked at both ends by regions called long terminal repeats (LTRs). In some embodiments, the LTRs are involved in proviral integration and transcription. In some embodiments, LTRs serve as enhancer-promoter sequences and can control the expression of the viral genes. In some embodiments, encapsidation of the retroviral RNAs occurs by virtue of a psi sequence located at the 5′ end of the viral genome.

In some embodiments, LTRs are similar sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

In some embodiments, for the viral genome, the site of transcription initiation is typically at the boundary between U3 and R in one LTR and the site of poly (A) addition (termination) is at the boundary between R and U5 in the other LTR. U3 contains most of the transcriptional control elements of the provirus, which include the promoter and multiple enhancer sequences responsive to cellular and in some cases, viral transcriptional activator proteins. In some embodiments, retroviruses comprise any one or more of the following genes that code for proteins that are involved in the regulation of gene expression: tat, rev, tax and rex.

In some embodiments, the structural genes gag, pol and env, gag encodes the internal structural protein of the virus. In some embodiments, Gag protein is proteolytically processed into the mature proteins MA (matrix), CA (capsid) and NC (nucleocapsid). In some embodiments, the pol gene encodes the reverse transcriptase (RT), which contains DNA polymerase, associated RNase H and integrase (IN), which mediate replication of the genome. In some embodiments, the env gene encodes the surface (SU) glycoprotein and the transmembrane (TM) protein of the virion, which form a complex that interacts specifically with cellular receptor proteins. In some embodiments, the interaction promotes infection by fusion of the viral membrane with the cell membrane.

In some embodiments, a replication-defective retroviral vector genome gag, pol and env may be absent or not functional. In some embodiments, the R regions at both ends of the RNA are typically repeated sequences. In some embodiments, U5 and U3 represent unique sequences at the 5′ and 3′ ends of the RNA genome respectively.

In some embodiments, retroviruses may also contain additional genes which code for proteins other than gag, pol and env. Examples of additional genes include (in HIV), one or more of vif, vpr, vpx, vpu, tat, rev and nef. EIAV has (amongst others) the additional gene S2. In some embodiments, proteins encoded by additional genes serve various functions, some of which may be duplicative of a function provided by a cellular protein. In EIAV, for example, tat acts as a transcriptional activator of the viral LTR (Derse and Newbold 1993 Virology 194:530-6; Maury et al. 1994 Virology 200:632-42). It binds to a stable, stem-loop RNA secondary structure referred to as TAR. Rev regulates and co-ordinates the expression of viral genes through rev-response elements (RRE) (Martarano et al. 1994 J. Virol. 68:3102-11).

In some embodiments, in addition to protease, reverse transcriptase and integrase, non-primate lentiviruses contain a fourth pol gene product which codes for a dUTPase. In some embodiments, this a role in the ability of these lentiviruses to infect certain non-dividing or slowly dividing cell types.

In embodiments, a recombinant lentiviral vector (RLV) is a vector with sufficient retroviral genetic information to allow packaging of an RNA genome, in the presence of packaging components, into a viral particle capable of infecting a target cell. In some embodiments, infection of the target cell can comprise reverse transcription and integration into the target cell genome. In some embodiments, the RLV typically carries non-viral coding sequences which are to be delivered by the vector to the target cell. In some embodiments, an RLV is incapable of independent replication to produce infectious retroviral particles within the target cell. In some embodiments, the RLV lacks a functional gag-pol and/or env gene and/or other genes involved in replication. In some embodiments, the vector may be configured as a split-intron vector, e.g., as described in PCT patent application WO 99/15683, which is herein incorporated by reference in its entirety.

In some embodiments, the lentiviral vector comprises a minimal viral genome, e.g., the viral vector has been manipulated so as to remove the non-essential elements and to retain the essential elements in order to provide the required functionality to infect, transduce and deliver a nucleotide sequence of interest to a target host cell, e.g., as described in WO 98/17815, which is herein incorporated by reference in its entirety.

In some embodiments, a minimal lentiviral genome may comprise, e.g., (5′)R-U5-one or more first nucleotide sequences-U3-R(3′). In some embodiments, the plasmid vector used to produce the lentiviral genome within a source cell can also include transcriptional regulatory control sequences operably linked to the lentiviral genome to direct transcription of the genome in a source cell. In some embodiments, the regulatory sequences may comprise the natural sequences associated with the transcribed retroviral sequence, e.g., the 5′ U3 region, or they may comprise a heterologous promoter such as another viral promoter, for example the CMV promoter. In some embodiments, lentiviral genomes comprise additional sequences to promote efficient virus production. In some embodiments, in the case of HIV, rev and RRE sequences may be included. In some embodiments, alternatively or combination, codon optimization may be used, e.g., the gene encoding the exogenous agent may be codon optimized, e.g., as described in WO 01/79518, which is herein incorporated by reference in its entirety. In some embodiments, alternative sequences which perform a similar or the same function as the rev/RRE system may also be used. In some embodiments, a functional analogue of the rev/RRE system is found in the Mason Pfizer monkey virus. In some embodiments, this is known as CTE and comprises an RRE-type sequence in the genome which is believed to interact with a factor in the infected cell. The cellular factor can be thought of as a rev analogue. In some embodiments, CTE may be used as an alternative to the rev/RRE system. In some embodiments, the Rex protein of HTLV-I can functionally replace the Rev protein of HIV-I. Rev and Rex have similar effects to IRE-BP.

In some embodiments, a retroviral nucleic acid (e.g., a lentiviral nucleic acid, e.g., a primate or non-primate lentiviral nucleic acid) (1) comprises a deleted gag gene wherein the deletion in gag removes one or more nucleotides downstream of about nucleotide 350 or 354 of the gag coding sequence; (2) has one or more accessory genes absent from the retroviral nucleic acid; (3) lacks the tat gene but includes the leader sequence between the end of the 5′ LTR and the ATG of gag; and (4) combinations of (1), (2) and (3). In an embodiment the lentiviral vector comprises all of features (1) and (2) and (3). This strategy is described in more detail in WO 99/32646, which is herein incorporated by reference in its entirety.

In some embodiments, a primate lentivirus minimal system requires none of the HIV/SIV additional genes vif, vpr, vpx, vpu, tat, rev and nef for either vector production or for transduction of dividing and non-dividing cells. In some embodiments, an EIAV minimal vector system does not require S2 for either vector production or for transduction of dividing and non-dividing cells.

In some embodiments, the deletion of additional genes may permit vectors to be produced without the genes associated with disease in lentiviral (e.g. HIV) infections. In some embodiments, tat is associated with disease. In some embodiments, the deletion of additional genes permits the vector to package more heterologous DNA. In some embodiments, genes whose function is unknown, such as S2, may be omitted, thus reducing the risk of causing undesired effects. Examples of minimal lentiviral vectors are disclosed in WO 99/32646 and in WO 98/17815.

In some embodiments, the retroviral nucleic acid is devoid of at least tat and S2 (if it is an EIAV vector system), and possibly also vif, vpr, vpx, vpu and nef. In some embodiments, the retroviral nucleic acid is also devoid of rev, RRE, or both.

In some embodiments the retroviral nucleic acid comprises vpx. The Vpx polypeptide binds to and induces the degradation of the SAMHD1 restriction factor, which degrades free dNTPs in the cytoplasm. In some embodiments, the concentration of free dNTPs in the cytoplasm increases as Vpx degrades SAMHD1 and reverse transcription activity is increased, thus facilitating reverse transcription of the retroviral genome and integration into the target cell genome.

In some embodiments, different cells differ in their usage of particular codons. In some embodiments, this codon bias corresponds to a bias in the relative abundance of particular tRNAs in the cell type. In some embodiments, by altering the codons in the sequence so that they are tailored to match with the relative abundance of corresponding tRNAs, it is possible to increase expression. In some embodiments, it is possible to decrease expression by deliberately choosing codons for which the corresponding tRNAs are known to be rare in the particular cell type. In some embodiments, an additional degree of translational control is available. An additional description of codon optimization is found, e.g., in WO 99/41397, which is herein incorporated by reference in its entirety.

In some embodiments viruses, including HIV and other lentiviruses, use a large number of rare codons and by changing these to correspond to commonly used mammalian codons, increased expression of the packaging components in mammalian producer cells can be achieved.

In some embodiments, codon optimization has a number of other advantages. In some embodiments, by virtue of alterations in their sequences, the nucleotide sequences encoding the packaging components may have RNA instability sequences (INS) reduced or eliminated from them. At the same time, the amino acid sequence coding sequence for the packaging components is retained so that the viral components encoded by the sequences remain the same, or at least sufficiently similar that the function of the packaging components is not compromised. In some embodiments, codon optimization also overcomes the Rev/RRE requirement for export, rendering optimized sequences Rev independent. In some embodiments, codon optimization also reduces homologous recombination between different constructs within the vector system (for example between the regions of overlap in the gag-pol and env open reading frames). In some embodiments, codon optimization leads to an increase in viral titer and/or improved safety.

In some embodiments, only codons relating to INS are codon optimized. In other embodiments, the sequences are codon optimized in their entirety, with the exception of the sequence encompassing the frameshift site of gag-pol.

The gag-pol gene comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimized. In some embodiments, retaining this fragment will enable more efficient expression of the gag-pol proteins. For EIAV, the beginning of the overlap is at nt 1262 (where nucleotide 1 is the A of the gag ATG). The end of the overlap is at nt 1461. In order to ensure that the frameshift site and the gag-pol overlap are preserved, the wild type sequence may be retained from nt 1156 to 1465.

In some embodiments, derivations from optimal codon usage may be made, for example, in order to accommodate convenient restriction sites, and conservative amino acid changes may be introduced into the gag-pol proteins.

In some embodiments, codon optimization is based on codons with poor codon usage in mammalian systems. The third and sometimes the second and third base may be changed.

In some embodiments, due to the degenerate nature of the genetic code, it will be appreciated that numerous gag-pol sequences can be achieved by a skilled worker. Also, there are many retroviral variants described which can be used as a starting point for generating a codon optimized gag-pol sequence. Lentiviral genomes can be quite variable. For example there are many quasi-species of HIV-I which are still functional. This is also the case for EIAV. These variants may be used to enhance particular parts of the transduction process. Examples of HIV-I variants may be found in the HIV databases maintained by Los Alamos National Laboratory. Details of EIAV clones may be found at the NCBI database maintained by the National Institutes of Health.

In some embodiments, the strategy for codon optimized gag-pol sequences can be used in relation to any retrovirus, e.g., EIAV, FIV, BIV, CAEV, VMR, SIV, HIV-I and HIV-2. In addition this method could be used to increase expression of genes from HTLV-I, HTLV-2, HFV, HSRV and human endogenous retroviruses (HERV), MLV and other retroviruses.

In embodiments, the retroviral vector comprises a packaging signal that comprises from 255 to 360 nucleotides of gag in vectors that still retain env sequences, or about 40 nucleotides of gag in a particular combination of splice donor mutation, gag and env deletions. In some embodiments, the retroviral vector includes a gag sequence which comprises one or more deletions, e.g., the gag sequence comprises about 360 nucleotides derivable from the N-terminus.

In some embodiments, the retroviral vector, helper cell, helper virus, or helper plasmid may comprise retroviral structural and accessory proteins, for example gag, pol, env, tat, rev, vif, vpr, vpu, vpx, or nef proteins or other retroviral proteins. In some embodiments the retroviral proteins are derived from the same retrovirus. In some embodiments the retroviral proteins are derived from more than one retrovirus, e.g. 2, 3, 4, or more retroviruses.

In some embodiments, the gag and pol coding sequences are generally organized as the Gag-Pol Precursor in native lentivirus. The gag sequence codes for a 55-kD Gag precursor protein, also called p55. The p55 is cleaved by the virally encoded protease4 (a product of the pol gene) during the process of maturation into four smaller proteins designated MA (matrix [p17]), CA (capsid [p24]), NC (nucleocapsid [p9]), and p6. The pol precursor protein is cleaved away from Gag by a virally encoded protease, and further digested to separate the protease (p10), RT (p50), RNase H (p15), and integrase (p31) activities.

In some embodiments, the lentiviral vector is integration-deficient. In some embodiments, the pol is integrase deficient, such as by encoding due to mutations in the integrase gene. For example, the pol coding sequence can contain an inactivating mutation in the integrase, such as by mutation of one or more of amino acids involved in catalytic activity, i.e. mutation of one or more of aspartic 64, aspartic acid 116 and/or glutamic acid 152. In some embodiments, the integrase mutation is a D64V mutation. In some embodiments, the mutation in the integrase allows for packaging of viral RNA into a lentivirus. In some embodiments, the mutation in the integrase allows for packaging of viral proteins into a lentivirus. In some embodiments, the mutation in the integrase reduces the possibility of insertional mutagenesis. In some embodiments, the mutation in the integrase decreases the possibility of generating replication-competent recombinants (RCRs) (Wanisch et al. 2009. Mol Ther. 1798):1316-1332).

In some embodiments, native Gag-Pol sequences can be utilized in a helper vector (e.g., helper plasmid or helper virus), or modifications can be made. These modifications include, chimeric Gag-Pol, where the Gag and Pol sequences are obtained from different viruses (e.g., different species, subspecies, strains, clades, etc.), and/or where the sequences have been modified to improve transcription and/or translation, and/or reduce recombination.

In some embodiments, the retroviral nucleic acid includes a polynucleotide encoding a 150-250 (e.g., 168) nucleotide portion of a gag protein that (i) includes a mutated INS1 inhibitory sequence that reduces restriction of nuclear export of RNA relative to wild-type INS1, (ii) contains two nucleotide insertion that results in frame shift and premature termination, and/or (iii) does not include INS2, INS3, and INS4 inhibitory sequences of gag.

In some embodiments, a vector described herein is a hybrid vector that comprises both retroviral (e.g., lentiviral) sequences and non-lentiviral viral sequences. In some embodiments, a hybrid vector comprises retroviral e.g., lentiviral, sequences for reverse transcription, replication, integration and/or packaging.

In some embodiments, most or all of the viral vector backbone sequences are derived from a lentivirus, e.g., HIV-1. However, it is to be understood that many different sources of retroviral and/or lentiviral sequences can be used or combined and numerous substitutions and alterations in certain of the lentiviral sequences may be accommodated without impairing the ability of a transfer vector to perform the functions described herein. A variety of lentiviral vectors are described in Naldini et al., (1996a, 1996b, and 1998); Zufferey et al., (1997); Dull et al., 1998, U.S. Pat. Nos. 6,013,516; and 5,994,136, many of which may be adapted to produce a retroviral nucleic acid.

In some embodiments, at each end of the provirus, long terminal repeats (LTRs) are typically found. An LTR typically comprises a domain located at the ends of retroviral nucleic acid which, in their natural sequence context, are direct repeats and contain U3, R and U5 regions. LTRs generally promote the expression of retroviral genes (e.g., promotion, initiation and polyadenylation of gene transcripts) and viral replication. The LTR can comprise numerous regulatory signals including transcriptional control elements, polyadenylation signals and sequences for replication and integration of the viral genome. The viral LTR is typically divided into three regions called U3, R and U5. The U3 region typically contains the enhancer and promoter elements. The U5 region is typically the sequence between the primer binding site and the R region and can contain the polyadenylation sequence. The R (repeat) region can be flanked by the U3 and U5 regions. The LTR is typically composed of U3, R and U5 regions and can appear at both the 5′ and 3′ ends of the viral genome. In some embodiments, adjacent to the 5′ LTR are sequences for reverse transcription of the genome (the tRNA primer binding site) and for efficient packaging of viral RNA into particles (the Psi site).

In some embodiments, a packaging signal can comprise a sequence located within the retroviral genome which mediate insertion of the viral RNA into the viral capsid or particle, see e.g., Clever et al., 1995. J. of Virology, Vol. 69, No. 4; pp. 2101-2109. Several retroviral vectors use a minimal packaging signal (a psi [Ψ] sequence) for encapsidation of the viral genome.

In various embodiments, retroviral nucleic acids comprise modified 5′ LTR and/or 3′ LTRs. Either or both of the LTR may comprise one or more modifications including, but not limited to, one or more deletions, insertions, or substitutions. Modifications of the 3′ LTR are often made to improve the safety of lentiviral or retroviral systems by rendering viruses replication-defective, e.g., virus that is not capable of complete, effective replication such that infective virions are not produced (e.g., replication-defective lentiviral progeny).

In some embodiments, a vector is a self-inactivating (SIN) vector, e.g., replication-defective vector, e.g., retroviral or lentiviral vector, in which the right (3′) LTR enhancer-promoter region, known as the U3 region, has been modified (e.g., by deletion or substitution) to prevent viral transcription beyond the first round of viral replication. This is because the right (3′) LTR U3 region can be used as a template for the left (5′) LTR U3 region during viral replication and, thus, absence of the U3 enhancer-promoter inhibits viral replication. In embodiments, the 3′ LTR is modified such that the U5 region is removed, altered, or replaced, for example, with an exogenous poly(A) sequence The 3′ LTR, the 5′ LTR, or both 3′ and 5′ LTRs, may be modified LTRs.

In some embodiments, the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. In some embodiments, promoters are able to drive high levels of transcription in a Tat-independent manner. In certain embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

In some embodiments, viral vectors comprise a TAR (trans-activation response) element, e.g., located in the R region of lentiviral (e.g., HIV) LTRs. This element interacts with the lentiviral trans-activator (tat) genetic element to enhance viral replication. However, this element is not required, e.g., in embodiments wherein the U3 region of the 5′ LTR is replaced by a heterologous promoter.

In some embodiments, the R region, e.g., the region within retroviral LTRs beginning at the start of the capping group (i.e., the start of transcription) and ending immediately prior to the start of the poly A tract can be flanked by the U3 and U5 regions. The R region plays a role during reverse transcription in the transfer of nascent DNA from one end of the genome to the other.

In some embodiments, the retroviral nucleic acid can also comprise a FLAP element, e.g., a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et al., 2000, Cell, 101:173, which are herein incorporated by reference in their entireties. During HIV-1 reverse transcription, central initiation of the plus-strand DNA at the central polypurine tract (cPPT) and central termination at the central termination sequence (CTS) can lead to the formation of a three-stranded DNA structure: the HIV-1 central DNA flap. In some embodiments, the retroviral or lentiviral vector backbones comprise one or more FLAP elements upstream or downstream of the gene encoding the exogenous agent. For example, in some embodiments a transfer plasmid includes a FLAP element, e.g., a FLAP element derived or isolated from HIV-1.

In embodiments, a retroviral or lentiviral nucleic acid comprises one or more export elements, e.g., a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423), and the hepatitis B virus post-transcriptional regulatory element (HPRE), which are herein incorporated by reference in their entireties. Generally, the RNA export element is placed within the 3′ UTR of a gene, and can be inserted as one or multiple copies.

In some embodiments, expression of heterologous sequences in viral vectors is increased by incorporating one or more of, e.g., all of, posttranscriptional regulatory elements, polyadenylation sites, and transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766), each of which is herein incorporated by reference in its entirety. In some embodiments, a retroviral nucleic acid described herein comprises a posttranscriptional regulatory element such as a WPRE or HPRE

In some embodiments, a retroviral nucleic acid described herein lacks or does not comprise a posttranscriptional regulatory element such as a WPRE or HPRE.

In some embodiments, elements directing the termination and polyadenylation of the heterologous nucleic acid transcripts may be included, e.g., to increases expression of the exogenous agent. Transcription termination signals may be found downstream of the polyadenylation signal. In some embodiments, vectors comprise a polyadenylation sequence 3′ of a polynucleotide encoding the exogenous agent. A polyA site may comprise a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Illustrative examples of polyA signals that can be used in a retroviral nucleic acid, include AATAAA, ATTAAA, AGTAAA, a bovine growth hormone polyA sequence (BGHpA), a rabbit β-globin polyA sequence (rpgpA), or another suitable heterologous or endogenous polyA sequence.

In some embodiments, a retroviral or lentiviral vector further comprises one or more insulator elements, e.g., an insulator element described herein.

In various embodiments, the vectors comprise a promoter operably linked to a polynucleotide encoding an exogenous agent. The vectors may have one or more LTRs, wherein either LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi (Ψ) packaging signal, RRE), and/or other elements that increase exogenous gene expression (e.g., poly (A) sequences), and may optionally comprise a WPRE or HPRE.

In some embodiments, a lentiviral nucleic acid comprises one or more of, e.g., all of, e.g., from 5′ to 3′, a promoter (e.g., CMV), an R sequence (e.g., comprising TAR), a U5 sequence (e.g., for integration), a PBS sequence (e.g., for reverse transcription), a DIS sequence (e.g., for genome dimerization), a psi packaging signal, a partial gag sequence, an RRE sequence (e.g., for nuclear export), a cPPT sequence (e.g., for nuclear import), a promoter to drive expression of the exogenous agent, a gene encoding the exogenous agent, a WPRE sequence (e.g., for efficient transgene expression), a PPT sequence (e.g., for reverse transcription), an R sequence (e.g., for polyadenylation and termination), and a U5 signal (e.g., for integration).

Some lentiviral vectors integrate inside active genes and possess strong splicing and polyadenylation signals that could lead to the formation of aberrant and possibly truncated transcripts.

Mechanisms of proto-oncogene activation may involve the generation of chimeric transcripts originating from the interaction of promoter elements or splice sites contained in the genome of the insertional mutagen with the cellular transcriptional unit targeted by integration (Gabriel et al. 2009. Nat Med 15: 1431-1436; Bokhoven, et al. J Virol 83:283-29). Chimeric fusion transcripts comprising vector sequences and cellular mRNAs can be generated either by read-through transcription starting from vector sequences and proceeding into the flanking cellular genes, or vice versa.

In some embodiments, a lentiviral nucleic acid described herein comprises a lentiviral backbone in which at least two of the splice sites have been eliminated, e.g., to improve the safety profile of the lentiviral vector. Species of such splice sites and methods of identification are described in WO2012156839A2, all of which is included by reference.

Large scale viral particle production is often useful to achieve a desired viral titer. Viral particles can be produced by transfecting (e.g., transducing) a transfer vector into a packaging cell line that comprises viral structural and/or accessory genes, e.g., gag, pol, env, tat, rev, vif, vpr, vpu, vpx, or nef genes or other retroviral genes.

In some embodiments, the packaging vector is an expression vector or viral vector that lacks a packaging signal and comprises a polynucleotide encoding one, two, three, four or more viral structural and/or accessory genes. Typically, the packaging vectors are included in a packaging cell, and are introduced into the cell via transduction, transduction or infection. A retroviral, e.g., lentiviral, transfer vector can be introduced into a packaging cell line, via transduction, transduction or infection, to generate a source cell or cell line. The packaging vectors can be introduced into human cells or cell lines by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones. A selectable marker gene can be linked physically to genes encoding by the packaging vector, e.g., by IRES or self-cleaving viral peptides.

In some embodiments, producer cell lines include cell lines that do not contain a packaging signal, but do stably or transiently express viral structural proteins and replication enzymes (e.g., gag, pol and env) which can package viral particles. Any suitable cell line can be employed, e.g., mammalian cells, e.g., human cells. Suitable cell lines which can be used include, for example, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells. In embodiments, the packaging cells are 293 cells, 293T cells, or A549 cells.

In some embodiments, a source cell line includes a cell line which is capable of producing recombinant retroviral particles, comprising a producer cell line and a transfer vector construct comprising a packaging signal. Methods of preparing viral stock solutions are illustrated by, e.g., Y. Soneoka et al. (1995) Nucl. Acids Res. 23:628-633, and N. R. Landau et al. (1992) J. Virol. 66:5110-5113, which are incorporated herein by reference. Infectious virus particles may be collected from the producer cells, e.g., by cell lysis, or collection of the supernatant of the cell culture. The collected virus particles may be enriched or purified.

In some embodiments, the source cell comprises one or more plasmids coding for viral structural proteins and replication enzymes (e.g., gag, pol and env) which can package viral particles. In some embodiments, the sequences coding for at least two of the gag, pol, and env precursors are on the same plasmid. In some embodiments, the sequences coding for the gag, pol, and env precursors are on different plasmids. In some embodiments, the sequences coding for the gag, pol, and env precursors have the same expression signal, e.g., promoter. In some embodiments, the sequences coding for the gag, pol, and env precursors have a different expression signal, e.g., different promoters. In some embodiments, expression of the gag, pol, and env precursors is inducible. In some embodiments, the plasmids coding for viral structural proteins and replication enzymes are transfected (e.g., transduced) at the same time or at different times. In some embodiments, the plasmids coding for viral structural proteins and replication enzymes are transfected (e.g., transduced) at the same time or at a different time from the packaging vector.

In some embodiments, the source cell line comprises one or more stably integrated viral structural genes. In some embodiments expression of the stably integrated viral structural genes is inducible.

In some embodiments, expression of the viral structural genes is regulated at the transcriptional level. In some embodiments, expression of the viral structural genes is regulated at the translational level. In some embodiments, expression of the viral structural genes is regulated at the post-translational level.

In some embodiments, expression of the viral structural genes is regulated by a tetracycline (Tet)-dependent system, in which a Tet-regulated transcriptional repressor (Tet-R) binds to DNA sequences included in a promoter and represses transcription by steric hindrance (Yao et al, 1998; Jones et al, 2005). Upon addition of doxycycline (dox), Tet-R is released, allowing transcription. Multiple other suitable transcriptional regulatory promoters, transcription factors, and small molecule inducers are suitable to regulate transcription of viral structural genes.

In some embodiments, the third-generation lentivirus components, human immunodeficiency virus type 1 (HIV) Rev, Gag/Pol, and an envelope under the control of Tet-regulated promoters and coupled with antibiotic resistance cassettes are separately integrated into the source cell genome. In some embodiments the source cell only has one copy of each of Rev, Gag/Pol, and an envelope protein integrated into the genome.

In some embodiments a nucleic acid encoding the exogenous agent (e.g., a retroviral nucleic acid encoding the exogenous agent) is also integrated into the source cell genome.

In some embodiments, a retroviral nucleic acid described herein is unable to undergo reverse transcription. Such a nucleic acid, in embodiments, is able to transiently express an exogenous agent. The retrovirus or VLP, may comprise a disabled reverse transcriptase protein, or may not comprise a reverse transcriptase protein. In embodiments, the retroviral nucleic acid comprises a disabled primer binding site (PBS) and/or att site. In embodiments, one or more viral accessory genes, including rev, tat, vif, nef, vpr, vpu, vpx and S2 or functional equivalents thereof, are disabled or absent from the retroviral nucleic acid. In embodiments, one or more accessory genes selected from S2, rev and tat are disabled or absent from the retroviral nucleic acid

In some embodiments, the retroviral vector systems described herein comprise viral genomes bearing cis-acting vector sequences for transcription, reverse-transcription, integration, translation and packaging of viral RNA into the viral particles, and (2) producer cells lines which express the trans-acting retroviral gene sequences (e.g., gag, pol and env) needed for production of virus particles. In some embodiments, by separating the cis- and trans-acting vector sequences completely, the virus is unable to maintain replication for more than one cycle of infection. Generation of live virus can be avoided by a number of strategies, e.g., by minimizing the overlap between the cis- and trans-acting sequences to avoid recombination.

In some embodiments, a viral vector particle which comprises a sequence that is devoid of or lacking viral RNA may be the result of removing or eliminating the viral RNA from the sequence. In one embodiment this may be achieved by using an endogenous packaging signal binding site on gag. In some embodiments, the endogenous packaging signal binding site is on pol. In this embodiment, the RNA which is to be delivered will contain a cognate packaging signal. In another embodiment, a heterologous binding domain (which is heterologous to gag) located on the RNA to be delivered, and a cognate binding site located on gag or pol, can be used to ensure packaging of the RNA to be delivered. In some embodiments, the heterologous sequence could be non-viral or it could be viral, in which case it may be derived from a different virus. In some embodiments, the vector particles are used to deliver therapeutic RNA, in which case functional integrase and/or reverse transcriptase is not required. In some embodiments, the vector particles could also be used to deliver a therapeutic gene of interest, in which case pol is typically included.

In some embodiments, gag-pol are altered, and the packaging signal is replaced with a corresponding packaging signal. In this embodiment, the particle can package the RNA with the new packaging signal. The advantage of this approach is that it is possible to package an RNA sequence which is devoid of viral sequence for example, RNAi.

In some embodiments, an alternative approach is to rely on over-expression of the RNA to be packaged. In one embodiment the RNA to be packaged is over-expressed in the absence of any RNA containing a packaging signal. This may result in a significant level of therapeutic RNA being packaged, and that this amount is sufficient to transduce a cell and have a biological effect.

In some embodiments, a polynucleotide comprises a nucleotide sequence encoding a viral gag protein or retroviral gag and pol proteins, wherein the gag protein or pol protein comprises a heterologous RNA binding domain capable of recognizing a corresponding sequence in an RNA sequence to facilitate packaging of the RNA sequence into a viral vector particle.

In some embodiments, the heterologous RNA binding domain comprises an RNA binding domain derived from a bacteriophage coat protein, a Rev protein, a protein of the U1 small nuclear ribonucleoprotein particle, a Nova protein, a TF111A protein, a TIS11 protein, a trp RNA-binding attenuation protein (TRAP) or a pseudouridine synthase.

When a recombinant plasmid and the retroviral LTR and packaging sequences are introduced into a producer cell line (e.g., by calcium phosphate precipitation for example), the packaging sequences may permit the RNA transcript of the recombinant plasmid to be packaged into viral particles, which then may be secreted into the culture media. The media containing the recombinant retroviruses in some embodiments is then collected, optionally concentrated, and used for gene transfer. For example, in some aspects, after cotransfection of the packaging plasmids and the transfer vector to the packaging cell line, the viral vector particles are recovered from the culture media and titered by standard methods used by those of skill in the art.

In some embodiments, a retroviral vector, such as a lentiviral vector, can be produced in a producer cell line, such as an exemplary HEK 293T cell line, by introduction of plasmids to allow generation of lentiviral particles. In some embodiments, a producer cell is transfected and/or contains a polynucleotide encoding gag and pol, and, in some cases, a polynucleotide encoding an exogenous agent. In some embodiments, the producer cell line is optionally and/or additionally transfected with and/or contains a polynucleotide encoding a rev protein. In some embodiments, the producer cell line is optionally and/or additionally transfected with and/or contains a polynucleotide encoding a non-native envelope glycoprotein, such as VSV-G. In some such embodiments, approximately two days after transfection of cells, e.g. HEK 293T cells, the cell supernatant contains recombinant lentiviral vectors, which can be recovered and titered.

In some embodiments, a method herein comprises detecting or confirming the absence of replication competent retrovirus. The methods may include assessing RNA levels of one or more target genes, such as viral genes, e.g. structural or packaging genes, from which gene products are expressed in certain cells infected with a replication-competent retrovirus, such as a gammaretrovirus or lentivirus, but not present in a viral vector used to transduce cells with a heterologous nucleic acid and not, or not expected to be, present and/or expressed in cells not containing replication-competent retrovirus. Replication competent retrovirus may be determined to be present if RNA levels of the one or more target genes is higher than a reference value, which can be measured directly or indirectly, e.g. from a positive control sample containing the target gene. For further disclosure, see WO2018023094A1.

### C. Targeting and Retargeting of Viral Vectors

In some embodiments, the viral vector further comprises a vector-surface targeting moiety which specifically binds to a target ligand on a target cell. It will be recognized by those skilled in the art that, the viral vectors provided herein harbor the attachment and/or fusion glycoproteins and are capable of binding to target cells and delivering the vehicle contents to the cytoplasm of the target cells. It will also be recognized by those skilled in the art that this is due to the natural viral entry mechanism that involves fusion of the viral membrane directly with the target cell plasma membrane.

It will further be recognized by those skilled in the art that many viruses such as paramyxoviruses bind to sialic acid receptors, and hence the corresponding derivative vehicles can deliver their contents generically to nearly any kind of cell that expresses sialic acid receptors. Other viruses such as Nipah virus and HIV bind to protein receptors, and hence the corresponding vehicles have a specificity that matches the natural tropisms for each virus and its surface proteins.

Furthermore, it will be recognized that technology exists to “re-target” attachment proteins, making it so that the vehicles only interact with particular cells or cell types that express a marker protein of interest (Msaouel et al., Meths Mol Biol 797: 141-162, 2012). Thus, vehicle surface glycoproteins proteins can be supplemented with or replaced by other targeting proteins, including but not necessarily limited to antibodies and antigen binding fragments thereof, receptor ligands, and other approaches that will be apparent to those skilled in the art given the benefit of the present disclosure. In some embodiments, the vector-surface targeting moiety is a polypeptide. In some embodiments, the polypeptide is a fusogen.

In some embodiments, the viral vector comprises one or more fusogens. In some embodiments, the fusogen facilitates the fusion of the viral vector to a cell membrane to deliver the exogenous agent into the cell. In some embodiments, the membrane is a plasma cell membrane. In some embodiments, the fusogen targets the viral vector to a target cell of interest. In some embodiments, the fusogen contains a targeting moiety that provides retargeting (compared to the natural tropism of the fusogen) to the target cell of interest.

Provided herein are methods of administration of viral vectors containing a fusogen disposed or embedded in the lipid bilayer. Exemplary fusogens are described in subsections below. In some embodiments, the fusogen is composed of one or more Paramyxovirus envelope protein or a biologically active portion thereof. In some embodiments, the Paramyxovirus envelope protein or a biologically active portion thereof harbors the attachment and/or fusion glycoproteins and are capable of binding to target cells and delivering the vehicle contents to the cytoplasm of the target cells.

### D. Viral Envelope Glycoproteins and Fusogens

In some embodiments, the viral vectors contain one or more fusogens. In some embodiments, the viral vector contains an exogenous or overexpressed fusogen. In some embodiments, the fusogen is disposed in the lipid bilayer. In some embodiments, the fusogen facilitates the fusion of the viral vector to a membrane. In some embodiments, the membrane is a plasma cell membrane of a target cell. In some embodiments, the viral vector comprising the fusogen integrates into the membrane into a lipid bilayer of a target cell. In some embodiments, the fusogen results in mixing between lipids in the viral vector and lipids in the target cell. In some embodiments, the fusogen results in formation of one or more pores between the interior of the non-cell particle and the cytosol of the target cell.

In some embodiments, fusogens are protein based, lipid based, and chemical based fusogens. In some embodiments, the viral vector, contain a first fusogen that is a protein fusogen and a second fusogen that is a lipid fusogen or chemical fusogen. In some embodiments, the fusogen binds a fusogen binding partner on a target cell surface. In some embodiments, the viral vector is pseudotyped with the fusogen. In some examples, a virus of viral-like particle has a modification to one or more of its envelope proteins, e.g., an envelope protein is substituted with an envelope protein from another virus. In some embodiments, retroviral envelope proteins, e.g. lentiviral envelope proteins, are pseudotyped with a fusogen.

In some embodiments, the fusogen is a protein fusogen, e.g., a mammalian protein or a homologue of a mammalian protein (e.g., having 50%, 60%, 70%, 80%, 85%, 90%, 95%, 96%, 97%, 98%, 99%, or greater identity), a non-mammalian protein such as a viral protein or a homologue of a viral protein (e.g., having 50%, 60%, 70%, 80%, 85%, 90%, 95%, 96%, 97%, 98%, 99%, or greater identity), a native protein or a derivative of a native protein, a synthetic protein, a fragment thereof, a variant thereof, a protein fusion comprising one or more of the fusogens or fragments, and any combination thereof.

In some embodiments, the fusogen may include a mammalian protein. Examples of mammalian fusogens may include, but are not limited to, a SNARE family protein such as vSNAREs and tSNAREs, a syncytin protein such as Syncytin-1 (DOI: 10.1128/JVI.76.13.6442-6452.2002), and Syncytin-2, myomaker (biorxiv.org/content/early/2017/04/02/123158, doi.org/10.1101/123158, doi: 10.1096/fj.201600945R, doi:10.1038/nature12343), myomixer (www.nature.com/nature/journal/v499/n7458/full/nature12343.html, doi:10.1038/nature12343), myomerger (science.sciencemag.org/content/early/2017/04/05/science.aam9361, DOI: 10.1126/science.aam9361), FGFRL1 (fibroblast growth factor receptor-like 1), Minion (doi.org/10.1101/122697), an isoform of glyceraldehyde-3-phosphate dehydrogenase (GAPDH) (e.g., as disclosed in U.S. Pat. No. 6,099,857A), a gap junction protein such as connexin 43, connexin 40, connexin 45, connexin 32 or connexin 37 (e.g., as disclosed in US 2007/0224176, Hap2, any protein capable of inducing syncytium formation between heterologous cells, any protein with fusogen properties, a homologue thereof, a fragment thereof, a variant thereof, and a protein fusion comprising one or more proteins or fragments thereof. In some embodiments, the fusogen is encoded by a human endogenous retroviral element (hERV) found in the human genome. Additional exemplary fusogens are disclosed in U.S. Pat. No. 6,099,857A and US 2007/0224176, the entire contents of which are hereby incorporated by reference.

In some embodiments, the fusogen may include a non-mammalian protein, e.g., a viral protein. In some embodiments, a viral fusogen is a Class I viral membrane fusion protein, a Class II viral membrane protein, a Class III viral membrane fusion protein, a viral membrane glycoprotein, or other viral fusion proteins, or a homologue thereof, a fragment thereof, a variant thereof, or a protein fusion comprising one or more proteins or fragments thereof.

In some embodiments, Class I viral membrane fusion proteins include, but are not limited to, Baculovirus F protein, e.g., F proteins of the nucleopolyhedrovirus (NPV) genera, e.g., Spodoptera exigua MNPV (SeMNPV) F protein and Lymantria dispar MNPV (LdMNPV), and paramyxovirus F proteins.

In some embodiments, Class II viral membrane proteins include, but are not limited to, tick bone encephalitis E (TBEV E), Semliki Forest Virus E1/E2.

In some embodiments, Class III viral membrane fusion proteins include, but are not limited to, rhabdovirus G (e.g., fusogenic protein G of the Vesicular Stomatitis Virus (VSV-G)), herpesvirus glycoprotein B (e.g., Herpes Simplex virus 1 (HSV-1) gB)), Epstein Barr Virus glycoprotein B (EBV gB), thogotovirus G, baculovirus gp64 (e.g., Autographa California multiple NPV (AcMNPV) gp64), Baboon endogenous retrovirus envelope glycoprotein (BaEV), and Borna disease virus (BDV) glycoprotein (BDV G).

Examples of other viral fusogens, e.g., membrane glycoproteins and viral fusion proteins, include, but are not limited to: viral syncytia proteins such as influenza hemagglutinin (HA) or mutants, or fusion proteins thereof; human immunodeficiency virus type 1 envelope protein (HIV-1 ENV), gp120 from HIV binding LFA-1 to form lymphocyte syncytium, HIV gp41, HIV gp160, or HIV Trans-Activator of Transcription (TAT); viral glycoprotein VSV-G, viral glycoprotein from vesicular stomatitis virus of the Rhabdoviridae family; glycoproteins gB and gH-gL of the varicella-zoster virus (VZV); murine leukemia virus (MLV)-10A1; Gibbon Ape Leukemia Virus glycoprotein (GaLV); type G glycoproteins in Rabies, Mokola, vesicular stomatitis virus and Togaviruses; murine hepatitis virus JHM surface projection protein; porcine respiratory coronavirus spike- and membrane glycoproteins; avian infectious bronchitis spike glycoprotein and its precursor; bovine enteric coronavirus spike protein; the F and H, HN or G genes of Measles virus; canine distemper virus, Newcastle disease virus, human parainfluenza virus 3, simian virus 41, Sendai virus and human respiratory syncytial virus; gH of human herpesvirus 1 and simian varicella virus, with the chaperone protein gL; human, bovine and cercopithicine herpesvirus gB; envelope glycoproteins of Friend murine leukemia virus and Mason Pfizer monkey virus; mumps virus hemagglutinin neuraminidase, and glycoproteins F1 and F2; membrane glycoproteins from Venezuelan equine encephalomyelitis; paramyxovirus F protein; SIV gp160 protein; Ebola virus G protein; or Sendai virus fusion protein, or a homologue thereof, a fragment thereof, a variant thereof, and a protein fusion comprising one or more proteins or fragments thereof.

Non-mammalian fusogens include viral fusogens, homologues thereof, fragments thereof, and fusion proteins comprising one or more proteins or fragments thereof. Viral fusogens include class I fusogens, class II fusogens, class III fusogens, and class IV fusogens. In embodiments, class I fusogens such as human immunodeficiency virus (HIV) gp41, have a characteristic post fusion conformation with a signature trimer of α-helical hairpins with a central coiled-coil structure. Class I viral fusion proteins include proteins having a central post fusion six-helix bundle. Class I viral fusion proteins include influenza HA, parainfluenza F, HIV Env, Ebola GP, hemagglutinins from orthomyxoviruses, F proteins from paramyxoviruses (e.g. Measles, (Katoh et al. BMC Biotechnology 2010, 10:37)), ENV proteins from retroviruses, and fusogens of filoviruses and coronaviruses. In embodiments, class II viral fusogens such as dengue E glycoprotein, have a structural signature of β-sheets forming an elongated ectodomain that refolds to result in a trimer of hairpins. In embodiments, the class II viral fusogen lacks the central coiled coil. Class II viral fusogen can be found in alphaviruses (e.g., E1 protein) and flaviviruses (e.g., E glycoproteins). Class II viral fusogens include fusogens from Semliki Forest virus, Sinbis, rubella virus, and dengue virus. In embodiments, class III viral fusogens such as the vesicular stomatitis virus G glycoprotein, combine structural signatures found in classes I and II. In embodiments, a class III viral fusogen comprises a helices (e.g., forming a six-helix bundle to fold back the protein as with class I viral fusogens), and R sheets with an amphiphilic fusion peptide at its end, reminiscent of class II viral fusogens. Class III viral fusogens can be found in rhabdoviruses and herpesviruses. In embodiments, class IV viral fusogens are fusion-associated small transmembrane (FAST) proteins (doi:10.1038/sj.emboj.7600767, Nesbitt, Rae L., “Targeted Intracellular Therapeutic Delivery Using Liposomes Formulated with Multifunctional FAST proteins” (2012). Electronic Thesis and Dissertation Repository. Paper 388), which are encoded by nonenveloped reoviruses. In embodiments, the class IV viral fusogens are sufficiently small that they do not form hairpins (doi: 10.1146/annurev-cellbio-101512-122422, doi:10.1016/j.devcel.2007.12.008).

Additional exemplary fusogens are disclosed in U.S. Pat. No. 9,695,446, US 2004/0028687, U.S. Pat. Nos. 6,416,997, 7,329,807, US 2017/0112773, US 2009/0202622, WO 2006/027202, and US 2004/0009604, the entire contents of all of which are hereby incorporated by reference.

In some embodiments, the fusogen is a poxviridae fusogen.

In some embodiments the fusogen is a paramyxovirus fusogen. In some embodiments, the fusogen may be an envelope glycoprotein G, H HN and/or an F protein of the Paramyxoviridae family. In some embodiments the fusogen contains a Nipah virus protein F, a measles virus F protein, a tupaia paramyxovirus F protein, a paramyxovirus F protein, a Hendra virus F protein, a Henipavirus F protein, a Morbillivirus F protein, a respirovirus F protein, a Sendai virus F protein, a rubulavirus F protein, or an avulavirus F protein. In some embodiments, the viral vector includes a henipavirus envelope attachment glycoprotein G (G protein) or a biologically active portion thereof and/or a henipavirus envelope fusion glycoprotein F (F protein) or a biologically active portion thereof.

In particular embodiments, the fusogen is glycoprotein GP64 of baculovirus, glycoprotein GP64 variant E45K/T259A.

In some embodiments, the fusogen is a hemagglutinin-neuraminidase (HN) and fusion (F) proteins (F/HN) from a respiratory paramyxovirus. In some embodiments, the respiratory paramyxovirus is a Sendai virus. The HN and F glycoproteins of Sendai viruses function to attach to sialic acids via the HN protein, and to mediate cell fusion for entry to cells via the F protein. In some embodiments, the fusogen is a F and/or HN protein from the murine parainfluenza virus type 1 (See e.g., U.S. patent Ser. No. 10/704,061).

In some embodiments, the viral vector is pseudotyped with viral glycoproteins as described herein such as a NiV-F and/or NiV-G protein.

In some embodiments, the viral vector further comprises a vector-surface targeting moiety which specifically binds to a target ligand. In some embodiments, the vector-surface targeting moiety is a polypeptide. In some embodiments, the nucleic acid encoding the one of the Paramyxovirus envelope protein (e.g. G protein) is modified with a targeting moiety to specifically bind to a target molecule on a target cells. In some embodiments, the targeting moiety can be any targeting protein, including but not necessarily limited to antibodies and antigen binding fragments thereof.

It has been reported that the henipavirus F proteins from various species exhibit compatibility with G proteins from other species to trigger fusion (Brandel-Tretheway et al. Journal of Virology. 2019. 93(13):e00577-19). In some aspects of the provided viral vector (e.g. lentiviral vector), the F protein is heterologous to the G protein, i.e. the F and G protein or biologically active portions are from different henipavirus species. For example, the G protein is from Hendra virus and the F protein is a NiV-F as described. In other aspects, the F and/or G protein can be a chimeric F and/or G protein containing regions of F and/or G proteins from different species of Henipavirus. In some embodiments, switching a region of amino acid residues of the F protein from one species of Henipavirus to another can result in fusion to the G protein of the species comprising the amino acid insertion. (Brandel-Tretheway et al. 2019). In some cases, the chimeric F and/or G protein contains an extracellular domain from one henipavirus species and a transmembrane and/or cytoplasmic domain from a different henipavirus species. For example, the F protein contains an extracellular domain of Hendra virus and a transmembrane/cytoplasmic domain of Nipah virus.

### 1. F Proteins

In some embodiments, the viral vectors comprises a protein with a hydrophobic fusion peptide domain. In some embodiments, the protein with a hydrophobic fusion peptide domain may be an envelope glycoprotein F protein of the Paramyxoviridae family (i.e., a paramyxovirus F protein). In some embodiments, the envelope glycoprotein F protein comprises a henipavirus F protein molecule or biologically active portion thereof. In some embodiments, the Henipavirus F protein is a Hendra (HeV) virus F protein, a Nipah (NiV) virus F-protein, a Cedar (CedPV) virus F protein, a Mojiang virus F protein or a bat Paramyxovirus F protein or a biologically active portion thereof.

In some embodiments, the fusogen comprises a protein with a hydrophobic fusion peptide domain. In some embodiments, the fusogen comprises a henipavirus F protein molecule or biologically active portion thereof. In some embodiments, the Henipavirus F protein is a Hendra (HeV) virus F protein, a Nipah (NiV) virus F-protein, a Cedar (CedPV) virus F protein, a Mojiang virus F protein or a bat Paramyxovirus F protein or a biologically active portion thereof.

F proteins of henipaviruses are encoded as F₀ precursors containing a signal peptide. Following cleavage of the signal peptide, the mature F₀ is transported to the cell surface, then endocytosed and cleaved by cathepsin L into the mature fusogenic subunits F1 and F2. For instance, with reference to NiV-F the NiV-F is encoded as F0 precursors containing a signal peptide (e.g. corresponding to amino acid residues 1-26 of the below). Following cleavage of the signal peptide, the mature F0 (SEQ ID NO:2 lacking the signal peptide, i.e. set forth in SEQ ID NO:7) is transported to the cell surface, then endocytosed and cleaved by cathepsin L (e.g. between amino acids 109-110 of NiV-F corresponding to amino acids set forth in SEQ ID NO:2) into the mature fusogenic subunits F1 (e.g. corresponding to amino acids 110-546 of NiV-F set forth in SEQ ID NO:2) and F2 (e.g. corresponding to amino acid residues 27-109 of NiV-F set forth in SEQ ID NO:2). The F1 and F2 subunits are associated by a disulfide bond and recycled back to the cell surface. The F1 subunit contains the fusion peptide domain located at the N terminus of the F1 subunit (e.g. corresponding to amino acids 110-129 of the below e.g. NiV-F set forth in SEQ ID NO:2), where it is able to insert into a cell membrane to drive fusion. Without wishing to be bound by theory, in some aspects, fusion is blocked by association of the F protein with G protein, until the G protein engages with a target molecule resulting in its disassociation from F and exposure of the fusion peptide to mediate membrane fusion.

In some embodiments, the F protein (e.g. NiV-F protein) of the viral vector, such as lentiviral vector, exhibits fusogenic activity. In some embodiments, the F protein (e.g. NiV-F) facilitates the fusion of the viral vector (e.g. lentiviral vector) to a membrane. In particular embodiments, the F protein or the functionally active variant or biologically active portion thereof retains fusogenic activity in conjunction with a Henipavirus G protein, such as a G protein set forth below. Fusogenic activity includes the activity of the F protein in conjunction with a G protein to promote or facilitate fusion of two membrane lumens, such as the lumen of the targeted viral vector e having embedded in its lipid bilayer a henipavirus F and G protein, and a cytoplasm of a target cell, e.g. a cell that contains a surface receptor or molecule that is recognized or bound by the targeted envelope protein. In some embodiments, the F protein and G protein are from the same Henipavirus species (e.g. NiV-G and NiV-F). In some embodiments, the F protein and G protein are from different Henipavirus species (e.g. NiV-G and HeV-F). In particular embodiments, the F protein of the functionally active variant or biologically active portion retains the cleavage site cleaved by cathepsin L (e.g. corresponding to the cleavage site between amino acids 109-110 of SEQ ID NO:2).

In some embodiments, the F protein or the functionally active variant or biologically active portion thereof comprises an F1 subunit or a fusogenic portion thereof. In some embodiments, the F1 subunit is a proteolytically cleaved portion of the F₀ precursor. In some embodiments, the F₀ precursor is inactive. In some embodiments, the cleavage of the F₀ precursor forms a disulfide-linked F1+F2 heterodimer. In some embodiments, the cleavage exposes the fusion peptide and produces a mature F protein. In some embodiments, the cleavage occurs at or around a single basic residue. In some embodiments, the cleavage occurs at Arginine 109 of NiV-F protein. In some embodiments, cleavage occurs at Lysine 109 of the Hendra virus F protein.

Table 2A provides non-limiting examples of F proteins. In some embodiments, the N-terminal hydrophobic fusion peptide domain of the F protein molecule or biologically active portion thereof is exposed on the outside of lipid bilayer.

Among different henipavirus species, the sequence and activity of the F protein is highly conserved. For examples, the F protein of NiV and HeV viruses share 89% amino acid sequence identity. Further, in some cases, the henipavirus F proteins exhibit compatibility with G proteins from other species to trigger fusion (Brandel-Tretheway et al. Journal of Virology. 2019. 93(13):e00577-19). In some aspects of the provided viral vector, the F protein is heterologous to the G protein, i.e. the F and G protein or biologically active portions are from different henipavirus species. For example, the F protein is from Hendra virus and the G protein is from Nipah virus. In other aspects, the F protein can be a chimeric F protein containing regions of F proteins from different species of Henipavirus. In some embodiments, switching a region of amino acid residues of the F protein from one species of Henipavirus to another can result in fusion to the G protein of the species comprising the amino acid insertion. (Brandel-Tretheway et al. 2019). In some cases, the chimeric F protein contains an extracellular domain from one henipavirus species and a transmembrane and/or cytoplasmic domain from a different henipavirus species. For example, the F protein contains an extracellular domain of Hendra virus and a transmembrane/cytoplasmic domain of Nipah virus. F protein sequences disclosed herein are predominantly disclosed as expressed sequences including an N-terminal signal sequence. As such N-terminal signal sequences are commonly cleaved co- or post-translationally, the mature protein sequences for all F protein sequences disclosed herein are also contemplated as lacking the N-terminal signal sequence.

TABLE 2A
Non-limiting Examples of F Proteins

| | | | | | SEQ ID |
|---|---|---|---|---|---|
| | Nucleo- | | | | NO |
| | tides | Full | | | (without |
| Genbank | of | Gene | | SEQ | signal |
| ID | CDS | Name | Sequence | ID | sequence) |
| AF017149 | 6618-8258 | gb: AF017149\| | MATQEVRLKCLLCGIIVLVLSLEGLGILH | 1 | 6 |
| | | Organism: | YEKLSKIGLVKGITRKYKIKSNPLTKDIVI | | |
| | | Hendra | KMIPNVSNVSKCTGTVMENYKSRLTGILS | | |
| | | virus\| | PIKGAIELYNNNTHDLVGDVKLAGVVMA | | |
| | | Strain | GIAIGIATAAQITAGVALYEAMKNADNIN | | |
| | | Name: | KLKSSIESTNEAVVKLQETAEKTVYVLTA | | |
| | | UNKNOWN- | LQDYINTNLVPTIDQISCKQTELALDLALS | | |
| | | AF017149\| | KYLSDLLFVFGPNLQDPVSNSMTIQAISQ | | |
| | | Protein | AFGGNYETLLRTLGYATEDFDDLLESDSI | | |
| | | Name: | AGQIVYVDLSSYYIIVRVYFPILTEIQQAY | | |
| | | fusion\|Gene | VQELLPVSFNNDNSEWISIVPNFVLIRNTL | | |
| | | Symbol: | ISNIEVKYCLITKKSVICNQDYATPMTAS | | |
| | | F | VRECLTGSTDKCPRELVVSSHVPRFALSG | | |
| | | | GVLFANCISVTCQCQTTGRAISQSGEQTL | | |
| | | | LMIDNTTCTTVVLGNIIISLGKYLGSINYN | | |
| | | | SESIAVGPPVYTDKVDISSQISSMNQSLQQ | | |
| | | | SKDYIKEAQKILDTVNPSLISMLSMIILYV | | |
| | | | LSIAALCIGLITFISFVIVEKKRGNYSRLDD | | |
| | | | RQVRPVSNGDLYYIGT | | |
| Q9IH63 | | Additional | MVVILDKRCYCNLLILILMISECSVGILHY | 2 | 7 |
| | | in | EKLSKIGLVKGVTRKYKIKSNPLTKDIVIK | | |
| | | cluster: | MIPNVSNMSQCTGSVMENYKTRLNGILT | | |
| | | sp/Q9IH63\| | PIKGALEIYKNNTHDLVGDVRLAGVIMA | | |
| | | FUS_NIPAV | GVAIGIATAAQITAGVALYEAMKNADNI | | |
| | | Fusion | NKLKSSIESTNEAVVKLQETAEKTVYVLT | | |
| | | glycoprotein | ALQDYINTNLVPTIDKISCKQTELSLDLAL | | |
| | | F0 | SKYLSDLLFVFGPNLQDPVSNSMTIQAIS | | |
| | | OS = Nipah | QAFGGNYETLLRTLGYATEDFDDLLESD | | |
| | | virus | SITGQIIYVDLSSYYIIVRVYFPILTEIQQA | | |
| | | | YIQELLPVSFNNDNSEWISIVPNFILVRNT | | |
| | | | LISNIEIGFCLITKRSVICNQDYATPMTNN | | |
| | | | MRECLTGSTEKCPRELVVSSHVPRFALSN | | |
| | | | GVLFANCISVTCQCQTTGRAISQSGEQTL | | |
| | | | LMIDNTTCPTAVLGNVIISLGKYLGSVNY | | |
| | | | NSEGIAIGPPVFTDKVDISSQISSMNQSLQ | | |
| | | | QSKDYIKEAQRLLDTVNPSLISMLSMIILY | | |
| | | | VLSIASLCIGLITFISFIIVEKKRNTYSRLED | | |
| | | | RRVRPTSSGDLYYIGT | | |
| JQ001776 | 6129-8166 | gb: | MSNKRTTVLIIISYTLFYLNNAAIVGFDFD | 3 | 8 |
| | | JQ001776: | KLNKIGVVQGRVLNYKIKGDPMTKDLVL | | |
| | | 6129-8166\| | KFIPNIVNITECVREPLSRYNETVRRLLLPI | | |
| | | Organism: | HNMLGLYLNNTNAKMTGLMIAGVIMGG | | |
| | | Cedar | IAIGIATAAQITAGFALYEAKKNTENIQKL | | |
| | | virus\| | TDSIMKTQDSIDKLTDSVGTSILILNKLQT | | |
| | | Strain | YINNQLVPNLELLSCRQNKIEFDLMLTKY | | |
| | | Name: CG1a\| | LVDLMTVIGPNINNPVNKDMTIQSLSLLF | | |
| | | Protein | DGNYDIMMSELGYTPQDFLDLIESKSITG | | |
| | | Name: | QIIYVDMENLYVVIRTYLPTLIEVPDAQIY | | |
| | | fusion | EFNKITMSSNGGEYLSTIPNFILIRGNYMS | | |
| | | glycoprotein\| | NIDVATCYMTKASVICNQDYSLPMSQNL | | |
| | | Gene | RSCYQGETEYCPVEAVIASHSPRFALTNG | | |
| | | Symbol: | VIFANCINTICRCQDNGKTITQNINQFVSM | | |
| | | F | IDNSTCNDVMVDKFTIKVGKYMGRKDIN | | |
| | | | NINIQIGPQIIIDKVDLSNEINKMNQSLKDS | | |
| | | | IFYLREAKRILDSVNISLISPSVQLFLIIISVL | | |
| | | | SFIILLIIIVYLYCKSKHSYKYNKFIDDPDY | | |
| | | | YNDYKRERINGKASKSNNIYYVGD | | |
| NC_025352 | 5950-8712 | gb: | MALNKNMFSSLFLGYLLVYATTVQSSIH | 4 | 9 |
| | | NC_025352: | YDSLSKVGVIKGLTYNYKIKGSPSTKLM | | |
| | | 5950-8712\| | VVKLIPNIDSVKNCTQKQYDEYKNLVRK | | |
| | | Organism: | ALEPVKMAIDTMLNNVKSGNNKYRFAG | | |
| | | Mojiang | AIMAGVALGVATAATVTAGIALHRSNEN | | |
| | | virus\| | AQAIANMKSAIQNTNEAVKQLQLANKQT | | |
| | | Strain | LAVIDTIRGEINNNIIPVINQLSCDTIGLSV | | |
| | | Name: | GIRLTQYYSEIITAFGPALQNPVNTRITIQA | | |
| | | Tongguan | ISSVFNGNFDELLKIMGYTSGDLYEILHSE | | |
| | | 1\|Protein | LIRGNIIDVDVDAGYIALEIEFPNLTL VPN | | |
| | | Name: | AVVQELMPISYNIDGDEWVTLVPRFVLT | | |
| | | fusion | RTTLLSNIDTSRCTITDSSVICDNDYALPM | | |
| | | protein\| | SHELIGCLQGDTSKCAREKVVSSYVPKFA | | |
| | | Gene | LSDGLVYANCLNTICRCMDTDTPISQSLG | | |
| | | Symbol: | ATVSLLDNKRCSVYQVGDVLISVGSYLG | | |
| | | F | DGEYNADNVELGPPIVIDKIDIGNQLAGI | | |
| | | | NQTLQEAEDYIEKSEEFLKGVNPSIITLGS | | |
| | | | MVVLYIFMILIAIVSVIALVLSIKLTVKGN | | |
| | | | VVRQQFTYTQHVPSMENINYVSH | | |
| NC_025256 | 6865-8853 | gb: | MKKKTDNPTISKRGHNHSRGIKSRALLRE | 5 | 10 |
| | | NC_025256: | TDNYSNGLIVENLVRNCHHPSKNNLNYT | | |
| | | 6865-8853\| | KTQKRDSTIPYRVEERKGHYPKIKHLIDK | | |
| | | Organism: | SYKHIKRGKRRNGHNGNIITIILLLILILKT | | |
| | | Bat | QMSEGAIHYETLSKIGLIKGITREYKVKG | | |
| | | Paramyxovirus | TPSSKDIVIKLIPNVTGLNKCTNISMENYK | | |
| | | Eid_hel/ | EQLDKILIPINNIIELYANSTKSAPGNARFA | | |
| | | GH-M74a/ | GVIIAGVALGVAAAAQITAGIALHEARQN | | |
| | | GHA/2009\| | AERINLLKDSISATNNAVAELQEATGGIV | | |
| | | Strain | NVITGMQDYINTNLVPQIDKLQCSQIKTA | | |
| | | Name: | LDISLSQYYSEILTVFGPNLQNPVTTSMSI | | |
| | | BatPV/ | QAISQSFGGNIDLLLNLLGYTANDLLDLL | | |
| | | Eid_hel/ | ESKSITGQITYINLEHYFMVIRVYYPIMTTI | | |
| | | GH-M74a/G | SNAYVQELIKISFNVDGSEWVSLVPSYILI | | |
| | | HA/2009\| | RNSYLSNIDISECLITKNSVICRHDFAMPM | | |
| | | Protein | SYTLKECLTGDTEKCPREAVVTSYVPRFA | | |
| | | Name: | ISGGVIYANCLSTTCQCYQTGKVIAQDGS | | |
| | | fusion | QTLMMIDNQTCSIVRIEEILISTGKYLGSQ | | |
| | | protein\| | EYNTMHVSVGNPVFTDKLDITSQISNINQ | | |
| | | Gene | SIEQSKFYLDKSKAILDKINLNLIGSVPISI | | |
| | | Symbol: | LFIIAILSLILSIITFVIVMIIVRRYNKYTPLI | | |
| | | F | NSDPSSRRSTIQDVYIIPNPGEHSIRSAARS | | |
| | | | IDRDRD | | |

In some embodiments, the F protein or the biologically active portion thereof is a wild-type Nipah virus F (NiV-F) protein or a Hendra virus F protein or is a functionally active variant or biologically active portion thereof. For instance, in some embodiments, the F protein or the biologically active portion thereof is a wild-type NiV-F protein or a functionally active variant or a biologically active portion thereof.

In some embodiments, the F protein has the sequence of amino acids set forth in SEQ ID NO:1, SEQ ID NO:2, SEQ ID NO:3, SEQ ID NO:4, or SEQ ID NO:5, or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:1, SEQ ID NO:2, SEQ ID NO:3, SEQ ID NO:4, or SEQ ID NO:5, and retains fusogenic activity in conjunction with a G protein, such as a variant NiV-G as provided herein. In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:1, SEQ ID NO:2, SEQ ID NO:3, SEQ ID NO:4, or SEQ ID NO:5.

In particular embodiments, the F protein has the sequence of amino acids set forth in SEQ ID NO:6, SEQ ID NO:7, SEQ ID NO:8, SEQ ID NO:9, or SEQ ID NO:10, or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:6, SEQ ID NO:7, SEQ ID NO:8, SEQ ID NO:9, or SEQ ID NO:1, and retains fusogenic activity in conjunction with a G protein, such as a variant NiV-G as provided herein. In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:6, SEQ ID NO:7, SEQ ID NO:8, SEQ ID NO:9, or SEQ ID NO:1.

Fusogenic activity includes the activity of the F protein in conjunction with a G protein to promote or facilitate fusion of two membrane lumens, such as the lumen of the targeted viral vector having embedded in its lipid bilayer a henipavirus F and G protein, and a cytoplasm of a target cell, e.g. a cell that contains a surface receptor or molecule that is recognized or bound by the targeted envelope protein. In some embodiments, the F protein and G protein are from the same Henipavirus species (e.g. NiV-G and NiV-F). In some embodiments, the F protein and G protein are from different Henipavirus species (e.g. NiV-G and HeV-F). In particular embodiments, the F protein of the functionally active variant or biologically active portion retains the cleavage site cleaved by cathepsin L (e.g. corresponding to the cleavage site between amino acids 109-110 of SEQ ID NO:2).

Reference to retaining fusogenic activity includes activity (in conjunction with a G protein, such as a variant G protein provided herein) that is between at or about 10% and at or about 150% or more of the level or degree of binding of the corresponding wild-type F protein, such as set forth in SEQ ID NO:1, SEQ ID NO:2, SEQ ID NO:3, SEQ ID NO:4, or SEQ ID NO:5, SEQ ID NO:6, SEQ ID NO:7, SEQ ID NO:8, SEQ ID NO:9, or SEQ ID NO:10 or a cathepsin L cleaved from thereof containing an F1 and F2 subunit. In some embodiments, the fusogenic activity is at least or at least about 10% of the level or degree of fusogenic activity of the corresponding wild-type F protein, such as at least or at least about 15% of the level or degree of fusogenic activity of the corresponding wild-type F protein, such as at least or at least about 20% of the level or degree of fusogenic activity of the corresponding wild-type F protein, such as at least or at least about 25% of the level or degree of fusogenic activity of the corresponding wild-type F protein, such as at least or at least about 30% of the level or degree of fusogenic activity of the corresponding wild-type F protein, such as at least or at least about 35% of the level or degree of fusogenic activity of the corresponding wild-type F protein, such as at least or at least about 40% of the level or degree of fusogenic activity of the corresponding wild-type F protein, such as at least or at least about 45% of the level or degree of fusogenic activity of the corresponding wild-type F protein, such as at least or at least about 50% of the level or degree of fusogenic activity of the corresponding wild-type F protein, such as at least or at least about 55% of the level or degree of fusogenic activity of the corresponding wild-type F protein, such as at least or at least about 60% of the level or degree of fusogenic activity of the corresponding wild-type F protein, such as at least or at least about 65% of the level or degree of fusogenic activity of the corresponding wild-type F protein, such as at least or at least about 70% of the level or degree of fusogenic activity of the corresponding wild-type F protein, such as at least or at least about 75% of the level or degree of fusogenic activity of the corresponding wild-type F protein, such as at least or at least about 80% of the level or degree of fusogenic activity of the corresponding wild-type F protein, such as at least or at least about 85% of the level or degree of fusogenic activity of the corresponding wild-type F protein, such as at least or at least about 90% of the level or degree of fusogenic activity of the corresponding wild-type F protein, such as at least or at least about 95% of the level or degree of fusogenic activity of the corresponding wild-type F protein, such as at least or at least about 100% of the level or degree of fusogenic activity of the corresponding wild-type F protein, or such as at least or at least about 120% of the level or degree of fusogenic activity of the corresponding wild-type F protein.

In some embodiments, the F protein is a mutant F protein that is a functionally active fragment or a biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference F protein sequence. In some embodiments, the reference F protein sequence is the wild-type sequence of an F protein or a biologically active portion thereof. In some embodiments, the mutant F protein or the biologically active portion thereof is a mutant of a wild-type Hendra (Hev) virus F protein, a Nipah (NiV) virus F-protein, a Cedar (CedPV) virus F protein, a Mojiang virus F protein or a bat Paramyxovirus F protein. In some embodiments, the wild-type F protein is encoded by a sequence of nucleotides that encodes any one of SEQ ID NO:1, SEQ ID NO:2, SEQ ID NO:3, SEQ ID NO:4, or SEQ ID NO:5, SEQ ID NO:6, SEQ ID NO:7, SEQ ID NO:8, SEQ ID NO:9, or SEQ ID NO:10 or a cathepsin L cleaved from thereof containing an F1 and F2 subunit.

In some embodiments, the mutant F protein is a biologically active portion that is truncated and lacks up to 22 contiguous amino acid residues at or near the C-terminus of the wild-type F protein, such as a wild-type F protein set forth in any one of SEQ ID NO:1, SEQ ID NO:2, SEQ ID NO:3, SEQ ID NO:4, or SEQ ID NO:5, SEQ ID NO:6, SEQ ID NO:7, SEQ ID NO:8, SEQ ID NO:9, or SEQ ID NO:10. In some embodiments, the mutant F protein is truncated and lacks up to 22 contiguous amino acids, such as up to 21, 20, 19, 18, 17, 16, 15, 14, 13, 12, 11, 10, 9, 8, 7, 6, 5, 4, 3, 2, or 1 contiguous amino acids at the C-terminus of the wild-type F protein.

In some embodiments, the NiV-F, of a provided viral vector includes the F0 precursor or a proteolytically cleaved form thereof containing the F1 and F2 subunits, such as resulting following proteolytic cleavage at the cleavage site (e.g. between amino acids corresponding to amino acids between amino acids 10⁹-110 of SEQ ID NO:2) to produce two chains that can be linked by disulfide bond. In some embodiments, the NiV-F, is produced or encoded as an F₀ precursor which then is able to be proteolytically cleaved to result in an F protein containing the F1 and F2 subunit linked by a disulfide bond. Hence, it is understood that reference to a particular sequence (SEQ ID NO) of a NiV-F herein is typically with reference to the F₀ precursor sequence but also is understood to include the proteolytically cleaved form or sequence thereof containing the two cleaved chains, F1 and F2. For instance, the NiV-F, such as a mutant or truncated NiV-F, contains an F1 subunit corresponding to amino acids 110-546 of NiV-F set forth in SEQ ID NO:2 or truncated or mutant sequence thereof, and an F2 corresponding to amino acid residues 27-109 of NiV-F set forth in SEQ ID NO:2.

In some embodiments, the mutant F protein is a biologically active portion that is truncated and lacks up to 22 contiguous amino acid residues at or near the C-terminus of the wild-type NiV-F protein, such as a wild-type NiV-F protein set forth in SEQ ID NO:2 or SEQ ID NO:7. In some embodiments, the mutant F protein is truncated and lacks up to 22 contiguous amino acids, such as up to 21, 20, 19, 18, 17, 16, 15, 14, 13, 12, 11, 10, 9, 8, 7, 6, 5, 4, 3, 2, or 1 contiguous amino acids at the C-terminus of the wild-type NiV-F protein, such as a wild-type NiV-F protein set forth in SEQ ID NO:2 or SEQ ID NO:7. In some embodiments, the mutant F protein contains an F1 subunit and an F2 subunit in which (1) the F1 subunit is truncated and lacks up to 22 contiguous amino acids at or near the C-terminus of the wild-type F1 subunit, such as lacks up to 22 contiguous amino acids at or near the C-terminus of the wild-type F1 subunit corresponding to amino acids 110-546 of NiV-F set forth in SEQ ID NO:2, and (2) the F2 subunit has the sequence corresponding to amino acid residues 27-10⁹ of NiV-F set forth in SEQ ID NO:2.

In some embodiments, the F protein is a mutant NiV-F protein that is a biologically active portion thereof that comprises a 22 amino acid truncation at or near the C-terminus of the wild-type NiV-F protein (SEQ ID NO:2 or SEQ ID NO:7). In some embodiments, the NiV-F protein is encoded by a nucleotide sequence that encodes the sequence set forth in SEQ ID NO: 11. In some embodiments, the NiV-F protein has at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 11. In particular embodiments, the F protein is a mutant NiV-F protein that has the sequence of amino acids set forth in SEQ ID NO:12. In some embodiments, the NiV-F protein has a sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 12. In some embodiments, the F protein molecule or biologically active portion thereof comprises the sequence set forth in SEQ ID NO: 12.

In some embodiments, the mutant F protein contains an F1 subunit and an F2 subunit in which (1) the F1 subunit is set forth as amino acids 110-524 of SEQ ID NO:11, and (2) the F2 subunit is set forth as amino acids 27-10⁹ of SEQ ID NO:11.

In some embodiments, the mutant F protein contains an F1 subunit and an F2 subunit in which (1) the F1 subunit is set forth as amino acids 84-498 of SEQ ID NO:12, and (2) the F2 subunit is set forth as amino acids 1-83 of SEQ ID NO:12.

2. Re-Targeted Fusogens (e.g. Re-Targeted G Proteins)

In some embodiments, the fusogen (e.g. F or G protein) is a targeted envelope protein that contains a vector-surface targeting moiety. In some embodiments, the vector-surface targeting moiety binds a target ligand. In some embodiments, the target ligand can be expressed on a target cell of interest, such as a target cell present as a leukocyte component. In some aspects, a fusogen can be retargeted to display altered tropism. In some embodiments, the binding confers re-targeted binding compared to the binding of a wild-type surface glycoprotein protein in which a new or different binding activity is conferred.

In some embodiments, a G protein (such as NiV-G) is further attached or linked to a binding domain that binds to a target molecule, such as a cell surface marker. For instance, provided in some aspects is a targeted viral vector (e.g. targeted lentiviral vector) that includes a re-targeted G protein containing any of the provided G proteins attached to a binding domain, in which the re-targeted G protein is exposed on the surface of the targeted viral vector (e.g. targeted lentiviral vector). In particular embodiments, the fusogen (e.g. G protein) is mutated to reduce binding for the native binding partner of the fusogen. In some embodiments, the fusogen is or contains a mutant G protein or a biologically active portion thereof that is a mutant of wild-type NiV-G and exhibits reduced binding to one or both of the native binding partners Ephrin B2 or Ephrin B3, including any as described above. In particular embodiments, the binding confers re-targeted binding compared to the binding of a wild-type G protein in which a new or different binding activity is conferred.

In some embodiments, the targeted envelope protein contains a G protein provided herein.

In some embodiments the G protein is any as described above, including NiV-G proteins with cytoplasmic domain modifications, truncated NiV-G cytoplasmic tails, or modified NiV-G cytoplasmic tails.

In some embodiments, the binding domain can be any agent that binds to a cell surface molecule on a target cells. In some embodiments, protein fusogens may be re-targeted by covalently conjugating a targeting-moiety to the fusion protein. In some embodiments, the fusogen and targeting moiety are covalently conjugated by expression of a chimeric protein comprising the fusogen linked to the targeting moiety. In some embodiments, a target includes any peptide (e.g. a receptor) that is displayed on a target cell. In some embodiments, the target is expressed at higher levels on a target cell than non-target cells. In some embodiments, a single-chain variable fragment (scFv) can be conjugated to fusogens to redirect fusion activity towards cells that display the scFv binding target (doi:10.1038/nbt1060, DOI 10.1182/blood-2012-11-468579, doi:10.1038/nmeth.1514, doi:10.1006/mthe.2002.0550, HUMAN GENE THERAPY 11:817-826, doi:10.1038/nbt942, doi:10.1371/journal.pone.0026381, DOI 10.1186/s12896-015-0142-z). In some embodiments, designed ankyrin repeat proteins (DARPin) can be conjugated to fusogens to redirect fusion activity towards cells that display the DARPin binding target (doi:10.1038/mt.2013.16, doi:10.1038/mt.2010.298, doi: 10.4049/jimmunol.1500956), as well as combinations of different DARPins (doi:10.1038/mto.2016.3). In some embodiments, receptor ligands and antigens can be conjugated to fusogens to redirect fusion activity towards cells that display the target receptor (DOI: 10.1089/hgtb.2012.054, DOI: 10.1128/JVI.76.7.3558-3563.2002). In some embodiments, a targeting protein can also include an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, protein fusogens may be re-targeted by non-covalently conjugating a targeting moiety to the fusion protein or targeting protein (e.g. the hemagglutinin protein). In some embodiments, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi:10.1038/nm1192). In some embodiments, altered and non-altered fusogens may be displayed on the same retroviral vector or VLP (doi: 10.1016/j.biomaterials.2014.01.051).

In some embodiments, a targeting moiety comprises a humanized antibody molecule, intact IgA, IgG, IgE or IgM antibody; bi- or multi-specific antibody (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s.

In some embodiments, the targeting moiety is a binding domain that can be an antibody or an antibody portion or fragment. In some embodiments, the binding domain is a single domain antibody (sdAb). In some embodiments, the binding domain is a single chain variable fragment (scFv). In some examples, the binding domain can be linked directly or indirectly to the G protein (e.g. NiV-G or a biologically active portion). In particular embodiments, the binding domain is linked to the C-terminus (C-terminal amino acid) of the G protein or the biologically active portion thereof. The linkage can be via a peptide linker, such as a flexible peptide linker.

The binding domain may be modulated to have different binding strengths. For example, scFvs and antibodies with various binding strengths may be used to alter the fusion activity of the chimeric attachment proteins towards cells that display high or low amounts of the target antigen. For example DARPins with different affinities may be used to alter the fusion activity towards cells that display high or low amounts of the target antigen. Binding domains may also be modulated to target different regions on the target ligand, which will affect the fusion rate with cells displaying the target.

The binding domain may comprise a humanized antibody molecule, intact IgA, IgG, IgE or IgM antibody; bi- or multi-specific antibody (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. A targeting moiety can also include an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs).

In some embodiments, the binding domain is a single chain molecule. In some embodiments, the binding domain is a single domain antibody. In some embodiments, the binding domain is a single chain variable fragment. In particular embodiments, the binding domain contains an antibody variable sequence (s) that is human or humanized.

In some embodiments, the binding domain is a single domain antibody. In some embodiments, the single domain antibody can be human or humanized. In some embodiments, the single domain antibody or portion thereof is naturally occurring. In some embodiments, the single domain antibody or portion thereof is synthetic.

In some embodiments, the single domain antibodies are antibodies whose complementary determining regions are part of a single domain polypeptide. In some embodiments, the single domain antibody is a heavy chain only antibody variable domain. In some embodiments, the single domain antibody does not include light chains.

In some embodiments, the heavy chain antibody devoid of light chains is referred to as VHH. In some embodiments, the single domain antibody antibodies have a molecular weight of 12-15 kDa. In some embodiments, the single domain antibody antibodies include camelid antibodies or shark antibodies. In some embodiments, the single domain antibody molecule is derived from antibodies raised in Camelidae species, for example in camel, llama, dromedary, alpaca, vicuna and guanaco. In some embodiments, the single domain antibody is referred to as immunoglobulin new antigen receptors (IgNARs) and is derived from cartilaginous fishes. In some embodiments, the single domain antibody is generated by splitting dimeric variable domains of human or mouse IgG into monomers and camelizing critical residues.

In some embodiments, the single domain antibody can be generated from phage display libraries. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, single domain antibodies a library of human single domain antibodies is synthetically generated by introducing diversity into one or more scaffolds.

In some embodiments, the C-terminus of the binding domain is attached to the C-terminus of the G protein or biologically active portion thereof. In some embodiments, the N-terminus of the binding domain is exposed on the exterior surface of the lipid bilayer. In some embodiments, the N-terminus of the binding domain binds to a cell surface molecule of a target cell. In some embodiments, the binding domain specifically binds to a cell surface molecule present on a target cell. In some embodiments, the cell surface molecule is a protein, glycan, lipid or low molecular weight molecule. In some embodiments, the binding domain is one of any binding domains as described above.

In embodiments, the re-targeted fusogen binds a cell surface marker on the target cell, e.g., a protein, glycoprotein, receptor, cell surface ligand, agonist, lipid, sugar, class I transmembrane protein, class II transmembrane protein, or class III transmembrane protein. In some embodiments, a binding domain (e.g. sdAb or one of any binding domains as described herein) binds to a cell surface antigen of a cell. In some embodiments, a cell surface antigen is characteristic of one type of cell. In some embodiments, a cell surface antigen is characteristic of more than one type of cell.

In some embodiments, the cell surface molecule of a target cell is an antigen or portion thereof. In some embodiments, the single domain antibody or portion thereof is an antibody having a single monomeric domain antigen binding/recognition domain that is able to bind selectively to a specific antigen. In some embodiments, the single domain antibody binds an antigen present on a target cell.

Exemplary target cells include cells present in a blood sample from a subject. In some embodiments, the cells include a leukocyte component. In some embodiments, the target cells include polymorphonuclear cells (also known as PMN, PML, PMNL, or granulocytes), In some embodiments, the target cells include lymphocytes, monoctyes, macrophages, dendritic cells, natural killer cells, T cells (e.g. CD4 or CD8 T cells including cytotoxic T lymphocytes) or B cells. In some embodiments, the target cells include hematopoietic stem cells (HSCs).

In some embodiments, the target cell is a CD3+ T cell, a CD4+ T cell, a CD8+ T cell.

In some embodiments, the target cell is an antigen presenting cell, an MHC class II+ cell, a professional antigen presenting cell, an atypical antigen presenting cell, a macrophage, a dendritic cell, a myeloid dendritic cell, a plasmacyteoid dendritic cell, a CD11c+ cell, a CD11b+ cell, or a B cell.

In some embodiments, the binding domain (e.g. sdAb) variable domain binds a cell surface molecule or antigen. In some embodiments, the cell surface molecule is ASGR1, ASGR2, TM4SF5, CD3, CD8, CD4, or low density lipoprotein receptor (LDL-R). In some embodiments, the cell surface molecule is ASGR1. In some embodiments, the cell surface molecule is ASGR2. In some embodiments, the cell surface molecule is TM4SF5. In some embodiments, the cell surface molecule is CD3. In some embodiments, the cell surface molecule is CD8. In some embodiments, the cell surface molecule is CD4. In some embodiments, the cell surface molecule is LDL-R.

The viral vectors disclosed herein include one or more CD4 binding agents. For example, a CD4 binding agent may be fused to or incorporated in a protein fusogen or viral envelope protein. In another embodiment, a CD4 binding agent may be incorporated into the viral envelope via fusion with a transmembrane domain.

Exemplary CD4 binding agents include antibodies and fragments thereof (e.g., scFv, VHH) that bind to CD4. Such antibodies may be derived from any species, and may be for example, mouse, rabbit, human, humanized, or camelid antibodies. Exemplary antibodies include ibalizumab, zanolimumab, tregalizumab, priliximab, cedelizumab, clenoliximab, keliximab, and anti-CD4 antibodies disclosed in WO2002102853, WO2004083247, WO2004067554, WO2007109052, WO2008134046, WO2010074266, WO2012113348, WO2013188870, WO2017104735, WO2018035001, WO2018170096, WO2019203497, WO2019236684, WO2020228824, U.S. Pat. Nos. 5,871,732, 7,338,658, 7,722,873, 8,399,621, 8,911,728, 9,005,963,U.S. Pat. Nos. 9,587,022, 9,745,552, U.S. provisional application No. 63/326,269, U.S. provisional application No. 63/341,681; as well as antibodies B486A1, RPA-T4, CE9.1 (Novus Biologicals); GK1.5, RM4-5, RPA-T4, OKT4, 4SM95, S3.5, N1UG0 (ThermoFisher); GTX50984, ST0488, 10B5, EP204 (GeneTex); GK1.3, 5A8, 10C12, W3/25, 8A5, 13B8.2, 6G5 (Absolute Antibody); VIT4, M-T466, M-T321, REA623, (Miltenyi); MEM115, MT310 (Enzo Life Sciences); H129.19, 5B4, 6A17, 18-46, A-1, C-1, OX68 (Santa Cruz); EP204, D2E6M (Cell Signaling Technology). Other exemplary binding agents include designed ankyrin repeat proteins (DARPins) (e.g., the anti-CD4 DARPin disclosed in WO2017182585) and binding agents based on fibronectin type III (Fn3) scaffolds. Each of U.S. Pat. No. 9,005,963, U.S. provisional application No. 63/326,269, and U.S. provisional application No. 63/341,681 is incorporated by reference herein in its entirety.

In some embodiments, protein fusogens or viral envelope proteins may be re-targeted by mutating amino acid residues in a fusion protein or a targeting protein (e.g. the hemagglutinin (H) protein or G protein). In particular embodiments, the fusogen (e.g. G protein) is mutated to reduce binding for the native binding partner of the fusogen. In some embodiments, the fusogen is or contains a mutant G protein or a biologically active portion thereof that is a mutant of wild-type Niv-G and exhibits reduced binding to one or both of the native binding partners Ephrin B2 or Ephrin B3, including any as described above. Thus, in some aspects, a fusogen can be retargeted to display altered tropism. In some embodiments, the binding confers re-targeted binding compared to the binding of a wild-type surface glycoprotein protein in which a new or different binding activity is conferred. In particular embodiments, the binding confers re-targeted binding compared to the binding of a wild-type G protein in which a new or different binding activity is conferred. In some embodiments the fusogen is randomly mutated. In some embodiments the fusogen is rationally mutated. In some embodiments the fusogen is subjected to directed evolution. In some embodiments the fusogen is truncated and only a subset of the peptide is used in the viral vector. In some embodiments, amino acid residues in the measles hemagglutinin protein may be mutated to alter the binding properties of the protein, redirecting fusion (doi:10.1038/nbt942, Molecular Therapy vol. 16 no. 8, 1427-1436 August 2008, doi:10.1038/nbt1060, DOI: 10.1128/JVI.76.7.3558-3563.2002, DOI: 10.1128/JVI.75.17.8016-8020.2001, doi: 10.1073pnas.0604993103).

In some embodiments, protein fusogens may be re-targeted by covalently conjugating a CD4 binding agent to the fusion protein or targeting protein (e.g. the attachment or hemagglutinin protein). In some embodiments, the fusogen and CD4 binding agent are covalently conjugated by expression of a chimeric protein comprising the fusogen linked to the CD4 binding agent. In some embodiments, a single-chain variable fragment (scFv) can be conjugated to fusogens to redirect fusion activity towards cells that display the scFv binding target (doi:10.1038/nbt1060, DOI 10.1182/blood-2012-11-468579, doi:10.1038/nmeth.1514, doi:10.1006/mthe.2002.0550, HUMAN GENE THERAPY 11:817-826, doi:10.1038/nbt942, doi:10.1371/journal.pone.0026381, DOI 10.1186/s12896-015-0142-z). In some embodiments, designed ankyrin repeat proteins (DARPin) can be conjugated to fusogens to redirect fusion activity towards cells that display the DARPin binding target (doi:10.1038/mt.2013.16, doi:10.1038/mt.2010.298, doi: 10.4049/jimmunol.1500956), as well as combinations of different DARPins (doi:10.1038/mto.2016.3). In some embodiments, receptor ligands and antigens can be conjugated to fusogens to redirect fusion activity towards cells that display the target receptor (DOI: 10.1089/hgtb.2012.054, DOI: 10.1128/JVI.76.7.3558-3563.2002). In some embodiments, a targeting protein can also include an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, protein fusogens may be re-targeted by non-covalently conjugating a CD4 binding agent to the fusion protein or targeting protein (e.g. the hemagglutinin protein). In some embodiments, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi:10.1038/nm1192). In some embodiments, altered and non-altered fusogens may be displayed on the same retroviral vector or VLP (doi: 10.1016/j.biomaterials.2014.01.051).

In some embodiments, a CD4 binding agent comprises a humanized antibody molecule, intact IgA, IgG, IgE or IgM antibody; bi- or multi-specific antibody (e.g., Zybodies®, etc); antibody fragments such as Fab fragments, Fab′ fragments, F(ab′)₂ fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); camelid antibodies; masked antibodies (e.g., Probodies®); Small Modular ImmunoPharmaceuticals (“SMIPs™”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s.

In some embodiments, the CD4 binding agent is a peptide. In some embodiments, the CD4 binding agent is an antibody, such as a single-chain variable fragment (scFv). In some embodiments, the CD4 binding agent is an antibody, such as a single domain antibody. In some embodiments, the antibody can be human or humanized. In some embodiments, the CD4 binding agent is a VHH. In some embodiments, the antibody or portion thereof is naturally occurring. In some embodiments, the antibody or portion thereof is synthetic.

In some embodiments, the CD4 binding agent comprises a CDR-H1, a CDR-H2, and a CDR-H3 comprising the amino acid sequence set forth in SEQ ID NO: 133, 134, and 135, respectively. In some embodiments, the CD4 binding agent comprises a CDR-L1, a CDR-L2, and a CDR-L3 comprising the amino acid sequence set forth in SEQ ID NO: 136, 137, and 138, respectively. In some embodiments, the CD4 binding agent comprises a CDR-H1, a CDR-H2, and a CDR-H3 comprising the amino acid sequence set forth in SEQ ID NO: 133, 134, and 135, respectively; and a CDR-L1, a CDR-L2, and a CDR-L3 comprising the amino acid sequence set forth in SEQ ID NO: 136, 137, and 138, respectively. In some embodiments, the CD4 binding agent comprises a CDR-H1, a CDR-H2, and a CDR-H3 comprising the amino acid sequence set forth in SEQ ID NO: 191, 192, and 193, respectively. In some embodiments, the CD4 binding agent comprises a CDR-L1, a CDR-L2, and a CDR-L3 comprising the amino acid sequence set forth in SEQ ID NO: 194, 195, and 138, respectively. In some embodiments, the CD4 binding agent comprises a CDR-H1, a CDR-H2, and a CDR-H3 comprising the amino acid sequence set forth in SEQ ID NO: 191, 192, and 193, respectively; and a CDR-L1, a CDR-L2, and a CDR-L3 comprising the amino acid sequence set forth in SEQ ID NO: 194, 195, and 138, respectively. In some embodiments, the CD4 binding agent comprises a CDR-H1, a CDR-H2, and a CDR-H3 comprising the amino acid sequence set forth in SEQ ID NO: 196, 197, and 193, respectively. In some embodiments, the CD4 binding agent comprises a CDR-L1, a CDR-L2, and a CDR-L3 comprising the amino acid sequence set forth in SEQ ID NO: 194, 195, and 138, respectively. In some embodiments, the CD4 binding agent comprises a CDR-H1, a CDR-H2, and a CDR-H3 comprising the amino acid sequence set forth in SEQ ID NO: 196, 197, and 193, respectively; and a CDR-L1, a CDR-L2, and a CDR-L3 comprising the amino acid sequence set forth in SEQ ID NO: 194, 195, and 138, respectively. In some embodiments, the CD4 binding agent comprises a heavy chain variable region (VH) comprising the amino acid sequence set forth in SEQ ID NO:139. In some embodiments, the CD4 binding agent comprises a light chain variable region (VL) comprising the amino acid sequence set forth in SEQ ID NO:140. In some embodiments, the CD4 binding agent comprises a heavy chain variable region (VH) comprising the amino acid sequence set forth in SEQ ID NO:139; and a light chain variable region (VL) comprising the amino acid sequence set forth in SEQ ID NO:140. In some embodiments, the VH and VL are joined by a linker. In some embodiments, the linker comprises the amino acid sequence set forth in SEQ ID NO:143. In some embodiments, the CD4 binding agent comprises the amino acid sequence set forth in SEQ ID NO:141.

In some embodiments, the CD4 binding agent comprises a CDR-H1, a CDR-H2, and a CDR-H3 comprising the amino acid sequence set forth in SEQ ID NO: 142, 143, and 144, respectively. In some embodiments, the CD4 binding agent comprises a CDR-L1, a CDR-L2, and a CDR-L3 comprising the amino acid sequence set forth in SEQ ID NO: 145, 146, and 147, respectively. In some embodiments, the CD4 binding agent comprises a CDR-H1, a CDR-H2, and a CDR-H3 comprising the amino acid sequence set forth in SEQ ID NO: 142, 143, and 144, respectively; and a CDR-L1, a CDR-L2, and a CDR-L3 comprising the amino acid sequence set forth in SEQ ID NO: 145, 146, and 147, respectively. In some embodiments, the CD4 binding agent comprises a CDR-H1, a CDR-H2, and a CDR-H3 comprising the amino acid sequence set forth in SEQ ID NO: 198, 199, and 200, respectively. In some embodiments, the CD4 binding agent comprises a CDR-L1, a CDR-L2, and a CDR-L3 comprising the amino acid sequence set forth in SEQ ID NO: 201, 202, and 147, respectively. In some embodiments, the CD4 binding agent comprises a CDR-H1, a CDR-H2, and a CDR-H3 comprising the amino acid sequence set forth in SEQ ID NO: 198, 199, and 200, respectively; and a CDR-L1, a CDR-L2, and a CDR-L3 comprising the amino acid sequence set forth in SEQ ID NO: 201, 202, and 147, respectively. In some embodiments, the CD4 binding agent comprises a CDR-H1, a CDR-H2, and a CDR-H3 comprising the amino acid sequence set forth in SEQ ID NO: 203, 204, and 200, respectively. In some embodiments, the CD4 binding agent comprises a CDR-L1, a CDR-L2, and a CDR-L3 comprising the amino acid sequence set forth in SEQ ID NO: 201, 202, and 147, respectively. In some embodiments, the CD4 binding agent comprises a CDR-H1, a CDR-H2, and a CDR-H3 comprising the amino acid sequence set forth in SEQ ID NO: 203, 204, and 200, respectively; and a CDR-L1, a CDR-L2, and a CDR-L3 comprising the amino acid sequence set forth in SEQ ID NO: 201, 202, and 147, respectively. In some embodiments, the CD4 binding agent comprises a heavy chain variable region (VH) comprising the amino acid sequence set forth in SEQ ID NO:148. In some embodiments, the CD4 binding agent comprises a light chain variable region (VL) comprising the amino acid sequence set forth in SEQ ID NO:149. In some embodiments, the CD4 binding agent comprises a heavy chain variable region (VH) comprising the amino acid sequence set forth in SEQ ID NO:148; and a light chain variable region (VL) comprising the amino acid sequence set forth in SEQ ID NO:149. In some embodiments, the VH and VL are joined by a linker. In some embodiments, the linker comprises the amino acid sequence set forth in SEQ ID NO:143. In some embodiments, the CD4 binding agent comprises the amino acid sequence set forth in SEQ ID NO:150.

In some embodiments, the CD4 binding agent comprises a CDR-H1, a CDR-H2, and a CDR-H3 comprising the amino acid sequence set forth in SEQ ID NO: 151, 152, and 153, respectively. In some embodiments, the CD4 binding agent comprises a CDR-L1, a CDR-L2, and a CDR-L3 comprising the amino acid sequence set forth in SEQ ID NO: 154, 155, and 156, respectively. In some embodiments, the CD4 binding agent comprises a CDR-H1, a CDR-H2, and a CDR-H3 comprising the amino acid sequence set forth in SEQ ID NO: 151, 152, and 153, respectively; and a CDR-L1, a CDR-L2, and a CDR-L3 comprising the amino acid sequence set forth in SEQ ID NO: 154, 155, and 156, respectively. In some embodiments, the CD4 binding agent comprises a CDR-H1, a CDR-H2, and a CDR-H3 comprising the amino acid sequence set forth in SEQ ID NO: 205, 206, 207, respectively. In some embodiments, the CD4 binding agent comprises a CDR-L1, a CDR-L2, and a CDR-L3 comprising the amino acid sequence set forth in SEQ ID NO: 208, 209, and 156, respectively. In some embodiments, the CD4 binding agent comprises a CDR-H1, a CDR-H2, and a CDR-H3 comprising the amino acid sequence set forth in SEQ ID NO: 205, 206, 207, respectively; and a CDR-L1, a CDR-L2, and a CDR-L3 comprising the amino acid sequence set forth in SEQ ID NO: 208, 209, and 156, respectively. In some embodiments, the CD4 binding agent comprises a CDR-H1, a CDR-H2, and a CDR-H3 comprising the amino acid sequence set forth in SEQ ID NO: 210, 211, 207, respectively. In some embodiments, the CD4 binding agent comprises a CDR-L1, a CDR-L2, and a CDR-L3 comprising the amino acid sequence set forth in SEQ ID NO: 208, 209, and 156, respectively. In some embodiments, the CD4 binding agent comprises a CDR-H1, a CDR-H2, and a CDR-H3 comprising the amino acid sequence set forth in SEQ ID NO: 210, 211, 207, respectively; and a CDR-L1, a CDR-L2, and a CDR-L3 comprising the amino acid sequence set forth in SEQ ID NO: 208, 209, and 156, respectively. In some embodiments, the CD4 binding agent comprises a heavy chain variable region (VH) comprising the amino acid sequence set forth in SEQ ID NO:157. In some embodiments, the CD4 binding agent comprises a light chain variable region (VL) comprising the amino acid sequence set forth in SEQ ID NO:158. In some embodiments, the CD4 binding agent comprises a heavy chain variable region (VH) comprising the amino acid sequence set forth in SEQ ID NO:157; and a light chain variable region (VL) comprising the amino acid sequence set forth in SEQ ID NO:158. In some embodiments, the VH and VL are joined by a linker. In some embodiments, the linker comprises the amino acid sequence set forth in SEQ ID NO:143. In some embodiments, the CD4 binding agent comprises the amino acid sequence set forth in SEQ ID NO:159.

In some embodiments, the CD4 binding agent comprises a CDR-H1, a CDR-H2, and a CDR-H3 comprising the amino acid sequence set forth in SEQ ID NO: 160, 161, and 162, respectively. In some embodiments, the CD4 binding agent comprises a CDR-L1, a CDR-L2, and a CDR-L3 comprising the amino acid sequence set forth in SEQ ID NO: 163, 164, and 165, respectively. In some embodiments, the CD4 binding agent comprises a CDR-H1, a CDR-H2, and a CDR-H3 comprising the amino acid sequence set forth in SEQ ID NO: 160, 161, and 162, respectively; and a CDR-L1, a CDR-L2, and a CDR-L3 comprising the amino acid sequence set forth in SEQ ID NO: 163, 164, and 165, respectively. In some embodiments, the CD4 binding agent comprises a CDR-H1, a CDR-H2, and a CDR-H3 comprising the amino acid sequence set forth in SEQ ID NO: 212, 213, 214, respectively. In some embodiments, the CD4 binding agent comprises a CDR-L1, a CDR-L2, and a CDR-L3 comprising the amino acid sequence set forth in SEQ ID NO: 215, 216, and 165, respectively. In some embodiments, the CD4 binding agent comprises a CDR-H1, a CDR-H2, and a CDR-H3 comprising the amino acid sequence set forth in SEQ ID NO: 212, 213, 214, respectively; and a CDR-L1, a CDR-L2, and a CDR-L3 comprising the amino acid sequence set forth in SEQ ID NO: 215, 216, and 165, respectively. In some embodiments, the CD4 binding agent comprises a CDR-H1, a CDR-H2, and a CDR-H3 comprising the amino acid sequence set forth in SEQ ID NO: 217, 218, 214, respectively. In some embodiments, the CD4 binding agent comprises a CDR-L1, a CDR-L2, and a CDR-L3 comprising the amino acid sequence set forth in SEQ ID NO: 215, 216, and 165, respectively. In some embodiments, the CD4 binding agent comprises a CDR-H1, a CDR-H2, and a CDR-H3 comprising the amino acid sequence set forth in SEQ ID NO: 217, 218, 214, respectively; and a CDR-L1, a CDR-L2, and a CDR-L3 comprising the amino acid sequence set forth in SEQ ID NO: 215, 216, and 165, respectively. In some embodiments, the CD4 binding agent comprises a heavy chain variable region (VH) comprising the amino acid sequence set forth in SEQ ID NO:166. In some embodiments, the CD4 binding agent comprises a light chain variable region (VL) comprising the amino acid sequence set forth in SEQ ID NO:167. In some embodiments, the CD4 binding agent comprises a heavy chain variable region (VH) comprising the amino acid sequence set forth in SEQ ID NO:166; and a light chain variable region (VL) comprising the amino acid sequence set forth in SEQ ID NO:167. In some embodiments, the VH and VL are joined by a linker. In some embodiments, the linker comprises the amino acid sequence set forth in SEQ ID NO:143. In some embodiments, the CD4 binding agent comprises the amino acid sequence set forth in SEQ ID NO:168.

In some embodiments, the CD4 binding agent comprises a CDR-H1, a CDR-H2, and a CDR-H3 comprising the amino acid sequence set forth in SEQ ID NO: 169, 170, and 171, respectively. In some embodiments, the CD4 binding agent comprises a CDR-L1, a CDR-L2, and a CDR-L3 comprising the amino acid sequence set forth in SEQ ID NO: 172, 155, and 173, respectively. In some embodiments, the CD4 binding agent comprises a CDR-H1, a CDR-H2, and a CDR-H3 comprising the amino acid sequence set forth in SEQ ID NO: 169, 170, and 171, respectively; and a CDR-L1, a CDR-L2, and a CDR-L3 comprising the amino acid sequence set forth in SEQ ID NO: 172, 155, and 173, respectively. In some embodiments, the CD4 binding agent comprises a CDR-H1, a CDR-H2, and a CDR-H3 comprising the amino acid sequence set forth in SEQ ID NO: 219, 220, and 221, respectively. In some embodiments, the CD4 binding agent comprises a CDR-L1, a CDR-L2, and a CDR-L3 comprising the amino acid sequence set forth in SEQ ID NO: 222, 223, and 173, respectively. In some embodiments, the CD4 binding agent comprises a CDR-H1, a CDR-H2, and a CDR-H3 comprising the amino acid sequence set forth in SEQ ID NO: 219, 220, and 221, respectively; and a CDR-L1, a CDR-L2, and a CDR-L3 comprising the amino acid sequence set forth in SEQ ID NO: 222, 223, and 173, respectively. In some embodiments, the CD4 binding agent comprises a CDR-H1, a CDR-H2, and a CDR-H3 comprising the amino acid sequence set forth in SEQ ID NO: 224, 225, and 221, respectively. In some embodiments, the CD4 binding agent comprises a CDR-L1, a CDR-L2, and a CDR-L3 comprising the amino acid sequence set forth in SEQ ID NO: 222, 223, and 173, respectively. In some embodiments, the CD4 binding agent comprises a CDR-H1, a CDR-H2, and a CDR-H3 comprising the amino acid sequence set forth in SEQ ID NO: 224, 225, and 221, respectively; and a CDR-L1, a CDR-L2, and a CDR-L3 comprising the amino acid sequence set forth in SEQ ID NO: 222, 223, and 173, respectively. In some embodiments, the CD4 binding agent comprises a heavy chain variable region (VH) comprising the amino acid sequence set forth in SEQ ID NO:174. In some embodiments, the CD4 binding agent comprises a light chain variable region (VL) comprising the amino acid sequence set forth in SEQ ID NO:175. In some embodiments, the CD4 binding agent comprises a heavy chain variable region (VH) comprising the amino acid sequence set forth in SEQ ID NO:174; and a light chain variable region (VL) comprising the amino acid sequence set forth in SEQ ID NO:175. In some embodiments, the VH and VL are joined by a linker. In some embodiments, the linker comprises the amino acid sequence set forth in SEQ ID NO:143. In some embodiments, the CD4 binding agent comprises the amino acid sequence set forth in SEQ ID NO:176.

In some embodiments, the CD4 binding agent comprises a CDR-H1, a CDR-H2, and a CDR-H3 comprising the amino acid sequence set forth in SEQ ID NO: 177, 178, and 179, respectively. In some embodiments, the CD4 binding agent comprises a CDR-L1, a CDR-L2, and a CDR-L3 comprising the amino acid sequence set forth in SEQ ID NO: 180, 181, and 182, respectively. In some embodiments, the CD4 binding agent comprises a CDR-H1, a CDR-H2, and a CDR-H3 comprising the amino acid sequence set forth in SEQ ID NO: 177, 178, and 179, respectively; and a CDR-L1, a CDR-L2, and a CDR-L3 comprising the amino acid sequence set forth in SEQ ID NO: 180, 181, and 182, respectively. In some embodiments, the CD4 binding agent comprises a CDR-H1, a CDR-H2, and a CDR-H3 comprising the amino acid sequence set forth in SEQ ID NO: 226, 227, and 228, respectively. In some embodiments, the CD4 binding agent comprises a CDR-L1, a CDR-L2, and a CDR-L3 comprising the amino acid sequence set forth in SEQ ID NO: 229, 230, and 182, respectively. In some embodiments, the CD4 binding agent comprises a CDR-H1, a CDR-H2, and a CDR-H3 comprising the amino acid sequence set forth in SEQ ID NO: 226, 227, and 228, respectively; and a CDR-L1, a CDR-L2, and a CDR-L3 comprising the amino acid sequence set forth in SEQ ID NO: 229, 230, and 182, respectively. In some embodiments, the CD4 binding agent comprises a CDR-H1, a CDR-H2, and a CDR-H3 comprising the amino acid sequence set forth in SEQ ID NO: 231, 232, and 228, respectively. In some embodiments, the CD4 binding agent comprises a CDR-L1, a CDR-L2, and a CDR-L3 comprising the amino acid sequence set forth in SEQ ID NO: 229, 230, and 182, respectively. In some embodiments, the CD4 binding agent comprises a CDR-H1, a CDR-H2, and a CDR-H3 comprising the amino acid sequence set forth in SEQ ID NO: 231, 232, and 228, respectively; and a CDR-L1, a CDR-L2, and a CDR-L3 comprising the amino acid sequence set forth in SEQ ID NO: 229, 230, and 182, respectively. In some embodiments, the CD4 binding agent comprises a heavy chain variable region (VH) comprising the amino acid sequence set forth in SEQ ID NO:183. In some embodiments, the CD4 binding agent comprises a light chain variable region (VL) comprising the amino acid sequence set forth in SEQ ID NO:184. In some embodiments, the CD4 binding agent comprises a heavy chain variable region (VH) comprising the amino acid sequence set forth in SEQ ID NO:183; and a light chain variable region (VL) comprising the amino acid sequence set forth in SEQ ID NO:184. In some embodiments, the VH and VL are joined by a linker. In some embodiments, the linker comprises the amino acid sequence set forth in SEQ ID NO:143. In some embodiments, the CD4 binding agent comprises the amino acid sequence set forth in SEQ ID NO:185.

In some embodiments, the CD4 binding agent is an antibody, such as a single domain antibody. In some embodiments, the antibody can be human or humanized. In some embodiments, the CD4 binding agent is a VHH. In some embodiments, the CD4 binding agent comprises a CDR-H1, a CDR-H2, and a CDR-H3 comprising the amino acid sequence set forth in SEQ ID NO: 129, 130, and 131, respectively. In some embodiments, the CD4 binding agent comprises a CDR-H1, a CDR-H2, and a CDR-H3 comprising the amino acid sequence set forth in SEQ ID NO: 186, 187, and 188, respectively. In some embodiments, the CD4 binding agent comprises a CDR-H1, a CDR-H2, and a CDR-H3 comprising the amino acid sequence set forth in SEQ ID NO: 189, 190, and 188, respectively. In some embodiments, the CD4 binding agent comprises the amino acid sequence set forth in SEQ ID NO:132.

In some embodiments, the antibody can be generated from phage display libraries to have specificity for a desired target ligand. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, a library of human single domain antibodies is synthetically generated by introducing diversity into one or more scaffolds.

In some embodiments, the C-terminus of the CD4 binding agent is attached to the C-terminus of the G protein (e.g., fusogen) or biologically active portion thereof. In some embodiments, the N-terminus of the CD4 binding agent is exposed on the exterior surface of the lipid bilayer.

In some embodiments, the CD4 binding agent is the only surface displayed non-viral sequence of the viral vector. In some embodiments, the CD4 binding agent is the only membrane bound non-viral sequence of the viral vector. In some embodiments, the viral vector does not contain a molecule that engages or stimulates T cells other than the CD4 binding agent.

In some embodiments, viral vectors may display CD4 binding agents that are not conjugated to protein fusogens in order to redirect the fusion activity towards a cell that is bound by the targeting moiety, or to affect homing.

In some embodiments, a protein fusogen derived from a virus or organism that do not infect humans does not have a natural fusion targets in patients, and thus has high specificity.

The viral vectors disclosed herein include one or more CD8 binding agents. For example, a CD8 binding agent may be fused to or incorporated in a protein fusogen or viral envelope protein. In another embodiment, a CD8 binding agent may be incorporated into the viral envelope via fusion with a transmembrane domain.

The viral vectors disclosed herein include one or more CD8 binding agents. For example, a CD8 binding agent may be fused to or incorporated in a protein fusogen or viral envelope protein. In another embodiment, a CD8 binding agent may be incorporated into the viral envelope via fusion with a transmembrane domain. In some of any of the provided embodiments, the CD8 binding agent is an anti-CD8 antibody or an antigen-binding fragment. In some of any of the provided embodiments, the anti-CD8 antibody or antigen-binding fragment is mouse, rabbit, human, or humanized. In some embodiments, the antigen-binding fragment is a single chain variable fragment (scFv). In some embodiments, the anti-CD8 antibody or antigen-binding fragment is a single domain antibody. In some embodiments, the anti-CD8 antibody or antigen-binding fragment is a camelid (e.g. llama, alpaca, camel) (e.g. VHH).

In some of any of the provided embodiments, the CD8 binding agent binds to a CD8 alpha chain and/or CD8 beta chain. In some of any of the provided embodiments, the CD8 binding agent binds to a CD8 alpha chain. In some of any of the provided embodiments, the CD8 binding agent binds to a CD8 beta chain. In some of any of the provided embodiments, the CD8 binding agent binds to a CD8 alpha chain and a CD8 beta chain. Exemplary CD8 binding agents are also disclosed in Ser. No. 17/715,253, which is hereby incorporated in its entirety.

Further exemplary CD8 binding agents include antibodies and fragments thereof (e.g., scFv, VHH) that bind to one or more of CD8 alpha and CD8 beta. Such antibodies may be derived from any species, and may be for example, mouse, rabbit, human, humanized, or camelid antibodies. Exemplary antibodies include those disclosed in WO2014025828, WO2014164553, WO2020069433, WO2015184203, US20160176969, WO2017134306, WO2017182585, WO2019032661, WO2020257412, WO2018170096, WO2020060924, U.S. Ser. No. 10/730,944, US20200172620, and the non-human antibodies OKT8; RPA-T8, 12.C7 (Novus); 17D8, 3B5, LT8, RIV11, SP16, YTC182.20, MEM-31, MEM-87, RAVB3, C8/144B (Thermo Fisher); 2ST8.5H7, Bu88, 3C39, Hit8a, SPM548, CA-8, SK1, RPA-T8 (GeneTex); UCHT4 (Absolute Antibody); BW135/80 (Miltenyi); G42-8 (BD Biosciences); C8/1779R, mAB 104 (Enzo Life Sciences); B-Z31 (Sapphire North America); 32-M4, 5F10, MCD8, UCH-T4, 5F2 (Santa Cruz); D8A8Y, RPA-T8 (Cell Signaling Technology). Further exemplary anti-CD8 binding agents and G proteins are described in U.S. provisional application No. 63/172,518, which is incorporated by reference herein. Other exemplary binding agents include designed ankyrin repeat proteins (DARPins) and binding agents based on fibronectin type III (Fn3) scaffolds.

In some embodiments, the CD8 binding agent is an scFv that contains a VH and VL set forth from any as below, in which the VH and VL are separated by linker. In some embodiments, the CD8 binding agent is a VHH having the sequence set forth below. In some embodiments, the CD8 binding agent is linked to the C-terminus of a truncated NiV-G set forth in SEQ ID NO: 19 to provide a re-targeted NiV-G. In some embodiments, the retargeted NiV-G is pseudotyped on a lentiviral vector with the a NiV-F (e.g. set forth in SEQ ID NO:12). In some embodiments, the lentiviral vector further contains a payload gene encoding an anti-CD19 CAR. In some embodiments, the anti-CD19 CAR contains an anti-CD19 FMC63 scFv binding domain set forth in SEQ ID NO:40, a CD8 hinge set forth in SEQ ID NO:27, a CD8 transmembrane domain set forth in SEQ ID NO: 33, a 4-1bb signaling domain set forth in SEQ ID NO:36. a CD3zeta signaling domain set forth in SEQ ID NO: 38.

CD8_1
VH (SEQ ID NO.: 120):
QVQLVQSGAEVKKPGASVKVSCKASGGTFSSYAISWVRQAPGQGLEWMGI
IDPSDGNTNYAQNFQGRVTMTRDTSTSTVYMELSSLRSEDTAVYYCAKER
AAAGYYYYMDVWGQGTTVTVSS
VL (SEQ ID NO.: 121:
DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPLTFGG
GTKVEIKR
CD8_2
VH (SEQ ID NO.: 122):
QVQLVQSGAEVKKPGASVKVSCKASGYTFTDYYIQWVRQAPGQGLEWMGW
INPNSGGTSYAQKFQGRVTMTRDTSTSTVYMELSSLRSEDTAVYYCAKEG
DYYYGMDAWGQGTMVTVSS
VL (SEQ ID NO.: 123):
DIVMTQSPLSLPVTPGEPASISCRSSQSLLHSNGYNYLDWYLQKPGQSPQ
LLIYLGSNRASGVPDRFSGSGSGTDFTLKISRVEAEDVGVYYCMQGLQTP
HTFGQGTKVEIKR
CD8_3
VH (SEQ ID NO.: 124):
QVQLVQSGAEVKKPGASVKVSCKASGYTFTSYYMHWVRQAPGQGLEWMGG
FDPEDGETIYAQKFQGRVTMTRDTSTSTVYMELSSLRSEDTAVYYCARDQ
GWGMDVWGQGTTVTVSS
VL (SEQ ID NO.: 125):
DIQMTQSPSSLSASVGDRVTITCRASQSISSYLNWYQQKPGKAPKLLIYA
ASSLQSGVPSRFSGSGSGTDFTLTISSLQPEDFATYYCQQTYSTPYTFGQ
GTKLEIKR
CD8_4
VH (SEQ ID NO.: 126):
QVQLVQSGAEVKKPGASVKVSCKASGYTFTNHYMHWVRQAPGQGLEWMGW
MNPNSGNTGYAQKFQGRVTMTRDTSTSTVYMELSSLRSEDTAVYYCASSE
SGSDLDYWGQGTLVTVSS
VL (SEQ ID NO.: 127):
DIQMTQSPSSLSASVGDRVTITCRASQTIGNYVNWYQQKPGKAPKLLIYG
ASNLHTGVPSRFSGSGSGTDFTLTISSLQPEDFATYYCQQTYSAPLTFGG
GTKVEIKR

In some embodiments, the CD8 binding agent is VHH set forth as:

VHH (SEQ ID NO.: 128):
QVQLVESGGGLVQAGGSLRLSCAASGRTFSGYVMGWFRQAPGKQRKFVAA
ISRGGLSTSYADSVKGRFTISRDNAKNTVFLQMNTLKPEDTAVYYCAADR
SDLYEITAASNIDSWGQGTLVTVSS

In some embodiments, protein fusogens or viral envelope proteins may be re-targeted by mutating amino acid residues in a fusion protein or a targeting protein (e.g. the hemagglutinin protein). In particular embodiments, the fusogen (e.g. G protein) is mutated to reduce binding for the native binding partner of the fusogen. In some embodiments, the fusogen is or contains a mutant G protein or a biologically active portion thereof that is a mutant of wild-type Niv-G and exhibits reduced binding to one or both of the native binding partners Ephrin B2 or Ephrin B3, including any as described above. Thus, in some aspects, a fusogen can be retargeted to display altered tropism. In some embodiments, the binding confers re-targeted binding compared to the binding of a wild-type surface glycoprotein protein in which a new or different binding activity is conferred. In particular embodiments, the binding confers re-targeted binding compared to the binding of a wild-type G protein in which a new or different binding activity is conferred. In some embodiments the fusogen is randomly mutated. In some embodiments the fusogen is rationally mutated. In some embodiments the fusogen is subjected to directed evolution. In some embodiments the fusogen is truncated and only a subset of the peptide is used in the viral vector. In some embodiments, amino acid residues in the measles hemagglutinin protein may be mutated to alter the binding properties of the protein, redirecting fusion (doi:10.1038/nbt942, Molecular Therapy vol. 16 no. 8, 1427-1436 August 2008, doi:10.1038/nbt1060, DOI: 10.1128/JVI.76.7.3558-3563.2002, DOI: 10.1128/JVI.75.17.8016-8020.2001, doi: 10.1073pnas.0604993103).

In some embodiments, protein fusogens may be re-targeted by covalently conjugating a CD8 binding agent to the fusion protein or targeting protein (e.g. the hemagglutinin protein). In some embodiments, the fusogen and CD8 binding agent are covalently conjugated by expression of a chimeric protein comprising the fusogen linked to the CD8 binding agent. In some embodiments, a single-chain variable fragment (scFv) can be conjugated to fusogens to redirect fusion activity towards cells that display the scFv binding target (doi:10.1038/nbt1060, DOI 10.1182/blood-2012-11-468579, doi:10.1038/nmeth.1514, doi:10.1006/mthe.2002.0550, HUMAN GENE THERAPY 11:817-826, doi:10.1038/nbt942, doi:10.1371/journal.pone.0026381, DOI 10.1186/s12896-015-0142-z). In some embodiments, designed ankyrin repeat proteins (DARPin) can be conjugated to fusogens to redirect fusion activity towards cells that display the DARPin binding target (doi:10.1038/mt.2013.16, doi:10.1038/mt.2010.298, doi: 10.4049/jimmunol.1500956), as well as combinations of different DARPins (doi:10.1038/mto.2016.3). In some embodiments, receptor ligands and antigens can be conjugated to fusogens to redirect fusion activity towards cells that display the target receptor (DOI: 10.1089/hgtb.2012.054, DOI: 10.1128/JVI.76.7.3558-3563.2002). In some embodiments, a targeting protein can also include an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, protein fusogens may be re-targeted by non-covalently conjugating a CD8 binding agent to the fusion protein or targeting protein (e.g. the hemagglutinin protein). In some embodiments, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi:10.1038/nm1192). In some embodiments, altered and non-altered fusogens may be displayed on the same retroviral vector or VLP (doi: 10.1016/j.biomaterials.2014.01.051).

In some embodiments, a CD8 binding agent comprises a humanized antibody molecule, intact IgA, IgG, IgE or IgM antibody; bi- or multi-specific antibody (e.g., Zybodies®, etc); antibody fragments such as Fab fragments, Fab′ fragments, F(ab′)₂ fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s.

In some embodiments, the CD8 binding agent is a peptide. In some embodiments, the CD8 binding agent is an antibody, such as a single-chain variable fragment (scFv). In some embodiments, the CD8 binding agent is an antibody, such as a single domain antibody. In some embodiments, the CD8 binding agent is a VHH. In some embodiments, the antibody can be human or humanized. In some embodiments, the antibody or portion thereof is naturally occurring. In some embodiments, the antibody or portion thereof is synthetic.

In some embodiments, the antibody can be generated from phage display libraries to have specificity for a desired target ligand. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, a library of human single domain antibodies is synthetically generated by introducing diversity into one or more scaffolds.

In some embodiments, the C-terminus of the CD8 binding agent is attached to the C-terminus of the G protein (e.g., fusogen) or biologically active portion thereof. In some embodiments, the N-terminus of the CD8 binding agent is exposed on the exterior surface of the lipid bilayer.

In some embodiments, the CD8 binding agent is the only surface displayed non-viral sequence of the viral vector. In some embodiments, the CD8 binding agent is the only membrane bound non-viral sequence of the viral vector. In some embodiments, the viral vector does not contain a molecule that engages or stimulates T cells other than the CD8 binding agent.

In some embodiments, viral vectors may display CD8 binding agents that are not conjugated to protein fusogens in order to redirect the fusion activity towards a cell that is bound by the targeting moiety, or to affect homing.

In some embodiments, a protein fusogen derived from a virus or organism that do not infect humans does not have a natural fusion targets in patients, and thus has high specificity.

In some embodiments, the G protein or functionally active variant or biologically active portion thereof is linked directly to the binding domain and/or variable domain thereof. In some embodiments, the targeted envelope protein is a fusion protein that has the following structure: (N′-single domain antibody-C′)-(C′-G protein-N′).

In some embodiments, the G protein or functionally active variant or biologically active portion thereof is linked indirectly via a linker to the binding domain and/or variable domain thereof. In some embodiments, the linker is a peptide linker. In some embodiments, the linker is a chemical linker.

In some embodiments, the linker is a peptide linker and the targeted envelope protein is a fusion protein containing the G protein or functionally active variant or biologically active portion thereof linked via a peptide linker to the sdAb variable domain. In some embodiments, the targeted envelope protein is a fusion protein that has the following structure: (N′-single domain antibody-C′)-Linker-(C′-G protein-N′).

In some embodiments, the peptide linker is up to 65 amino acids in length. In some embodiments, the peptide linker comprises from or from about 2 to 65 amino acids, 2 to 60 amino acids, 2 to 56 amino acids, 2 to 52 amino acids, 2 to 48 amino acids, 2 to 44 amino acids, 2 to 40 amino acids, 2 to 36 amino acids, 2 to 32 amino acids, 2 to 28 amino acids, 2 to 24 amino acids, 2 to 20 amino acids, 2 to 18 amino acids, 2 to 14 amino acids, 2 to 12 amino acids, 2 to 10 amino acids, 2 to 8 amino acids, 2 to 6 amino acids, 6 to 65 amino acids, 6 to 60 amino acids, 6 to 56 amino acids, 6 to 52 amino acids, 6 to 48 amino acids, 6 to 44 amino acids, 6 to 40 amino acids, 6 to 36 amino acids, 6 to 32 amino acids, 6 to 28 amino acids, 6 to 24 amino acids, 6 to 20 amino acids, 6 to 18 amino acids, 6 to 14 amino acids, 6 to 12 amino acids, 6 to 10 amino acids, 6 to 8 amino acids, 8 to 65 amino acids, 8 to 60 amino acids, 8 to 56 amino acids, 8 to 52 amino acids, 8 to 48 amino acids, 8 to 44 amino acids, 8 to 40 amino acids, 8 to 36 amino acids, 8 to 32 amino acids, 8 to 28 amino acids, 8 to 24 amino acids, 8 to 20 amino acids, 8 to 18 amino acids, 8 to 14 amino acids, 8 to 12 amino acids, 8 to 10 amino acids, 10 to 65 amino acids, 10 to 60 amino acids, 10 to 56 amino acids, 10 to 52 amino acids, 10 to 48 amino acids, 10 to 44 amino acids, 10 to 40 amino acids, 10 to 36 amino acids, 10 to 32 amino acids, 10 to 28 amino acids, 10 to 24 amino acids, 10 to 20 amino acids, 10 to 18 amino acids, 10 to 14 amino acids, 10 to 12 amino acids, 12 to 65 amino acids, 12 to 60 amino acids, 12 to 56 amino acids, 12 to 52 amino acids, 12 to 48 amino acids, 12 to 44 amino acids, 12 to 40 amino acids, 12 to 36 amino acids, 12 to 32 amino acids, 12 to 28 amino acids, 12 to 24 amino acids, 12 to 20 amino acids, 12 to 18 amino acids, 12 to 14 amino acids, 14 to 65 amino acids, 14 to 60 amino acids, 14 to 56 amino acids, 14 to 52 amino acids, 14 to 48 amino acids, 14 to 44 amino acids, 14 to 40 amino acids, 14 to 36 amino acids, 14 to 32 amino acids, 14 to 28 amino acids, 14 to 24 amino acids, 14 to 20 amino acids, 14 to 18 amino acids, 18 to 65 amino acids, 18 to 60 amino acids, 18 to 56 amino acids, 18 to 52 amino acids, 18 to 48 amino acids, 18 to 44 amino acids, 18 to 40 amino acids, 18 to 36 amino acids, 18 to 32 amino acids, 18 to 28 amino acids, 18 to 24 amino acids, 18 to 20 amino acids, 20 to 65 amino acids, 20 to 60 amino acids, 20 to 56 amino acids, 20 to 52 amino acids, 20 to 48 amino acids, 20 to 44 amino acids, 20 to 40 amino acids, 20 to 36 amino acids, 20 to 32 amino acids, 20 to 28 amino acids, 20 to 26 amino acids, 20 to 24 amino acids, 24 to 65 amino acids, 24 to 60 amino acids, 24 to 56 amino acids, 24 to 52 amino acids, 24 to 48 amino acids, 24 to 44 amino acids, 24 to 40 amino acids, 24 to 36 amino acids, 24 to 32 amino acids, 24 to 30 amino acids, 24 to 28 amino acids, 28 to 65 amino acids, 28 to 60 amino acids, 28 to 56 amino acids, 28 to 52 amino acids, 28 to 48 amino acids, 28 to 44 amino acids, 28 to 40 amino acids, 28 to 36 amino acids, 28 to 34 amino acids, 28 to 32 amino acids, 32 to 65 amino acids, 32 to 60 amino acids, 32 to 56 amino acids, 32 to 52 amino acids, 32 to 48 amino acids, 32 to 44 amino acids, 32 to 40 amino acids, 32 to 38 amino acids, 32 to 36 amino acids, 36 to 65 amino acids, 36 to 60 amino acids, 36 to 56 amino acids, 36 to 52 amino acids, 36 to 48 amino acids, 36 to 44 amino acids, 36 to 40 amino acids, 40 to 65 amino acids, 40 to 60 amino acids, 40 to 56 amino acids, 40 to 52 amino acids, 40 to 48 amino acids, 40 to 44 amino acids, 44 to 65 amino acids, 44 to 60 amino acids, 44 to 56 amino acids, 44 to 52 amino acids, 44 to 48 amino acids, 48 to 65 amino acids, 48 to 60 amino acids, 48 to 56 amino acids, 48 to 52 amino acids, 50 to 65 amino acids, 50 to 60 amino acids, 50 to 56 amino acids, 50 to 52 amino acids, 54 to 65 amino acids, 54 to 60 amino acids, 54 to 56 amino acids, 58 to 65 amino acids, 58 to 60 amino acids, or 60 to 65 amino acids. In some embodiments, the peptide linker is a polypeptide that is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 amino acids in length.

In particular embodiments, the linker is a flexible peptide linker. In some such embodiments, the linker is 1-20 amino acids, such as 1-20 amino acids predominantly composed of glycine. In some embodiments, the linker is 1-20 amino acids, such as 1-20 amino acids predominantly composed of glycine and serine. In some embodiments, the linker is a flexible peptide linker containing amino acids Glycine and Serine, referred to as GS-linkers. In some embodiments, the peptide linker includes the sequences GS, GGS, GGGGS (SEQ ID NO:20), GGGGGS (SEQ ID NO:21) or combinations thereof. In some embodiments, the polypeptide linker has the sequence (GGS)n, wherein n is 1 to 10. In some embodiments, the polypeptide linker has the sequence (GGGGS)n, (SEQ ID NO:22) wherein n is 1 to 10. In some embodiments, the polypeptide linker has the sequence (GGGGGS)n (SEQ ID NO:23), wherein n is 1 to 6.

### 3. G Proteins

In some embodiments, the one or more paramyxovirus fusogen includes a paramyxovirus attachment glycoprotein (e.g. G protein). Paramyxoviral attachment proteins are type II transmembrane glycoproteins that are designated as hemagglutinin-neuraminidase (HN), hemagglutinin (H), or glycoproteins (G), depending on two characteristics; the ability to agglutinate erythrocytes (hemagglutination) and the presence or absence of neuraminidase activity (cleavage of sialic acid). Specifically, the HN attachment glycoprotein is characteristic of the Avulavirus, Respirovirus, and Rubulavirus genera, the H attachment glycoproteins are found in members of the Morbillivirus genus, while the G attachment glycoproteins are utilized by the viruses of the genus Henipavirus and the Pneumovirinae subfamily. The geometries of HN, H, or G glycoproteins possess high structural similarity, however although H and G glycoproteins are capable of recognizing protein receptors, they lack neuraminidase activity.

Paramyxoviral attachment glycoproteins contain a short N-terminal cytoplasmic tail, a transmembrane domain, and an extracellular domain containing an extracellular stalk and a globular head. The N-terminal cytoplasmic domain is within the inner lumen of the lipid bilayer and the C-terminal portion is the extracellular domain that is exposed on the outside of the lipid bilayer. The receptor binding and antigenic sites reside on the extracellular domain. Regions of the stalk in the C-terminal region have been shown to be involved in interactions with the F protein and triggering of fusion with a target cell membrane (Liu et al. 2015 J of Virology 89:1838). The F protein undergoes significant conformational change that facilitates the insertion of the fusion peptide into target membranes, bringing the two HR regions together in the formation of a six-helix bundle structure or trimer-of-hairpins during or immediately following fusion of virus and cell membranes (Bishop et al. 2008. J of Virology 82(22): 11398-11409). The cytoplasmic tails play a role in particle formation, incorporation into packaged particles, and serves as a signal peptide to modulate protein maturation and surface transport (Sawatsky et al. 2016. J of Virology 97:1066-1076).

In some embodiments, any of the provided viral vector (lentiviral vectors) that contains a paramyxovirus attachment glycoprotein (e.g. G protein, such as NiV-G) or a biologically active portion thereof may also contain an F protein, such as a NiV-F protein, such as a full-length NiV-F protein or a biologically active portion thereof or a variant thereof.

In some embodiments, the envelope protein contains a henipavirus envelope attachment glycoprotein G (G protein) or a biologically active portion thereof. In some embodiments, the G protein may be retargeted by linkage to a targeting moiety, such as a binding molecule (e.g. antibody or antigen-binding fragment, e.g. sdAb or scFv) that binds to a target cell. In some embodiments, the G protein and the NiV-F protein provided herein together exhibit fusogenic activity to a target cell, such as to deliver an exogenous agent or nucleic acid exogenous agent to the target cell.

The attachment G proteins are type II transmembrane glycoproteins containing an N-terminal cytoplasmic tail (e.g. corresponding to amino acids 1-49 of SEQ ID NO:14), a transmembrane domain (e.g. corresponding to amino acids 50-70 of SEQ ID NO:14), and an extracellular domain containing an extracellular stalk (e.g. corresponding to amino acids 71-187 of SEQ ID NO:14), and a globular head (corresponding to amino acids 188-602 of SEQ ID NO:14). The N-terminal cytoplasmic domain is within the inner lumen of the lipid bilayer and the C-terminal portion is the extracellular domain that is exposed on the outside of the lipid bilayer. Regions of the stalk in the C-terminal region (e.g. corresponding to amino acids 71-187 of SEQ ID NO: 14) have been shown to be involved in interactions with F protein and triggering of F protein fusion (Liu et al. 2015 J of Virology 89:1838). In wild-type G protein, the globular head mediates receptor binding to henipavirus entry receptors Ephrin B2 and Ephrin B3, but is dispensable for membrane fusion (Brandel-Tretheway et al. Journal of Virology. 2019. 93(13)e00577-19). In some embodiments herein, tropism of the G protein is altered by linkage of the G protein or biologically active fragment thereof (e.g. cytoplasmic truncation) to a sdAb variable domain. Binding of the G protein to a binding partner can trigger fusion mediated by a compatible F protein or biologically active portion thereof. G protein sequences disclosed herein are predominantly disclosed as expressed sequences including an N-terminal methionine required for start of translation. As such N-terminal methionines are commonly cleaved co- or post-translationally, the mature protein sequences for all G protein sequences disclosed herein are also contemplated as lacking the N-terminal methionine.

G glycoproteins are highly conserved between henipavirus species. For example, the G protein of NiV and HeV viruses share 79% amino acid identity. Studies have shown a high degree of compatibility among G proteins with F proteins of different species as demonstrated by heterotypic fusion activation (Brandel-Tretheway et al. Journal of Virology. 2019). As described, a viral vector can contain heterologous G and F proteins from different species. In particular embodiments, the F protein or the functionally active variant or biologically active portion thereof retains fusogenic activity in conjunction with a G protein as provided, such as any set forth below. Fusogenic activity includes the activity of the F protein in conjunction with a G protein to promote or facilitate fusion of two membrane lumens, such as the lumen of the viral vector provided herein (e.g. having embedded in its lipid bilayer, such as exposed on its surface, a G protein and a F protein), and a cytoplasm of a target cell, e.g. a cell that contains a surface receptor or molecule that is recognized or bound by the G protein.

Exemplary Henipavirus protein G sequences are provided in Table 3

TABLE 3
Henipavirus protein G sequence clusters. Column 1, Genbank ID includes the Genbank ID
of the whole genome sequence of the virus that is the centroid sequence of the cluster.
Column 2,nucleotides of CDS provides the nucleotides corresponding to the CDS of the
gene in the whole genome. Column 3, Full Gene Name, provides the full name of the gene
including Genbank ID, virus species, strain, and protein name. Column 4, Sequence,
provides the amino acid sequence of the gene. Column 5, #Sequences/Cluster, provides
the number of sequences that cluster with this centroid sequence. Column 6
provides the SEQ ID numbers for the described sequences.

| | Nucleotides | | | SEQ |
|---|---|---|---|---|
| Genbank | of | Full sequence | | ID |
| ID | CDS | ID | Sequence | NO |
| AF017149 | 8913-10727 | gb: AF017149\| | MMADSKLVSLNNNLSGKIKDQGKVIKNY | 13 |
| | | Organism: | YGTMDIKKINDGLLDSKILGAFNTVIALL | |
| | | Hendra | GSIIIIVMNIMIIQNYTRTTDNQALIKESLQ | |
| | | virus\|Strain | SVQQQIKALTDKIGTEIGPKVSLIDTSSTIT | |
| | | Name: UNKNOWN- | IPANIGLLGSKISQSTSSINENVNDKCKFTL | |
| | | AF017149\| | PPLKIHECNISCPNPLPFREYRPISQGVSDL | |
| | | Protein | VGLPNQICLQKTTSTILKPRLISYTLPINTR | |
| | | Name: | EGVCITDPLLAVDNGFFAYSHLEKIGSCT | |
| | | glycoprotein\| | RGIAKQRIIGVGEVLDRGDKVPSMFMTN | |
| | | Gene | VWTPPNPSTIHHCSSTYHEDFYYTLCAVS | |
| | | Symbol: G | HVGDPILNSTSWTESLSLIRLAVRPKSDSG | |
| | | | DYNQKYIAITKVERGKYDKVMPYGPSGI | |
| | | | KQGDTLYFPAVGFLPRTEFQYNDSNCPII | |
| | | | HCKYSKAENCRLSMGVNSKSHYILRSGL | |
| | | | LKYNLSLGGDIILQFIEIADNRLTIGSPSKI | |
| | | | YNSLGQPVFYQASYSWDTMIKLGDVDTV | |
| | | | DPLRVQWRNNSVISRPGQSQCPRFNVCPE | |
| | | | VCWEGTYNDAFLIDRLNWVSAGVYLNS | |
| | | | NQTAENPVFAVFKDNEILYQVPLAEDDT | |
| | | | NAQKTITDCFLLENVIWCISLVEIYDTGDS | |
| | | | VIRPKLFAVKIPAQCSES | |
| AF212302 | 8943-10751 | gb: AF212302\| | MPAENKKVRFENTTSDKGKIPSKVIKSYY | 14 |
| | | Organism: | GTMDIKKINEGLLDSKILSAFNTVIALLGS | |
| | | Nipah virus\| | IVIIVMNIMIIQNYTRSTDNQAVIKDALQG | |
| | | Strain | IQQQIKGLADKIGTEIGPKVSLIDTSSTITIP | |
| | | Name: UNKNOWN- | ANIGLLGSKISQSTASINENVNEKCKFTLP | |
| | | AF212302\| | PLKIHECNISCPNPLPFREYRPQTEGVSNL | |
| | | Protein | VGLPNNICLQKTSNQILKPKLISYTLPVVG | |
| | | Name: | QSGTCITDPLLAMDEGYFAYSHLERIGSC | |
| | | attachment | SRGVSKQRIIGVGEVLDRGDEVPSLFMTN | |
| | | glycoprotein\| | VWTPPNPNTVYHCSAVYNNEFYYVLCA | |
| | | Gene | VSTVGDPILNSTYWSGSLMMTRLAVKPK | |
| | | Symbol: G | SNGGGYNQHQLALRSIEKGRYDKVMPY | |
| | | | GPSGIKQGDTLYFPAVGFLVRTEFKYNDS | |
| | | | NCPITKCQYSKPENCRLSMGIRPNSHYILR | |
| | | | SGLLKYNLSDGENPKVVFIEISDQRLSIGS | |
| | | | PSKIYDSLGQPVFYQASFSWDTMIKFGDV | |
| | | | LTVNPLVVNWRNNTVISRPGQSQCPRFNT | |
| | | | CPEICWEGVYNDAFLIDRINWISAGVFLD | |
| | | | SNQTAENPVFTVFKDNEILYRAQLASEDT | |
| | | | NAQKTITNCFLLKNKIWCISLVEIYDTGD | |
| | | | NVIRPKLFAVKIPEQCT | |
| JQ001776 | 8170-10275 | gb: JQ001776: | MLSQLQKNYLDNSNQQGDKMNNPDKKL | 15 |
| | | 8170-10275\| | SVNFNPLELDKGQKDLNKSYYVKNKNY | |
| | | Organism: | NVSNLLNESLHDIKFCIYCIFSLLIIITIINIIT | |
| | | Cedar | ISIVITRLKVHEENNGMESPNLQSIQDSLSS | |
| | | virus\|Strain | LTNMINTEITPRIGILVTATSVTLSSSINYV | |
| | | Name: CG1a\| | GTKTNQLVNELKDYITKSCGFKVPELKLH | |
| | | Protein | ECNISCADPKISKSAMYSTNAYAELAGPP | |
| | | Name: | KIFCKSVSKDPDFRLKQIDYVIPVQQDRSI | |
| | | attachment | CMNNPLLDISDGFFTYIHYEGINSCKKSDS | |
| | | glycoprotein\| | FKVLLSHGEIVDRGDYRPSLYLLSSHYHP | |
| | | Gene | YSMQVINCVPVTCNQSSFVFCHISNNTKT | |
| | | Symbol: G | LDNSDYSSDEYYITYFNGIDRPKTKKIPIN | |
| | | | NMTADNRYIHFTFSGGGGVCLGEEFIIPV | |
| | | | TTVINTDVFTHDYCESFNCSVQTGKSLKE | |
| | | | ICSESLRSPTNSSRYNLNGIMIISQNNMTD | |
| | | | FKIQLNGITYNKLSFGSPGRLSKTLGQVL | |
| | | | YYQSSMSWDTYLKAGFVEKWKPFTPNW | |
| | | | MNNTVISRPNQGNCPRYHKCPEICYGGT | |
| | | | YNDIAPLDLGKDMYVSVILDSDQLAENPE | |
| | | | ITVFNSTTILYKERVSKDELNTRSTTTSCF | |
| | | | LFLDEPWCISVLETNRFNGKSIRPEIYSYKI | |
| | | | PKYC | |
| NC_025256 | 9117-11015 | gb: NC_025256: | MPQKTVEFINMNSPLERGVSTLSDKKTLN | 16 |
| | | 9117-11015\| | QSKITKQGYFGLGSHSERNWKKQKNQND | |
| | | Organism: | HYMTVSTMILEILVVLGIMENLIVLTMVY | |
| | | Bat | YQNDNINQRMAELTSNITVLNLNLNQLT | |
| | | Paramyxovirus | NKIQREIIPRITLIDTATTITIPSAITYILATL | |
| | | Eid_hel/GH- | TTRISELLPSINQKCEFKTPTLVLNDCRINC | |
| | | M74a/GHA/ | TPPLNPSDGVKMSSLATNLVAHGPSPCRN | |
| | | 2009\|Strain | FSSVPTIYYYRIPGLYNRTALDERCILNPR | |
| | | Name: BatPV/ | LTISSTKFAYVHSEYDKNCTRGFKYYELM | |
| | | Eid_hel/GH- | TFGEILEGPEKEPRMFSRSFYSPTNAVNY | |
| | | M74a/GHA/ | HSCTPIVTVNEGYFLCLECTSSDPLYKAN | |
| | | 2009\|Protein | LSNSTFHLVILRHNKDEKIVSMPSFNLSTD | |
| | | Name: | QEYVQIIPAEGGGTAESGNLYFPCIGRLLH | |
| | | glycoprotein\| | KRVTHPLCKKSNCSRTDDESCLKSYYNQ | |
| | | Gene | GSPQHQVVNCLIRIRNAQRDNPTWDVITV | |
| | | Symbol: G | DLTNTYPGSRSRIFGSFSKPMLYQSSVSW | |
| | | | HTLLQVAEITDLDKYQLDWLDTPYISRPG | |
| | | | GSECPFGNYCPTVCWEGTYNDVYSLTPN | |
| | | | NDLFVTVYLKSEQVAENPYFAIFSRDQIL | |
| | | | KEFPLDAWISSARTTTISCFMFNNEIWCIA | |
| | | | ALEITRLNDDIIRPIYYSFWLPTDCRTPYP | |
| | | | HTGKMTRVPLRSTYNY | |
| NC_025352 | 8716-11257 | gb: NC_025352: | MATNRDNTITSAEVSQEDKVKKYYGVET | 17 |
| | | 8716-11257\| | AEKVADSISGNKVFILMNTLLILTGAIITIT | |
| | | Organism: | LNITNLTAAKSQQNMLKIIQDDVNAKLE | |
| | | Mojiang | MFVNLDQLVKGEIKPKVSLINTAVSVSIP | |
| | | virus\|Strain | GQISNLQTKFLQKYVYLEESITKQCTCNP | |
| | | Name: Tongguan | LSGIFPTSGPTYPPTDKPDDDTTDDDKVD | |
| | | 1\|Protein | TTIKPIEYPKPDGCNRTGDHFTMEPGANF | |
| | | Name: | YTVPNLGPASSNSDECYTNPSFSIGSSIYM | |
| | | attachment | FSQEIRKTDCTAGEILSIQIVLGRIVDKGQ | |
| | | glycoprotein\| | QGPQASPLLVWAVPNPKIINSCAVAAGDE | |
| | | Gene | MGWVLCSVTLTAASGEPIPHMFDGFWLY | |
| | | Symbol: G | KLEPDTEVVSYRITGYAYLLDKQYDSVFI | |
| | | | GKGGGIQKGNDLYFQMYGLSRNRQSFKA | |
| | | | LCEHGSCLGTGGGGYQVLCDRAVMSFGS | |
| | | | EESLITNAYLKVNDLASGKPVIIGQTFPPS | |
| | | | DSYKGSNGRMYTIGDKYGLYLAPSSWNR | |
| | | | YLRFGITPDISVRSTTWLKSQDPIMKILST | |
| | | | CTNTDRDMCPEICNTRGYQDIFPLSEDSE | |
| | | | YYTYIGITPNNGGTKNFVAVRDSDGHIAS | |
| | | | IDILQNYYSITSATISCFMYKDEIWCIAITE | |
| | | | GKKQKDNPQRIYAHSYKIRQMCYNMKS | |
| | | | ATVTVGNAKNITIRRY | |

In some embodiments, the G protein has a sequence set forth in any of SEQ ID NOS: 14, 13, 15, 16 or 17 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NOS: 14, 13, 15, 16 or 17.

In particular embodiments, the G protein or functionally active variant or biologically active portion is a protein that retains fusogenic activity in conjunction with a Henipavirus F protein, such as a NiV-F protein described herein. Fusogenic activity includes the activity of the G protein in conjunction with a Henipavirus F protein to promote or facilitate fusion of two membrane lumens, such as the lumen of the targeted viral vector having embedded in its lipid bilayer a henipavirus F and G protein, and a cytoplasm of a target cell, e.g. a cell that contains a surface receptor or molecule that is recognized or bound by the targeted envelope protein. In some embodiments, the F protein and G protein are from the same Henipavirus species (e.g. NiV-G and NiV-F).

In some embodiments the G protein is a mutant G protein that is a functionally active variant or biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference G protein sequence. In some embodiments, the reference G protein sequence is the wild-type sequence of a G protein or a biologically active portion thereof. In some embodiments, the functionally active variant or the biologically active portion thereof is a mutant of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein or biologically active portion thereof. In some embodiments, the wild-type G protein has the sequence set forth in any one of SEQ ID NOS: 14, 13, 15, 16 or 17.

In some embodiments, the G protein is a mutant G protein that is a biologically active portion that is an N-terminally and/or C-terminally truncated fragment of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein. In particular embodiments, the truncation is an N-terminal truncation of all or a portion of the cytoplasmic domain. In some embodiments, the mutant G protein is a biologically active portion that is truncated and lacks up to 49 contiguous amino acid residues at or near the N-terminus of the wild-type G protein, such as a wild-type G protein set forth in any one of SEQ ID NOS: 14, 13, 15, 16 or 17. In some embodiments, the mutant G protein is truncated and lacks up to 49 contiguous amino acids, such as up to 49, 48, 47, 46, 45, 44, 43, 42, 41, 40, 30, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 contiguous amino acids at the N-terminus of the wild-type G protein.

In some embodiments, the G protein is a wild-type Nipah virus G (NiV-G) protein or a Hendra virus G protein, or is a functionally active variant or biologically active portion thereof. In some embodiments, the G protein is a NiV-G protein that has the sequence set forth in SEQ ID NO:14, or is a functional variant or a biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, at least at or about 99% sequence identity to SEQ ID NO:14.

In some embodiments, the G protein is a mutant NiV-G protein that is a biologically active portion of a wild-type NiV-G. In some embodiments, the biologically active portion is an N-terminally truncated fragment. In some embodiments, the mutant NiV-G protein is truncated and lacks up to 5 contiguous amino acid residues at or near the N-terminus of the wild-type NiV-G protein, such as compared to wild-type NiV-G set forth in SEQ ID NO: 14. In some embodiments, the mutant NiV-G protein is truncated and lacks up to 10 contiguous amino acid residues at or near the N-terminus of the wild-type NiV-G protein, such as compared to wild-type NiV-G set forth in SEQ ID NO: 14. In some embodiments, the mutant NiV-G protein is truncated and lacks up to 15 contiguous amino acid residues at or near the N-terminus of the wild-type NiV-G protein, such as compared to wild-type NiV-G set forth in SEQ ID NO: 14. n some embodiments, the mutant NiV-G protein is truncated and lacks up to 20 contiguous amino acid residues at or near the N-terminus of the wild-type NiV-G protein, such as compared to wild-type NiV-G set forth in SEQ ID NO: 14. In some embodiments, the mutant NiV-G protein is truncated and lacks up to 25 contiguous amino acid residues at or near the N-terminus of the wild-type NiV-G protein, such as compared to wild-type NiV-G set forth in SEQ ID NO: 14. In some embodiments, the mutant NiV-G protein is truncated and lacks up to 30 contiguous amino acid residues at or near the N-terminus of the wild-type NiV-G protein, such as compared to wild-type NiV-G set forth in SEQ ID NO: 14. In some embodiments, the mutant NiV-G protein is truncated and lacks up to 35 contiguous amino acid residues at or near the N-terminus of the wild-type NiV-G protein, such as compared to wild-type NiV-G set forth in SEQ ID NO: 14. In some embodiments, the mutant NiV-G protein (also called variant NiV-G) contains an N-terminal methionine.

In some embodiments, the mutant NiV-G protein is truncated and lacks up to amino acid 34 at or near the N-terminus of the wild-type NiV-G protein, such as compared to wild-type NiV-G set forth in SEQ ID NO: 14. In some embodiments, the mutant NiV-G protein (also called variant NiV-G) contains an N-terminal methionine. In some embodiments, the mutant NiV-G protein lacks amino acids 2-34 as compared to wild-type NiV-G set forth in SEQ ID NO: 14.

In some embodiments, the G protein or the functionally active variant or biologically active portion thereof binds to Ephrin B2 or Ephrin B3. In some embodiments, the G protein is a mutant G protein, such as a truncated G protein as described and retains binding to Ephrin B2 or B3. Reference to retaining binding to Ephrin B2 or B3 includes binding that is similar to the level or degree of binding of the corresponding wild-type G protein, such as set forth in SEQ ID NO: 14, 13, 15, 16 or 17., such as at least 50%, at least 60%, at least 70%, at least 80% or at least 90% of the binding of the wild-type G protein.

In some embodiments, the G protein or the biologically thereof is a mutant G protein that exhibits reduced binding for the native binding partner of a wild-type G protein. In some embodiments, the mutant G protein or the biologically active portion thereof is a mutant of wild-type Niv-G and exhibits reduced binding to one or both of the native binding partners Ephrin B2 or Ephrin B3. In some embodiments, the mutant G-protein or the biologically active portion, such as a mutant NiV-G protein, exhibits reduced binding to the native binding partner. In some embodiments, the reduced binding to Ephrin B2 or Ephrin B3 is reduced by greater than at or about 5%, at or about 10%, at or about 15%, at or about 20%, at or about 25%, at or about 30%, at or about 40%, at or about 50%, at or about 60%, at or about 70%, at or about 80%, at or about 90%, or at or about 100%.

In some embodiments, the mutations can improve transduction efficiency. In some embodiments, the mutations allow for specific targeting of other desired cell types that are not Ephrin B2 or Ephrin B3. In some embodiments, the mutations result in at least the partial inability to bind at least one natural receptor, such as reduce the binding to at least one of Ephrin B2 or Ephrin B3. In some embodiments, the mutations described herein interfere with natural receptor recognition.

In some embodiments, the G protein contains one or more amino acid substitutions in a residue that is involved in the interaction with one or both of Ephrin B2 and Ephrin B3. In some embodiments, the amino acid substitutions correspond to mutations E501A, W504A, Q530A and E533A with reference to numbering set forth in SEQ ID NO:14. In some embodiments, the G protein is a mutant G protein containing one or more amino acid substitutions selected from the group consisting of E501A, W504A, Q530A and E533A with reference to numbering set forth in SEQ ID NO:14. In some embodiments, the G protein is a mutant G protein that contains one or more amino acid substitutions elected from the group consisting of E501A, W504A, Q530A and E533A with reference to SEQ ID NO:14 and is a biologically active portion thereof containing an N-terminal truncation.

In particular embodiments, the G protein has the sequence of amino acids set forth in SEQ ID NO: 19, or is a functionally active variant thereof or a biologically active portion thereof that retains binding and/or fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 19 and retains fusogenic activity in conjunction with a NiV-F protein as described.

Reference to retaining fusogenic activity includes activity of a viral vector (e.g. lentiviral vector) containing a variant NiV-F protein as described or biologically active portion or functionally active variant of the F protein (in conjunction with a G protein, such as a NiV-G protein as described) that is between at or about 10% and at or about 150% or more of the level or degree of binding of a reference viral vector (e.g. lentiviral vector) that is similar, such as contains the same variant NiV-F, but that contains the corresponding wild-type G protein, such as set forth in SEQ ID NO: 14. For instance, viral vector (e.g. lentiviral vector) that retains fusogenic activity has at least or at least about 10% of the level or degree of fusogenic activity of the reference viral vector that is similar (such as contains the same variant NiV-F) but that contains the corresponding wild-type G protein, such as at least or at least about 15% of the level or degree of fusogenic activity, at least or at least about 20% of the level or degree of fusogenic activity, at least or at least about 25% of the level or degree of fusogenic activity, at least or at least about 30% of the level or degree of fusogenic activity, at least or at least about 35% of the level or degree of fusogenic activity, at least or at least about 40% of the level or degree of fusogenic activity, at least or at least about 45% of the level or degree of fusogenic activity, at least or at least about 50% of the level or degree of fusogenic activity, at least or at least about 55% of the level or degree of fusogenic activity, at least or at least about 60% of the level or degree of fusogenic activity, at least or at least about 65% of the level or degree of fusogenic activity, at least or at least about 70% of the level or degree of fusogenic activity, at least or at least about 75% of the level or degree of fusogenic activity, at least or at least about 80% of the level or degree of fusogenic activity, at least or at least about 85% of the level or degree of fusogenic activity, at least or at least about 90% of the level or degree of fusogenic activity, at least or at least about 95% of the level or degree of fusogenic activity, at least or at least about 100% of the level or degree of fusogenic activity, or at least or at least about 120% of the level or degree of fusogenic activity.

### E. Payload Gene

In some embodiments, the viral vector comprises an exogenous agent that is a nucleic acid encoding a payload gene. For example, the viral vector may comprise a nucleic acid that is or encodes an RNA to enhance expression of an endogenous protein, or a siRNA or miRNA that inhibits protein expression of an endogenous protein. For example, the endogenous protein may modulate structure or function in the target cells. In some embodiments, the viral vector may comprise a nucleic acid that is or encodes an engineered protein that modulates structure or function in the target cells. In some embodiments, the viral vector may comprise a nucleic acid that is or encodes a transcriptional activator that modulate structure or function in the target cells.

In some embodiments, the viral vector herein comprises a nucleic acid, e.g., RNA or DNA. In some embodiments, the nucleic acid is, comprises, or consists of one or more natural nucleic acid residues. In some embodiments, the nucleic acid is, comprises, or consists of one or more nucleic acid analogs. In some embodiments, the nucleic acid has a nucleotide sequence that encodes a functional gene product such as an RNA or protein. In some embodiments, the nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, the nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, the nucleic acid is partly or wholly single stranded; in some embodiments, the nucleic acid is partly or wholly double stranded. In some embodiments the nucleic acid has a nucleotide sequence comprising at least one element that encodes, or is the complement of a sequence that encodes, a polypeptide.

In some embodiments, the viral vector contains a nucleic acid that encodes a payload gene (also referred to as a “heterologous, recombinant, exogenous, or therapeutic gene.”).

In some embodiments, the exogenous agent is a payload gene that encodes a protein that comprises a cytosolic protein, e.g., a protein that is produced in the recipient cell and localizes to the recipient cell cytoplasm. In some embodiments, the exogenous agent is a payload gene that encodes a protein that comprises a secreted protein, e.g., a protein that is produced and secreted by the recipient cell. In some embodiments, the exogenous agent is a payload gene that encodes a protein that is a nuclear protein, e.g., a protein that is produced in the recipient cell and is imported to the nucleus of the recipient cell. In some embodiments, the exogenous agent is a payload gene that encodes s a protein that comprises an organellar protein (e.g., a mitochondrial protein), e.g., a protein that is produced in the recipient cell and is imported into an organelle (e.g., a mitochondrial) of the recipient cell.

In some embodiments, the exogenous agent is a payload gene that encodes a protein that comprises a membrane protein. In some embodiments, the membrane protein comprises a chimeric antigen receptor (CAR), a T cell receptor, an integrin, an ion channel, a pore forming protein, a Toll-Like Receptor, an interleukin receptor, a cell adhesion protein, or a transport protein.

In some embodiments, the exogenous agent is a payload gene that encodes a protein that is a nuclease for use in gene editing methods. In some embodiments, the nuclease is a zinc-finger nucleases (ZFNs), transcription-activator like effector nucleases (TALENs), or a CRISPR-associated protein-nuclease (Cas). In some embodiments, the Cas is Cas9 from Streptococcus pyogenes. In some embodiments, the Cas is a Cas12a (also known as cpf1) from a Prevotella or Francisella bacteria, or the Cas is a Cas12b from a Bacillus, optionally Bacillus hisashii. In some of any embodiments, the Cas is a Cas3, Cas13, CasMini, or any other Cas protein known in the art. See for example, Wang et al., Biosensors and Bioelectronics (165) 1: 2020, and Wu et al. Nature Reviews Chemistry (4) 441: 2020)

In some embodiments, the provided the viral vector contains a exogenous agent that is a payload gene that encodes a protein that is a nuclease protein. In some embodiments, the provided the viral vector contains a protein that is a nuclease protein and the nuclease protein is directly delivered to a target cell Methods of delivering a nuclease protein include those as described, for example, in Cai et al. Elife, 2014, 3:e01911 and International patent publication No. WO2017068077. For instance, the provided viral vector comprises one or more Cas protein(s), such as Cas9. In some embodiments, the nuclease protein (e.g. Cas, such as Cas 9) is engineered as a chimeric nuclease protein with a viral structural protein (e.g. GAG) for packaging into the viral vector (e.g. paramyxovirus viral vector). For instance, a chimeric Cas9-protein fusion with the structural GAG protein can be packaged inside a paramyxovirus viral vector. In some embodiments, the fusion protein is a cleavable fusion protein between (i) a viral structural protein (e.g. GAG) and (ii) a nuclease protein (e.g. Cas protein, such as Cas 9). Similar such systems for delivery of a Cas protein are disclosed in U.S. patent Ser. No. 10/968,253 and International Application No. WO2020102709, the contents of which are incorporated in their entirety.

In some embodiments, the viral vector is a vector which further comprises an exogenous agent that is an encapsulated polypeptide or polynucleotide encoding a payload gene, a therapeutic gene, an exogenous gene, and/or a recombinant gene, such as any recombinant gene, particularly a therapeutic gene.

In some embodiments, the payload gene comprises a nucleic acid (i.e., a heterologous, recombinant, exogenous, or therapeutic gene) that encodes a cytosolic protein, e.g., a protein that is produced in the recipient cell and localizes to the recipient cell cytoplasm. In some embodiments, the payload gene comprises a nucleic acid that encodes a secreted protein, e.g., a protein that is produced and secreted by the recipient cell. In some embodiments, the payload gene comprises a nucleic acid that encodes a nuclear protein, e.g., a protein that is produced in the recipient cell and is imported to the nucleus of the recipient cell. In some embodiments, the payload gene comprises a nucleic acid that encodes an organellar protein (e.g., a mitochondrial protein), e.g., a protein that is produced in the recipient cell and is imported into an organelle (e.g., a mitochondrial) of the recipient cell.

In some embodiments, the payload gene comprises a nucleic acid (i.e., a heterologous, recombinant, exogenous, or therapeutic gene) that encodes a membrane protein. In some embodiments, the membrane protein comprises a nucleic acid that encodes a chimeric antigen receptor (CAR), a T cell receptor, an integrin, an ion channel, a pore forming protein, a Toll-Like Receptor, an interleukin receptor, a cell adhesion protein, or a transport protein. In some embodiments, delivery of the nuclease is by a provided vector encoding the nuclease (e.g. Cas).

In some embodiments, the payload gene is a globin gene. In some embodiments, the payload gene is ADA, IL2RG, JAK3, IL7R, HBB, F8, F9, WAS, CYBA, CYBB, NCF1, NCF2, NCF4, UROS, TCIRG1, CLCN7, MPL, ITGA2B, ITGB3, ITGB2, PKLR, SLC25, A38, RAG1, RAG2, FANCA, FANCC, FANCG, ABCD1, MAN2B1, AGA, LYST, CTNS, LAMP2, GLA, CTSA, GBA, GAA, IDS, IDUA, ISSD, ARSB, GALNS, GLB1, NEU1, GNPTA, SUMF1, SMPD1, NPC1, NPC2, CTSK, GNS, HGSNAT, NAGLU, SGSH, NAGA, GUSB, PSAP, LAL. In some embodiment, the payload gene can be a gene for delivery to a hematopoietic stem cell (HSC). Exemplary payload genes are described in WO2020102485, which is incorporated by reference.

For example, the payload gene can be, but is not limited to antisense ras, antisense myc, antisense raf, antisense erb, antisense src, antisense fins, antisense jun, antisense trk, antisense ret, antisense gsp, antisense hst, antisense bcl, antisense abl, Rb, CFTR, pi 6, p21, p27, p57, p73, C-CAM, APC, CTS-I, zac1, scFV ras, DCC, NF-I, NF-2, WT-I, MEN-I, MEN-II, BRCA1, VHL, MMAC1, FCC, MCC, BRCA2, IL-I, IL-2, IL-3, IL-4, IL-5, IL-6, IL-7, IL-8, IL-9, IL-10, IL-Il IL-12, GM-CSF, G-CSF, thymidine kinase, mda7, fus-1, interferon α, interferon β, interferon γ, ADP, p53, ABLI, BLCl, BLC6, CBFAl, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIMl, PML, RET, SRC, TALI, TCL3, YES, MADH4, RBl, TP53, WT1, TNF, BDNF, CNTF, NGF, IGF, GMF, aFGF, bFGF, NT3, NT5, ApoAI, ApoAIV, ApoE, RaplA, cytosine deaminase, Fab, ScFv, BRCA2, zac1, ATM, HIC-I, DPC-4, FHIT, PTEN, INGl, NOEY1, NOEY2, OVCAl, MADR2, 53BP2, IRF-I, Rb, zac1, DBCCR-I, rks-3, COX-I, TFPI, PGS, Dp, E2F, ras, myc, neu, raf, erb, fins, trk, ret, gsp, hst, abl, E1A, p300, VEGF, FGF, thrombospondin, BAI-I, GDAIF, or MCC. In further embodiments of the present invention, the payload gene is a gene encoding an ACP desaturase, an ACP hydroxylase, an ADP-glucose pyrophorylase, an ATPase, an alcohol dehydrogenase, an amylase, an amyloglucosidase, a catalase, a cellulase, a cyclooxygenase, a decarboxylase, a dextrinase, an esterase, a DNA polymerase, an RNA polymerase, a hyaluron synthase, a galactosidase, a glucanase, a glucose oxidase, a GTPase, a helicase, a hemicellulase, a hyaluronidase, an integrase, an invertase, an isomerase, a kinase, a lactase, a lipase, a lipoxygenase, a lyase, a lysozyrne, a pectinesterase, a peroxidase, a phosphatase, a phospholipase, a phosphorylase, a polygalacturonase, a proteinase, a peptidease, a pullanase, a recombinase, a reverse transcriptase, a topoisomerase, a xylanase, a reporter gene, an interleukin, or a cytokine. In other embodiments of the present invention, the payload gene is a gene encoding carbamoyl synthetase I, ornithine transcarbamylase, arginosuccinate synthetase, arginosuccinate lyase, arginase, fumarylacetoacetate hydrolase, phenylalanine hydroxylase, alpha-1 antitrypsin, gmcose-6-phosphatase, low-density-lipoprotein receptor, porphobilinogen deaminase, factor VIII, factor IX, cystathione α-synthase, branched chain ketoacid decarboxylase, albumin, isovaleryl-CoA dehydrogenase, propionyl CoA carboxylase, methyl malonyl CoA mutase, glutaryl CoA dehydrogenase, insulin, beta.-glucosidase, pyruvate carboxylase, hepatic phosphorylase, phosphorylase kinase, glycine decarboxylase, H-protein, T-protein, Menkes disease copper-transporting ATPase, Wilson's disease copper-transporting ATPase, cytosine deaminase, hypoxanthine-guanine phosphoribosyltransferase. galactose-1-phosphate uridyltransferase, phenylalanine hydroxylase, glucocerbrosidase, sphingomyelinase, α-L-iduronidase, glucose-6-phosphate dehydrogenase, HSV thymidine kinase, or human thymidine kinase. Alternatively, the recombinant gene may encode growth hormone, prolactin, placental lactogen, luteinizing hormone, follicle-stimulating hormone, chorionic gonadotropin, thyroid-stimulating hormone, leptin, adrenocorticotropin, angiotensin I, angiotensin II, β-endorphin, β-melanocyte stimulating hormone, cholecystokinin, endothelin I, galanin, gastric inhibitory peptide, glucagon, insulin, lipotropins, neurophysins, somatostatin, calcitonin, calcitonin gene related peptide, (3-calcitonin gene related peptide, hypercalcemia of malignancy factor, parathyroid hormone-related protein, parathyroid hormone-related protein, glucagon-like peptide, pancreastatin, pancreatic peptide, peptide YY, PHM, secretin, vasoactive intestinal peptide, oxytocin, vasopressin, vasotocin, enkephalinamide, metorphinamide, alpha melanocyte stimulating hormone, atrial natriuretic factor, amylin, amyloid P component, corticotropin releasing hormone, growth hormone releasing factor, luteinizing hormone-releasing hormone, neuropeptide Y, substance K, substance P, or thyrotropin releasing hormone.

### 1. Chimeric Antigen Receptors

In certain embodiments, the payload gene may comprise an exogenous polynucleotide encoding a CAR. CARs (also known as chimeric immunoreceptors, chimeric T cell receptors, or artificial T cell receptors) are receptor proteins that have been engineered to give host cells (e.g., T cells) the new ability to target a specific protein. The receptors are chimeric because they combine both antigen-binding and T cell activating functions into a single receptor. The polycistronic vector of the present disclosure may be used to express one or more CARs in a host cell (e.g., a T cell) for use in cell-based therapies against various target antigens. The CARs expressed by the one or more expression cassettes may be the same or different. In these embodiments, the CAR may comprise an extracellular binding domain (also referred to as a “binder”) that specifically binds a target antigen, a transmembrane domain, and an intracellular signaling domain. In certain embodiments, the CAR may further comprise one or more additional elements, including one or more signal peptides, one or more extracellular hinge domains, and/or one or more intracellular costimulatory domains. Domains may be directly adjacent to one another, or there may be one or more amino acids linking the domains. The nucleotide sequence encoding a CAR may be derived from a mammalian sequence, for example, a mouse sequence, a primate sequence, a human sequence, or combinations thereof. In the cases where the nucleotide sequence encoding a CAR is non-human, the sequence of the CAR may be humanized. The nucleotide sequence encoding a CAR may also be codon-optimized for expression in a mammalian cell, for example, a human cell. In any of these embodiments, the nucleotide sequence encoding a CAR may be at least 80% identical (e.g., at least 80%, at least 85%, at least 90%, at least 95%, at least 96%, at least 97%, at least 98%, at least 99%, or 100% identical) to any of the nucleotide sequences disclosed herein. The sequence variations may be due to codon-optimization, humanization, restriction enzyme-based cloning scars, and/or additional amino acid residues linking the functional domains, etc.

In certain embodiments, the CAR may comprise a signal peptide at the N-terminus. Non-limiting examples of signal peptides include CD8a signal peptide, IgK signal peptide, and granulocyte-macrophage colony-stimulating factor receptor subunit alpha (GMCSFR-α, also known as colony stimulating factor 2 receptor subunit alpha (CSF2RA)) signal peptide, and variants thereof, the amino acid sequences of which are provided in Table 4 below.

TABLE 4
Exemplary sequences of signal peptides

| SEQ ID | | |
|---|---|---|
| NO: | Sequence | Description |
| 24 | MALPVTALLLPLALLLHAARP | CD8α signal peptide |
| 25 | METDTLLLWVLLLWVPGSTG | IgK signal peptide |
| 26 | MLLLVTSLLLCELPHPAFLLIP | GMCSFR-α (CSF2RA) signal peptide |

In certain embodiments, the extracellular binding domain of the CAR may comprise one or more antibodies specific to one target antigen or multiple target antigens. The antibody may be an antibody fragment, for example, an scFv, or a single-domain antibody fragment, for example, a VHH. In certain embodiments, the scFv may comprise a heavy chain variable region (V_{H}) and a light chain variable region (V_{L}) of an antibody connected by a linker. The V_{H} and the V_{L} may be connected in either order, i.e., V_{H}-linker-V_{L} or V_{L}-linker-V_{H}. Non-limiting examples of linkers include Whitlow linker, (G₄S)˜(n can be a positive integer, e.g., 1, 2, 3, 4, 5, 6, etc.) linker, and variants thereof. In certain embodiments, the antigen may be an antigen that is exclusively or preferentially expressed on tumor cells, or an antigen that is characteristic of an autoimmune or inflammatory disease. Exemplary target antigens include, but are not limited to, CD5, CD19, CD20, CD22, CD23, CD30, CD70, Kappa, Lambda, and B cell maturation agent (BCMA), G-protein coupled receptor family C group 5 member D (GPRC5D) (associated with leukemias); CS1/SLAMF7, CD38, CD138, GPRC5D, TA, and BCMA (associated with myelomas); GD2, HER2, EGFR, EGFRvIII, 1B71H3, PSMA, PSCA, CAIX, CD171, CEA, CSPG4, EPHA2, FAP, FRα, IL-13Rα, Mesothelin, MUC1, MUC16, and ROR1 (associated with solid tumors). In any of these embodiments, the extracellular binding domain of the CAR can be codon-optimized for expression in a host cell or have variant sequences to increase functions of the extracellular binding domain.

In certain embodiments, the CAR may comprise a hinge domain, also referred to as a spacer. The terms “hinge” and “spacer” may be used interchangeably in the present disclosure. Non-limiting examples of hinge domains include CD8a hinge domain, CD28 hinge domain, IgG4 hinge domain, IgG4 hinge-CH2-CH3 domain, and variants thereof, the amino acid sequences of which are provided in Table 5 below.

TABLE 5
Exemplary sequences of hinge domains

| SEQ ID | | |
|---|---|---|
| NO: | Sequence | Description |
| 27 | TTTPAPRPPTPAPTIASQPLSLRPEACRPAAGGAVH | CD8α hinge domain |
| | TRGLDFACD | |
| 28 | IEVMYPPPYLDNEKSNGTIIHVKGKHLCPSPLFPGP | CD28 hinge domain |
| | SKP | |
| 29 | AAAIEVMYPPPYLDNEKSNGTIIHVKGKHLCPSPLF | CD28 hinge domain |
| | PGPSKP | |
| 30 | ESKYGPPCPPCP | IgG4 hinge domain |
| 31 | ESKYGPPCPSCP | IgG4 hinge domain |
| 32 | ESKYGPPCPPCPAPEFLGGPSVFLFPPKPKDTLMISR | IgG4 hinge-CH2-CH3 |
| | TPEVTCVVVDVSQEDPEVQFNWYVDGVEVHNAK | domain |
| | TKPREEQFNSTYRVVSVLTVLHQDWLNGKEYKCK | |
| | VSNKGLPSSIEKTISKAKGQPREPQVYTLPPSQEEM | |
| | TKNQVSLTCLVKGFYPSDIAVEWESNGQPENNYK | |
| | TTPPVLDSDGSFFLYSRLTVDKSRWQEGNVFSCSV | |
| | MHEALHNHYTQKSLSLSLGK | |

In certain embodiments, the transmembrane domain of the CAR may comprise a transmembrane region of the alpha, beta, or zeta chain of a T cell receptor, CD28, CD3E, CD45, CD4, CD5, CD8, CD9, CD16, CD22, CD33, CD37, CD64, CD80, CD86, CD134, CD137, CD154, or a functional variant thereof, including the human versions of each of these sequences. In other embodiments, the transmembrane domain may comprise a transmembrane region of CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B, or a functional variant thereof, including the human versions of each of these sequences. Table 6 provides the amino acid sequences of a few exemplary transmembrane domains.

TABLE 6
Exemplary sequences of transmembrane domains

| SEQ ID | | |
|---|---|---|
| NO: | Sequence | Description |
| 33 | IYIWAPLAGTCGVLLLSLVITLYC | CD8α transmembrane domain |
| 34 | FWVLVVVGGVLACYSLLVTVAFIIFWV | CD28 transmembrane domain |
| 35 | MFWVLVVVGGVLACYSLLVTVAFIIFWV | CD28 transmembrane domain |

In certain embodiments, the intracellular signaling domain and/or intracellular costimulatory domain of the CAR may comprise one or more signaling domains selected from B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, PDCD6, 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNFβ, OX40/TNFRSF4, OX40 Ligand/TNFSF4, RELT/TNFRSF19L, TACI/TNFRSF13B, TL1A/TNFSFi5, TNFα, TNF RII/TNFRSF1B, 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, SLAM/CD150, CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), NKG2C, CD3ζ, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, and a functional variant thereof including the human versions of each of these sequences. In some embodiments, the intracellular signaling domain and/or intracellular costimulatory domain comprises one or more signaling domains selected from a CD3ζ domain, an ITAM, a CD28 domain, 4-1BB domain, or a functional variant thereof. Table 7 provides the amino acid sequences of a few exemplary intracellular costimulatory and/or signaling domains. In certain embodiments, as in the case of tisagenlecleucel as described below, the CD3ζ signaling domain of SEQ ID NO:38 may have a mutation, e.g., a glutamine (Q) to lysine (K) mutation, at amino acid position 14 (see SEQ ID NO:39).

TABLE 7
Exemplary sequences of intracellular costimulatory and/or signaling domains

| SEQ ID | | |
|---|---|---|
| NO: | Sequence | Description |
| 36 | KRGRKKLLYIFKQPFMRPVQTTQEEDGCSCR | 4-1BB costimulatory domain |
| | FPEEEEGGCEL | |
| 37 | RSKRSRLLHSDYMNMTPRRPGPTRKHYQPY | CD28 costimulatory domain |
| | APPRDFAAYRS | |
| 38 | RVKFSRSADAPAYQQGQNQLYNELNLGRRE | CD32 signaling domain |
| | EYDVLDKRRGRDPEMGGKPRRKNPQEGLYN | |
| | ELQKDKMAEAYSEIGMKGERRRGKGHDGLY | |
| | QGLSTATKDTYDALHMQALPPR | |
| 39 | RVKFSRSADAPAYKQGQNQLYNELNLGRRE | CD35 signaling domain (with Q |
| | EYDVLDKRRGRDPEMGGKPRRKNPQEGLYN | to K mutation at position 14) |
| | ELQKDKMAEAYSEIGMKGERRRGKGHDGLY | |
| | QGLSTATKDTYDALHMQALPPR | |

In certain embodiments where the polycistronic vector encodes two or more CARs, the two or more CARs may comprise the same functional domains, or one or more different functional domains, as described. For example, the two or more CARs may comprise different signal peptides, extracellular binding domains, hinge domains, transmembrane domains, costimulatory domains, and/or intracellular signaling domains, in order to minimize the risk of recombination due to sequence similarities. Or, alternatively, the two or more CARs may comprise the same domains. In the cases where the same domain(s) and/or backbone are used, it is optional to introduce codon divergence at the nucleotide sequence level to minimize the risk of recombination.

a. CD19 CAR

In some embodiments, the CAR is a CD19 CAR (“CD19-CAR”), and in these embodiments, the polycistronic vector comprises an expression cassette that contains a nucleotide sequence encoding a CD19 CAR. In some embodiments, the CD19 CAR may comprise a signal peptide, an extracellular binding domain that specifically binds CD19, a hinge domain, a transmembrane domain, an intracellular costimulatory domain, and/or an intracellular signaling domain in tandem.

In some embodiments, the signal peptide of the CD19 CAR comprises a CD8a signal peptide. In some embodiments, the CD8a signal peptide comprises or consists of an amino acid sequence set forth in SEQ ID NO:24 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:24. In some embodiments, the signal peptide comprises an IgK signal peptide. In some embodiments, the IgK signal peptide comprises or consists of an amino acid sequence set forth in SEQ ID NO:25 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:25. In some embodiments, the signal peptide comprises a GMCSFR-a or CSF2RA signal peptide. In some embodiments, the GMCSFR-a or CSF2RA signal peptide comprises or consists of an amino acid sequence set forth in SEQ ID NO:26 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:26.

In some embodiments, the extracellular binding domain of the CD19 CAR is specific to CD19, for example, human CD19. The extracellular binding domain of the CD19 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD19 CAR comprises an scFv derived from the FMC63 monoclonal antibody (FMC63), which comprises the heavy chain variable region (V_{H}) and the light chain variable region (V_{L}) of FMC63 connected by a linker. FMC63 and the derived scFv have been described in Nicholson et al., Mol. Immun. 34(16-17):1157-1165 (1997) and PCT Application Publication No. WO2018/213337, the entire contents of each of which are incorporated by reference herein. In some embodiments, the amino acid sequences of the entire FMC63-derived scFv (also referred to as FMC63 scFv) and its different portions are provided in Table 8 below. In some embodiments, the CD19-specific scFv comprises or consists of an amino acid sequence set forth in SEQ ID NO:40, 41, or 46, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:40, 41, or 46. In some embodiments, the CD19-specific scFv may comprise one or more CDRs having amino acid sequences set forth in SEQ ID NOs: 42-44 and 48-50. In some embodiments, the CD19-specific scFv may comprise a light chain with one or more CDRs having amino acid sequences set forth in SEQ ID NOs: 42-44. In some embodiments, the CD19-specific scFv may comprise a heavy chain with one or more CDRs having amino acid sequences set forth in SEQ ID NOs: 48-50. In any of these embodiments, the CD19-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD19 CAR comprises or consists of the one or more CDRs as described herein.

In some embodiments, the linker linking the V_{H} and the V_{L} portions of the scFv is a Whitlow linker having an amino acid sequence set forth in SEQ ID NO:45. In some embodiments, the Whitlow linker may be replaced by a different linker, for example, a 3×G₄S linker having an amino acid sequence set forth in SEQ ID NO:51, which gives rise to a different FMC63-derived scFv having an amino acid sequence set forth in SEQ ID NO:50. In certain of these embodiments, the CD19-specific scFv comprises or consists of an amino acid sequence set forth in SEQ ID NO:50 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:50.

TABLE 8
Exemplary sequences of anti-CD19 scFv and components

| SEQ ID NO: | Amino Acid Sequence | Description |
|---|---|---|
| 40 | DIQMTQTTSSLSASLGDRVTISCRASQDI | Anti-CD19 FMC63 scFv |
| | SKYLNWYQQKPDGTVKLLIYHTSRLHS | entire sequence, with |
| | GVPSRFSGSGSGTDYSLTISNLEQEDIAT | Whitlow linker |
| | YFCQQGNTLPYTFGGGTKLEITGSTSGS | |
| | GKPGSGEGSTKGEVKLQESGPGLVAPS | |
| | QSLSVTCTVSGVSLPDYGVSWIRQPPRK | |
| | GLEWLGVIWGSETTYYNSALKSRLTIIK | |
| | DNSKSQVFLKMNSLQTDDTAIYYCAKH | |
| | YYYGGSYAMDYWGQGTSVTVSS | |
| 41 | DIQMTQTTSSLSASLGDRVTISCRASQDI | Anti-CD19 FMC63 scFv |
| | SKYLNWYQQKPDGTVKLLIYHTSRLHS | light chain variable region |
| | GVPSRFSGSGSGTDYSLTISNLEQEDIAT | |
| | YFCQQGNTLPYTFGGGTKLEIT | |
| 42 | QDISKY | Anti-CD19 FMC63 scFv |
| | | light chain CDR1 |
| 43 | HTS | Anti-CD19 FMC63 scFv |
| | | light chain CDR2 |
| 44 | QQGNTLPYT | Anti-CD19 FMC63 scFv |
| | | light chain CDR3 |
| 45 | GSTSGSGKPGSGEGSTKG | Whitlow linker |
| 46 | EVKLQESGPGLVAPSQSLSVTCTVSGVS | Anti-CD19 FMC63 scFv |
| | LPDYGVSWIRQPPRKGLEWLGVIWGSE | heavy chain variable region |
| | TTYYNSALKSRLTIIKDNSKSQVFLKMN | |
| | SLQTDDTAIYYCAKHYYYGGSYAMDY | |
| | WGQGTSVTVSS | |
| 47 | GVSLPDYG | Anti-CD19 FMC63 scFv |
| | | heavy chain CDR1 |
| 48 | IWGSETT | Anti-CD19 FMC63 scFv |
| | | heavy chain CDR2 |
| 49 | AKHYYYGGSYAMDY | Anti-CD19 FMC63 scFv |
| | | heavy chain CDR3 |
| 50 | DIQMTQTTSSLSASLGDRVTISCRASQDI | Anti-CD19 FMC63 scFv |
| | SKYLNWYQQKPDGTVKLLIYHTSRLHS | entire sequence, with 3xG₄S |
| | GVPSRFSGSGSGTDYSLTISNLEQEDIAT | linker |
| | YFCQQGNTLPYTFGGGTKLEITGGGGS | |
| | GGGGSGGGGSEVKLQESGPGLVAPSQS | |
| | LSVTCTVSGVSLPDYGVSWIRQPPRKGL | |
| | EWLGVIWGSETTYYNSALKSRLTIIKDN | |
| | SKSQVFLKMNSLQTDDTAIYYCAKHYY | |
| | YGGSYAMDYWGQGTSVTVSS | |
| 51 | GGGGSGGGGSGGGGS | 3xG₄S linker |

In some embodiments, the extracellular binding domain of the CD19 CAR is derived from an antibody specific to CD19, including, for example, SJ25C1 (Bejcek et al., Cancer Res. 55:2346-2351 (1995)), HD37 (Pezutto et al., J. Immunol. 138(9):2793-2799 (1987)), 4G7 (Meeker et al., Hybridoma 3:305-320 (1984)), B43 (Bejcek (1995)), BLY3 (Bejcek (1995)), B4 (Freedman et al., 70:418-427 (1987)), B4 HB12b (Kansas & Tedder, J. Immunol. 147:4094-4102 (1991); Yazawa et al., Proc. Natl. Acad. Sci. USA 102:15178-15183 (2005); Herbst et al., J. Pharmacol. Exp. Ther. 335:213-222 (2010)), BU12 (Callard et al., J. Immunology, 148(10): 2983-2987 (1992)), and CLB-CD19 (De Rie Cell. Immunol. 118:368-381(1989)). In any of these embodiments, the extracellular binding domain of the CD19 CAR can comprise or consist of the V_{H}, the V_{L}, and/or one or more CDRs of any of the antibodies.

In some embodiments, the hinge domain of the CD19 CAR comprises a CD8a hinge domain, for example, a human CD8a hinge domain. In some embodiments, the CD8a hinge domain comprises or consists of an amino acid sequence set forth in SEQ ID NO:27 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:27. In some embodiments, the hinge domain comprises a CD28 hinge domain, for example, a human CD28 hinge domain. In some embodiments, the CD28 hinge domain comprises or consists of an amino acid sequence set forth in SEQ ID NO:28 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:28. In some embodiments, the CD28 hinge domain comprises or consists of an amino acid sequence set forth in SEQ ID NO:29 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:29. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:31 or SEQ ID NO:30, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:31 or SEQ ID NO:30. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:32 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:32.

In some embodiments, the transmembrane domain of the CD19 CAR comprises a CD8a transmembrane domain, for example, a human CD8a transmembrane domain. In some embodiments, the CD8a transmembrane domain comprises or consists of an amino acid sequence set forth in SEQ ID NO:33 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:33. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:34 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:34. In some embodiments, the CD28 transmembrane domain comprises or consists of an amino acid sequence set forth in SEQ ID NO:35 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:35.

In some embodiments, the intracellular costimulatory domain of the CD19 CAR comprises a 4-1BB costimulatory domain. 4-1BB, also known as CD137, transmits a potent costimulatory signal to T cells, promoting differentiation and enhancing long-term survival of T lymphocytes. In some embodiments, the 4-1BB costimulatory domain is human. In some embodiments, the 4-1BB costimulatory domain comprises or consists of an amino acid sequence set forth in SEQ ID NO:36 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:36. In some embodiments, the intracellular costimulatory domain comprises a CD28 costimulatory domain. CD28 is another co-stimulatory molecule on T cells. In some embodiments, the CD28 costimulatory domain is human. In some embodiments, the CD28 costimulatory domain comprises or consists of an amino acid sequence set forth in SEQ ID NO:37 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:37. In some embodiments, the intracellular costimulatory domain of the CD19 CAR comprises a 4-1BB costimulatory domain and a CD28 costimulatory domain as described.

In some embodiments, the intracellular signaling domain of the CD19 CAR comprises a CD3 zeta (ζ) signaling domain. CD3ζ associates with T cell receptors (TCRs) to produce a signal and contains immunoreceptor tyrosine-based activation motifs (ITAMs). The CD3ζ signaling domain refers to amino acid residues from the cytoplasmic domain of the zeta chain that are sufficient to functionally transmit an initial signal necessary for T cell activation. In some embodiments, the CD3ζ signaling domain is human. In some embodiments, the CD3ζ signaling domain comprises or consists of an amino acid sequence set forth in SEQ ID NO:38 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:38.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:40 or SEQ ID NO:41, the CD8α hinge domain of SEQ ID NO:27, the CD8α transmembrane domain of SEQ ID NO:33, the 4-1BB costimulatory domain of SEQ ID NO:36, the CD3ζ signaling domain of SEQ ID NO:38, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:40 or SEQ ID NO:41, the IgG4 hinge domain of SEQ ID NO:30 or SEQ ID NO:31, the CD28 transmembrane domain of SEQ ID NO:34, the 4-1BB costimulatory domain of SEQ ID NO:36, the CD3ζ signaling domain of SEQ ID NO:38, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:40 or SEQ ID NO:41, the CD28 hinge domain of SEQ ID NO:28, the CD28 transmembrane domain of SEQ ID NO:34, the CD28 costimulatory domain of SEQ ID NO:37, the CD3ζ signaling domain of SEQ ID NO:38, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR as set forth in SEQ ID NO:52 or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:52 (see Table 10). The encoded CD19 CAR has a corresponding amino acid sequence set forth in SEQ ID NO:53 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:53, with the following components: CD8α signal peptide, FMC63 scFv (V_{L}—Whitlow linker-V_{H}), CD8α hinge domain, CD8α transmembrane domain, 4-1BB costimulatory domain, and CD3ζ signaling domain.

In some embodiments, the polycistronic vector comprises an expression cassette that contains a nucleotide sequence encoding a commercially available embodiment of CD19 CAR. Non-limiting examples of commercially available embodiments of CD19 CARs expressed and/or encoded by T cells include tisagenlecleucel, lisocabtagene maraleucel, axicabtagene ciloleucel, and brexucabtagene autoleucel.

In some embodiments, the polycistronic vector comprises an expression cassette that contains a nucleotide sequence encoding tisagenlecleucel or portions thereof. Tisagenlecleucel comprises a CD19 CAR with the following components: CD8α signal peptide, FMC63 scFv (V_{L}-3×G₄S linker-V_{H}), CD8α hinge domain, CD8α transmembrane domain, 4-1BB costimulatory domain, and CD3ζ signaling domain. The nucleotide and amino acid sequence of the CD19 CAR in tisagenlecleucel are provided in Table 10, with annotations of the sequences provided in Table 10.

In some embodiments, the polycistronic vector comprises an expression cassette that contains a nucleotide sequence encoding lisocabtagene maraleucel or portions thereof. Lisocabtagene maraleucel comprises a CD19 CAR with the following components: GMCSFR-a or CSF2RA signal peptide, FMC63 scFv (V_{L}—Whitlow linker-V_{H}), IgG4 hinge domain, CD28 transmembrane domain, 4-1BB costimulatory domain, and CD3ζ signaling domain. The nucleotide and amino acid sequence of the CD19 CAR in lisocabtagene maraleucel are provided in Table 9, with annotations of the sequences provided in Table 11.

In some embodiments, the polycistronic vector comprises an expression cassette that contains a nucleotide sequence encoding axicabtagene ciloleucel or portions thereof. Axicabtagene ciloleucel comprises a CD19 CAR with the following components: GMCSFR-a or CSF2RA signal peptide, FMC63 scFv (V_{L}-Whitlow linker-V_{H}), CD28 hinge domain, CD28 transmembrane domain, CD28 costimulatory domain, and CD3ζ signaling domain. The nucleotide and amino acid sequence of the CD19 CAR in axicabtagene ciloleucel are provided in Table 9, with annotations of the sequences provided in Table 12.

In some embodiments, the polycistronic vector comprises an expression cassette that contains a nucleotide sequence encoding brexucabtagene autoleucel or portions thereof. Brexucabtagene autoleucel comprises a CD19 CAR with the following components: GMCSFR-α signal peptide, FMC63 scFv, CD28 hinge domain, CD28 transmembrane domain, CD28 costimulatory domain, and CD3ζ signaling domain.

In some embodiments, the polycistronic vector comprises an expression cassette that contains a nucleotide sequence encoding a CD19 CAR as set forth in SEQ ID NO: 54, 56, or 58, or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 54, 56, or 58. The encoded CD19 CAR has a corresponding amino acid sequence set forth in SEQ ID NO: 55, 57, or 59, respectively, or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 55, 57, or 59, respectively.

TABLE 9
Exemplary sequences of CD19 CARs

| SEQ ID NO: | Sequence | Description |
|---|---|---|
| 52 | atggccttaccagtgaccgccttgctcctgccgctggccttgctgctccac | Exemplary CD19 |
| | gccgccaggccggacatccagatgacacagactacatcctccctgtctgc | CAR nucleotide |
| | ctctctgggagacagagtcaccatcagttgcagggcaagtcaggacatta | sequence |
| | gtaaatatttaaattggtatcagcagaaaccagatggaactgttaaactcct | |
| | gatctaccatacatcaagattacactcaggagtcccatcaaggttcagtgg | |
| | cagtgggtctggaacagattattctctcaccattagcaacctggagcaaga | |
| | agatattgccacttacttttgccaacagggtaatacgcttccgtacacgttcg | |
| | gaggggggaccaagctggagatcacaggctccacctctggatccggca | |
| | agcccggatctggcgagggatccaccaagggcgaggtgaaactgcag | |
| | gagtcaggacctggcctggtggcgccctcacagagcctgtccgtcacat | |
| | gcactgtctcaggggtctcattacccgactatggtgtaagctggattcgcc | |
| | agcctccacgaaagggtctggagtggctgggagtaatatggggtagtga | |
| | aaccacatactataattcagctctcaaatccagactgaccatcatcaagga | |
| | caactccaagagccaagttttcttaaaaatgaacagtctgcaaactgatga | |
| | cacagccatttactactgtgccaaacattattactacggtggtagctatgcta | |
| | tggactactggggccaaggaacctcagtcaccgtctcctcaaccacgac | |
| | gccagcgccgcgaccaccaacaccggcgcccaccatcgcgtcgcagc | |
| | ccctgtccctgcgcccagaggcgtgccggccagcggcggggggcgca | |
| | gtgcacacgagggggctggacttcgcctgtgatatctacatctgggcgcc | |
| | cttggccgggacttgtggggtccttctcctgtcactggttatcaccctttact | |
| | gcaaacggggcagaaagaaactcctgtatatattcaaacaaccatttatga | |
| | gaccagtacaaactactcaagaggaagatggctgtagctgccgatttcca | |
| | gaagaagaagaaggaggatgtgaactgagagtgaagttcagcaggagc | |
| | gcagacgcccccgcgtaccagcagggccagaaccagctctataacgag | |
| | ctcaatctaggacgaagagaggagtacgatgttttggacaagagacgtgg | |
| | ccgggaccctgagatggggggaaagccgagaaggaagaaccctcagg | |
| | aaggcctgtacaatgaactgcagaaagataagatggcggaggcctacag | |
| | tgagattgggatgaaaggcgagcgccggaggggcaaggggcacgatg | |
| | gcctttaccagggtctcagtacagccaccaaggacacctacgacgccctt | |
| | cacatgcaggccctgccccctcgc | |
| 53 | MALPVTALLLPLALLLHAARPDIQMTQTTSSLS | Exemplary CD19 |
| | ASLGDRVTISCRASQDISKYLNWYQQKPDGTV | CAR amino acid |
| | KLLIYHTSRLHSGVPSRFSGSGSGTDYSLTISNL | sequence |
| | EQEDIATYFCQQGNTLPYTFGGGTKLEITGSTS | |
| | GSGKPGSGEGSTKGEVKLQESGPGLVAPSQSLS | |
| | VTCTVSGVSLPDYGVSWIRQPPRKGLEWLGVI | |
| | WGSETTYYNSALKSRLTIIKDNSKSQVFLKMNS | |
| | LQTDDTAIYYCAKHYYYGGSYAMDYWGQGT | |
| | SVTVSSTTTPAPRPPTPAPTIASQPLSLRPEACRP | |
| | AAGGAVHTRGLDFACDIYIWAPLAGTCGVLLL | |
| | SLVITLYCKRGRKKLLYIFKQPFMRPVQTTQEE | |
| | DGCSCRFPEEEEGGCELRVKFSRSADAPAYQQ | |
| | GQNQLYNELNLGRREEYDVLDKRRGRDPEMG | |
| | GKPRRKNPQEGLYNELQKDKMAEAYSEIGMK | |
| | GERRRGKGHDGLYQGLSTATKDTYDALHMQA | |
| | LPPR | |
| 54 | atggccttaccagtgaccgccttgctcctgccgctggccttgctgctccac | Tisagenlecleucel |
| | gccgccaggccggacatccagatgacacagactacatcctccctgtctgc | CD19 CAR |
| | ctctctgggagacagagtcaccatcagttgcagggcaagtcaggacatta | nucleotide |
| | gtaaatatttaaattggtatcagcagaaaccagatggaactgttaaactcct | sequence |
| | gatctaccatacatcaagattacactcaggagtcccatcaaggttcagtgg | |
| | cagtgggtctggaacagattattctctcaccattagcaacctggagcaaga | |
| | agatattgccacttacttttgccaacagggtaatacgcttccgtacacgttcg | |
| | gaggggggaccaagctggagatcacaggtggcggtggctcgggcggt | |
| | ggtgggtcgggtggcggcggatctgaggtgaaactgcaggagtcagga | |
| | cctggcctggtggcgccctcacagagcctgtccgtcacatgcactgtctc | |
| | aggggtctcattacccgactatggtgtaagctggattcgccagcctccacg | |
| | aaagggtctggagtggctgggagtaatatggggtagtgaaaccacatact | |
| | ataattcagctctcaaatccagactgaccatcatcaaggacaactccaaga | |
| | gccaagttttcttaaaaatgaacagtctgcaaactgatgacacagccattta | |
| | ctactgtgccaaacattattactacggtggtagctatgctatggactactgg | |
| | ggccaaggaacctcagtcaccgtctcctcaaccacgacgccagcgccg | |
| | cgaccaccaacaccggcgcccaccatcgcgtcgcagcccctgtccctgc | |
| | gcccagaggcgtgccggccagcggcggggggcgcagtgcacacgag | |
| | ggggctggacttcgcctgtgatatctacatctgggcgcccttggccggga | |
| | cttgtggggtccttctcctgtcactggttatcaccctttactgcaaacggggc | |
| | agaaagaaactcctgtatatattcaaacaaccatttatgagaccagtacaaa | |
| | ctactcaagaggaagatggctgtagctgccgatttccagaagaagaagaa | |
| | ggaggatgtgaactgagagtgaagttcagcaggagcgcagacgccccc | |
| | gcgtacaagcagggccagaaccagctctataacgagctcaatctaggac | |
| | gaagagaggagtacgatgttttggacaagagacgtggccgggaccctga | |
| | gatggggggaaagccgagaaggaagaaccctcaggaaggcctgtaca | |
| | atgaactgcagaaagataagatggcggaggcctacagtgagattgggat | |
| | gaaaggcgagcgccggaggggcaaggggcacgatggcctttaccagg | |
| | gtctcagtacagccaccaaggacacctacgacgcccttcacatgcaggc | |
| | cctgccccctcgc | |
| 55 | MALPVTALLLPLALLLHAARPDIQMTQTTSSLS | Tisagenlecleucel |
| | ASLGDRVTISCRASQDISKYLNWYQQKPDGTV | CD19 CAR amino |
| | KLLIYHTSRLHSGVPSRFSGSGSGTDYSLTISNL | acid sequence |
| | EQEDIATYFCQQGNTLPYTFGGGTKLEITGGGG | |
| | SGGGGSGGGGSEVKLQESGPGLVAPSQSLSVT | |
| | CTVSGVSLPDYGVSWIRQPPRKGLEWLGVIWG | |
| | SETTYYNSALKSRLTIIKDNSKSQVFLKMNSLQ | |
| | TDDTAIYYCAKHYYYGGSYAMDYWGQGTSV | |
| | TVSSTTTPAPRPPTPAPTIASQPLSLRPEACRPA | |
| | AGGAVHTRGLDFACDIYIWAPLAGTCGVLLLS | |
| | LVITLYCKRGRKKLLYIFKQPFMRPVQTTQEED | |
| | GCSCRFPEEEEGGCELRVKFSRSADAPAYKQG | |
| | QNQLYNELNLGRREEYDVLDKRRGRDPEMGG | |
| | KPRRKNPQEGLYNELQKDKMAEAYSEIGMKG | |
| | ERRRGKGHDGLYQGLSTATKDTYDALHMQAL | |
| | PPR | |
| 56 | atgctgctgctggtgaccagcctgctgctgtgcgagctgccccaccccgc | Lisocabtagene |
| | ctttctgctgatccccgacatccagatgacccagaccacctccagcctgag | maraleucel CD19 |
| | cgccagcctgggcgaccgggtgaccatcagctgccgggccagccagg | CAR nucleotide |
| | acatcagcaagtacctgaactggtatcagcagaagcccgacggcaccgt | sequence |
| | caagctgctgatctaccacaccagccggctgcacagcggcgtgcccagc | |
| | cggtttagcggcagcggctccggcaccgactacagcctgaccatctcca | |
| | acctggaacaggaagatatcgccacctacttttgccagcagggcaacaca | |
| | ctgccctacacctttggcggcggaacaaagctggaaatcaccggcagca | |
| | cctccggcagcggcaagcctggcagcggcgagggcagcaccaaggg | |
| | cgaggtgaagctgcaggaaagcggccctggcctggtggcccccagcca | |
| | gagcctgagcgtgacctgcaccgtgagcggcgtgagcctgcccgacta | |
| | cggcgtgagctggatccggcagccccccaggaagggcctggaatggct | |
| | gggcgtgatctggggcagcgagaccacctactacaacagcgccctgaa | |
| | gagccggctgaccatcatcaaggacaacagcaagagccaggtgttcctg | |
| | aagatgaacagcctgcagaccgacgacaccgccatctactactgcgcca | |
| | agcactactactacggcggcagctacgccatggactactggggccaggg | |
| | caccagcgtgaccgtgagcagcgaatctaagtacggaccgccctgcccc | |
| | ccttgccctatgttctgggtgctggtggtggtcggaggcgtgctggcctgc | |
| | tacagcctgctggtcaccgtggccttcatcatcttttgggtgaaacggggc | |
| | agaaagaaactcctgtatatattcaaacaaccatttatgagaccagtacaaa | |
| | ctactcaagaggaagatggctgtagctgccgatttccagaagaagaagaa | |
| | ggaggatgtgaactgcgggtgaagttcagcagaagcgccgacgcccct | |
| | gcctaccagcagggccagaatcagctgtacaacgagctgaacctgggc | |
| | agaagggaagagtacgacgtcctggataagcggagaggccgggaccc | |
| | tgagatgggcggcaagcctcggcggaagaacccccaggaaggcctgta | |
| | taacgaactgcagaaagacaagatggccgaggcctacagcgagatcgg | |
| | catgaagggcgagcggaggcggggcaagggccacgacggcctgtatc | |
| | agggcctgtccaccgccaccaaggatacctacgacgccctgcacatgca | |
| | ggccctgcccccaagg | |
| 57 | MLLLVTSLLLCELPHPAFLLIPDIQMTQTTSSLS | Lisocabtagene |
| | ASLGDRVTISCRASQDISKYLNWYQQKPDGTV | maraleucel CD19 |
| | KLLIYHTSRLHSGVPSRFSGSGSGTDYSLTISNL | CAR amino acid |
| | EQEDIATYFCQQGNTLPYTFGGGTKLEITGSTS | sequence |
| | GSGKPGSGEGSTKGEVKLQESGPGLVAPSQSLS | |
| | VTCTVSGVSLPDYGVSWIRQPPRKGLEWLGVI | |
| | WGSETTYYNSALKSRLTIIKDNSKSQVFLKMNS | |
| | LQTDDTAIYYCAKHYYYGGSYAMDYWGQGT | |
| | SVTVSSESKYGPPCPPCPMFWVLVVVGGVLAC | |
| | YSLLVTVAFIIFWVKRGRKKLLYIFKQPFMRPV | |
| | QTTQEEDGCSCRFPEEEEGGCELRVKFSRSADA | |
| | PAYQQGQNQLYNELNLGRREEYDVLDKRRGR | |
| | DPEMGGKPRRKNPQEGLYNELQKDKMAEAYS | |
| | EIGMKGERRRGKGHDGLYQGLSTATKDTYDA | |
| | LHMQALPPR | |
| 58 | atgcttctcctggtgacaagccttctgctctgtgagttaccacacccagcatt | Axicabtagene |
| | cctcctgatcccagacatccagatgacacagactacatcctccctgtctgc | ciloleucel CD19 |
| | ctctctgggagacagagtcaccatcagttgcagggcaagtcaggacatta | CAR nucleotide |
| | gtaaatatttaaattggtatcagcagaaaccagatggaactgttaaactcct | sequence |
| | gatctaccatacatcaagattacactcaggagtcccatcaaggttcagtgg | |
| | cagtgggtctggaacagattattctctcaccattagcaacctggagcaaga | |
| | agatattgccacttacttttgccaacagggtaatacgcttccgtacacgttcg | |
| | gaggggggactaagttggaaataacaggctccacctctggatccggcaa | |
| | gcccggatctggcgagggatccaccaagggcgaggtgaaactgcagg | |
| | agtcaggacctggcctggtggcgccctcacagagcctgtccgtcacatg | |
| | cactgtctcaggggtctcattacccgactatggtgtaagctggattcgcca | |
| | gcctccacgaaagggtctggagtggctgggagtaatatggggtagtgaa | |
| | accacatactataattcagctctcaaatccagactgaccatcatcaaggac | |
| | aactccaagagccaagttttcttaaaaatgaacagtctgcaaactgatgac | |
| | acagccatttactactgtgccaaacattattactacggtggtagctatgctat | |
| | ggactactggggtcaaggaacctcagtcaccgtctcctcagcggccgca | |
| | attgaagttatgtatcctcctccttacctagacaatgagaagagcaatggaa | |
| | ccattatccatgtgaaagggaaacacctttgtccaagtcccctatttcccgg | |
| | accttctaagcccttttgggtgctggtggtggttgggggagtcctggcttgc | |
| | tatagcttgctagtaacagtggcctttattattttctgggtgaggagtaagag | |
| | gagcaggctcctgcacagtgactacatgaacatgactccccgccgcccc | |
| | gggcccacccgcaagcattaccagccctatgccccaccacgcgacttcg | |
| | cagcctatcgctccagagtgaagttcagcaggagcgcagacgcccccg | |
| | cgtaccagcagggccagaaccagctctataacgagctcaatctaggacg | |
| | aagagaggagtacgatgttttggacaagagacgtggccgggaccctgag | |
| | atggggggaaagccgagaaggaagaaccctcaggaaggcctgtacaat | |
| | gaactgcagaaagataagatggcggaggcctacagtgagattgggatga | |
| | aaggcgagcgccggaggggcaaggggcacgatggcctttaccagggt | |
| | ctcagtacagccaccaaggacacctacgacgcccttcacatgcaggccc | |
| | tgccccctcgc | |
| 59 | MLLLVTSLLLCELPHPAFLLIPDIQMTQTTSSLS | Axicabtagene |
| | ASLGDRVTISCRASQDISKYLNWYQQKPDGTV | ciloleucel CD19 |
| | KLLIYHTSRLHSGVPSRFSGSGSGTDYSLTISNL | CAR amino acid |
| | EQEDIATYFCQQGNTLPYTFGGGTKLEITGSTS | sequence |
| | GSGKPGSGEGSTKGEVKLQESGPGLVAPSQSLS | |
| | VTCTVSGVSLPDYGVSWIRQPPRKGLEWLGVI | |
| | WGSETTYYNSALKSRLTIIKDNSKSQVFLKMNS | |
| | LQTDDTAIYYCAKHYYYGGSYAMDYWGQGT | |
| | SVTVSSAAAIEVMYPPPYLDNEKSNGTIIHVKG | |
| | KHLCPSPLFPGPSKPFWVLVVVGGVLACYSLL | |
| | VTVAFIIFWVRSKRSRLLHSDYMNMTPRRPGPT | |
| | RKHYQPYAPPRDFAAYRSRVKFSRSADAPAYQ | |
| | QGQNQLYNELNLGRREEYDVLDKRRGRDPEM | |
| | GGKPRRKNPQEGLYNELQKDKMAEAYSEIGM | |
| | KGERRRGKGHDGLYQGLSTATKDTYDALHMQ | |
| | ALPPR | |

TABLE 10
Annotation of tisagenlecleucel CD19 CAR sequences

| | Nucleotide | Amino Acid |
|---|---|---|
| | Sequence | Sequence |
| Feature | Position | Position |
| CD8α signal peptide | 1-63 | 1-21 |
| FMC63 scFv | 64-789 | 22-263 |
| (V_{L}-3xG₄S linker-V_{H}) | | |
| CD8α hinge domain | 790-924 | 264-308 |
| CD8α transmembrane domain | 925-996 | 309-332 |
| 4-1BB costimulatory domain | 997-1122 | 333-374 |
| CD3ζ signaling domain | 1123-1458 | 375-486 |

TABLE 11
Annotation of lisocabtagene maraleucel CD19 CAR sequences

| | Nucleotide | Amino Acid |
|---|---|---|
| | Sequence | Sequence |
| Feature | Position | Position |
| GMCSFR-α signal peptide | 1-66 | 1-22 |
| FMC63 scFv | 67-801 | 23-267 |
| (V_{L}-Whitlow linker-V_{H}) | | |
| IgG4 hinge domain | 802-837 | 268-279 |
| CD28 transmembrane domain | 838-921 | 280-307 |
| 4-1BB costimulatory domain | 922-1047 | 308-349 |
| CD3ζ signaling domain | 1048-1383 | 350-461 |

TABLE 12
Annotation of axicabtagene ciloleucel CD19 CAR sequences

| | Nucleotide | Amino Acid |
|---|---|---|
| | Sequence | Sequence |
| Feature | Position | Position |
| CSF2RA signal peptide | 1-66 | 1-22 |
| FMC63 scFv | 67-801 | 23-267 |
| (V_{L}-Whitlow linker-V_{H}) | | |
| CD28 hinge domain | 802-927 | 268-309 |
| CD28 transmembrane domain | 928-1008 | 310-336 |
| CD28 costimulatory domain | 1009-1131 | 337-377 |
| CD3ζ signaling domain | 1132-1467 | 378-489 |

In some embodiments, the polycistronic vector comprises an expression cassette that contains a nucleotide sequence encoding CD19 CAR as set forth in SEQ ID NO: 54, 56, or 58, or at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 54, 56, or 58. The encoded CD19 CAR has a corresponding amino acid sequence set forth in SEQ ID NO: 55, 57, or 59, respectively, is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 55, 57, or 59, respectively.

b. CD20 CAR

In some embodiments, the CAR is a CD20 CAR (“CD20-CAR”), and in these embodiments, the polycistronic vector comprises an expression cassette that contains a nucleotide sequence encoding a CD20 CAR. CD20 is an antigen found on the surface of B cells as early at the pro-B phase and progressively at increasing levels until B cell maturity, as well as on the cells of most B-cell neoplasms. CD20 positive cells are also sometimes found in cases of Hodgkins disease, myeloma, and thymoma. In some embodiments, the CD20 CAR may comprise a signal peptide, an extracellular binding domain that specifically binds CD20, a hinge domain, a transmembrane domain, an intracellular costimulatory domain, and/or an intracellular signaling domain in tandem.

In some embodiments, the signal peptide of the CD20 CAR comprises a CD8α signal peptide. In some embodiments, the CD8α signal peptide comprises or consists of an amino acid sequence set forth in SEQ ID NO:24 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:24. In some embodiments, the signal peptide comprises an IgK signal peptide. In some embodiments, the IgK signal peptide comprises or consists of an amino acid sequence set forth in SEQ ID NO:25 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:25. In some embodiments, the signal peptide comprises a GMCSFR-a or CSF2RA signal peptide. In some embodiments, the GMCSFR-α or CSF2RA signal peptide comprises or consists of an amino acid sequence set forth in SEQ ID NO:26 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:26.

In some embodiments, the extracellular binding domain of the CD20 CAR is specific to CD20, for example, human CD20. The extracellular binding domain of the CD20 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD20 CAR is derived from an antibody specific to CD20, including, for example, Leu16, IFS, 1.5.3, rituximab, obinutuzumab, ibritumomab, ofatumumab, tositumumab, odronextamab, veltuzumab, ublituximab, and ocrelizumab. In any of these embodiments, the extracellular binding domain of the CD20 CAR can comprise or consist of the V_{H}, the V_{L}, and/or one or more CDRs of any of the antibodies.

In some embodiments, the extracellular binding domain of the CD20 CAR comprises an scFv derived from the Leu16 monoclonal antibody, which comprises the heavy chain variable region (V_{H}) and the light chain variable region (V_{L}) of Leu16 connected by a linker. See Wu et al., Protein Engineering. 14(12):1025-1033 (2001). In some embodiments, the linker is a 3×G₄S linker. In other embodiments, the linker is a Whitlow linker as described herein. In some embodiments, the amino acid sequences of different portions of the entire Leu16-derived scFv (also referred to as Leu16 scFv) and its different portions are provided in Table 13 below. In some embodiments, the CD20-specific scFv comprises or consists of an amino acid sequence set forth in SEQ ID NO:60, 61, or 65, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:60, 61, or 65 In some embodiments, the CD20-specific scFv may comprise one or more CDRs having amino acid sequences set forth in SEQ ID NOs: 62-64, 66, 67, and 68. In some embodiments, the CD20-specific scFv may comprise a light chain with one or more CDRs having amino acid sequences set forth in SEQ ID NOs: 62-64. In some embodiments, the CD20-specific scFv may comprise a heavy chain with one or more CDRs having amino acid sequences set forth in SEQ ID NOs: 66, 67, and 68. In any of these embodiments, the CD20-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD20 CAR comprises or consists of the one or more CDRs as described herein.

TABLE 13
Exemplary sequences of anti-CD20 scFv and components

| SEQ ID NO: | Amino Acid Sequence | Description |
|---|---|---|
| 60 | DIVLTQSPAILSASPGEKVTMTCRASSS | Anti-CD20 Leu16 scFv |
| | VNYMDWYQKKPGSSPKPWIYATSNLA | entire sequence, with |
| | SGVPARFSGSGSGTSYSLTISRVEAEDA | Whitlow linker |
| | ATYYCQQWSFNPPTFGGGTKLEIKGSTS | |
| | GSGKPGSGEGSTKGEVQLQQSGAELVK | |
| | PGASVKMSCKASGYTFTSYNMHWVKQ | |
| | TPGQGLEWIGAIYPGNGDTSYNQKFKG | |
| | KATLTADKSSSTAYMQLSSLTSEDSAD | |
| | YYCARSNYYGSSYWFFDVWGAGTTVT | |
| | VSS | |
| 61 | DIVLTQSPAILSASPGEKVTMTCRASSS | Anti-CD20 Leu16 scFv |
| | VNYMDWYQKKPGSSPKPWIYATSNLA | light chain variable |
| | SGVPARFSGSGSGTSYSLTISRVEAEDA | region |
| | ATYYCQQWSFNPPTFGGGTKLEIK | |
| 62 | RASSSVNYMD | Anti-CD20 Leu16 scFv |
| | | light chain CDR1 |
| 63 | ATSNLAS | Anti-CD20 Leu16 scFv |
| | | light chain CDR2 |
| 64 | QQWSFNPPT | Anti-CD20 Leu16 scFv |
| | | light chain CDR3 |
| 65 | EVQLQQSGAELVKPGASVKMSCKASG | Anti-CD20 Leu16 scFv |
| | YTFTSYNMHWVKQTPGQGLEWIGAIYP | heavy chain |
| | GNGDTSYNQKFKGKATLTADKSSSTAY | |
| | MQLSSLTSEDSADYYCARSNYYGSSYW | |
| | FFDVWGAGTTVTVSS | |
| 66 | SYNMH | Anti-CD20 Leu16 scFv |
| | | heavy chain CDR1 |
| 67 | AIYPGNGDTSYNQKFKG | Anti-CD20 Leu16 scFv |
| | | heavy chain CDR2 |
| 68 | SNYYGSSYWFFDV | Anti-CD20 Leu16 scFv |
| | | heavy chain CDR3 |

In some embodiments, the hinge domain of the CD20 CAR comprises a CD8α hinge domain, for example, a human CD8α hinge domain. In some embodiments, the CD8α hinge domain comprises or consists of an amino acid sequence set forth in SEQ ID NO:27 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:27. In some embodiments, the hinge domain comprises a CD28 hinge domain, for example, a human CD28 hinge domain. In some embodiments, the CD28 hinge domain comprises or consists of an amino acid sequence set forth in SEQ ID NO:28 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:28. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:30 or SEQ ID NO:31, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:30 or SEQ ID NO:31. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:32 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:32.

In some embodiments, the transmembrane domain of the CD20 CAR comprises a CD8α transmembrane domain, for example, a human CD8α transmembrane domain. In some embodiments, the CD8α transmembrane domain comprises or consists of an amino acid sequence set forth in SEQ ID NO:33 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:33. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:35 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:35.

In some embodiments, the intracellular costimulatory domain of the CD20 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:36 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:36. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:37 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:37.

In some embodiments, the intracellular signaling domain of the CD20 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:38 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:38.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:60, the CD8α hinge domain of SEQ ID NO:27, the CD8α transmembrane domain of SEQ ID NO:33, the 4-1BB costimulatory domain of SEQ ID NO:36, the CD3ζ signaling domain of SEQ ID NO:38, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:60, the CD28 hinge domain of SEQ ID NO:27, the CD8α transmembrane domain of SEQ ID NO:33, the 4-1BB costimulatory domain of SEQ ID NO:36, the CD3ζ signaling domain of SEQ ID NO:38, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:60, the IgG4 hinge domain of SEQ ID NO:30 or SEQ ID NO:31, the CD8α transmembrane domain of SEQ ID NO:33, the 4-1BB costimulatory domain of SEQ ID NO:36, the CD3ζ signaling domain of SEQ ID NO:37, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:60, the CD8α hinge domain of SEQ ID NO:27, the CD28 transmembrane domain of SEQ ID NO:29, the 4-1BB costimulatory domain of SEQ ID NO:36, the CD3ζ signaling domain of SEQ ID NO:37, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:60, the CD28 hinge domain of SEQ ID NO:29, the CD28 transmembrane domain of SEQ ID NO:35, the 4-1BB costimulatory domain of SEQ ID NO:36, the CD3ζ signaling domain of SEQ ID NO:37, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:60, the IgG4 hinge domain of SEQ ID NO:30 or SEQ ID NO:31, the CD28 transmembrane domain of SEQ ID NO:34, the 4-1BB costimulatory domain of SEQ ID NO:36, the CD3ζ signaling domain of SEQ ID NO:37, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

c. CD22 CAR

In some embodiments, the CAR is a CD22 CAR (“CD22-CAR”), and in these embodiments, the polycistronic vector comprises an expression cassette that contains a nucleotide sequence encoding a CD22 CAR. CD22, which is a transmembrane protein found mostly on the surface of mature B cells that functions as an inhibitory receptor for B cell receptor (BCR) signaling. CD22 is expressed in 60-70% of B cell lymphomas and leukemias (e.g., B-chronic lymphocytic leukemia, hairy cell leukemia, acute lymphocytic leukemia (ALL), and Burkitt's lymphoma) and is not present on the cell surface in early stages of B cell development or on stem cells. In some embodiments, the CD22 CAR may comprise a signal peptide, an extracellular binding domain that specifically binds CD22, a hinge domain, a transmembrane domain, an intracellular costimulatory domain, and/or an intracellular signaling domain in tandem.

In some embodiments, the signal peptide of the CD22 CAR comprises a CD8α signal peptide. In some embodiments, the CD8α signal peptide comprises or consists of an amino acid sequence set forth in SEQ ID NO:85 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:85. In some embodiments, the signal peptide comprises an IgK signal peptide. In some embodiments, the IgK signal peptide comprises or consists of an amino acid sequence set forth in SEQ ID NO:86 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:86. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:87 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:87.

In some embodiments, the extracellular binding domain of the CD22 CAR is specific to CD22, for example, human CD22. The extracellular binding domain of the CD22 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD22 CAR is derived from an antibody specific to CD22, including, for example, SM03, inotuzumab, epratuzumab, moxetumomab, and pinatuzumab. In any of these embodiments, the extracellular binding domain of the CD22 CAR can comprise or consist of the V_{H}, the V_{L}, and/or one or more CDRs of any of the antibodies.

In some embodiments, the extracellular binding domain of the CD22 CAR comprises an scFv derived from the m971 monoclonal antibody (m971), which comprises the heavy chain variable region (V_{H}) and the light chain variable region (V_{L}) of m971 connected by a linker. In some embodiments, the linker is a 3×G₄S linker. In other embodiments, the Whitlow linker may be used instead. In some embodiments, the amino acid sequences of the entire m971-derived scFv (also referred to as m971 scFv) and its different portions are provided in Table 15 below. In some embodiments, the CD22-specific scFv comprises or consists of an amino acid sequence set forth in SEQ ID NO:128, 129, or 133, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:128, 129, or 133. In some embodiments, the CD22-specific scFv may comprise one or more CDRs having amino acid sequences set forth in SEQ ID NOs: 130-132 and 134-136. In some embodiments, the CD22-specific scFv may comprise a heavy chain with one or more CDRs having amino acid sequences set forth in SEQ ID NOs: 130-132. In some embodiments, the CD22-specific scFv may comprise a light chain with one or more CDRs having amino acid sequences set forth in SEQ ID NOs: 134-136. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

In some embodiments, the extracellular binding domain of the CD22 CAR comprises an scFv derived from m971-L7, which is an affinity matured variant of m971 with significantly improved CD22 binding affinity compared to the parental antibody m971 (improved from about 2 nM to less than 50 μM). In some embodiments, the scFv derived from m971-L7 comprises the V_{H} and the V_{L} of m971-L7 connected by a 3×G₄S linker. In other embodiments, the Whitlow linker may be used instead. In some embodiments, the amino acid sequences of the entire m971-L7-derived scFv (also referred to as m971-L7 scFv) and its different portions are provided in Table 14 below. In some embodiments, the CD22-specific scFv comprises or consists of an amino acid sequence set forth in SEQ ID NO:137, 138, or 142, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:137, 138, or 142. In some embodiments, the CD22-specific scFv may comprise one or more CDRs having amino acid sequences set forth in SEQ ID NOs: 139-141 143-145. In some embodiments, the CD22-specific scFv may comprise a heavy chain with one or more CDRs having amino acid sequences set forth in SEQ ID NOs: 139-141. In some embodiments, the CD22-specific scFv may comprise a light chain with one or more CDRs having amino acid sequences set forth in SEQ ID NOs: 143-145. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

TABLE 14
Exemplary sequences of anti-CD22 scFv and components

| SEQ ID NO: | Amino Acid Sequence | Description |
|---|---|---|
| 69 | QVQLQQSGPGLVKPSQTLSLTCAISGDS | Anti-CD22 m971 scFv |
| | VSSNSAAWNWIRQSPSRGLEWLGRTYY | entire sequence, with |
| | RSKWYNDYAVSVKSRITINPDTSKNQFS | 3xG₄S linker |
| | LQLNSVTPEDTAVYYCAREVTGDLEDA | |
| | FDIWGQGTMVTVSSGGGGSGGGGSGG | |
| | GGSDIQMTQSPSSLSASVGDRVTITCRA | |
| | SQTIWSYLNWYQQRPGKAPNLLIYAAS | |
| | SLQSGVPSRFSGRGSGTDFTLTISSLQAE | |
| | DFATYYCQQSYSIPQTFGQGTKLEIK | |
| 70 | QVQLQQSGPGLVKPSQTLSLTCAISGDS | Anti-CD22 m971 scFv |
| | VSSNSAAWNWIRQSPSRGLEWLGRTYY | heavy chain variable |
| | RSKWYNDYAVSVKSRITINPDTSKNQFS | region |
| | LQLNSVTPEDTAVYYCAREVTGDLEDA | |
| | FDIWGQGTMVTVSS | |
| 71 | GDSVSSNSAA | Anti-CD22 m971 scFv |
| | | heavy chain CDR1 |
| 72 | TYYRSKWYN | Anti-CD22 m971 scFv |
| | | heavy chain CDR2 |
| 73 | AREVTGDLEDAFDI | Anti-CD22 m971 scFv |
| | | heavy chain CDR3 |
| 74 | DIQMTQSPSSLSASVGDRVTITCRASQTI | Anti-CD22 m971 scFv |
| | WSYLNWYQQRPGKAPNLLIYAASSLQS | light chain |
| | GVPSRFSGRGSGTDFTLTISSLQAEDFAT | |
| | YYCQQSYSIPQTFGQGTKLEIK | |
| 75 | QTIWSY | Anti-CD22 m971 scFv |
| | | light chain CDR1 |
| 76 | AAS | Anti-CD22 m971 scFv |
| | | light chain CDR2 |
| 77 | QQSYSIPQT | Anti-CD22 m971 scFv |
| | | light chain CDR3 |
| 78 | QVQLQQSGPGMVKPSQTLSLTCAISGD | Anti-CD22 m971-L7 scFv |
| | SVSSNSVAWNWIRQSPSRGLEWLGRTY | entire sequence, with |
| | YRSTWYNDYAVSMKSRITINPDTNKNQ | 3xG₄S linker |
| | FSLQLNSVTPEDTAVYYCAREVTGDLE | |
| | DAFDIWGQGTMVTVSSGGGGSGGGGS | |
| | GGGGSDIQMIQSPSSLSASVGDRVTITC | |
| | RASQTIWSYLNWYRQRPGEAPNLLIYA | |
| | ASSLQSGVPSRFSGRGSGTDFTLTISSLQ | |
| | AEDFATYYCQQSYSIPQTFGQGTKLEIK | |
| 79 | QVQLQQSGPGMVKPSQTLSLTCAISGD | Anti-CD22 m971-L7 scFv |
| | SVSSNSVAWNWIRQSPSRGLEWLGRTY | heavy chain variable |
| | YRSTWYNDYAVSMKSRITINPDTNKNQ | region |
| | FSLQLNSVTPEDTAVYYCAREVTGDLE | |
| | DAFDIWGQGTMVTVSS | |
| 80 | GDSVSSNSVA | Anti-CD22 m971-L7 scFv |
| | | heavy chain CDR1 |
| 81 | TYYRSTWYN | Anti-CD22 m971-L7 scFv |
| | | heavy chain CDR2 |
| 82 | AREVTGDLEDAFDI | Anti-CD22 m971-L7 scFv |
| | | heavy chain CDR3 |
| 83 | DIQMIQSPSSLSASVGDRVTITCRASQTI | Anti-CD22 m971-L7 scFv |
| | WSYLNWYRQRPGEAPNLLIYAASSLQS | light chain variable |
| | GVPSRFSGRGSGTDFTLTISSLQAEDFAT | region |
| | YYCQQSYSIPQTFGQGTKLEIK | |
| 84 | QTIWSY | Anti-CD22 m971-L7 scFv |
| | | light chain CDR1 |
| 85 | AAS | Anti-CD22 m971-L7 scFv |
| | | light chain CDR2 |
| 86 | QQSYSIPQT | Anti-CD22 m971-L7 scFv |
| | | light chain CDR3 |

In some embodiments, the extracellular binding domain of the CD22 CAR comprises immunotoxins HA22 or BL22. Immunotoxins BL22 and HA22 are therapeutic agents that comprise an scFv specific for CD22 fused to a bacterial toxin, and thus can bind to the surface of the cancer cells that express CD22 and kill the cancer cells. BL22 comprises a dsFv of an anti-CD22 antibody, RFB4, fused to a 38-kDa truncated form of Pseudomonas exotoxin A (Bang et al., Clin. Cancer Res., 11:1545-50 (2005)). HA22 (CAT8015, moxetumomab pasudotox) is a mutated, higher affinity version of BL22 (Ho et al., J. Biol. Chem., 280(1): 607-17 (2005)). Suitable sequences of antigen binding domains of HA22 and BL22 specific to CD22 are disclosed in, for example, U.S. Pat. Nos. 7,541,034; 7,355,012; and 7,982,011, which are hereby incorporated by reference in their entirety.

In some embodiments, the hinge domain of the CD22 CAR comprises a CD8α hinge domain, for example, a human CD8α hinge domain. In some embodiments, the CD8α hinge domain comprises or consists of an amino acid sequence set forth in SEQ ID NO:88 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:88. In some embodiments, the hinge domain comprises a CD28 hinge domain, for example, a human CD28 hinge domain. In some embodiments, the CD28 hinge domain comprises or consists of an amino acid sequence set forth in SEQ ID NO:89 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:89. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:91 or SEQ ID NO:92, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:91 or SEQ ID NO:92. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:93 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:93.

In some embodiments, the transmembrane domain of the CD22 CAR comprises a CD8α transmembrane domain, for example, a human CD8α transmembrane domain. In some embodiments, the CD8α transmembrane domain comprises or consists of an amino acid sequence set forth in SEQ ID NO:94 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:94. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:95 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:95.

In some embodiments, the intracellular costimulatory domain of the CD22 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:97 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:97. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:98 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:98.

In some embodiments, the intracellular signaling domain of the CD22 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:99 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:99.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:128 or SEQ ID NO:137, the CD8α hinge domain of SEQ ID NO:88, the CD8α transmembrane domain of SEQ ID NO:94, the 4-1BB costimulatory domain of SEQ ID NO:97, the CD3ζ signaling domain of SEQ ID NO:99, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:128 or SEQ ID NO:137, the CD28 hinge domain of SEQ ID NO:89, the CD8α transmembrane domain of SEQ ID NO:94, the 4-1BB costimulatory domain of SEQ ID NO:97, the CD3ζ signaling domain of SEQ ID NO:99, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:128 or SEQ ID NO:137, the IgG4 hinge domain of SEQ ID NO:91 or SEQ ID NO:92, the CD8α transmembrane domain of SEQ ID NO:94, the 4-1BB costimulatory domain of SEQ ID NO:97, the CD3ζ signaling domain of SEQ ID NO:99, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:128 or SEQ ID NO:137, the CD8α hinge domain of SEQ ID NO:8, the CD28 transmembrane domain of SEQ ID NO:95, the 4-1BB costimulatory domain of SEQ ID NO:97, the CD3ζ signaling domain of SEQ ID NO:99, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:128 or SEQ ID NO:137, the CD28 hinge domain of SEQ ID NO:89, the CD28 transmembrane domain of SEQ ID NO:95, the 4-1BB costimulatory domain of SEQ ID NO:97, the CD3ζ signaling domain of SEQ ID NO:99, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:128 or SEQ ID NO:137, the IgG4 hinge domain of SEQ ID NO:91 or SEQ ID NO:92, the CD28 transmembrane domain of SEQ ID NO:95, the 4-1BB costimulatory domain of SEQ ID NO:97, the CD3ζ signaling domain of SEQ ID NO:99, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

d. BCMA CAR

In some embodiments, the CAR is a BCMA CAR (“BCMA-CAR”), and in these embodiments, the polycistronic vector comprises an expression cassette that contains a nucleotide sequence encoding a BCMA CAR. BCMA is a tumor necrosis family receptor (TNFR) member expressed on cells of the B cell lineage, with the highest expression on terminally differentiated B cells or mature B lymphocytes. BCMA is involved in mediating the survival of plasma cells for maintaining long-term humoral immunity. The expression of BCMA has been recently linked to a number of cancers, such as multiple myeloma, Hodgkin's and non-Hodgkin's lymphoma, various leukemias, and glioblastoma. In some embodiments, the BCMA CAR may comprise a signal peptide, an extracellular binding domain that specifically binds BCMA, a hinge domain, a transmembrane domain, an intracellular costimulatory domain, and/or an intracellular signaling domain in tandem.

In some embodiments, the signal peptide of the BCMA CAR comprises a CD8α signal peptide. In some embodiments, the CD8α signal peptide comprises or consists of an amino acid sequence set forth in SEQ ID NO:85 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:85. In some embodiments, the signal peptide comprises an IgK signal peptide. In some embodiments, the IgK signal peptide comprises or consists of an amino acid sequence set forth in SEQ ID NO:86 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:86. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:87 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:87.

In some embodiments, the extracellular binding domain of the BCMA CAR is specific to BCMA, for example, human BCMA. The extracellular binding domain of the BCMA CAR can be codon-optimized for expression in a host cell or to have variant sequences to increase functions of the extracellular binding domain.

In some embodiments, the extracellular binding domain comprises an immunogenically active portion of an immunoglobulin molecule, for example, an scFv. In some embodiments, the extracellular binding domain of the BCMA CAR is derived from an antibody specific to BCMA, including, for example, belantamab, erlanatamab, teclistamab, LCAR-B38M, and ciltacabtagene. In any of these embodiments, the extracellular binding domain of the BCMA CAR can comprise or consist of the V_{H}, the V_{L}, and/or one or more CDRs of any of the antibodies.

In some embodiments, the extracellular binding domain of the BCMA CAR comprises an scFv derived from C11D5.3, a murine monoclonal antibody as described in Carpenter et al., Clin. Cancer Res. 19(8):2048-2060 (2013). See also PCT Application Publication No. WO2010/104949. The C 11D5.3-derived scFv may comprise the heavy chain variable region (V_{H}) and the light chain variable region (V_{L}) of C11D5.3 connected by the Whitlow linker, the amino acid sequences of which is provided in Table 16 below. In some embodiments, the BCMA-specific extracellular binding domain comprises or consists of an amino acid sequence set forth in SEQ ID NO:146, 147, or 151, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:146, 147, or 151. In some embodiments, the BCMA-specific extracellular binding domain may comprise one or more CDRs having amino acid sequences set forth in SEQ ID NOs: 148-150 and 152-154. In some embodiments, the BCMA-specific extracellular binding domain may comprise a light chain with one or more CDRs having amino acid sequences set forth in SEQ ID NOs: 148-150. In some embodiments, the BCMA-specific extracellular binding domain may comprise a heavy chain with one or more CDRs having amino acid sequences set forth in SEQ ID NOs: 152-154. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from another murine monoclonal antibody, C12A3.2, as described in Carpenter et al., Clin. Cancer Res. 19(8):2048-2060 (2013) and PCT Application Publication No. WO2010/104949, the amino acid sequence of which is also provided in Table 15 below. In some embodiments, the BCMA-specific extracellular binding domain comprises or consists of an amino acid sequence set forth in SEQ ID NO:155, 156, or 160, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:155, 156, or 160. In some embodiments, the BCMA-specific extracellular binding domain may comprise one or more CDRs having amino acid sequences set forth in SEQ ID NOs: 157-159 and 161-163. In some embodiments, the BCMA-specific extracellular binding domain may comprise a light chain with one or more CDRs having amino acid sequences set forth in SEQ ID NOs: 157-159. In some embodiments, the BCMA-specific extracellular binding domain may comprise a heavy chain with one or more CDRs having amino acid sequences set forth in SEQ ID NOs: 161-163. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises a murine monoclonal antibody with high specificity to human BCMA, referred to as BB2121 in Friedman et al., Hum. Gene Ther. 29(5):585-601 (2018)). See also, PCT Application Publication No. WO2012163805.

In some embodiments, the extracellular binding domain of the BCMA CAR comprises single variable fragments of two heavy chains (VHH) that can bind to two epitopes of BCMA as described in Zhao et al., J. Hematol. Oncol. 11(1):141 (2018), also referred to as LCAR-B38M. See also, PCT Application Publication No. WO2018/028647.

In some embodiments, the extracellular binding domain of the BCMA CAR comprises a fully human heavy-chain variable domain (FHVH) as described in Lam et al., Nat. Commun. 11(1):283 (2020), also referred to as FHVH33. See also, PCT Application Publication No. WO2019/006072. The amino acid sequences of FHVH33 and its CDRs are provided in Table 15 below. In some embodiments, the BCMA-specific extracellular binding domain comprises or consists of an amino acid sequence set forth in SEQ ID NO:164 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:164. In some embodiments, the BCMA-specific extracellular binding domain may comprise one or more CDRs having amino acid sequences set forth in SEQ ID NOs: 165-167. In any of these embodiments, the BCMA-specific extracellular binding domain may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from CT103A (or CAR0085) as described in U.S. Pat. No. 11,026,975 B2, the amino acid sequence of which is provided in Table 15 below. In some embodiments, the BCMA-specific extracellular binding domain comprises or consists of an amino acid sequence set forth in SEQ ID NO:168, 169, or 173, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 168, 169, or 173. In some embodiments, the BCMA-specific extracellular binding domain may comprise one or more CDRs having amino acid sequences set forth in SEQ ID NOs: 170-172 and 174-176. In some embodiments, the BCMA-specific extracellular binding domain may comprise a light chain with one or more CDRs having amino acid sequences set forth in SEQ ID NOs: 170-172. In some embodiments, the BCMA-specific extracellular binding domain may comprise a heavy chain with one or more CDRs having amino acid sequences set forth in SEQ ID NOs: 174-176. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

Additionally, CARs and binders directed to BCMA have been described in U.S. Application Publication Nos. 2020/0246381 A1 and 2020/0339699 A1, the entire contents of each of which are incorporated by reference herein.

TABLE 15
Exemplary sequences of anti-BCMA binder and components

| SEQ ID NO: | Amino Acid Sequence | Description |
|---|---|---|
| 87 | DIVLTQSPASLAMSLGKRATISCRASES | Anti-BCMA C11D5.3 |
| | VSVIGAHLIHWYQQKPGQPPKLLIYLAS | scFv entire sequence, with |
| | NLETGVPARFSGSGSGTDFTLTIDPVEE | Whitlow linker |
| | DDVAIYSCLQSRIFPRTFGGGTKLEIKGS | |
| | TSGSGKPGSGEGSTKGQIQLVQSGPELK | |
| | KPGETVKISCKASGYTFTDYSINWVKR | |
| | APGKGLKWMGWINTETREPAYAYDFR | |
| | GRFAFSLETSASTAYLQINNLKYEDTAT | |
| | YFCALDYSYAMDYWGQGTSVTVSS | |
| 88 | DIVLTQSPASLAMSLGKRATISCRASES | Anti-BCMA C11D5.3 |
| | VSVIGAHLIHWYQQKPGQPPKLLIYLAS | scFv light chain variable |
| | NLETGVPARFSGSGSGTDFTLTIDPVEE | region |
| | DDVAIYSCLQSRIFPRTFGGGTKLEIK | |
| 89 | RASESVSVIGAHLIH | Anti-BCMA C11D5.3 |
| | | scFv light chain CDR1 |
| 90 | LASNLET | Anti-BCMA C11D5.3 |
| | | scFv light chain CDR2 |
| 91 | LQSRIFPRT | Anti-BCMA C11D5.3 |
| | | scFv light chain CDR3 |
| 92 | QIQLVQSGPELKKPGETVKISCKASGYT | Anti-BCMA C11D5.3 |
| | FTDYSINWVKRAPGKGLKWMGWINTE | scFv heavy chain variable |
| | TREPAYAYDFRGRFAFSLETSASTAYLQ | region |
| | INNLKYEDTATYFCALDYSYAMDYWG | |
| | QGTSVTVSS | |
| 93 | DYSIN | Anti-BCMA C11D5.3 |
| | | scFv heavy chain CDR1 |
| 94 | WINTETREPAYAYDFRG | Anti-BCMA C11D5.3 |
| | | scFv heavy chain CDR2 |
| 95 | DYSYAMDY | Anti-BCMA C11D5.3 |
| | | scFv heavy chain CDR3 |
| 96 | DIVLTQSPPSLAMSLGKRATISCRASESV | Anti-BCMA C12A3.2 |
| | TILGSHLIYWYQQKPGQPPTLLIQLASN | scFv entire sequence, with |
| | VQTGVPARFSGSGSRTDFTLTIDPVEED | Whitlow linker |
| | DVAVYYCLQSRTIPRTFGGGTKLEIKGS | |
| | TSGSGKPGSGEGSTKGQIQLVQSGPELK | |
| | KPGETVKISCKASGYTFRHYSMNWVK | |
| | QAPGKGLKWMGRINTESGVPIYADDFK | |
| | GRFAFSVETSASTAYLVINNLKDEDTAS | |
| | YFCSNDYLYSLDFWGQGTALTVSS | |
| 97 | DIVLTQSPPSLAMSLGKRATISCRASESV | Anti-BCMA C12A3.2 |
| | TILGSHLIYWYQQKPGQPPTLLIQLASN | scFv light chain variable |
| | VQTGVPARFSGSGSRTDFTLTIDPVEED | region |
| | DVAVYYCLQSRTIPRTFGGGTKLEIK | |
| 98 | RASESVTILGSHLIY | Anti-BCMA C12A3.2 |
| | | scFv light chain CDR1 |
| 99 | LASNVQT | Anti-BCMA C12A3.2 |
| | | scFv light chain CDR2 |
| 100 | LQSRTIPRT | Anti-BCMA C12A3.2 |
| | | scFv light chain CDR3 |
| 101 | QIQLVQSGPELKKPGETVKISCKASGYT | Anti-BCMA C12A3.2 |
| | FRHYSMNWVKQAPGKGLKWMGRINTE | scFv heavy chain variable |
| | SGVPIYADDFKGRFAFSVETSASTAYLV | region |
| | INNLKDEDTASYFCSNDYLYSLDFWGQ | |
| | GTALTVSS | |
| 102 | HYSMN | Anti-BCMA C12A3.2 |
| | | scFv heavy chain CDR1 |
| 103 | RINTESGVPIYADDFKG | Anti-BCMA C12A3.2 |
| | | scFv heavy chain CDR2 |
| 104 | DYLYSLDF | Anti-BCMA C12A3.2 |
| | | scFv heavy chain CDR3 |
| 105 | EVQLLESGGGLVQPGGSLRLSCAASGF | Anti-BCMA FHVH33 |
| | TFSSYAMSWVRQAPGKGLEWVSSISGS | entire sequence |
| | GDYIYYADSVKGRFTISRDISKNTLYLQ | |
| | MNSLRAEDTAVYYCAKEGTGANSSLA | |
| | DYRGQGTLVTVSS | |
| 106 | GFTFSSYA | Anti-BCMA FHVH33 |
| | | CDR1 |
| 107 | ISGSGDYI | Anti-BCMA FHVH33 |
| | | CDR2 |
| 108 | AKEGTGANSSLADY | Anti-BCMA FHVH33 |
| | | CDR3 |
| 109 | DIQMTQSPSSLSASVGDRVTITCRASQSI | Anti-BCMA CT103A |
| | SSYLNWYQQKPGKAPKLLIYAASSLQS | scFv entire sequence, with |
| | GVPSRFSGSGSGTDFTLTISSLQPEDFAT | Whitlow linker |
| | YYCQQKYDLLTFGGGTKVEIKGSTSGS | |
| | GKPGSGEGSTKGQLQLQESGPGLVKPS | |
| | ETLSLTCTVSGGSISSSSYYWGWIRQPP | |
| | GKGLEWIGSISYSGSTYYNPSLKSRVTIS | |
| | VDTSKNQFSLKLSSVTAADTAVYYCAR | |
| | DRGDTILDVWGQGTMVTVSS | |
| 110 | DIQMTQSPSSLSASVGDRVTITCRASQSI | Anti-BCMA CT103A |
| | SSYLNWYQQKPGKAPKLLIYAASSLQS | scFv light chain variable |
| | GVPSRFSGSGSGTDFTLTISSLQPEDFAT | region |
| | YYCQQKYDLLTFGGGTKVEIK | |
| 111 | QSISSY | Anti-BCMA CT103A |
| | | scFv light chain CDR1 |
| 112 | AAS | Anti-BCMA CT103A |
| | | scFv light chain CDR2 |
| 113 | QQKYDLLT | Anti-BCMA CT103A |
| | | scFv light chain CDR3 |
| 114 | QLQLQESGPGLVKPSETLSLTCTVSGGS | Anti-BCMA CT103A |
| | ISSSSYYWGWIRQPPGKGLEWIGSISYS | scFv heavy chain variable |
| | GSTYYNPSLKSRVTISVDTSKNQFSLKL | region |
| | SSVTAADTAVYYCARDRGDTILDVWG | |
| | QGTMVTVSS | |
| 115 | GGSISSSSYY | Anti-BCMA CT103A |
| | | scFv heavy chain CDR1 |
| 116 | ISYSGST | Anti-BCMA CT103A |
| | | scFv heavy chain CDR2 |
| 117 | ARDRGDTILDV | Anti-BCMA CT103A |
| | | scFv heavy chain CDR3 |

In some embodiments, the hinge domain of the BCMA CAR comprises a CD8α hinge domain, for example, a human CD8α hinge domain. In some embodiments, the CD8α hinge domain comprises or consists of an amino acid sequence set forth in SEQ ID NO:88 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:88. In some embodiments, the hinge domain comprises a CD28 hinge domain, for example, a human CD28 hinge domain. In some embodiments, the CD28 hinge domain comprises or consists of an amino acid sequence set forth in SEQ ID NO: 89 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:89. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:91 or SEQ ID NO:92, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:91 or SEQ ID NO:92. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:93 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:93.

In some embodiments, the transmembrane domain of the BCMA CAR comprises a CD8α transmembrane domain, for example, a human CD8α transmembrane domain. In some embodiments, the CD8α transmembrane domain comprises or consists of an amino acid sequence set forth in SEQ ID NO:94 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:94. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:95 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:95.

In some embodiments, the intracellular costimulatory domain of the BCMA CAR comprises a 4-1BB costimulatory domain, for example, a human 4-1BB costimulatory domain. In some embodiments, the 4-1BB costimulatory domain comprises or consists of an amino acid sequence set forth in SEQ ID NO:97 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:97. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:98 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:98.

In some embodiments, the intracellular signaling domain of the BCMA CAR comprises a CD3 zeta (ζ) signaling domain, for example, a human CD3ζ signaling domain. In some embodiments, the CD3ζ signaling domain comprises or consists of an amino acid sequence set forth in SEQ ID NO:99 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:99.

In some embodiments, the polycistronic vector comprises an expression cassette that contains a nucleotide sequence encoding a BCMA CAR, including, for example, a BCMA CAR comprising any of the BCMA-specific extracellular binding domains as described, the CD8α hinge domain of SEQ ID NO:88, the CD8α transmembrane domain of SEQ ID NO:94, the 4-1BB costimulatory domain of SEQ ID NO:97, the CD3ζ signaling domain of SEQ ID NO:99, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide (e.g., a CD8α signal peptide) as described.

In some embodiments, the polycistronic vector comprises an expression cassette that contains a nucleotide sequence encoding a BCMA CAR, including, for example, a BCMA CAR comprising any of the BCMA-specific extracellular binding domains as described, the CD8α hinge domain of SEQ ID NO:88, the CD8α transmembrane domain of SEQ ID NO:94, the CD28 costimulatory domain of SEQ ID NO:98, the CD3ζ signaling domain of SEQ ID NO:99, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide as described.

In some embodiments, the polycistronic vector comprises an expression cassette that contains a nucleotide sequence encoding a BCMA CAR as set forth in SEQ ID NO:177 or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:177 (see Table 16). The encoded BCMA CAR has a corresponding amino acid sequence set forth in SEQ ID NO:178 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:178, with the following components: CD8α signal peptide, CT103A scFv (V_{L}—Whitlow linker-V_{H}), CD8α hinge domain, CD8α transmembrane domain, 4-1BB costimulatory domain, and CD3ζ signaling domain.

In some embodiments, the polycistronic vector comprises an expression cassette that contains a nucleotide sequence encoding a commercially available embodiment of BCMA CAR, including, for example, idecabtagene vicleucel (ide-cel, also called bb212I). In some embodiments, the polycistronic vector comprises an expression cassette that contains a nucleotide sequence encoding idecabtagene vicleucel or portions thereof. Idecabtagene vicleucel comprises a BCMA CAR with the following components: the BB2121 binder, CD8α hinge domain, CD8α transmembrane domain, 4-11B1 costimulatory domain, and CD3 signaling domain.

TABLE 16
Exemplary sequences of BCMA CARs

| SEQ ID NO: | Sequence | Description |
|---|---|---|
| 118 | atggccttaccagtgaccgccttgctcctgccgctggccttgctgctcca | Exemplary BCMA |
| | cgccgccaggccggacatccagatgacccagtctccatcctccctgtct | CAR nucleotide |
| | gcatctgtaggagacagagtcaccatcacttgccgggcaagtcagagc | sequence |
| | attagcagctatttaaattggtatcagcagaaaccagggaaagcccctaa | |
| | gctcctgatctatgctgcatccagtttgcaaagtggggtcccatcaaggtt | |
| | cagtggcagtggatctgggacagatttcactctcaccatcagcagtctgc | |
| | aacctgaagattttgcaacttactactgtcagcaaaaatacgacctcctca | |
| | cttttggcggagggaccaaggttgagatcaaaggcagcaccagcggct | |
| | ccggcaagcctggctctggcgagggcagcacaaagggacagctgca | |
| | gctgcaggagtcgggcccaggactggtgaagccttcggagaccctgtc | |
| | cctcacctgcactgtctctggtggctccatcagcagtagtagttactactg | |
| | gggctggatccgccagcccccagggaaggggctggagtggattggg | |
| | agtatctcctatagtgggagcacctactacaacccgtccctcaagagtcg | |
| | agtcaccatatccgtagacacgtccaagaaccagttctccctgaagctga | |
| | gttctgtgaccgccgcagacacggcggtgtactactgcgccagagatc | |
| | gtggagacaccatactagacgtatggggtcagggtacaatggtcaccgt | |
| | cagctcattcgtgcccgtgttcctgcccgccaaacctaccaccacccctg | |
| | cccctagacctcccaccccagccccaacaategccagccagcctctgt | |
| | ctctgcggcccgaagcctgtagacctgctgccggcggagccgtgcaca | |
| | ccagaggcctggacttcgcctgcgacatctacatctgggcccctctggc | |
| | cggcacctgtggcgtgctgctgctgagcctggtgatcaccctgtactgc | |
| | aaccaccggaacaaacggggcagaaagaaactcctgtatatattcaaa | |
| | caaccatttatgagaccagtacaaactactcaagaggaagatggctgta | |
| | gctgccgatttccagaagaagaagaaggaggatgtgaactgagagtga | |
| | agttcagcagatccgccgacgcccctgcctaccagcagggacagaac | |
| | cagctgtacaacgagctgaacctgggcagacgggaagagtacgacgt | |
| | gctggacaagcggagaggccgggaccccgagatgggggaaagcc | |
| | cagacggaagaacccccaggaaggcctgtataacgaactgcagaaag | |
| | acaagatggccgaggcctacagcgagatcggcatgaagggcgagcg | |
| | gaggcgcggcaagggccacgatggcctgtaccagggcctgagcacc | |
| | gccaccaaggacacctacgacgccctgcacatgcaggccctgccccc | |
| | caga | |
| 119 | MALPVTALLLPLALLLHAARPDIQMTQSPSSL | Exemplary BCMA |
| | SASVGDRVTITCRASQSISSYLNWYQQKPGKA | CAR amino acid |
| | PKLLIYAASSLQSGVPSRFSGSGSGTDFTLTISS | sequence |
| | LQPEDFATYYCQQKYDLLTFGGGTKVEIKGST | |
| | SGSGKPGSGEGSTKGQLQLQESGPGLVKPSET | |
| | LSLTCTVSGGSISSSSYYWGWIRQPPGKGLEWI | |
| | GSISYSGSTYYNPSLKSRVTISVDTSKNQFSLK | |
| | LSSVTAADTAVYYCARDRGDTILDVWGQGT | |
| | MVTVSSFVPVFLPAKPTTTPAPRPPTPAPTIAS | |
| | QPLSLRPEACRPAAGGAVHTRGLDFACDIYIW | |
| | APLAGTCGVLLLSLVITLYCNHRNKRGRKKLL | |
| | YIFKQPFMRPVQTTQEEDGCSCRFPEEEEGGC | |
| | ELRVKFSRSADAPAYQQGQNQLYNELNLGRR | |
| | EEYDVLDKRRGRDPEMGGKPRRKNPQEGLY | |
| | NELQKDKMAEAYSEIGMKGERRRGKGHDGL | |
| | YQGLSTATKDTYDALHMQALPPR | |

### V. METHODS OF USE

In some embodiments, the viral vectors and payload genes provided herein or pharmaceutical compositions containing same can be administered to a subject, e.g. a mammal, e.g. a human. In some embodiments, the viral vectors and payload genes are administered by the provided system of ex vivo dosing and administration. In some embodiments, the methods and uses involve dosing the therapy in-line in a closed fluid circuit attached or operably connected to the subject being treated. In some embodiments, the fluid pathway from the whole blood sample from the subject to the reinfusion of the transduction mixture containing the viral vector and PBMCs or subset, such as a leukapheresis or apheresis cell composition, is closed so that the entire process occurs while the system is connected to the subject or patient.

In such embodiments, the subject may be at risk of, may have a symptom of, or may be diagnosed with or identified as having, a particular disease or condition. In one embodiment, the subject has cancer. In one embodiment, the subject has an infectious disease. In some embodiments, the viral vectors, such as a targeted viral vector, contains nucleic acid sequences encoding the payload agent (also interchangeably called an exogenous agent or in some cases “cargo”) for treating the disease or condition in the subject. Thus, in some embodiments, the disease or condition that is treated is any that may be treatable by the encoded payload agent. For instance, in some embodiments the payload agent encodes a chimeric antigen receptor (CAR) that specifically binds to an antigen, and the disease or condition to be treated can be any in which expression of the antigen is associated with and/or involved in the etiology of a disease condition or disorder, e.g. causes, exacerbates or otherwise is involved in such disease, condition, or disorder. Exemplary diseases and conditions can include diseases or conditions associated with malignancy or transformation of cells (e.g. cancer), autoimmune or inflammatory disease, or an infectious disease, e.g. caused by bacterial, viral or other pathogens. Exemplary antigens, which include antigens associated with various diseases and conditions that can be treated, include any of antigens described herein.

For example, the exogenous agent is one that targets or is specific for a protein of a neoplastic cells and the viral vector is administered to a subject for treating a tumor or cancer in the subject. In another example, the exogenous agent is an inflammatory mediator or immune molecule, such as a cytokine, and viral vector is administered to a subject for treating any condition in which it is desired to modulate (e.g. increase) the immune response, such as a cancer or infectious disease. In some embodiments, the viral vector is administered in an effective amount or dose to effect treatment of the disease, condition or disorder.

Provided herein are uses of any of the provided viral vectors in such methods and treatments, and in the preparation of a medicament in order to carry out such therapeutic methods. In some embodiments, the methods are carried out by administering the viral vector or compositions comprising the same, to the subject having, having had, or suspected of having the disease or condition or disorder. In some embodiments, the methods thereby treat the disease or condition or disorder in the subject. Also provided herein are uses of any of the compositions, such as pharmaceutical compositions provided herein, for the treatment of a disease, condition or disorder associated with a particular gene or protein targeted by or provided by the exogenous agent.

In some embodiments, the viral vector or compositions described herein can be administered to a subject, e.g., a mammal, e.g., a human. In some of any embodiments, the subject may be at risk of, may have a symptom of, or may be diagnosed with or identified as having, a particular disease or condition (e.g., a disease or condition described herein). In some embodiments, the disease is a disease or disorder.

In some embodiments, the payload agent is a chimeric antigen receptor, including any as described in Section IV.E and/or that specifically binds an antigen described in Section IV.E. Exemplary target antigens include, but are not limited to, CD5, CD19, CD20, CD22, CD23, CD30, CD70, Kappa, Lambda, and B cell maturation agent (BCMA), G-protein coupled receptor family C group 5 member D (GPRC5D) (associated with leukemias); CS1/SLAMF7, CD38, CD138, GPRC5D, TACI, and BCMA (associated with myelomas); GD2, HER2, EGFR, EGFRvIII, B7H3, PSMA, PSCA, CAIX, CD171, CEA, CSPG4, EPHA2, FAP, FRα, IL-13Ra, Mesothelin, MUC1, MUC16, and ROR1 (associated with solid tumors).

In some embodiments, the disease or condition is a B cell malignancy and the antigen targeted by the CAR is expressed by cells associated with the B cell malignancy. In some embodiments, the antigen is CD19. In some embodiments, the antigen is CD20. In some embodiments, the antigen is CD22. In some embodiments, the antigen is BCMA. In some embodiments, the B cell malignancy is a Large B-cell Lymphoma (LBCL). In some embodiments, the disease or condition has relapsed or the subject is refractory to treatment of the disease or condition. For instance, in some embodiments, the disease or condition is relapsed and/or refractory Large B-cell Lymphoma (LBCL). In some embodiments, LBCL include Non-Hodgkin's lymphoma, including diffuse large B-cell lymphoma (DLBCL) not otherwise specified (including DLBCL arising from indolent lymphoma), primary mediastinal large B-cell lymphoma, high grade B-cell lymphoma, follicular lymphoma, and marginal zone lymphoma. In some embodiments, the subject has received or is receiving prior to the ex vivo dosing provided herein prior therapies, such as two or more lines of systemic therapy for treating the disease or condition. In some embodiments, the subject has relapsed and/or is refractory to the prior therapies. In some embodiments, the prior therapies include two or more prior therapies from a chemotherapy containing regimen, such as with anthracycline, or an anti-CD20 mAb (unless CD20 negative), or after autologous stem cell transplant (ASCT). In some embodiments, the subject has one or more measurable PET-positive lesion, such as measured per Lugano classification. In some embodiments, the subject as an ECOG performance status of 0 or 1. In some embodiments, the subject has adequate organ function.

In some embodiments, the disease or condition is a multiple myeloma and the antigen targeted by the CAR is expressed by cells associated with the multiple myeloma. In some embodiments, the antigen is BCMA. In some embodiments, the subject has or is suspected of having a multiple myeloma that is associated with expression of B cell maturation antigen (BCMA). In some embodiments, the multiple myeloma is a relapsed and/or refractory multiple myeloma.

In some aspects, response rates in subjects, such as subjects with LBCL, are based on the Lugano criteria. (Cheson et al., (2014) JCO., 32(27):3059-3067; Johnson et al., (2015) Radiology 2:323-338; Cheson, B. D. (2015) Chin. Clin. Oncol. 4(1):5). In some aspects, response assessment utilizes any of clinical, hematologic, and/or molecular methods. In some aspects, response assessed using the Lugano criteria involves the use of positron emission tomography (PET)-computed tomography (CT) and/or CT as appropriate. PET-CT evaluations may further comprise the use of fluorodeoxyglucose (FDG) for FDG-avid lymphomas. In some aspects, where PET-CT will be used to assess response in FDG-avid histologies, a 5-point scale may be used. In some respects, the 5-point scale comprises the following criteria: 1, no uptake above background; 2, uptake <mediastinum; 3, uptake >mediastinum but ≤liver; 4, uptake moderately >liver; 5, uptake markedly higher than liver and/or new lesions; X, new areas of uptake unlikely to be related to lymphoma.

In some embodiments, response is based on lack of detectable minimal residual disease (MRD negativity) which means that no disease is detected. Methods for assessing MRD include, but are not limited to flow cytometry, polymerase chain reaction (PCR) and next-generation sequencing. In some embodiments, a sample of bone marrow cells and/or peripheral blood cells is assessed for disease. For instance, certain mutations or genetic abnormalities can be assessed that are known to be associated with the cancer. A skilled artisan is familiar with methods to assess MRD.

In some cases, the pharmacokinetics of cells expressing the payload agent (e.g. CAR) are determined to assess the bioavailability of the engineered cells in vivo. Methods for determining the pharmacokinetics of engineered cells in vivo may include drawing peripheral blood from subjects that have received the ex vivo dosing and determining the number of engineered cells in the blood based on detection of the engineered payload agent (CAR) expressed by the cells. For example, an anti-idiotypic antibody against the CAR may be used for detection of the CAR-expressing cells.

In some embodiments, the regimen of administration may affect what constitutes an effective amount. In some embodiments, the therapeutic formulations may be administered to the subject either prior to or after a diagnosis of disease. In some embodiments, several divided dosages, as well as staggered dosages may be administered daily or sequentially, or the dose may be continuously infused, or may be a bolus injection. In some embodiments, the dosages of the therapeutic formulations may be proportionally increased or decreased as indicated by the exigencies of the therapeutic or prophylactic situation.

In some embodiments, an effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. In some embodiments, the dosage regimens may be adjusted to provide the optimum therapeutic response. In some embodiments, several divided doses may be administered daily or the dose may be proportionally reduced as indicated by the exigencies of the therapeutic situation. One of ordinary skill in the art would be able to study the relevant factors and make the determination regarding the effective amount of the therapeutic compound without undue experimentation.

A medical doctor, e.g., physician or veterinarian, having ordinary skill in the art may readily determine and prescribe the effective amount of the pharmaceutical composition required. In some embodiments, the physician or veterinarian could start doses of the compounds of the invention employed in the pharmaceutical composition at levels lower than that required in order to achieve the desired therapeutic effect and gradually increase the dosage until the desired effect is achieved.

In some embodiments, routes of administration of any of the compositions disclosed herein include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration. In some embodiments, the administration is by infusion such as by intravenous infusion.

In some embodiments, the provided embodiments do not involve a lymphodepletion therapy prior to the ex vivo administration of the viral vector. Thus, the provided methods do not involve administration of lymphopleting regimens, such as those including cyclophosphamide and/or fludarabine and/or bendamustine, or other lymphodepleting regimens or protocols, prior to receiving administration of the viral vector. It is understood that the exclusion of lymphodepleting therapies in accord to the provided methods does not exclude that the subject may have previous in time (e.g. months to years earlier) may have received a lymphodepleting therapy. Rather, the provided embodiments include those in which the subject has not in accord with the present dosing methods received a lymphodepleting therapy, such as within 60 days or 30 days, prior to the ex vivo administration of the viral vector of the present methods.

In some embodiments, the viral vector composition comprising an exogenous agent or cargo, may be used to deliver such exogenous agent or cargo to a cell tissue or subject. In some embodiments, delivery of a cargo by administration of a viral vector composition described herein may modify cellular protein expression levels. In certain embodiments, the administered composition directs upregulation of (via expression in the cell, delivery in the cell, or induction within the cell) of one or more cargo (e.g., a polypeptide or mRNA) that provide a functional activity which is substantially absent or reduced in the cell in which the polypeptide is delivered. In some embodiments, the missing functional activity may be enzymatic, structural, or regulatory in nature. In some embodiments, the administered composition directs up-regulation of one or more polypeptides that increases (e.g., synergistically) a functional activity which is present but substantially deficient in the cell in which the polypeptide is upregulated. In some of any embodiments, the administered composition directs downregulation of (via expression in the cell, delivery in the cell, or induction within the cell) of one or more cargo (e.g., a polypeptide, siRNA, or miRNA) that repress a functional activity which is present or upregulated in the cell in which the polypeptide, siRNA, or miRNA is delivered. In some of any embodiments, the upregulated functional activity may be enzymatic, structural, or regulatory in nature. In some embodiments, the administered composition directs down-regulation of one or more polypeptides that decreases (e.g., synergistically) a functional activity which is present or upregulated in the cell in which the polypeptide is downregulated. In some embodiments, the administered composition directs upregulation of certain functional activities and downregulation of other functional activities.

In some of any embodiments, the viral vector composition (e.g., one comprising mitochondria or DNA) mediates an effect on a target cell, and the effect lasts for at least 1, 2, 3, 4, 5, 6, or 7 days, 2, 3, or 4 weeks, or 1, 2, 3, 6, or 12 months. In some embodiments (e.g., wherein the viral vector composition comprises an exogenous protein), the effect lasts for less than 1, 2, 3, 4, 5, 6, or 7 days, 2, 3, or 4 weeks, or 1, 2, 3, 6, or 12 months.

In some of any embodiments, the viral vector composition described herein is delivered for ex-vivo administration to a cell or tissue, e.g., a human cell or tissue. In embodiments, the composition improves function of a cell or tissue ex-vivo, e.g., improves cell viability, respiration, or other function (e.g., another function described herein).

In some embodiments, the composition is delivered for ex vivo administration to a tissue that is in an injured state (e.g., from trauma, disease, hypoxia, ischemia or other damage).

In some embodiments, the composition is delivered for ex-vivo transplant (e.g., a tissue explant or tissue for transplantation, e.g., a human vein, a musculoskeletal graft such as bone or tendon, cornea, skin, heart valves, nerves; or an isolated or cultured organ, e.g., an organ to be transplanted into a human, e.g., a human heart, liver, lung, kidney, pancreas, intestine, thymus, eye). In some embodiments, the composition is delivered to the tissue or organ before, during and/or after transplantation.

In some embodiments, the viral vector compositions described herein can be administered to a subject, e.g., a mammal, e.g., a human. In such embodiments, the subject may be at risk of, may have a symptom of, or may be diagnosed with or identified as having, a particular disease or condition (e.g., a disease or condition described herein).

In some embodiments, the source of viral vector are from the same subject that is administered a viral vector composition. In other embodiments, they are different. In some embodiments, the source of viral vector and recipient tissue may be autologous (from the same subject) or heterologous (from different subjects). In some embodiments, the donor tissue for viral vector compositions described herein may be a different tissue type than the recipient tissue. In some embodiments, the donor tissue may be muscular tissue and the recipient tissue may be connective tissue (e.g., adipose tissue). In other embodiments, the donor tissue and recipient tissue may be of the same or different type, but from different organ systems.

In some embodiments, the viral vector composition described herein may be administered to a subject having a cancer, an autoimmune disease, an infectious disease, a metabolic disease, a neurodegenerative disease, or a genetic disease (e.g., enzyme deficiency). In some embodiments, the subject is in need of regeneration.

In some embodiments, the viral vector is co-administered with an inhibitor of a protein that inhibits membrane fusion. For example, Suppressyn is a human protein that inhibits cell-cell fusion (Sugimoto et al., “A novel human endogenous retroviral protein inhibits cell-cell fusion” Scientific Reports 3: 1462 (DOI: 10.1038/srep01462)). In some embodiments, the viral vector particles is co-administered with an inhibitor of sypressyn, e.g., a siRNA or inhibitory antibody.

### VI. EXEMPLARY EMBODIMENTS

Among the provided embodiments are

1. A method of transducing cells in subject, the method comprising:
(a) administering to a subject an inhibitor of the mammalian target of rapamycin (mTOR), and
(b) administering to the subject a viral vector comprising a viral fusogen embedded in the lipid bilayer.

2. The method of embodiment 2, wherein the viral vector comprises an exogenous agent.

3. A method of delivering an exogenous agent to a subject, the method comprising:
(a) administering to a subject an inhibitor of mTOR, and
(b) administering to the subject a viral vector comprising an exogenous agent, wherein the viral vector comprises a fusogen embedded in the lipid bilayer.

4. A method of transducing cells in a subject, the method comprising
(a) administering to a subject an inhibitor of the mammalian target of rapamycin (mTOR), and
(b) administering to the subject a lentiviral vector comprising one or more Nipah envelope proteins or functional variants thereof wherein the vector comprises a polynucleotide encoding a chimeric antigen receptor (CAR), and
(c) administering to the subject IL-7 or a functional variant thereof.

5. A method of transducing cells in a subject, the method comprising
(a) administering to a subject an inhibitor of the mammalian target of rapamycin (mTOR), and
(b) administering to the subject a lentiviral vector comprising one or more Nipah envelope proteins or functional variants thereof wherein the vector comprises
(i) a viral VPX protein, and
(ii) a polynucleotide encoding a chimeric antigen receptor (CAR).

6. The method of any of embodiments 1-5, wherein the inhibitor of mTOR and the viral vector are administered separately or in the same composition.

7. The method of any of embodiments 1-6, wherein the inhibitor of mTOR and the viral vector are administered separately.

8. The method of any of embodiments 1-5 and 7, wherein the inhibitor of mTOR is administered prior to, consecutively, or after administering the viral vector.

9. The method of any of embodiments 1-8, wherein the time period between the administration of the inhibitor of mTOR and viral vector is no more than three days.

10. The method of any of embodiments 1-9, wherein the time period between the administration of the inhibitor of mTOR and viral vector is no more than one day.

11. The method of any of embodiments 1-10, wherein the time period between the administration of the inhibitor of mTOR and viral vector is no more than 12, 6, or 3 hours.

12. The method of any of embodiments 1-11, wherein the inhibitor of mTOR and the viral vector are administered on the same day.

13. The method of any of embodiments 1-12, wherein the method further comprises administering to the subject an inhibitor of an antiviral restriction factor.

14. A method of transducing cells in subject, the method comprising:
(a) administering to a subject an inhibitor of an antiviral restriction factor, and
(b) administering to the subject a viral vector comprising a viral fusogen embedded in the lipid bilayer.

15. The method of embodiment 13 and 14, wherein the inhibitor of an antiviral restriction factor is an inhibitor of SAMHD1, an inhibitor of IFITM1, and/or an inhibitor of IFITM3.

16. The method of embodiment 13, 14, or 15, wherein the inhibitor of an antiviral restriction factor is an inhibitor of SAMHD1, optionally wherein the inhibitor increases phosphorylation and/or degradation of SAMHD1.

17. The method of embodiments 13, 14 or 15, wherein the inhibitor of an antiviral restriction factor is an inhibitor of IFITM1 and/or an inhibitor of IFITM3, optionally wherein the inhibitor reduces expression of IFITM1.

18. The method of embodiments 13-17, wherein the inhibitor of an antiviral restriction factor is an oligonucleotide, optionally wherein the inhibitor of an antiviral restriction factor is an anti-sense oligonucleotide complementary to an RNA encoding said cellular restriction factor.

19. The method of any of embodiments 13, 14, 15, or 17, wherein the inhibitor of an antiviral restriction factor is a resveratrol cyclotrimer, optionally caraphenol A, a-viniferin or resveratrol, or an analog compound thereof.

20. The method of any of embodiments 13-17, wherein the inhibitor of an antiviral restrictions factor is an antifungal agent.

21. The method of any embodiments 13-20, wherein the inhibitor of an antiviral restriction factor is a polyene antifungal agent, optionally nystatin, pimaricin, or amphotericin B.

22. The method of any of embodiment 13-21, wherein the inhibitor of an antiviral restriction factor is amphotericin B.

23. The method of any of embodiments 13-22, wherein the time period between the administration of the inhibitor of the antiviral restriction factor and viral vector is no more than one day.

24. The method of any of embodiments 13-23, wherein the time period between the administration of the inhibitor of the antiviral restriction factor and viral vector is no more than 12 hours.

25. The method of any of embodiments 13-24, wherein the time period between the administration of the inhibitor of the antiviral restriction factor and viral vector is no more than 1, 2, 3, 4, or 5 hours.

26. The method of any of embodiments 13-25, wherein the inhibitor of an antiviral restriction factor is administered at a dose of at or about 0.1-10 mg, 1-10 mg, 10-20 mg, 20-30 mg, 30-40 mg, or 40-50, or any value between the foregoing.

27. A method for administering a viral vector to a subject, the method comprising:
(a) contacting peripheral blood mononuclear cells (PBMCs) or a subset thereof from a subject with a composition comprising a viral vector and an inhibitor of mTOR to create a transduction mixture; and
(b) administering the transduction mixture to the subject, thereby administering the viral vector to the subject.

28. A method for administering a viral vector to a subject, the method comprising:
(a) contacting peripheral blood mononuclear cells (PBMCs) or a subset thereof from a subject with a composition comprising a viral vector and an inhibitor of mTOR that is temsirolimus to create a transduction mixture; and
(b) administering the transduction mixture to the subject, thereby administering the viral vector to the subject.

29. A method for administering a viral vector to a subject, the method comprising:
(a) contacting peripheral blood mononuclear cells (PBMCs) or a subset thereof from a subject with a composition comprising a viral vector and an inhibitor of an antiviral restriction factor to create a transduction mixture; and
(b) administering the transduction mixture to the subject, thereby administering the viral vector to the subject.

30. A method for administering a viral vector to a subject, the method comprising:
(a) contacting peripheral blood mononuclear cells (PBMCs) or a subset thereof from a subject with a composition comprising a viral vector and a polyene antifungal agent to create a transduction mixture; and
(b) administering the transduction mixture to the subject, thereby administering the viral vector to the subject.

31. The method of embodiments 23-30, wherein the contacting and administering is performed in a closed fluid circuit.

32. The method of embodiment 27, 28, or 31, wherein an inhibitor of mTOR is administered prior to contacting in step (A).

33. The method of embodiments 29, wherein the inhibitor of an antiviral restriction factor is administered prior to contacting in step (A).

34. The method of embodiments 30, wherein the polyene antifungal agent is administered prior to contacting in step (A).

35. The method of embodiment 30 or 34, wherein the polyene antifungal agent is selected from the group comprising nystatin, pimaricin, or amphotericin B.

36. A method for administering a viral vector to a subject, the method comprising:
(a) obtaining whole blood from a subject;
(b) collecting from the whole blood a fraction of blood containing PBMCs or a subset thereof;
(c) contacting the fraction of blood containing PBMCs or a subset thereof with a viral vector and an inhibitor of mTOR to create a transduction mixture; and
(d) reinfusing the transduction mixture to the subject, thereby administering the viral vector to the subject,
wherein steps (a)-(d) are performed in-line in a closed fluid circuit.

37. A method for administering a viral vector to a subject, the method comprising:
(a) obtaining whole blood from a subject;
(b) collecting from the whole blood a fraction of blood containing PBMCs or a subset thereof;
(c) contacting the fraction of blood containing PBMCs or a subset thereof with a viral vector and an inhibitor of an antiviral restriction factor to create a transduction mixture; and
(d) reinfusing the transduction mixture to the subject, thereby administering the viral vector to the subject,
wherein steps (a)-(d) are performed in-line in a closed fluid circuit.

38. A method for administering a viral vector to a subject, the method comprising:
(a) administering to a subject an inhibitor of mTOR;
(b) obtaining whole blood from a subject;
(c) collecting from the whole blood a fraction of blood containing PBMCs or a subset thereof;
(d) contacting the fraction of blood containing PBMCs or a subset thereof with a viral vector to create a transduction mixture; and
(e) reinfusing the transduction mixture to the subject, thereby administering the viral vector to the subject,
wherein steps (a)-(e) are performed in-line in a closed fluid circuit.

39. A method for administering a viral vector to a subject, the method comprising:
(a) administering to a subject an inhibitor of an antiviral restriction factor;
(b) obtaining whole blood from a subject;
(c) collecting from the whole blood a fraction of blood containing PBMCs or a subset thereof;
(d) contacting the fraction of blood containing PBMCs or a subset thereof with a viral vector to create a transduction mixture; and
(e) reinfusing the transduction mixture to the subject, thereby administering the viral vector to the subject, wherein steps (a)-(e) are performed in-line in a closed fluid circuit.

40. The method of embodiment 38, wherein the method further comprises contacting the fraction of blood containing PBMCs or a subset thereof with a viral vector and an inhibitor of mTOR to create a transduction mixture in step (d).

41. The method of embodiment 39, wherein the method further comprises contacting the fraction of blood containing PBMCs or a subset thereof with a viral vector and an inhibitor of an antiviral restriction factor to create a transduction mixture in step (d).

42. The method of embodiment 27-41, wherein the viral vector comprises an exogenous agent.

43. A method for delivering an exogenous agent to a subject, the method comprising:
(a) contacting PBMCs or a subset thereof from a subject with a viral vector and an inhibitor of mTOR to create a transduction mixture, wherein the viral vector comprises an exogenous agent; and
(b) administering the transduction mixture to the subject, thereby administering the exogenous agent to the subject.

44. A method for delivering an exogenous agent to a subject, the method comprising:
(a) contacting PBMCs or a subset thereof from a subject with a viral vector and an inhibitor of mTOR that is temsirolimus to create a transduction mixture, wherein the viral vector comprises an exogenous agent; and
(b) administering the transduction mixture to the subject, thereby administering the exogenous agent to the subject.

45. A method for delivering an exogenous agent to a subject, the method comprising:
(a) contacting PBMCs or a subset thereof from a subject with a viral vector and an inhibitor of an antiviral restriction factor to create a transduction mixture, wherein the viral vector comprises an exogenous agent; and
(b) administering the transduction mixture to the subject, thereby administering the exogenous agent to the subject.

46. A method for delivering an exogenous agent to a subject, the method comprising:
(a) contacting PBMCs or a subset thereof from a subject with a viral vector and a polyene antifungal agent to create a transduction mixture, wherein the viral vector comprises an exogenous agent; and
(b) administering the transduction mixture to the subject, thereby administering the exogenous agent to the subject.

47. The method of embodiment 43-46, wherein the contacting and administering is performed in a closed fluid circuit.

48. The method of embodiment 43, 44, or 47, wherein an inhibitor of mTOR is administered prior to contacting in step (A).

49. The method of embodiments 45, wherein the inhibitor of an antiviral restriction factor is administered prior to contacting in step (A).

50. The method of embodiments 46, wherein the polyene antifungal agent is administered prior to contacting in step (A).

51. The method of embodiment 46 or 50, wherein the polyene antifungal agent is selected from the group comprising nystatin, pimaricin, or amphotericin B.

52. A method for delivering an exogenous agent to a subject, the method comprising:
(a) obtaining whole blood from a subject;
(b) collecting from the whole blood a fraction of blood containing peripheral blood mononuclear cells (PBMCs) or a subset thereof;
(c) contacting the fraction of blood containing peripheral blood mononuclear cells (PBMCs) or a subset thereof with a viral vector and an inhibitor of mTOR to create a transduction mixture,
wherein the viral vector comprises an exogenous agent; and
(d) reinfusing the transduction mixture to the subject, thereby administering the exogenous agent to the subject,
wherein steps (a)-(d) are performed in-line in a closed fluid circuit.

53. A method for delivering an exogenous agent to a subject, the method comprising:
(a) obtaining whole blood from a subject;
(b) collecting from the whole blood a fraction of blood containing peripheral blood mononuclear cells (PBMCs) or a subset thereof;
(c) contacting the fraction of blood containing peripheral blood mononuclear cells (PBMCs) or a subset thereof with a viral vector and an inhibitor of an antiviral restriction factor to create a transduction mixture, wherein the viral vector comprises an exogenous agent; and
(d) reinfusing the transduction mixture to the subject, thereby administering the exogenous agent to the subject,
wherein steps (a)-(d) are performed in-line in a closed fluid circuit.

54. A method for delivering an exogenous agent to a subject, the method comprising:
(a) administering to the subject inhibitor of mTOR;
(b) obtaining whole blood from a subject;
(c) collecting from the whole blood a fraction of blood containing PBMCs or a subset thereof;
(d) contacting the fraction of blood containing PBMCs or a subset thereof with a viral vector to create a transduction mixture; and
(e) reinfusing the transduction mixture to the subject, thereby administering the viral vector to the subject,
wherein steps (a)-(e) are performed in-line in a closed fluid circuit.

55. A method for delivering an exogenous agent to a subject, the method comprising:
(a) administering to the subject inhibitor of an antiviral restriction factor;
(b) obtaining whole blood from a subject;
(c) collecting from the whole blood a fraction of blood containing PBMCs or a subset thereof;
(d) contacting the fraction of blood containing PBMCs or a subset thereof with a viral vector to create a transduction mixture; and
(e) reinfusing the transduction mixture to the subject, thereby administering the viral vector to the subject,
wherein steps (a)-(e) are performed in-line in a closed fluid circuit.

56. The method of embodiment 54, wherein the method further comprises contacting the fraction of blood containing PBMCs or a subset thereof with a viral vector and an inhibitor of mTOR to create a transduction mixture in step (d).

57. The method of embodiment 55, wherein the method further comprises contacting the fraction of blood containing PBMCs or a subset thereof with a viral vector and an inhibitor of an antiviral restriction factor to create a transduction mixture in step (d).

58. A method for administering a viral vector to a subject, the method comprising:
(a) contacting peripheral blood mononuclear cells (PBMCs) or a subset thereof from a subject with a composition comprising a lentiviral vector comprising one or more Nipah envelope proteins or functional variants thereof, an inhibitor of an antiviral restriction factor, and Il-7 or a functional variant thereof to create a transduction mixture; and
(b) administering the transduction mixture to the subject, thereby administering the viral vector to the subject, wherein the vector comprises a chimeric antigen receptor (CAR).

59. A method for administering a viral vector to a subject, the method comprising:
(a) contacting peripheral blood mononuclear cells (PBMCs) or a subset thereof from a subject with a composition comprising a lentiviral vector comprising one or more Nipah envelope proteins or functional variants thereof and an inhibitor of an antiviral restriction factor, to create a transduction mixture; and
(b) administering the transduction mixture to the subject, thereby administering the viral vector to the subject, wherein the vector comprises a viral VPX protein and a chimeric antigen receptor (CAR).

60. A method for transducing cells in a subject, the method comprising:
(a) contacting peripheral blood mononuclear cells (PBMCs) or a subset thereof from a subject with a composition comprising a lentiviral vector comprising one or more Nipah envelope proteins or functional variants thereof, and an inhibitor of an antiviral restriction factor to create a transduction mixture; and
(b) administering the transduction mixture to the subject, thereby administering the viral vector to the subject, wherein the vector comprises a viral VPX protein and a chimeric antigen receptor (CAR).

61. A method for transducing cells in a subject, the method comprising:
(a) contacting peripheral blood mononuclear cells (PBMCs) or a subset thereof from a subject with a composition comprising a lentiviral vector comprising one or more Nipah envelope proteins or functional variants thereof, an inhibitor of an antiviral restriction factor, and Il-7 or a functional variant thereof to create a transduction mixture; and
(b) administering the transduction mixture to the subject, thereby administering the viral vector to the subject, wherein the vector comprises a chimeric antigen receptor (CAR).

62. A method for administering a viral vector to a subject, the method comprising:
(a) contacting peripheral blood mononuclear cells (PBMCs) or a subset thereof from a subject with a composition comprising a lentiviral vector comprising one or more Nipah envelope proteins or functional variants thereof, an inhibitor of mTOR, and Il-7 or a functional variant thereof to create a transduction mixture; and
(b) administering the transduction mixture to the subject, thereby administering the viral vector to the subject, wherein the vector comprises a chimeric antigen receptor (CAR).

63. A method for administering a viral vector to a subject, the method comprising:
(a) contacting peripheral blood mononuclear cells (PBMCs) or a subset thereof from a subject with a composition comprising a lentiviral vector comprising one or more Nipah envelope proteins or functional variants thereof and an inhibitor of mTOR, to create a transduction mixture; and
(b) administering the transduction mixture to the subject, thereby administering the viral vector to the subject, wherein the vector comprises a viral VPX protein and a chimeric antigen receptor (CAR).

64. A method for transducing cells in a subject, the method comprising:
(a) contacting peripheral blood mononuclear cells (PBMCs) or a subset thereof from a subject with a composition comprising a lentiviral vector comprising one or more Nipah envelope proteins or functional variants thereof, and an inhibitor of mTOR to create a transduction mixture; and
(b) administering the transduction mixture to the subject, thereby administering the viral vector to the subject, wherein the vector comprises a viral VPX protein and a chimeric antigen receptor (CAR).

65. A method for transducing cells in a subject, the method comprising:
(a) contacting peripheral blood mononuclear cells (PBMCs) or a subset thereof from a subject with a composition comprising a lentiviral vector comprising one or more Nipah envelope proteins or functional variants thereof, an inhibitor of mTOR, and Il-7 or a functional variant thereof to create a transduction mixture; and
(b) administering the transduction mixture to the subject, thereby administering the viral vector to the subject, wherein the vector comprises a chimeric antigen receptor (CAR).

66. The method of any of embodiments 27-65, wherein the PBMCs or subset are further contacted with an inhibitor of an antiviral restriction factor.

67. The method of any of embodiments 27-65, wherein the transduction mixture further comprises an inhibitor of an antiviral restriction factor.

68. The method of embodiment 57-61, 66 or 67, wherein the inhibitor of an antiviral restriction factor is an inhibitor of SAMHD1, an inhibitor of IFITM1, and/or an inhibitor of IFITM3.

69. The method of embodiment 68, wherein the inhibitor of an antiviral restriction factor is an inhibitor of SAMHD1, optionally wherein the inhibitor increases phosphorylation and/or degradation of SAMHD1.

70. The method of embodiment 68, wherein the inhibitor of an antiviral restriction factor is an inhibitor of IFITM1, optionally wherein the inhibitor reduces expression of IFITM1.

71. The method of embodiments 57-61, 66-70, wherein the inhibitor of an antiviral restriction factor is an oligonucleotide, optionally wherein the inhibitor of an antiviral restriction factor is an anti-sense oligonucleotide complementary to an RNA encoding said cellular restriction factor.

72. The method of any of embodiments 57-61, 66-68, or 70, wherein the inhibitor of an antiviral restriction factor is a resveratrol cyclotrimer, optionally caraphenol A, a-viniferin or resveratrol, or an analog compound thereof.

73. The method of any of embodiments 57-61, 66-70, wherein the inhibitor of an antiviral restriction factor is an antifungal agent, optionally a polyene antifungal agent, further optionally amphotericin B.

74. The method of any of embodiments 57-61, 66-73, wherein the inhibitor of an antiviral restriction factor is contacted with PBMCs at a dose of at or about 0.1-10 μM, 1-10 μM, 10-20 μM, 20-30 μM, 30-40 μM, 40-50 μM, 50-60 μM, 60-70 μM, 70-80 μM, 80-90 μM, or 90-100 M, or any value between the foregoing.

75. The method of any of embodiments 27-74, wherein the method is carried out in a single in-line procedure to maintain a closed or functionally closed fluid circuit.

76. The method of any of embodiments 27-75, wherein the method is characterized by the whole blood, PBMCs or subset thereof, and transduction mixture having not been subjected to cryopreservation or freezing.

77. The method of any of embodiments 27-76, wherein the PBMCs or subset thereof, and transfection mixture are not formulated with a cryoprotectant (e.g. DMSO).

78. The method of any of embodiments 27-77, wherein the transduction mixture is directly reinfused to the subject, optionally without any further processing or washing steps.

79. The method of any of embodiments 31-42, or 47-56, or 75, wherein the closed fluid circuit comprises one or more of a blood processing set for obtaining the whole blood from the subject, a separation chamber for the separating the PBMCS or subset from the blood to collect the PBMCs or subset, a contacting container for the contacting the collected PBMCs or subset thereof with the composition comprising lipid particles (e.g. lentiviral vector), and a transfer container containing the contacted PBMCs or subset thereof and/or the transfection mixture for reinfusion to the subject.

80. The method of embodiment 79, wherein the closed fluid circuit further comprises a collection container operably connected to the separation chamber to collect the PBMCs or subset, optionally wherein the collection container is a bag, more optionally a sterile bag.

81. The method of any of embodiments 27-80, wherein during at least a portion of the contacting the method comprises mixing the transduction mixture comprising the PBMCs or subset and the composition comprising the viral vector.

82. The method of embodiment 81, wherein the mixing is by physical manipulation and/or centrifugation.

83. The method of any of embodiments 26-29, 33-55, wherein the collected fraction of blood contains PBMCs or subset thereof separated from other blood components.

84. The method of any of embodiments 36-42, 52-83, wherein collecting the fraction of blood is by apheresis.

85. The method of embodiment 84, wherein the apheresis device comprises membrane apheresis or centrifugal apheresis.

86. The method of any of embodiments 36-42, 52-85, wherein the collected fraction comprises leukocytes or precursors thereof.

87. The method of embodiment 86, wherein the precursors thereof comprise hematopoietic stem cells.

88. The method of any of embodiments 36-42, 52-87, wherein collecting the fraction of blood is by leukapheresis.

89. The method of embodiment 88, wherein the collected fraction of blood contains leukocytes.

90. The method of embodiment 13-26, 66-89, wherein the inhibitor of an antiviral restriction factor is a cytokine.

91. The method of embodiment 90, wherein the cytokine comprises IL-7, IL-15, or both IL-7 and IL-15.

92. The method of embodiments 13-26, 66-89, wherein the inhibitor of an antiviral restriction factor is an antifungal agent, optionally a polyene antifungal agent, further optionally amphotericin B.

93. The method of any of embodiments 1-92, wherein the inhibitor of mTOR is rapamycin or a rapamycin analogue.

94. The method of any of embodiments 1-93, wherein the inhibitor of mTOR is selected from the group comprising rapamycin, everolimus, temsirolimus, or ridaforolimus.

95. The method of any of embodiments 1-94, wherein the inhibitor of mTOR is rapamycin.

96. The method of any of embodiments 1-26 and 93-95, wherein the inhibitor of mTOR is administered at a dose of 1 mg to 1000 mg per day or 1 mg/m2/day to 500 mg/m2/day, or as a single dose of 1 mg to 1000 mg or 1 mg/m2 to 500 mg/m2/dose.

97. The method of embodiment 96, wherein the inhibitor of mTOR is administered as a single dose of 2 mg to 50 mg.

98. The method of embodiment 96 or embodiment 97, wherein the inhibitor of mTOR is administered as a single dose of 25 mg.

99. The method of embodiment 96, wherein the inhibitor of mTOR is administered as a single dose of 100 mg/m2 to 300 mg/m2/dose.

100. The method of embodiment 99, wherein the inhibitor of mTOR is administered at a dose of 220 mg/m2/dose.

101. The method of embodiment 96, wherein administration of the inhibitor of mTOR further comprising a loading dose.

102. The method of embodiment 101, wherein the loading dose is administered at a dose of 1 mg to 1000 mg per day or 1 mg/m2/day to 500 mg/m2/day, or as a single dose of 1 mg to 1000 mg or 1 mg/m2 to 500 mg/m2/dose.

103. The method of embodiments 101 or 102, wherein the loading dose is administered at a dose of 25 mg per day, 50 mg per day, or 500 mg per day. 104. The method of any of embodiments 1-26 and 93-103, wherein the inhibitor of mTOR is administered orally or intravenously, optionally wherein the inhibitor of mTOR is administered intravenously.

105. The method of any of embodiments 27-95, wherein the inhibitor of mTOR is contacted with the PBMCs or the subset thereof in an amount from 1 μM to 50 μM.

106. The method of any of embodiments 27-95, and 105, wherein the inhibitor of mTOR is contacted with the PBMCs or the subset thereof in an amount of at or about 5 μM, 10 μM, 15 μM, 20 μM, 25 μM, 30 μM, 35 μM, or 40 μM, or any value between any of the foregoing.

107. The method of any of embodiments 1-26, and 95-106 wherein the method further comprises administration of one or more recombinant cytokine to the subject.

108. The method of any of embodiments 25-65, wherein the PBMCs or subset are further contacted with one or more recombinant cytokine.

109. The method of embodiments 25-89, wherein the transduction mixture further comprises one or more recombinant cytokines.

110. The method of any of embodiments 107-109, wherein the one or more recombinant cytokines comprise recombinant IL-7, recombinant IL-15, or both recombinant IL-7 and recombinant IL-15.

111. The method of embodiment 109 or embodiment 110, wherein the one or more recombinant cytokine further comprises IL-2.

112. The method of embodiment 107, 108, 110, or 111, wherein the time period between the administration of the recombinant cytokine and viral vector is no more than one day.

113. The method of any of embodiments 107, 108, or 110-112, wherein the time period between the administration of the cytokine and viral vector is no more than 12 hours.

114. The method of any of embodiments 107, 108, or 110-113, wherein the time period between the administration of the cytokine and viral vector is no more than 1, 2, 3, 4, or 5 hours.

115. The method of any of embodiments 107-114, wherein the recombinant cytokine is administered at a dose of from at or about 0.001 mg/kg to at or about 0.1 mg/kg, at or about 0.001 mg/kg to at or about 0.05 mg/kg, at or about 0.001 mg/kg to at or about 0.01 mg/kg, at or about 0.01 mg/kg to at or about 0.1 mg/kg, at or about 0.01 mg/kg to at or about 0.05 mg/kg or at or about 0.05 mg/kg to at or about 0.1 mg/kg.

116. The method of embodiment 115, wherein the recombinant cytokine is administered at a dose of from or from about 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.02 mg/kg, 0.03 mg/kg, 0.04 mg/kg, or 0.05 mg/kg, or any value between any of the foregoing.

117. The method of any of embodiments 1-4, 6-62, 65-116, wherein the viral vector further comprises a viral accessory protein, optionally wherein the viral accessory protein is a viral kinase.

118. The method of embodiment 117, wherein the viral accessory protein is an inhibitor of an antiviral restriction factor, optionally wherein the viral accessory protein is an inhibitor of SAMHD1 activity.

119. The method of embodiments 117 or embodiment 118, wherein the viral accessory protein directly or indirectly phosphorylates SAMHD1.

120. The method of embodiments 117-119, wherein the viral accessory protein is selected from the group consisting of: BLG4, UL97, and U69.

121. The method of embodiments 117 or 118, wherein the viral accessory protein degrades SAMHD1.

122. The method of any of embodiments 117-121, wherein the viral accessory protein is a a fusion protein, optionally a fusion protein with VPX and/or Vpr.

123. The method of any of embodiments 1-3, 6-61, 66-122, wherein the viral vector is a retroviral vector.

124. The method of any of embodiments 1-3, 6-61, 66-123, wherein the viral vector is a lentiviral vector.

125. The method of any of embodiments 1-3, 6-61, 66-124, wherein the viral vector is pseudotyped with the fusogen.

126. The method of any of embodiments 1-3, 6-61, 66-125, wherein the viral fusogen is selected from a Class I viral membrane fusion protein, a Class II viral membrane protein, a Class II viral membrane fusion protein, a viral membrane glycoprotein, or a viral envelope protein.

127. The method of any of embodiments 1-3, 6-61, 66-126, wherein the viral fusogen comprises a viral envelope protein or a functional variant thereof.

128. The method of any of embodiments 1-3, 6-61, 66-127, wherein the viral fusogen is a vesicular stomatitis virus envelope glycoprotein (VSV-G).

129. The method of 1-3, 6-61, 66-127, wherein the viral fusogen is a baboon endogenous virus (BaEV) envelope glycoprotein.

130. The method of 1-3, 6-61, 66-127, wherein the viral fusogen is a Cocal virus envelope glycoprotein.

131. The method of 1-3, 6-61, 66-127, wherein the viral fusogen is an Alphavirus class II fusion protein or a functional variant thereof, optionally wherein the Alphavirus is a Sindbis virus.

132. The method of 1-3, 6-61, 66-127, wherein the viral fusogen comprises a Paramyxovirus fusion (F) protein or a biologically active portion thereof, optionally wherein the Paramyxovirus is a Morbillivirus or a Henipavirus.

133. The method of any of embodiments 1-3, 6-61, 66-127, and 132, wherein the viral fusogen comprises a Morbillivirus fusion (F) protein.

134. The method of embodiment 133, wherein the Morbillivirus F protein is from a measles virus (MeV), canine distemper virus, Cetacean morbillivirus, Peste-des-petits-ruminants virus, Phocine distemper virus, Rinderpest virus or a biologically active portion or functional variant thereof of any of the foregoing.

135. The method of any of embodiments 1-3, 6-61, 66-127, and 132, wherein the viral fusogen comprises a Henipavirus F protein from a Nipah virus, Hendra virus, Cedar virus, Kumasi virus, Mojiang virus or a biologically active portion or functional variant thereof.

136. The method of any of embodiments 1-3, 6-61, 66-127, 132 and 135, wherein the viral fusogen comprises a Nipah virus F protein or a biologically active portion or functional variant thereof.

137. The method of any of embodiments 132-136, wherein the fusogen comprises a paramyxovirus G, paramyxovirus H, or paramyxovirus HN protein, or a biologically active portion or functional variant thereof.

138. The method of embodiment 137, wherein the paramyxovirus G, paramyxovirus H, or paramyxovirus HN protein further comprises a targeting moiety that binds to a molecule on a target cell.

139. The method of any of embodiments 1-3, 6-61, 66-127, and 132-138, wherein the viral fusogen comprises an F protein molecule or a biologically active portion thereof from a Paramyxovirus and a glycoprotein G (G protein) or a biologically active portion thereof from a Paramyxovirus.

140. The method of embodiment 139, wherein the Paramyxovirus is a henipavirus.

141. The method of embodiment 139 or embodiment 140, wherein the Paramyxovirus is Nipah virus.

142. The method of any of embodiments 1-127 and 132-141, wherein the viral fusogen and/or Nipah envelope protein comprises a Nipah virus F glycoprotein (NiV-F) or a biologically active portion or functional variant thereof and a Nipah virus G glycoprotein (NiV-G) or a biologically active portion or functional variant thereof.

143. The method of embodiment 139 or embodiment 140, wherein the Paramyxovirus is Hendra virus.

144. The method of any of embodiments 139-142, wherein the G protein or the biologically active portion thereof is a mutant NiV-G protein or biologically active portion thereof that exhibits reduced binding to Ephrin B2 or Ephrin B3.

145. The method of embodiment 144, wherein the mutant NiV-G protein comprises one or more amino acid substitutions corresponding to amino acid substitutions selected from the group consisting of E501A, W504A, Q530A and E533A with reference to numbering set forth in SEQ ID NO:14.

146. The method of any of embodiments 139-142, 144 and 145, wherein the G protein or biologically active portion is a biologically active portion of wild-type NiV-G that has a deletion of up to 40 amino acids at or near the N-terminus, optionally not including the initial methionine.

147. The method of any of embodiments 139-142, and 144-146, wherein the G protein is a biologically active portion that is a truncated NiV-G that has a deletion of amino acids 2-34 at or near the N-terminus of wild-type NiV-G set forth in SEQ ID NO:14.

148. The method of any of embodiments 139-142, and 144-147, wherein the G protein or the biologically active portion has the amino acid sequence set forth in SEQ ID NO: 19 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:19.

149. The method of any of embodiments 139-142, and 144-146, wherein the F protein or the biologically active portion thereof is a NiV-F protein or a biologically active portion thereof.

150. The method of embodiment 149, wherein the F protein or the biologically active portion is a truncated NiV-F that is truncated by at least or at 22 amino acids or at least or at 20 amino acids at or near the C-terminus of wild-type NiV-F set forth in SEQ ID NO:2, optionally not including the initial methionine.

151. The method of any of embodiments 139-142, and 144-150, wherein the F protein or the biologically active portion is a truncated NiV-F that lacks amino acids 525-546 of SEQ ID NO:2.

152. The method of any of embodiments 139-142, and 144-151, wherein the F protein or the biologically active portion has the amino acid sequence set forth in SEQ ID NO: 12 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:12.

153. The method of any of embodiments 139-142, and 144-151, wherein the NiV-G protein comprises the amino acid sequence set forth in SEQ ID NO: 19, and the NiV-F protein comprises the amino acid sequence set forth in SEQ ID NO:12.

154. The method of any of embodiments 138-153, wherein the targeted moiety is specific for a cell surface receptor on a target cell.

155. The method of any of embodiment 138-154, wherein the targeting domain is a Design ankyrin repeat proteins (DARPin), a single domain antibody (sdAb), a VHH fragment, a single chain variable fragment (scFv), or an antigen-binding fibronectin type III (Fn3) scaffold.

156. The method of any one of embodiments 138-155, wherein the fusogen and the targeting moiety are directly linked.

157. The method of any one of embodiments 138-156, wherein the fusogen and targeting moiety are indirectly linked via a linker.

158. The method of embodiment 157, wherein the linker is a peptide linker.

159. The method of embodiment 158, wherein the peptide linker is (GmS)n (SEQ ID NO: 11), wherein each of m and n is an integer between 1 to 4, inclusive.

160. The method of any of embodiments 2-3, 6-26, 42-56, 66-159, wherein the exogenous agent is a nucleic acid or a polypeptide.

161. The method of embodiment 160, wherein the exogenous agent is a nucleic acid encoding a payload gene, optionally wherein the nucleic acid encodes a chimeric antigen receptor.

162. The method of embodiment 154, wherein the target cell is one or more of a monocyte, macrophage, neutrophil, dendritic cell, eosinophil, mast cell, platelet, large granular lymphocyte, Langerhans' cell, natural killer (NK) cell, T lymphocyte (e.g., T cell), a Gamma delta T cell, B lymphocyte (e.g., B cell), CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, a CD30+ lung epithelial cell, a liver sinusoidal endothelial cell or myocyte.

163. The method of embodiment 162, wherein the target cell is a T cell, optionally wherein the target cell is a resting T cell.

164. The method of embodiment 162 or 163, wherein the target cell is a T cell that has not been activated.

165. The method of any of embodiments 139-142 and 144-164, wherein the viral vector comprises
(i) a re-targeted Nipah virus G glycoprotein (NiV-G) that is a truncated NiV-G set forth in SEQ ID NO:19 linked to a targeting moiety that binds to a T cell, and
(ii) a truncated Nipah virus F glycoprotein (NiV-F) set forth in SEQ ID NO:12.

166. The method of embodiment 139-165, wherein the targeting moiety that binds to a T cell is a CD8 binding agent, CD4 binding agent or CD3 binding agent

167. The method of embodiment 165 or embodiment 166, wherein the targeting moiety is an sdAb or an ScFv.

168. A composition, comprising the transduction mixture of the method of any of embodiments 27-167.

169. A composition comprising a leukapheresis composition for delivering a viral vector to a subject, wherein the leukapheresis composition comprises peripheral blood mononuclear cells (PBMCs) or a subset thereof from the subject, a viral vector, and an inhibitor of mTOR.

170. A composition comprising a leukapheresis composition for delivering a viral vector to a subject, wherein the leukapheresis composition comprises peripheral blood mononuclear cells (PBMCs) or a subset thereof from the subject, a viral vector, and an inhibitor of an antiviral restriction factor.

171. The composition of embodiment 169 or embodiment 170, wherein the delivery to the subject is with an apheresis device.

172. A method of treating a disease or condition in a subject comprising administering a viral vector or exogenous agent by the method of any of embodiments 1-167 to a subject in need thereof.

173. A method of treating a disease or condition comprising infusing the composition of any of embodiments 168-170 into a subject in need thereof.

174. The method of embodiment 172 or embodiment 173, wherein the disease or disorder is treatable by administration of the viral vector or the exogenous agent.

175. The method of any of embodiments 172-174, wherein the disease or condition is a cancer.

176. The lipid particle therapy or method of any of embodiments 172-175, wherein the cancer is a solid tumor, a lymphoma or a leukemia.

### VII. EXAMPLES

The following examples are included for illustrative purposes only and are not intended to limit the scope of the invention.

### Example 1 Inhibition of Restriction Factors for Increased Transduction of Cells by Paramyxovirus-Pseudotyped Fusosomes

The effect of inhibiting certain host cellular restriction factors, such as interferon induced transmembrane protein 1 (IFITM 1) or Sam-domain and HD-domain containing protein 1 (SAMHD1), on transduction efficiency of exemplary target T cells with an exemplary lentiviral vectors was assessed. The studies were carried out on activated and non-activated T cells as described below.

### 1. Knockout (KO) of IFITMs-3 and MxB in Activated T-Cells

CD8+ T cells were activated via anti-CD3/anti-CD28 antibody bead stimulation for three days at a ratio of 3:1 (bead:cell) before electroporation with a CRISPR ribonucleoprotein complex (cRNP) containing CRISPR/Cas9 complexed with 1-3 sgRNAs targeting a gene encoding a target cellular restriction factor for knock-out of target gene expression. Target cellular restriction factor genes for knockout in this assay included MxB, IFITM1, and IFITM2. In addition, a single sgRNA with the ability to target both IFITM1 and IFITM3 was used to doubly knockout IFITM1 and IFITM3. As controls for the processes of knock-out and subsequent repair, genes encoding host cell proteins CD4, CD8, and IFNAR1 also were knocked out in cells.

Cells were transduced with an exemplary lentiviral vector carrying an eGFP reporter gene. The lentiviral vectors were pseudotyped either with VSV-G, or a CD8-retargeted Nipah fusogen composed of a Nipah F protein and a CD8-retargeted Nipah G protein (see US 2019/0144885, incorporated by reference herein). The CD8-retargeted Nipah G (NiV-G) protein contained an anti-CD8 scFv as a fusion with the exemplary NiV-G sequence GcA34 (Bender et al. 2016 PLoS Pathol 12(6):e1005641; set forth in SEQ ID NO:19), and the Nipah F (NiV-F) protein was the exemplary NiV-F sequence NivFdel22 (SEQ ID NO:11; or SEQ ID NO:12 without a signal sequence; Bender et al. 2016 PLoS).

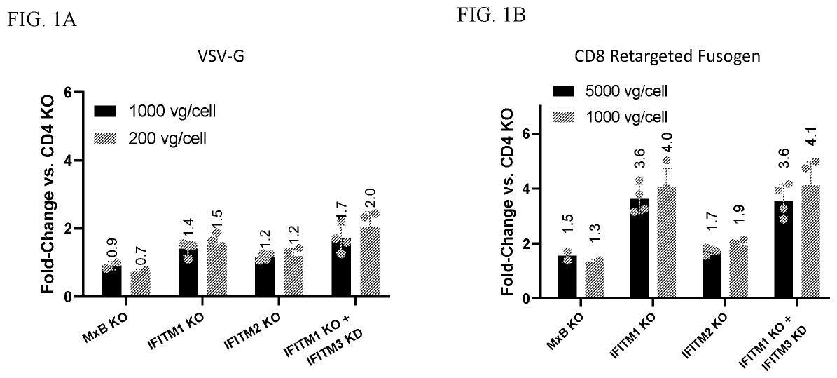

Knock-out of IFITM1 did not substantially improve transduction efficiency of VSV-G pseudotyped lentiviral vectors, as compared to other tested conditions (FIG. 1A). In contrast, knock-out of IFITM1 was observed to result in about a 3 to 4-fold increase in transduction levels of a lentiviral vector pseudotyped with the exemplary CD8-retargeted Nipah fusogen (FIG. 1B). The effect of additional known restriction factor knockouts on activated T-cell transduction efficiency by fusosomes is shown in Table E1.

TABLE E1
Effect of known restriction factor knockouts
on activated T cell transduction

| | Fold change vs. benign KO control | | | |
|---|---|---|---|---|
| Knockout | | | CD4-targeted | CD8-targeted |
| Pseudovirus | VSV-G | | fusosome | fusosome |
| T-cell subset | CD4+ | CD8+ | CD4+ | CD8+ |
| CD4 | 0.9 | 1.0 | 0.1 | 1.0 |
| CD8 | 1.0 | 1.1 | 1.0 | 0.1 |
| IFITM1 | 1.3 | 1.4 | 3.2 | 3.6 |
| IFITM2 | 1.0 | 1.2 | 1.0 | 1.7 |
| IFITM1/3 | 1.4 | 1.7 | 3.4 | 3.6 |
| MxB | ND | 0.9 | ND | 1.5 |
| CTNNBL1 | 1.2 | ND | 0.5 | ND |
| LEDGF | 0.8 | ND | 0.4 | ND |

### 2. Effect of IFITM1 and SAMHD1 on Transduction of Non-Activated T-Cells

Cryopreserved donor CD4+ and CD8+ cells were thawed and electroporated with cRNPs for CRISPR/Cas9 mediated knock out of target genes, including CD4, CD8, SAMHD1, IFITM1, and a combination of both SAMHD1 and IFITM1 using methods described above. In a complementary experiment, CD4+ and CD8+ T-cells were negatively selected for from fresh PBMCs isolated from apheresis. Cells were maintained in conditions such that they were unactivated, and then electroporated with cRNPs for CRISPR/Cas9 mediated knock out of target genes. Thawed and freshly isolated T cells were then transduced with the exemplary CD8-retargeted fusogen pseudotyped lentiviral vector encoding a reporter gene enhanced GFP (eGFP) as described above. Transduction was assessed on Day 36 for thawed cells, and Day 38 for those isolated following apheresis.

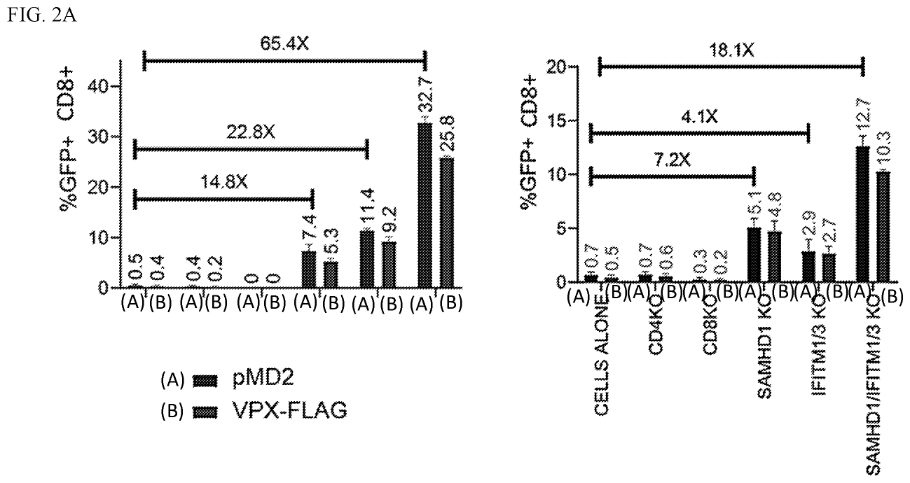

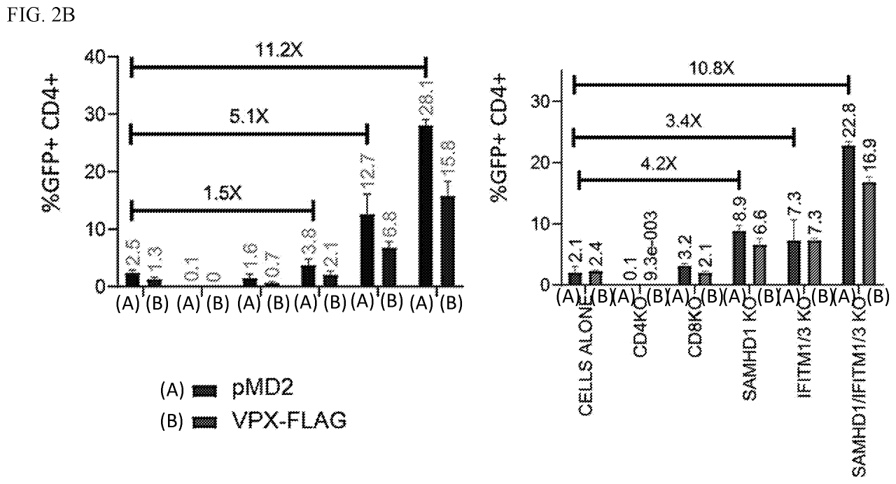

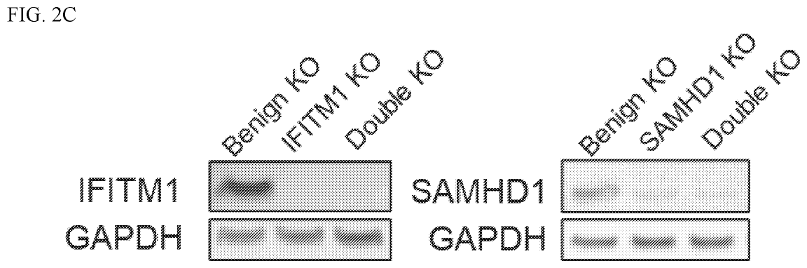

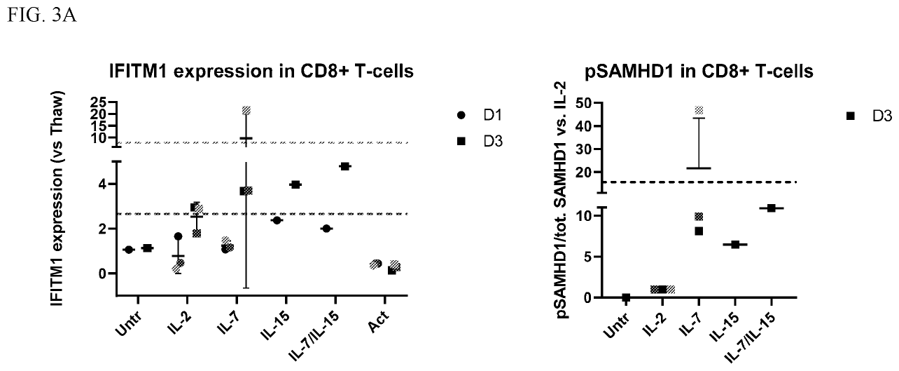

Results are shown in FIG. 2A (thawed cells) and FIG. 2B (freshly isolated cells). Knockout is confirmed by FIG. 2C. It was observed that knockout of both SAMHD1 and IFITM1 potentiated transduction via the exemplary CD8-retargeted fusogen in unactivated T-cells. In some aspects, a synergistic effect between IFITM1 and SAMHD1 knockout was observed to increase transduction efficiency >10-fold.

Together, these results support a finding that inhibition of a cellular restriction factor can potentiate transduction efficiency of T cells, both activated or non-activated, with a lentiviral vector pseudotyped with a paramyxovirus fusogen, such as a Nipah fusogen.

### Example 2 Effect of IL-7 and IL-15 on Expression of Restriction Factors

The effect of IL-7 and IL-15 on phosphorylated SAMHD1 (pSAMHD1) and IFITM1 expression was assessed. CD4+ and CD8+ T cells were treated with either IL-2 (100 U/mL), IL-15 (15 ng/mL), IL-7 (15 ng/mL), or a composition comprising both IL-7 and IL-15. Cells were activated with anti-CD3/anti-CD8 antibody coated beads at a ratio of 3:1, and then cells were assessed for expression of IFITM1 or pSAMHD1. Aggregate densitometry across multiple donors are shown in FIG. 3A (CD8+) and FIG. 3B (CD4+), while representative western blots are also depicted in FIG. 3C (CD8+) and FIG. 3D (CD4+).

Pre-treatment with IL-7 results in increased levels of pSAMHD1 which increases over time. It was also observed that the proportion of pSAMHD1 to total SAMHD1 was increased in the presence of IL-7, IL-15, or both IL-7 and IL-15. Further, IFITM1 expression was similarly increased in these cytokine conditions, although some donor-to-donor variability was observed. Without wishing to be bound by theory, these results support that cytokine treatment increases the levels of pSAMHD1, which is an inactive form of SAMHD1. Thus, while SAMHD1 is a known restriction factor to lentiviral infection, the results are consistent with a finding that treatment of cells with cytokines can be used to inactivate SAMHD1 to improve transduction efficiency.

### Example 3 Assessment of Transduction with an mTOR Inhibitor and Cytokines

The effects of an mTOR inhibitor in the presence or absence of cytokines such as IL-7 and IL-15 on transduction efficiency of exemplary target T cells with Nipah fusogen pseudotyped lentiviral vectors was assessed. mTOR inhibitors, such as rapamycin, are known to down-regulate the cellular restriction factor IFITM3.

Pan T cells comprising CD4+ and CD8+ T cells were thawed on Day 0, and divided into treatment groups. Cells were either treated with anti-CD3/anti-CD8 antibody coated beads at a ratio of 3:1 or left untreated, and then cultured in the presence of either IL-2 (100 U/mL), IL-7 (15 ng/mL), IL-15 (15 ng/mL), or both IL-7 and IL-15 on Days 0-3. Activated cells were debeaded on Day 3, and all cells were then treated with either a CD4-retargeted Nipah fusogen pseudotyped lentiviral vector or a CD8-retargeted Nipah fusogen pseudotyped lentiviral vector via spinfection, each carrying an eGFP reporter gene substantially as described in Example 1. The lentiviral vector was added to the cells at titrated amounts of 15 IU/cell, 5 IU/cell, 1.7 IU/cell or 0.57 IU/cell in the presence or absence of 30 μM rapamycin for two hours, and then cells were transduced by a further 2 hour spinoculation. After spinoculation, the cell media was changed to remove rapamycin and to add fresh media with the respective cytokines as described (I.e., IL-2, IL-7, IL-15, or IL-7/IL-15). The percent GFP+ cells was monitored 3 days post transduction as an marker of transduction efficiency.

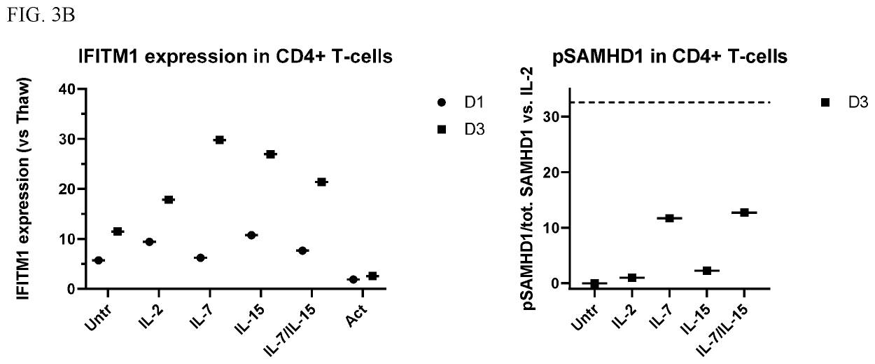

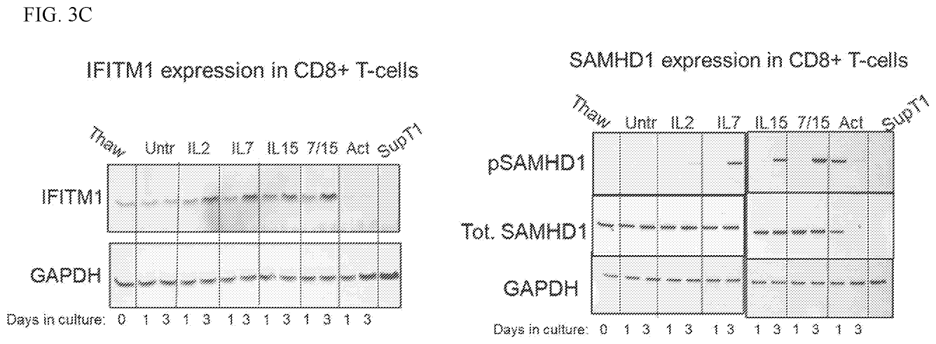

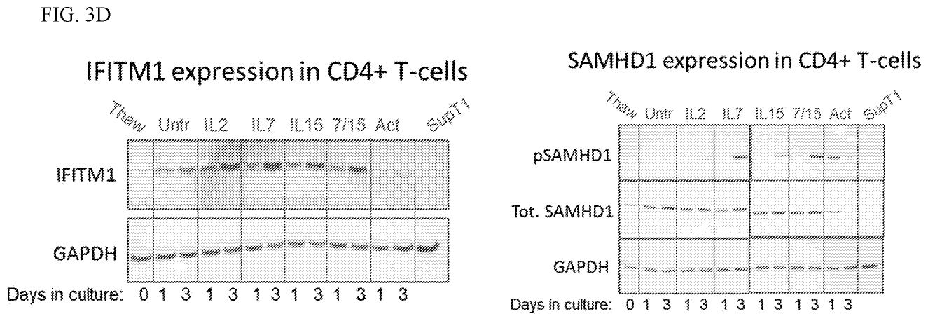

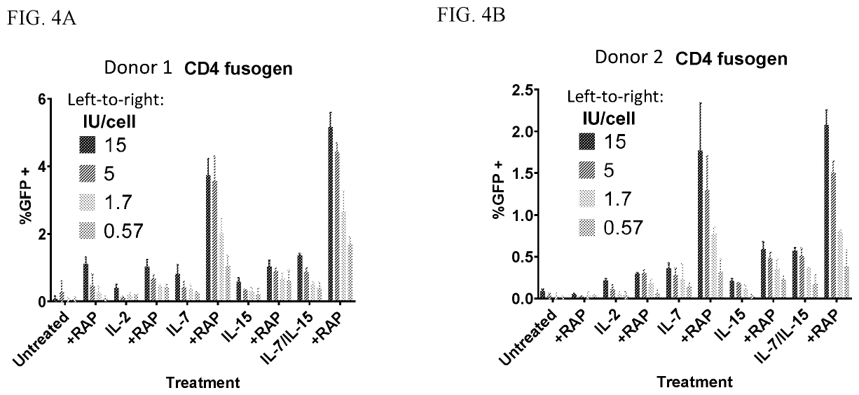

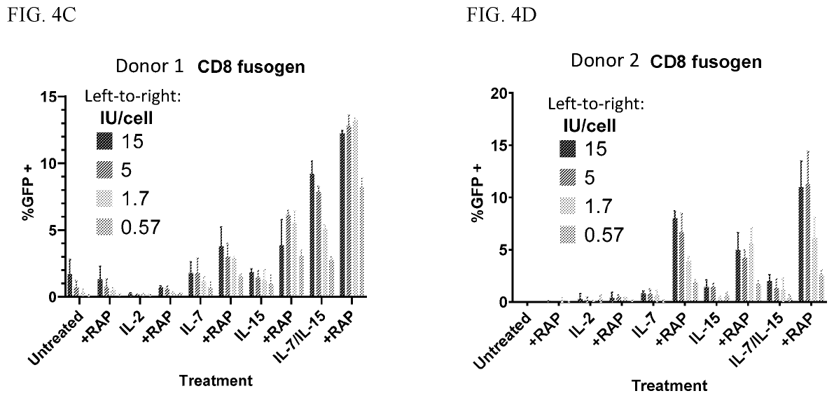

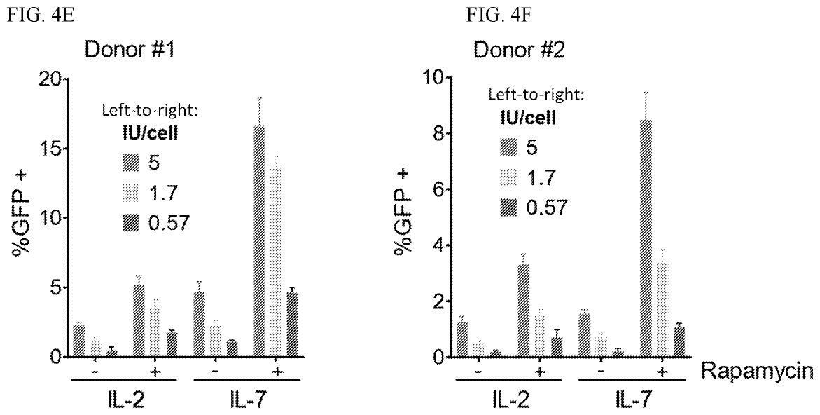

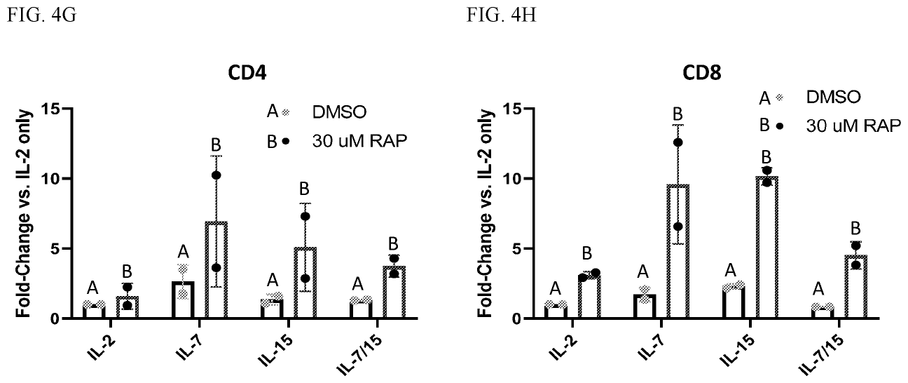

Transduction efficiency of T cells with a CD4-retargeted Nipah fusogen pseudotyped lentiviral vector from two donors is shown in FIGS. 4A and 4B, and transduction efficiency of T cells with a CD8-retargeted Nipah fusogen pseudotyped lentiviral vector from two donors is shown in FIGS. 4C and 4D. In a similar experiment, combination of IL-7 and rapamycin was shown to result in a significant increase in transduction efficiency (FIGS. 4E and 4F). Aggregate data across multiple donors are also shown for a CD4-retargeted Nipah fusogen pseudotyped lentiviral vector in FIG. 4G, and for a CD8-retargeted Nipah fusogen pseudotyped lentiviral vector in FIG. 4H.

In addition to transduction efficiency, expression of certain restriction factors was also assessed in the presence of the respective cytokines as described (I.e., IL-2, IL-7, IL-15, or IL-7/IL-15) with or without 30 μM rapamycin following transduction with either a CD4-retargeted Nipah fusogen pseudotyped lentiviral vector or a CD8-retargeted Nipah fusogen pseudotyped lentiviral vector via spinfection. As shown in FIG. 5A, 4 hour total treatment with rapamycin resulted in a downregulation of IFITM 1 expression of roughly 20-35% across the cytokine treatment conditions. Similar results are shown for another donor in FIG. 5B, where a 5-40% reduction was observed.

These data support that rapamycin increases transduction efficiency of T-cells and also inhibits antiviral restriction factor IFITM1. Further, it was observed the potentiation of transduction via mTOR inhibition was particularly pronounced in the presence of IL-7, or the combination of IL-7 and IL-15.

### Example 4 Assessment of Transduction with Temsirolimus and IL-2 or IL-7

The effects of temsirolimus (TEM), a rapamycin analog, in combination with IL-2 or IL-7 on transduction efficiency and protein expression of pSAMHD1 and IFITM1 expression was assessed.

### A. Transduction Efficiency

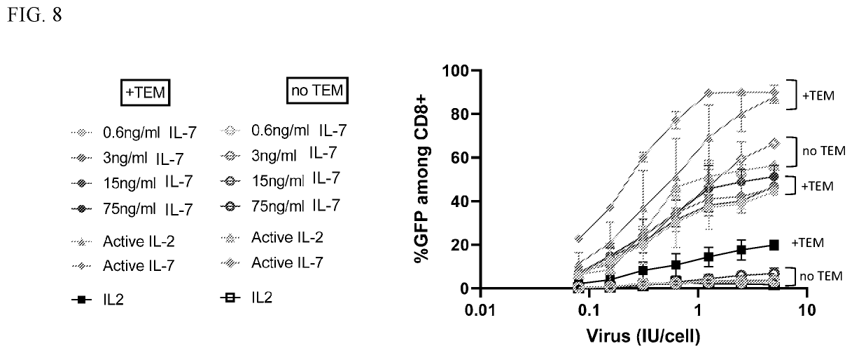

Resting pan-T cells from a donor were pre-treated with 100 U/mL of IL-2 or a range of IL-7 (0.6-75 ng/mL) for three days. Pre-activated pan-T cells were included as controls (designated “active”). After three days, cells were incubated for 2 hours with 30 μM of TEM and transduced by spinfection for 2 hours with a lentiviral vector carrying an eGFP reporter gene and pseudotyped with a CD8-retargeted NiV-G protein and NiV-F as described in Example 1 at a range of 0.08-5 IU/cell. Cells were incubated with TEM for a total of 4 hrs. Cells not treated with TEM were included as controls. Cells were bead-activated a day later and 6 days thereafter examined by flow for % GFP. The combined treatment with TEM and IL-7 increased transduction efficiency. Varying the pre-treatment IL-7 dose from 0.6-75 ng/mL did not lead to dose-dependent differences in transduction efficiency (FIG. 8).

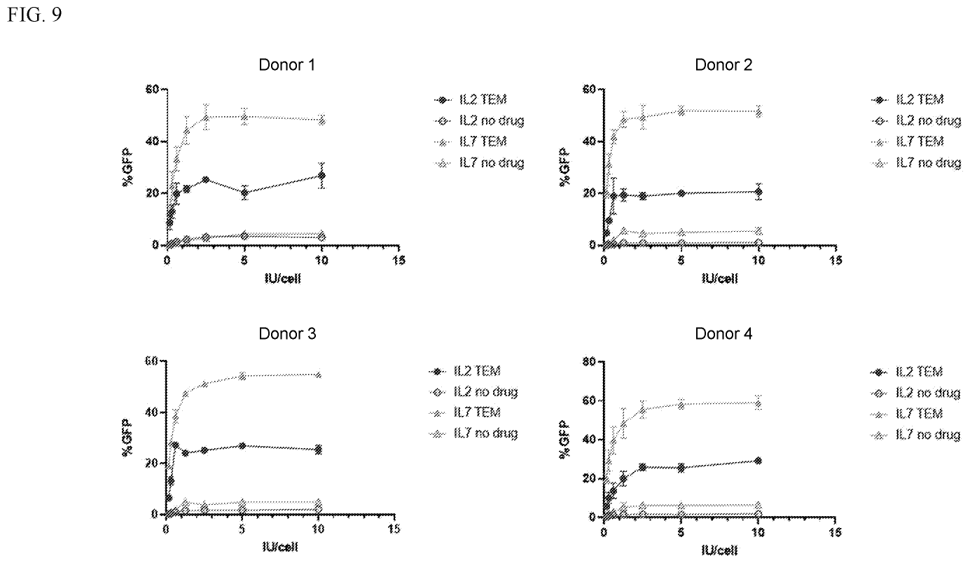

In a separate experiment, resting pan-T cells from 4 donors were pre-treated with 15 ng/mL IL-7 or 100 U/mL of IL-2 for three days. After three days, cells were incubated for 2 hours with 30 μM of TEM and then transduced by spinfection for 2 hours with a lentiviral vector carrying an eGFP reporter gene and pseudotyped with a CD8-retargeted NiV-G protein and NiV-F as described in Example 1 at range of 0.16-10 IU/cell. Cells were incubated with TEM for a total of 4 hrs. Cells not treated with TEM were included as controls. Cells were bead-activated a day later and 6 days thereafter examined by flow for % GFP. IL-7 and TEM synergistically improved transduction efficiencies and GFP expression up to 11 times compared to cells incubated with either components alone (FIG. 9).

### B. Effect of IL-2 or IL-7 and TEM on IFITM1 and SAMHD1 Expression

Since IL-7 and temsirolimus (TEM) synergistically increased transduction efficiencies of resting pan-T cells, protein expression of pSAMHD1 and IFITM1 expression was determined.

Resting pan-T cells from 1 donor were pre-treated with 100 U/mL of IL-2 or a range of IL-7 (0.6-75 ng/mL) for three days. Pre-activated pan-T cells were included as controls. After three days, cells were incubated for 2 hours with 30 μM of TEM and then transduced by spinfection for 2 hours with a lentiviral vector carrying an eGFP reporter gene and pseudotyped with a CD8-retargeted NiV-G protein and NiV-F as described in Example 1 at range of 0.08-5 IU/cell. Cells were incubated with TEM for a total of 4 hrs. Cells were bead-activated a day later. Cells were harvested at various timepoints for protein expression (day of thaw (d-3), day of transduction before adding drug (d0), 2 hours after drug treatment (d0+2h), 4 hours after drug treatment (0+4h), and 1 day after transduction. IFITM1 and pSAMHD1 protein levels were assessed by Western Blot.

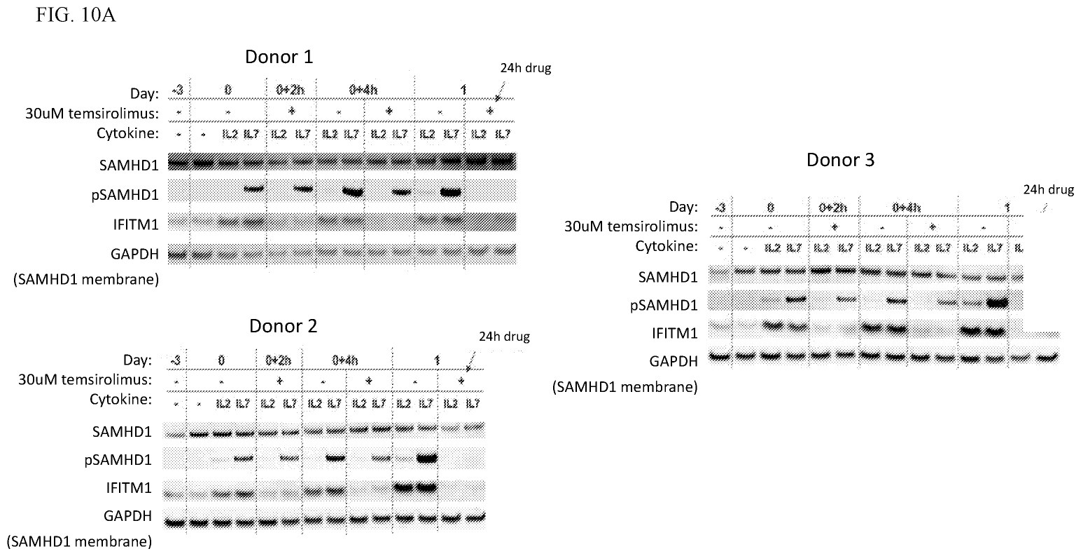

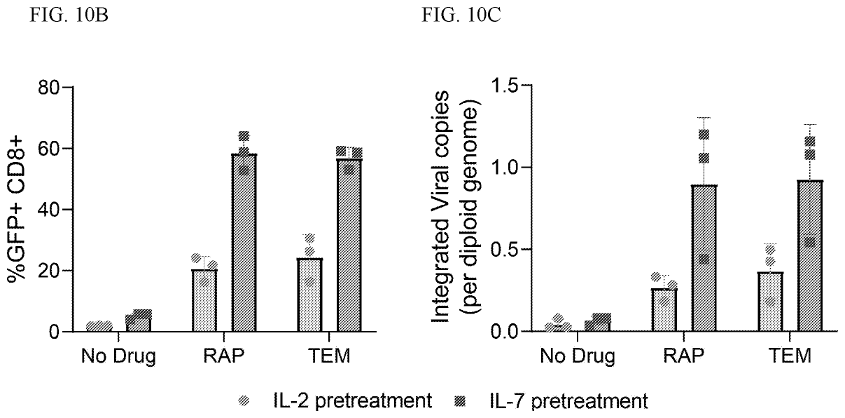

Western blot analysis of IFITM1 and pSAMHD1 protein levels as shown in FIG. 10A highlighted a specific effect of IL-7 and TEM combination on relevant restriction factors. A pronounced loss of IFITM1 protein expression after 2 hours of TEM treatment and an increase in pSAMHD1 levels after 3 days of IL-7 pre-treatment was observed across multiple donors (FIG. 10A). FIG. 10B depicts increased CAR expression in cells treated with RAP+IL-7 and TEM+IL-7. Increased transduction efficiency was observed in T cells treated with RAP+IL-7 and TEM+IL-7 (FIG. 10C).

### Example 5 Assessment of Transduction with Rapamycin Analogs

The effects of rapamycin (RAP), everolimus (EVO), or temsirolimus (TEM), in combination with IL-2 or IL-7 on transduction efficiency was assessed.

Resting pan-T cells from 3 donors were pre-treated with 15 ng/mL IL-7 or 100 U/mL of IL-2 for 3 days. After three days, cells were incubated for 2 hours with various doses of RAP, EVO, or TEM, and transduced by spinfection with 5 IU/cell of a lentiviral vector carrying a CD19 CAR gene and pseudotyped with a CD8-retargeted NiV-G protein and NiV-F as described in Example 1 for 2 hrs. Cells were incubated with RAP, EVO or TEM for a total of 4 hrs. Cells not treated with RAP, EVO or TEM were included as controls. Cells were bead-activated a day later and 6 days thereafter examined by flow for % CAR and viral copy number (VCN) was also measured.

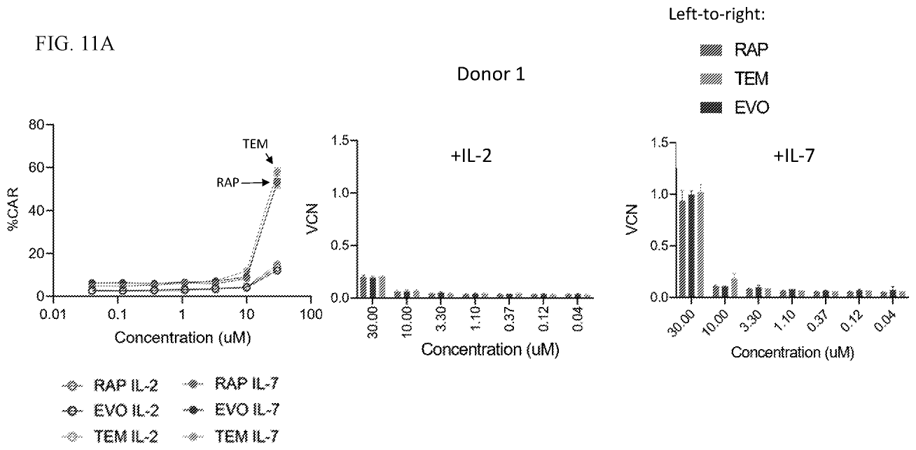

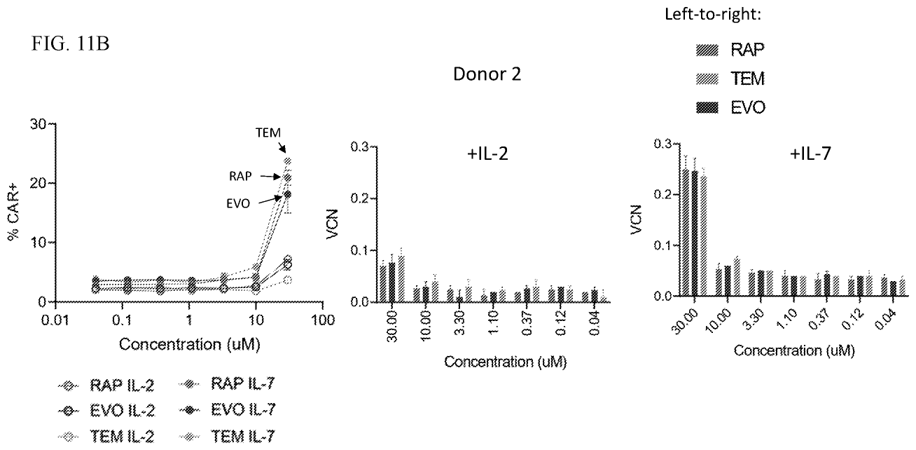

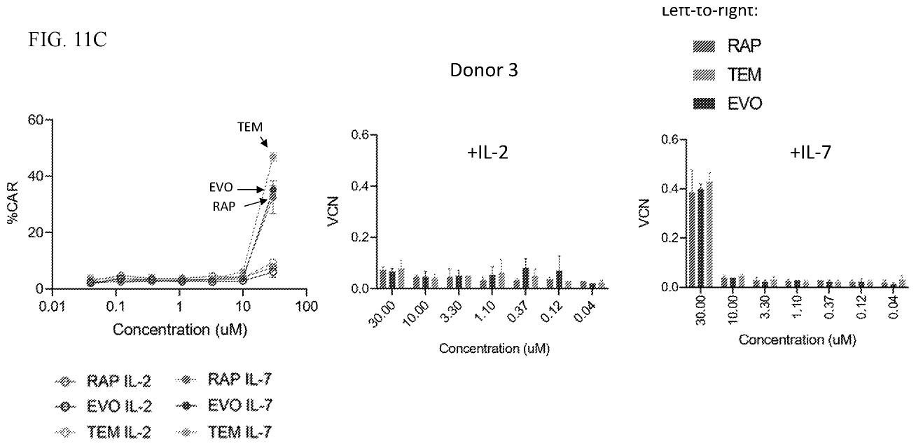

Enhanced transduction with rapamycin analogs (rapalogs) occurred at concentrations 10 μM or greater. 30 μM of TEM in combination with IL-7 pre-treatment led to the greatest dose-dependent increase in transduction efficiencies in resting T cells in all donors (FIGS. 11A-11C).

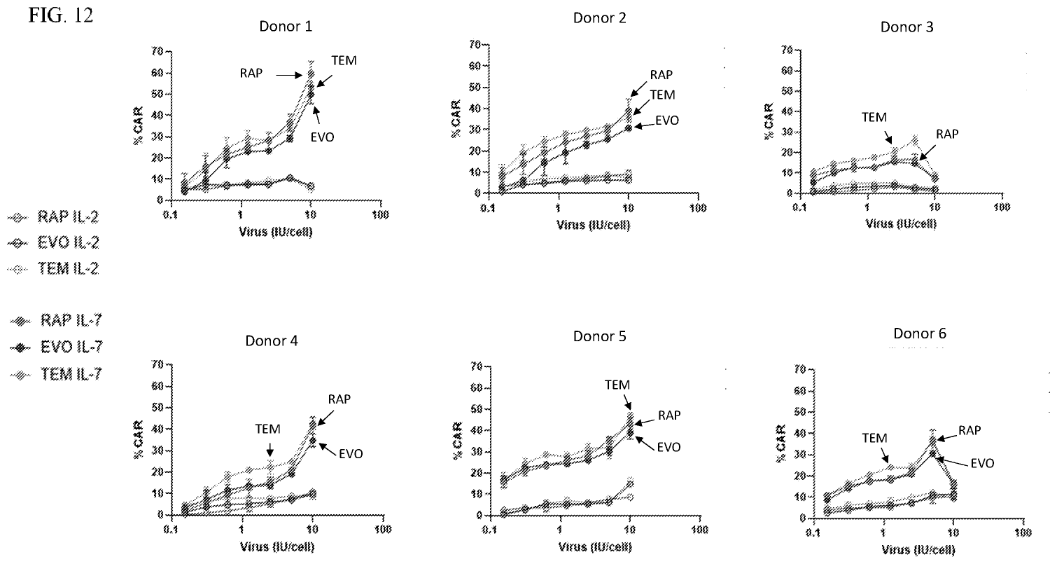

In a separate experiment, the effects of RAP, EVO, or TEM in combination with IL-2 or IL-7 on transduction efficiency was assessed. Briefly, resting pan-T cells from 6 donors were pre-treated with 15 ng/mL IL-7 or 100 U/mL of IL-2 for 3 days. After three days, cells were incubated for 2 hours with 30 μM of RAP, TEM or EVO and transduced by spinfection with a lentiviral vector carrying a CD19 CAR gene and pseudotyped with a CD8-retargeted NiV-G protein and NiV-F as described in Example 1 at a range of 0.16-10 IU/cell for 2 hrs. Cells were incubated with RAP, EVO or TEM for a total of 4 hrs. Cells not treated with RAP, EVO or TEM were included as controls. Cells were bead-activated a day later and 6 days thereafter examined by flow for % CAR. CAR expression was up to five times higher with IL-7+ TEM compared to IL-2+ TEM treatment (FIG. 12). Increased transfection efficiency also was observed with other rapalogs.

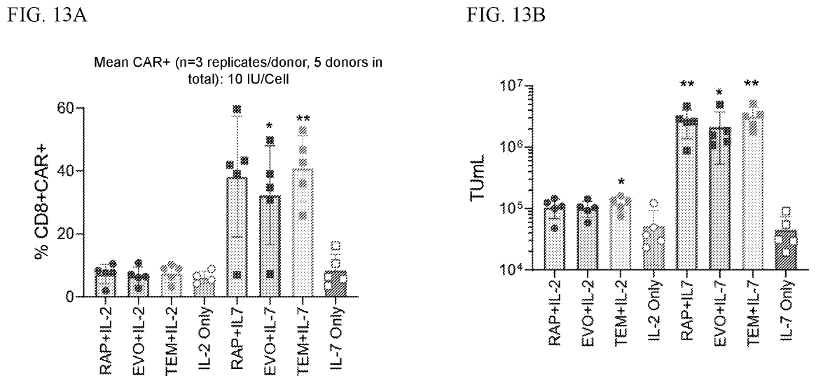

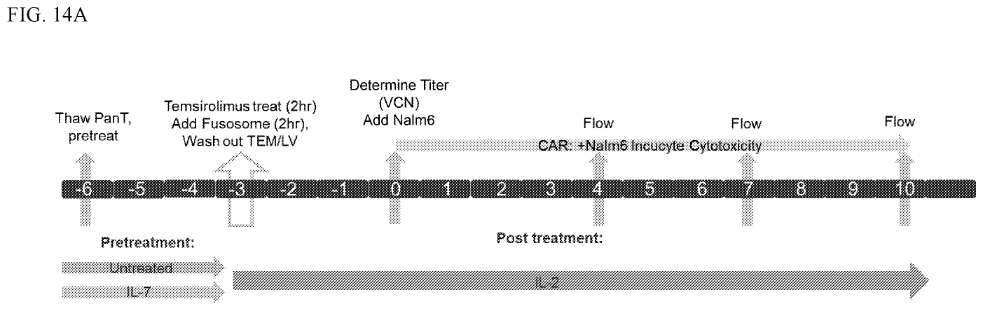

In a further experiment, the effects of 30 μM TEM on % CAR expression, % CD8+CAR+ cells, and TU/mL transduction of resting T cells was assessed. Briefly, cells were treated for 72 hours with IL-2 or IL-7 and replated in a 96 well plate at 50,000 cells per well. Cells were incubated for 2 hours with 30 μM of RAP, TEM or EVO and transduced by spinfection with a range of a lentiviral vector carrying a CD19 CAR gene and pseudotyped with a CD8-retargeted NiV-G protein and NiV-F as described in Example 1 (max dose of 10 IU/cell) for 2 hrs. Rapamycin analogs+IL-7 increased CAR expression compared to rapamycin analogs+IL-2. Rapamycin analogs also increased the percentage of CD8+ CAR expressing cells (FIG. 13A) and the number of functional viral particles (FIG. 13B).

These data show that transduction potency and efficiency is significantly increased in resting T cells following IL-7 and TEM (or other rapalog) treatment in a transgene-independent manner. This increase in potency and efficiency is consistent with the inactivation or loss of two critical restriction factors, SAMDH1 and IFITM1, as demonstrated by western blot analysis. Collectively, these results suggest a strategy toward a combination in vivo vector-based therapy that is supplemented with pharmaceutical agents to improve vector potency.

### Example 6 Assessment of Cytotoxic Activity of CD19-CAR Fusosome Transduced Cells

Cytotoxic activity of CD8+ cells transduced with CD8-retargeting lentiviral vectors (fusosomes) carrying a CD19 CAR transgene was assessed.

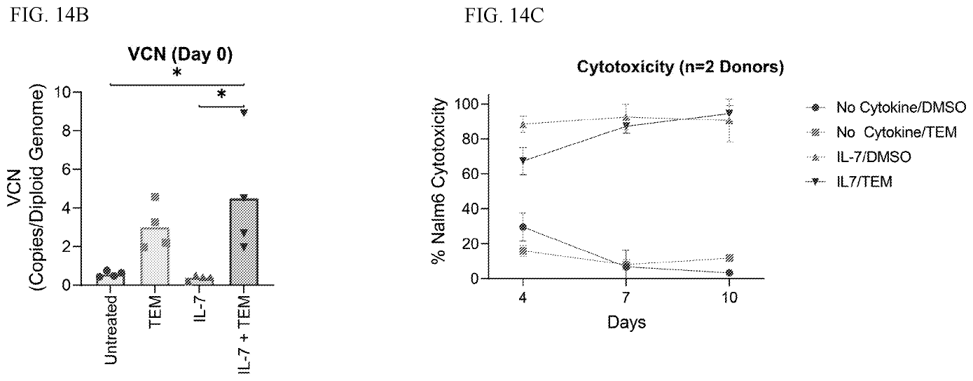

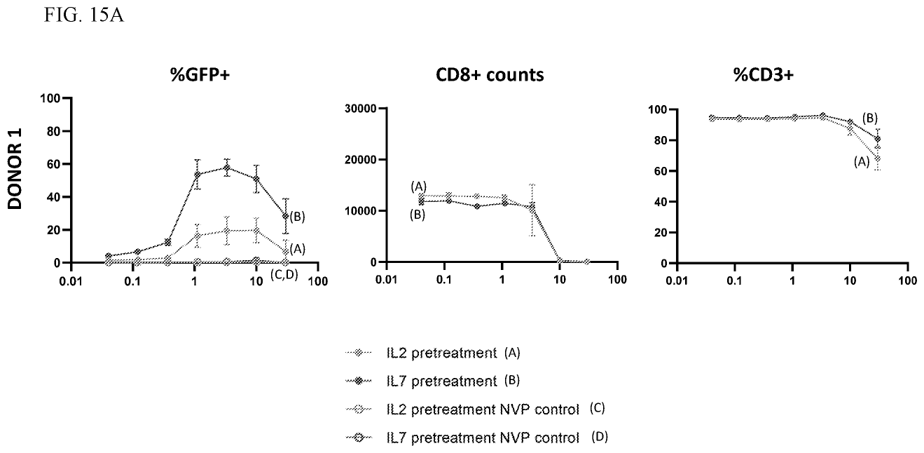

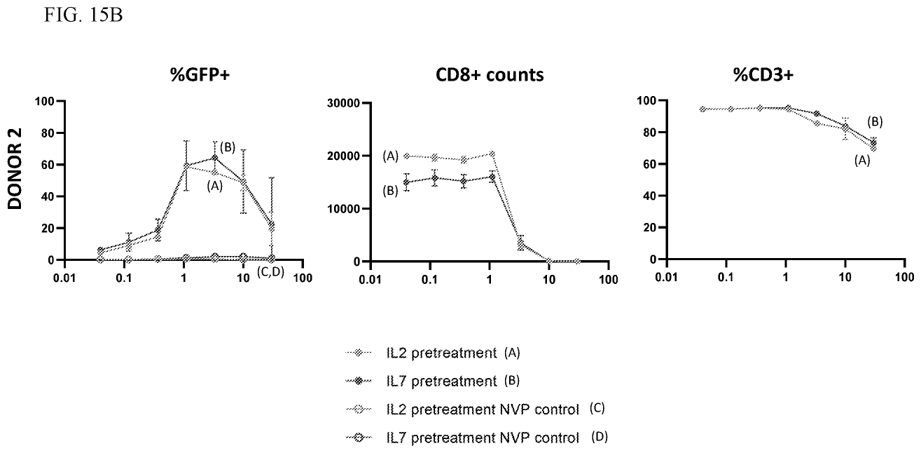

The cytotoxic assay was performed according to FIG. 14A. Briefly, resting pan-T cells from 2 donors were pre-treated with 15 ng/mL IL-7 or were left untreated for 3 days. After three days, cells were incubated for 2 hours with 30 μM TEM and transduced by spinfection with a lentiviral vector carrying a CD19 CAR gene and pseudotyped with a CD8-retargeted NiV-G protein and NiV-F as described in Example 1 at a range of 0.16-10 IU/cell for 2 hrs. Cells were concurrently treated with IL-2 on day 3 before the experiment. Transduction efficiency was assessed on Day 0 of the experiment and Nalm6 cells were added. Nalm6 killing was determined by flow cytometry on Day 4, 7 and 10 of the experiment. Cytotoxicity is presented as Nalm6 loss related to untransduced controls.

Increased transduction efficiency was observed in T cells treated with TEM and IL-7+ TEM (FIG. 14B). Increased cytotoxicity was observed in T cells treated with IL-7 and IL-7+ TEM (FIG. 14C).

### Example 7 Transduction Efficiency with Antifungal Agent Amphotericin B

The effects of temsirolimus (TEM) and antifungal agent Amphotericin B (AmphoB), in combination with IL-2 or IL-7 on transduction efficiency was assessed.

Resting pan-T cells from two donors were pre-treated with 15 ng/mL IL-7 or 100 U/mL of IL-2 for three days. After three days, cells were incubated for two hours with various doses of TEM or AmphoB.

Cells were then transduced by spinfection with 5 IU/cell of a lentiviral vector carrying a GFP gene and pseudotyped with a CD8-retargeted NiV-G protein and NiV-F as described in Example 1 for two hours before cells were washed and placed into cytokine containing media (e.g., IL-7 or IL-2) for up to seven days. Cells not treated were included as controls.

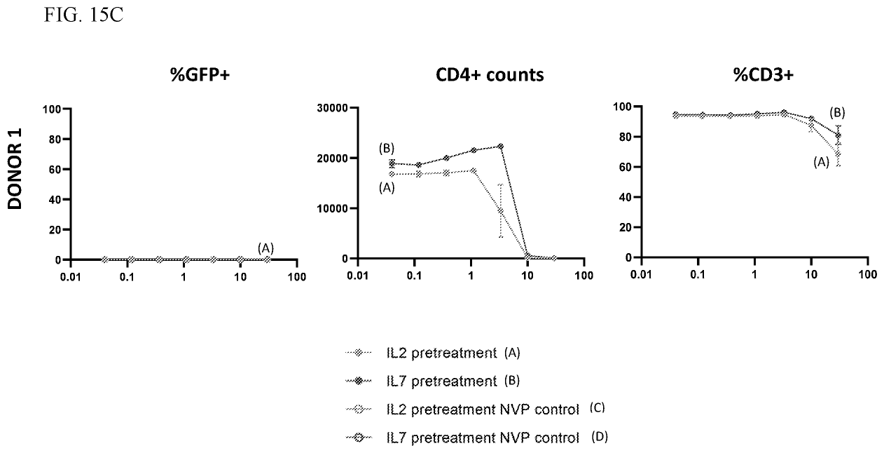

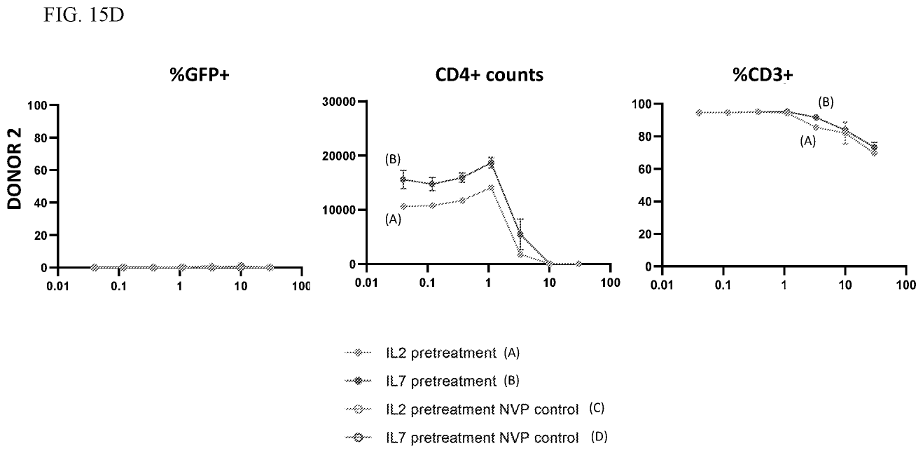

Results for experiments conducted with cells from two donors using AmphoB are shown in FIG. 15 (FIG. 15A and FIG. 15B represent Donor 1, and FIG. 15C and FIG. 15D represent Donor 2). As shown in percentage of cells expressing the transgene GFP, AmphoB increased transduction efficiency at the 1 μM dose for both CD4 and CD8 cells, where IL-7 pretreatment was also seen to positively impact transduction for several donors. CD8 and CD4 cell number was observed to be slightly reduced at the higher doses tested, depending on donor and cytokine pretreatment (FIG. 15A and FIG. 15C, donor 1; FIG. 15B and FIG. 15D, donor 2). Additionally, CD3 number was assessed as an approximate of T cell health and was observed to gradually decrease at the highest dose of AmphoB for both donors. CD3 number was observed to be consistent at those AmphoB concentrations associated with the highest GFP expression.

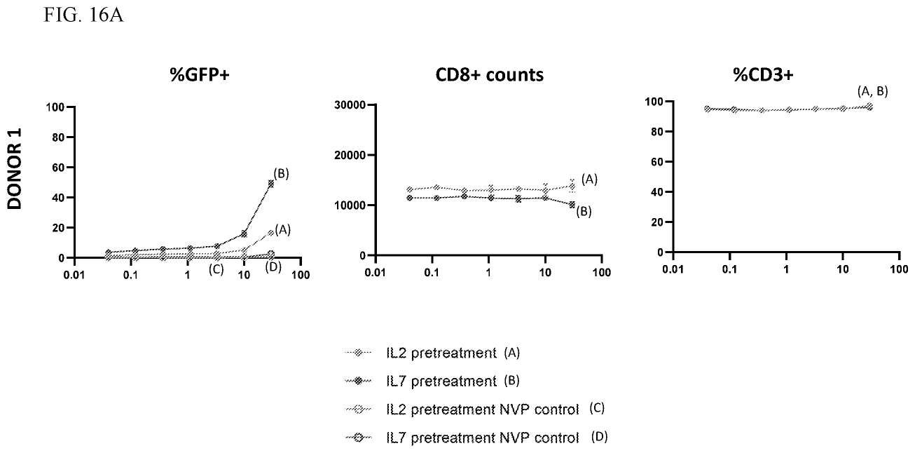

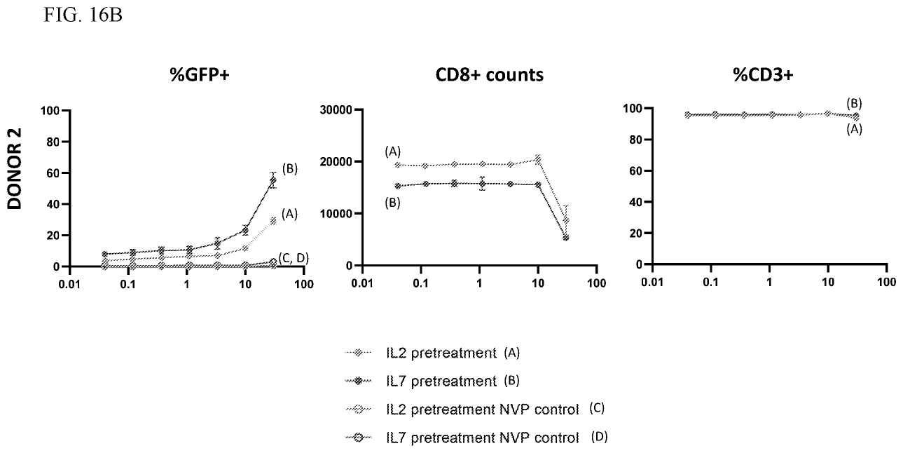

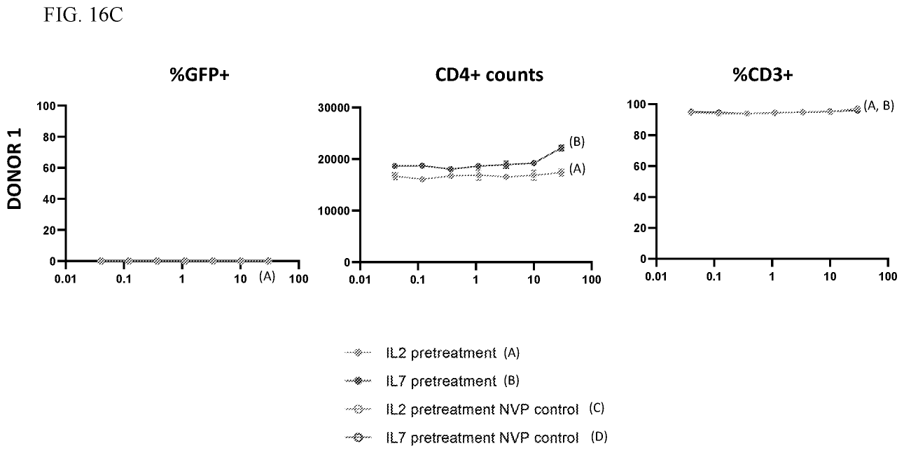

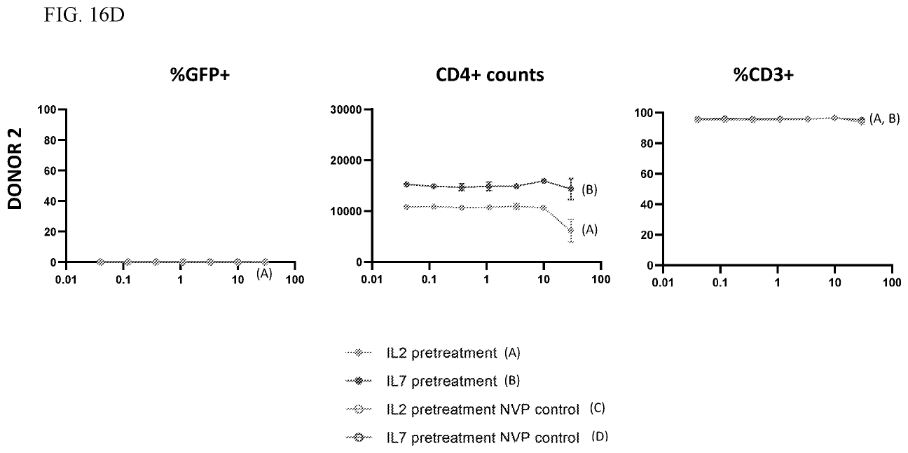

Results for experiments conducted with cells from two donors using TEM are shown in FIG. 16 (FIG. 16A and FIG. 16B represent Donor 1, and FIG. 16C and FIG. 16D represent Donor 2). As shown in percentage of cells expressing the transgene GFP, TEM showed an increase in transduction efficiency at a higher dose across donors as compared to AmphoB, 30 μM was observed to enhance transduction of the GFP transgene in both CD4 and CD8 cells. IL7 pretreatment also increased transduction efficiency. CD8 and CD4 T-cells were reduced at the 30 μM dose of TEM in Donor 2, regardless of cytokine pretreatment (FIG. 16B and FIG. 16D). CD3 number remained generally consistent across TEM dosing for both donors.

Enhanced transduction of CD4 and CD8 T cells with the antifungal agent (AmphoB) occurred at a broader dose range and lower concentrations lower than that seen with TEM. For example, gain in transduction efficiency is seen for AmphoB treated cells at concentrations as low as 0.1 μM (FIG. 15A and FIG. 15B).

These data show that transduction efficiency is increased in resting T cells following treatment with an antifungal agent, AmphoB. This increase in potency and efficiency is consistent with the inactivation or loss of critical restriction factors IFITM1, as demonstrated by western blot analysis (Data not shown).

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

### VIII. SEQUENCES

| SEQ | | |
|---|---|---|
| ID | | |
| NO | SEQUENCE | DESCRIPTION |
| 1 | MATQEVRLKCLLCGIIVLVLSLEGLGILHYEKLSKIGLVKGITRKY | Hendra F |
| | KIKSNPLTKDIVIKMIPNVSNVSKCTGTVMENYKSRLTGILSPIKG | |
| | AIELYNNNTHDLVGDVKLAGVVMAGIAIGIATAAQITAGVALYE | |
| | AMKNADNINKLKSSIESTNEAVVKLQETAEKTVYVLTALQDYIN | |
| | TNLVPTIDQISCKQTELALDLALSKYLSDLLFVFGPNLQDPVSNSM | |
| | TIQAISQAFGGNYETLLRTLGYATEDFDDLLESDSIAGQIVYVDLS | |
| | SYYIIVRVYFPILTEIQQAYVQELLPVSFNNDNSEWISIVPNFVLIRN | |
| | TLISNIEVKYCLITKKSVICNQDYATPMTASVRECLTGSTDKCPRE | |
| | LVVSSHVPRFALSGGVLFANCISVTCQCQTTGRAISQSGEQTLLMI | |
| | DNTTCTTVVLGNIIISLGKYLGSINYNSESIAVGPPVYTDKVDISSQ | |
| | ISSMNQSLQQSKDYIKEAQKILDTVNPSLISMLSMIILYVLSIAALCI | |
| | GLITFISFVIVEKKRGNYSRLDDRQVRPVSNGDLYYIGT | |
| 2 | MVVILDKRCYCNLLILILMISECSVGILHYEKLSKIGLVKGVTRKY | Nipah F0 |
| | KIKSNPLTKDIVIKMIPNVSNMSQCTGSVMENYKTRLNGILTPIKG | |
| | ALEIYKNNTHDLVGDVRLAGVIMAGVAIGIATAAQITAGVALYE | |
| | AMKNADNINKLKSSIESTNEAVVKLQETAEKTVYVLTALQDYIN | |
| | TNLVPTIDKISCKQTELSLDLALSKYLSDLLFVFGPNLQDPVSNSM | |
| | TIQAISQAFGGNYETLLRTLGYATEDFDDLLESDSITGQIIYVDLSS | |
| | YYIIVRVYFPILTEIQQAYIQELLPVSFNNDNSEWISIVPNFILVRNT | |
| | LISNIEIGFCLITKRSVICNQDYATPMTNNMRECLTGSTEKCPRELV | |
| | VSSHVPRFALSNGVLFANCISVTCQCQTTGRAISQSGEQTLLMIDN | |
| | TTCPTAVLGNVIISLGKYLGSVNYNSEGIAIGPPVFTDKVDISSQIS | |
| | SMNQSLQQSKDYIKEAQRLLDTVNPSLISMLSMIILYVLSIASLCIG | |
| | LITFISFIIVEKKRNTYSRLEDRRVRPTSSGDLYYIGT | |
| 3 | MSNKRTTVLIIISYTLFYLNNAAIVGFDFDKLNKIGVVQGRVLNY | CedV F |
| | KIKGDPMTKDLVLKFIPNIVNITECVREPLSRYNETVRRLLLPIHN | |
| | MLGLYLNNTNAKMTGLMIAGVIMGGIAIGIATAAQITAGFALYE | |
| | AKKNTENIQKLTDSIMKTQDSIDKLTDSVGTSILILNKLQTYINNQ | |
| | LVPNLELLSCRQNKIEFDLMLTKYLVDLMTVIGPNINNPVNKDMT | |
| | IQSLSLLFDGNYDIMMSELGYTPQDFLDLIESKSITGQIIYVDMENL | |
| | YVVIRTYLPTLIEVPDAQIYEFNKITMSSNGGEYLSTIPNFILIRGNY | |
| | MSNIDVATCYMTKASVICNQDYSLPMSQNLRSCYQGETEYCPVE | |
| | AVIASHSPRFALTNGVIFANCINTICRCQDNGKTITQNINQFVSMID | |
| | NSTCNDVMVDKFTIKVGKYMGRKDINNINIQIGPQIIIDKVDLSNE | |
| | INKMNQSLKDSIFYLREAKRILDSVNISLISPSVQLFLIIISVLSFIILLI | |
| | IIVYLYCKSKHSYKYNKFIDDPDYYNDYKRERINGKASKSNNIYY | |
| | VGD | |
| 4 | MALNKNMFSSLFLGYLLVYATTVQSSIHYDSLSKVGVIKGLTYN | Mojiang F |
| | YKIKGSPSTKLMVVKLIPNIDSVKNCTQKQYDEYKNLVRKALEPV | |
| | KMAIDTMLNNVKSGNNKYRFAGAIMAGVALGVATAATVTAGIA | |
| | LHRSNENAQAIANMKSAIQNTNEAVKQLQLANKQTLAVIDTIRG | |
| | EINNNIIPVINQLSCDTIGLSVGIRLTQYYSEIITAFGPALQNPVNTRI | |
| | TIQAISSVENGNFDELLKIMGYTSGDLYEILHSELIRGNIIDVDVDA | |
| | GYIALEIEFPNLTLVPNAVVQELMPISYNIDGDEWVTLVPRFVLTR | |
| | TTLLSNIDTSRCTITDSSVICDNDYALPMSHELIGCLQGDTSKCAR | |
| | EKVVSSYVPKFALSDGLVYANCLNTICRCMDTDTPISQSLGATVS | |
| | LLDNKRCSVYQVGDVLISVGSYLGDGEYNADNVELGPPIVIDKID | |
| | IGNQLAGINQTLQEAEDYIEKSEEFLKGVNPSIITLGSMVVLYIFMI | |
| | LIAIVSVIALVLSIKLTVKGNVVRQQFTYTQHVPSMENINYVSH | |
| 5 | MKKKTDNPTISKRGHNHSRGIKSRALLRETDNYSNGLIVENLVRN | Bat PV F |
| | CHHPSKNNLNYTKTQKRDSTIPYRVEERKGHYPKIKHLIDKSYKH | |
| | IKRGKRRNGHNGNIITIILLLILILKTQMSEGAIHYETLSKIGLIKGIT | |
| | REYKVKGTPSSKDIVIKLIPNVTGLNKCTNISMENYKEQLDKILIPI | |
| | NNIIELYANSTKSAPGNARFAGVIIAGVALGVAAAAQITAGIALHE | |
| | ARQNAERINLLKDSISATNNAVAELQEATGGIVNVITGMQDYINT | |
| | NLVPQIDKLQCSQIKTALDISLSQYYSEILTVFGPNLQNPVTTSMSI | |
| | QAISQSFGGNIDLLLNLLGYTANDLLDLLESKSITGQITYINLEHYF | |
| | MVIRVYYPIMTTISNAYVQELIKISFNVDGSEWVSLVPSYILIRNSY | |
| | LSNIDISECLITKNSVICRHDFAMPMSYTLKECLTGDTEKCPREAV | |
| | VTSYVPRFAISGGVIYANCLSTTCQCYQTGKVIAQDGSQTLMMID | |
| | NQTCSIVRIEEILISTGKYLGSQEYNTMHVSVGNPVFTDKLDITSQI | |
| | SNINQSIEQSKFYLDKSKAILDKINLNLIGSVPISILFIIAILSLILSIITF | |
| | VIVMIIVRRYNKYTPLINSDPSSRRSTIQDVYIIPNPGEHSIRSAARSI | |
| | DRDRD | |
| 6 | ILHYEKLSKIGLVKGITRKYKIKSNPLTKDIVIKMIPNVSNVSKCTG | Hendra virus F |
| | TVMENYKSRLTGILSPIKGAIELYNNNTHDLVGDVKLAGVVMAG | Protein, Without |
| | IAIGIATAAQITAGVALYEAMKNADNINKLKSSIESTNEAVVKLQE | signal sequence |
| | TAEKTVYVLTALQDYINTNLVPTIDQISCKQTELALDLALSKYLS | |
| | DLLFVFGPNLQDPVSNSMTIQAISQAFGGNYETLLRTLGYATEDF | |
| | DDLLESDSIAGQIVYVDLSSYYIIVRVYFPILTEIQQAYVQELLPVS | |
| | FNNDNSEWISIVPNFVLIRNTLISNIEVKYCLITKKSVICNQDYATP | |
| | MTASVRECLTGSTDKCPRELVVSSHVPRFALSGGVLFANCISVTC | |
| | QCQTTGRAISQSGEQTLLMIDNTTCTTVVLGNIIISLGKYLGSINYN | |
| | SESIAVGPPVYTDKVDISSQISSMNQSLQQSKDYIKEAQKILDTVN | |
| | PSLISMLSMIILYVLSIAALCIGLITFISFVIVEKKRGNYSRLDDRQV | |
| | RPVSNGDLYYIGT | |
| 7 | ILHYEKLSKIGLVKGVTRKYKIKSNPLTKDIVIKMIPNVSNMSQCT | Nipah virus F |
| | GSVMENYKTRLNGILTPIKGALEIYKNNTHDLVGDVRLAGVIMA | Protein, without |
| | GVAIGIATAAQITAGVALYEAMKNADNINKLKSSIESTNEAVVKL | signal sequence |
| | QETAEKTVYVLTALQDYINTNLVPTIDKISCKQTELSLDLALSKYL | |
| | SDLLFVFGPNLQDPVSNSMTIQAISQAFGGNYETLLRTLGYATED | |
| | FDDLLESDSITGQIIYVDLSSYYIIVRVYFPILTEIQQAYIQELLPVSF | |
| | NNDNSEWISIVPNFILVRNTLISNIEIGFCLITKRSVICNQDYATPMT | |
| | NNMRECLTGSTEKCPRELVVSSHVPRFALSNGVLFANCISVTCQC | |
| | QTTGRAISQSGEQTLLMIDNTTCPTAVLGNVIISLGKYLGSVNYNS | |
| | EGIAIGPPVFTDKVDISSQISSMNQSLQQSKDYIKEAQRLLDTVNPS | |
| | LISMLSMIILYVLSIASLCIGLITFISFIIVEKKRNTYSRLEDRRVRPT | |
| | SSGDLYYIGT | |
| 8 | TVLIIISYTLFYLNNAAIVGFDFDKLNKIGVVQGRVLNYKIKGDPM | Cedar Virus F |
| | TKDLVLKFIPNIVNITECVREPLSRYNETVRRLLLPIHNMLGLYLN | Protein, without |
| | NTNAKMTGLMIAGVIMGGIAIGIATAAQITAGFALYEAKKNTENI | signal sequence |
| | QKLTDSIMKTQDSIDKLTDSVGTSILILNKLQTYINNQLVPNLELLS | |
| | CRQNKIEFDLMLTKYLVDLMTVIGPNINNPVNKDMTIQSLSLLFD | |
| | GNYDIMMSELGYTPQDFLDLIESKSITGQIIYVDMENLYVVIRTYL | |
| | PTLIEVPDAQIYEFNKITMSSNGGEYLSTIPNFILIRGNYMSNIDVA | |
| | TCYMTKASVICNQDYSLPMSQNLRSCYQGETEYCPVEAVIASHSP | |
| | RFALTNGVIFANCINTICRCQDNGKTITQNINQFVSMIDNSTCNDV | |
| | MVDKFTIKVGKYMGRKDINNINIQIGPQIIIDKVDLSNEINKMNQS | |
| | LKDSIFYLREAKRILDSVNISLISPSVQLFLIIISVLSFIILLIIIVYLYC | |
| | KSKHSYKYNKFIDDPDYYNDYKRERINGKASKSNNIYYVGD | |
| 9 | IHYDSLSKVGVIKGLTYNYKIKGSPSTKLMVVKLIPNIDSVKNCTQ | Mojiang virus, |
| | KQYDEYKNLVRKALEPVKMAIDTMLNNVKSGNNKYRFAGAIMA | Tongguan 1 F |
| | GVALGVATAATVTAGIALHRSNENAQAIANMKSAIQNTNEAVKQ | Protein, without |
| | LQLANKQTLAVIDTIRGEINNNIIPVINQLSCDTIGLSVGIRLTQYYS | signal sequence |
| | EIITAFGPALQNPVNTRITIQAISSVENGNFDELLKIMGYTSGDLYEI | |
| | LHSELIRGNIIDVDVDAGYIALEIEFPNLTLVPNAVVQELMPISYNI | |
| | DGDEWVTLVPRFVLTRTTLLSNIDTSRCTITDSSVICDNDYALPMS | |
| | HELIGCLQGDTSKCAREKVVSSYVPKFALSDGLVYANCLNTICRC | |
| | MDTDTPISQSLGATVSLLDNKRCSVYQVGDVLISVGSYLGDGEY | |
| | NADNVELGPPIVIDKIDIGNQLAGINQTLQEAEDYIEKSEEFLKGV | |
| | NPSIITLGSMVVLYIFMILIAIVSVIALVLSIKLTVKGNVVRQQFTY | |
| | TQHVPSMENINYVSH | |
| 10 | SRALLRETDNYSNGLIVENLVRNCHHPSKNNLNYTKTQKRDSTIP | Bat Paramyxovirus |
| | YRVEERKGHYPKIKHLIDKSYKHIKRGKRRNGHNGNIITIILLLILI | F Protein, without |
| | LKTQMSEGAIHYETLSKIGLIKGITREYKVKGTPSSKDIVIKLIPNV | signal sequence |
| | TGLNKCTNISMENYKEQLDKILIPINNIIELYANSTKSAPGNARFA | |
| | GVIIAGVALGVAAAAQITAGIALHEARQNAERINLLKDSISATNN | |
| | AVAELQEATGGIVNVITGMQDYINTNLVPQIDKLQCSQIKTALDIS | |
| | LSQYYSEILTVFGPNLQNPVTTSMSIQAISQSFGGNIDLLLNLLGYT | |
| | ANDLLDLLESKSITGQITYINLEHYFMVIRVYYPIMTTISNAYVQE | |
| | LIKISFNVDGSEWVSLVPSYILIRNSYLSNIDISECLITKNSVICRHD | |
| | FAMPMSYTLKECLTGDTEKCPREAVVTSYVPRFAISGGVIYANCL | |
| | STTCQCYQTGKVIAQDGSQTLMMIDNQTCSIVRIEEILISTGKYLG | |
| | SQEYNTMHVSVGNPVFTDKLDITSQISNINQSIEQSKFYLDKSKAI | |
| | LDKINLNLIGSVPISILFIIAILSLILSIITFVIVMIIVRRYNKYTPLINS | |
| | DPSSRRSTIQDVYIIPNPGEHSIRSAARSIDRDRD | |
| 11 | MVVILDKRCY CNLLILILMI SECSVGILHY EKLSKIGLVK | Truncated NiV |
| | GVTRKYKIKS NPLTKDIVIK MIPNVSNMSQ CTGSVMENYK | fusion glycoprotein |
| | TRLNGILTPI KGALEIYKNN THDLVGDVRL AGVIMAGVAI | (FcDelta22) at |
| | GIATAAQITA GVALYEAMKN ADNINKLKSS IESTNEAVVK | cytoplasmic tail |
| | LQETAEKTVY VLTALQDYIN TNLVPTIDKI SCKQTELSLD | (with signal |
| | LALSKYLSDL LFVFGPNLQD PVSNSMTIQA ISQAFGGNYE | sequence) |
| | TLLRTLGYAT EDFDDLLESD SITGQIIYVD LSSYYIIVRV | |
| | YFPILTEIQQ AYIQELLPVS FNNDNSEWIS IVPNFILVRN | |
| | TLISNIEIGF CLITKRSVIC NQDYATPMTN NMRECLTGST | |
| | EKCPRELVVS SHVPRFALSN GVLFANCISV TCQCQTTGRA | |
| | ISQSGEQTLL MIDNTTCPTA VLGNVIISLG KYLGSVNYNS | |
| | EGIAIGPPVF TDKVDISSQI SSMNQSLQQS KDYIKEAQRL | |
| | LDTVNPSLIS MLSMIILYVL SIASLCIGLI TFISFIIVEK KRNT | |
| 12 | ILHY EKLSKIGLVK GVTRKYKIKS NPLTKDIVIK MIPNVSNMSQ | Truncated mature |
| | CTGSVMENYK TRLNGILTPI KGALEIYKNN THDLVGDVRL | NiV fusion |
| | AGVIMAGVAI GIATAAQITA GVALYEAMKN ADNINKLKSS | glycoprotein |
| | IESTNEAVVK LQETAEKTVY VLTALQDYIN TNLVPTIDKI | (FcDelta22) at |
| | SCKQTELSLD LALSKYLSDL LFVFGPNLQD PVSNSMTIQA | cytoplasmic tail |
| | ISQAFGGNYE TLLRTLGYAT EDFDDLLESD SITGQIIYVD | |
| | LSSYYIIVRV YFPILTEIQQ AYIQELLPVS FNNDNSEWIS | |
| | IVPNFILVRN TLISNIEIGF CLITKRSVIC NQDYATPMTN | |
| | NMRECLTGST EKCPRELVVS SHVPRFALSN GVLFANCISV | |
| | TCQCQTTGRA ISQSGEQTLL MIDNTTCPTA VLGNVIISLG | |
| | KYLGSVNYNS EGIAIGPPVF TDKVDISSQI SSMNQSLQQS | |
| | KDYIKEAQRL LDTVNPSLIS MLSMIILYVL SIASLCIGLI | |
| | TFISFIIVEK KRNT | |
| 13 | MMADSKLVSLNNNLSGKIKDQGKVIKNYYGTMDIKKINDGLLDS | Hendra virus G |
| | KILGAFNTVIALLGSIIIIVMNIMIIQNYTRTTDNQALIKESLQSVQQ | protein Uniprot |
| | QIKALTDKIGTEIGPKVSLIDTSSTITIPANIGLLGSKISQSTSSINEN | O89343 |
| | VNDKCKFTLPPLKIHECNISCPNPLPFREYRPISQGVSDLVGLPNQI | |
| | CLQKTTSTILKPRLISYTLPINTREGVCITDPLLAVDNGFFAYSHLE | |
| | KIGSCTRGIAKQRIIGVGEVLDRGDKVPSMFMTNVWTPPNPSTIH | |
| | HCSSTYHEDFYYTLCAVSHVGDPILNSTSWTESLSLIRLAVRPKSD | |
| | SGDYNQKYIAITKVERGKYDKVMPYGPSGIKQGDTLYFPAVGFL | |
| | PRTEFQYNDSNCPIIHCKYSKAENCRLSMGVNSKSHYILRSGLLK | |
| | YNLSLGGDIILQFIEIADNRLTIGSPSKIYNSLGQPVFYQASYSWDT | |
| | MIKLGDVDTVDPLRVQWRNNSVISRPGQSQCPRFNVCPEVCWEG | |
| | TYNDAFLIDRLNWVSAGVYLNSNQTAENPVFAVFKDNEILYQVP | |
| | LAEDDTNAQKTITDCFLLENVIWCISLVEIYDTGDSVIRPKLFAVKI | |
| | PAQCSES | |
| 14 | MPAENKKVRFENTTSDKGKIPSKVIKSYYGTMDIKKINEGLLDSKI | gb: AF212302\|Organ |
| | LSAFNTVIALLGSIVIIVMNIMIIQNYTRSTDNQAVIKDALQGIQQQ | ism: Nipah |
| | IKGLADKIGTEIGPKVSLIDTSSTITIPANIGLLGSKISQSTASINENV | virus\|Strain |
| | NEKCKFTLPPLKIHECNISCPNPLPFREYRPQTEGVSNLVGLPNNIC | Name: UNKNOWN- |
| | LQKTSNQILKPKLISYTLPVVGQSGTCITDPLLAMDEGYFAYSHLE | AF212302\|Protein |
| | RIGSCSRGVSKQRIIGVGEVLDRGDEVPSLFMTNVWTPPNPNTVY | Name: attachment |
| | HCSAVYNNEFYYVLCAVSTVGDPILNSTYWSGSLMMTRLAVKP | glycoprotein\|Gene |
| | KSNGGGYNQHQLALRSIEKGRYDKVMPYGPSGIKQGDTLYFPAV | Symbol: G |
| | GFLVRTEFKYNDSNCPITKCQYSKPENCRLSMGIRPNSHYILRSGL | Uniprot Q9IH62 |
| | LKYNLSDGENPKVVFIEISDQRLSIGSPSKIYDSLGQPVFYQASFS | |
| | WDTMIKFGDVLTVNPLVVNWRNNTVISRPGQSQCPRFNTCPEIC | |
| | WEGVYNDAFLIDRINWISAGVFLDSNQTAENPVFTVFKDNEILYR | |
| | AQLASEDTNAQKTITNCFLLKNKIWCISLVEIYDTGDNVIRPKLFA | |
| | VKIPEQCT | |
| 15 | MLSQLQKNYLDNSNQQGDKMNNPDKKLSVNFNPLELDKGQKDL | gb: JQ001776: 8170- |
| | NKSYYVKNKNYNVSNLLNESLHDIKFCIYCIFSLLIIITIINIITISIVIT | 10275\|Organism: |
| | RLKVHEENNGMESPNLQSIQDSLSSLTNMINTEITPRIGILVTATSV | Cedar virus\|Strain |
| | TLSSSINYVGTKTNQLVNELKDYITKSCGFKVPELKLHECNISCAD | Name: CG1a\|Protein |
| | PKISKSAMYSTNAYAELAGPPKIFCKSVSKDPDFRLKQIDYVIPVQ | Name: attachment |
| | QDRSICMNNPLLDISDGFFTYIHYEGINSCKKSDSFKVLLSHGEIV | glycoprotein\|Gene |
| | DRGDYRPSLYLLSSHYHPYSMQVINCVPVTCNQSSFVFCHISNNT | Symbol: G |
| | KTLDNSDYSSDEYYITYFNGIDRPKTKKIPINNMTADNRYIHFTFS | |
| | GGGGVCLGEEFIIPVTTVINTDVFTHDYCESFNCSVQTGKSLKEIC | |
| | SESLRSPTNSSRYNLNGIMIISQNNMTDFKIQLNGITYNKLSFGSPG | |
| | RLSKTLGQVLYYQSSMSWDTYLKAGFVEKWKPFTPNWMNNTVI | |
| | SRPNQGNCPRYHKCPEICYGGTYNDIAPLDLGKDMYVSVILDSDQ | |
| | LAENPEITVENSTTILYKERVSKDELNTRSTTTSCFLFLDEPWCISV | |
| | LETNRFNGKSIRPEIYSYKIPKYC | |
| 16 | MPQKTVEFINMNSPLERGVSTLSDKKTLNQSKITKQGYFGLGSHS | gb: NC_025256: |
| | ERNWKKQKNQNDHYMTVSTMILEILVVLGIMENLIVLTMVYYQ | 9117- |
| | NDNINQRMAELTSNITVLNLNLNQLTNKIQREIIPRITLIDTATTITI | 11015\|Organism: Bat |
| | PSAITYILATLTTRISELLPSINQKCEFKTPTLVLNDCRINCTPPLNP | Paramyxovirus |
| | SDGVKMSSLATNLVAHGPSPCRNFSSVPTIYYYRIPGLYNRTALD | Eid_hel/GH- |
| | ERCILNPRLTISSTKFAYVHSEYDKNCTRGFKYYELMTFGEILEGP | M74a/GHA/2009\| |
| | EKEPRMFSRSFYSPTNAVNYHSCTPIVTVNEGYFLCLECTSSDPLY | Strain |
| | KANLSNSTFHLVILRHNKDEKIVSMPSFNLSTDQEYVQIIPAEGGG | Name: BatPV/Eid_hel/ |
| | TAESGNLYFPCIGRLLHKRVTHPLCKKSNCSRTDDESCLKSYYNQ | GH- |
| | GSPQHQVVNCLIRIRNAQRDNPTWDVITVDLTNTYPGSRSRIFGSF | M74a/GHA/2009\| |
| | SKPMLYQSSVSWHTLLQVAEITDLDKYQLDWLDTPYISRPGGSEC | Protein |
| | PFGNYCPTVCWEGTYNDVYSLTPNNDLFVTVYLKSEQVAENPYF | Name: glycoprotein\| |
| | AIFSRDQILKEFPLDAWISSARTTTISCFMFNNEIWCIAALEITRLN | Gene Symbol: G |
| | DDIIRPIYYSFWLPTDCRTPYPHTGKMTRVPLRSTYNY | |
| 17 | MATNRDNTITSAEVSQEDKVKKYYGVETAEKVADSISGNKVFIL | gb:NC_025352: |
| | MNTLLILTGAIITITLNITNLTAAKSQQNMLKIIQDDVNAKLEMFV | 8716- |
| | NLDQLVKGEIKPKVSLINTAVSVSIPGQISNLQTKFLQKYVYLEESI | 11257\|Organism: |
| | TKQCTCNPLSGIFPTSGPTYPPTDKPDDDTTDDDKVDTTIKPIEYP | Mojiang virus\|Strain |
| | KPDGCNRTGDHFTMEPGANFYTVPNLGPASSNSDECYTNPSFSIG | Name: Tongguan 1\| |
| | SSIYMFSQEIRKTDCTAGEILSIQIVLGRIVDKGQQGPQASPLLVW | Protein |
| | AVPNPKIINSCAVAAGDEMGWVLCSVTLTAASGEPIPHMFDGFW | Name: attachment |
| | LYKLEPDTEVVSYRITGYAYLLDKQYDSVFIGKGGGIQKGNDLYF | glycoprotein\|Gene |
| | QMYGLSRNRQSFKALCEHGSCLGTGGGGYQVLCDRAVMSFGSE | Symbol: G |
| | ESLITNAYLKVNDLASGKPVIIGQTFPPSDSYKGSNGRMYTIGDKY | |
| | GLYLAPSSWNRYLRFGITPDISVRSTTWLKSQDPIMKILSTCTNTD | |
| | RDMCPEICNTRGYQDIFPLSEDSEYYTYIGITPNNGGTKNFVAVRD | |
| | SDGHIASIDILQNYYSITSATISCFMYKDEIWCIAITEGKKQKDNPQ | |
| | RIYAHSYKIRQMCYNMKSATVTVGNAKNITIRRY | |
| 18 | MLSQLQKNYLDNSNQQGDKMNNPDKKLSVNFNPLELDKGQKDL | gb: JQ001776: 8170- |
| | NKSYYVKNKNYNVSNLLNESLHDIKFCIYCIFSLLIIITIINIITISIVIT | 10275\|Organism: |
| | RLKVHEENNGMESPNLQSIQDSLSSLTNMINTEITPRIGILVTATSV | Cedar virus\|Strain |
| | TLSSSINYVGTKTNQLVNELKDYITKSCGFKVPELKLHECNISCAD | Name: CG1a\|Protein |
| | PKISKSAMYSTNAYAELAGPPKIFCKSVSKDPDFRLKQIDYVIPVQ | Name: attachment |
| | QDRSICMNNPLLDISDGFFTYIHYEGINSCKKSDSFKVLLSHGEIV | glycoprotein\|Gene |
| | DRGDYRPSLYLLSSHYHPYSMQVINCVPVTCNQSSFVFCHISNNT | Symbol: G |
| | KTLDNSDYSSDEYYITYFNGIDRPKTKKIPINNMTADNRYIHFTFS | |
| | GGGGVCLGEEFIIPVTTVINTDVFTHDYCESFNCSVQTGKSLKEIC | |
| | SESLRSPTNSSRYNLNGIMIISQNNMTDFKIQLNGITYNKLSFGSPG | |
| | RLSKTLGQVLYYQSSMSWDTYLKAGFVEKWKPFTPNWMNNTVI | |
| | SRPNQGNCPRYHKCPEICYGGTYNDIAPLDLGKDMYVSVILDSDQ | |
| | LAENPEITVFNSTTILYKERVSKDELNTRSTTTSCFLFLDEPWCISV | |
| | LETNRFNGKSIRPEIYSYKIPKYC | |
| 19 | MKKINE GLLDSKILSA FNTVIALLGS IVIIVMNIMI IQNYTRSTDN | (E501A, W504A, |
| | QAVIKDALQG IQQQIKGLAD KIGTEIGPKV SLIDTSSTIT | Q530A, E533A) |
| | IPANIGLLGS KISQSTASIN ENVNEKCKFT LPPLKIHECN | NiV G protein (Gc |
| | ISCPNPLPFR EYRPQTEGVS NLVGLPNNIC LQKTSNQILK | Δ 34) |
| | PKLISYTLPV VGQSGTCITD PLLAMDEGYF AYSHLERIGS | |
| | CSRGVSKQRI IGVGEVLDRG DEVPSLFMTN VWTPPNPNTV | |
| | YHCSAVYNNE FYYVLCAVST VGDPILNSTY WSGSLMMTRL | |
| | AVKPKSNGGG YNQHQLALRS IEKGRYDKVM PYGPSGIKQG | |
| | DTLYFPAVGF LVRTEFKYND SNCPITKCQY SKPENCRLSM | |
| | GIRPNSHYIL RSGLLKYNLS DGENPKVVFI EISDQRLSIG | |
| | SPSKIYDSLG QPVFYQASFS WDTMIKFGDV LTVNPLVVNW | |
| | RNNTVISRPG QSQCPRENTC PAICAEGVYN DAFLIDRINW | |
| | ISAGVFLDSN ATAANPVFTV FKDNEILYRA QLASEDTNAQ | |
| | KTITNCFLLK NKIWCISLVE IYDTGDNVIR PKLFAVKIPE QC | |
| 20 | GGGGS | Linker |
| 21 | GGGGGS | Linker |
| 22 | (GGGGS)n | Linker |
| 23 | (GGGGGS)n | Linker |
| 24 | MALPVTALLLPLALLLHAARP | CD8α signal peptide |
| 25 | METDTLLLWVLLLWVPGSTG | IgK signal peptide |
| 26 | MLLLVTSLLLCELPHPAFLLIP | GMCSFR-α |
| | | (CSF2RA) signal |
| | | peptide |
| 27 | TTTPAPRPPTPAPTIASQPLSLRPEACRPAAGGAVHTRGLDFACD | CD8α hinge domain |
| 28 | IEVMYPPPYLDNEKSNGTIIHVKGKHLCPSPLFPGPSKP | CD28 hinge domain |
| 29 | AAAIEVMYPPPYLDNEKSNGTIIHVKGKHLCPSPLFPGPSKP | CD28 hinge domain |
| 30 | ESKYGPPCPPCP | IgG4 hinge domain |
| 31 | ESKYGPPCPSCP | IgG4 hinge domain |
| 32 | ESKYGPPCPPCPAPEFLGGPSVFLFPPKPKDTLMISRTPEVTCVVV | IgG4 hinge-CH2- |
| | DVSQEDPEVQFNWYVDGVEVHNAKTKPREEQFNSTYRVVSVLT | CH3 domain |
| | VLHQDWLNGKEYKCKVSNKGLPSSIEKTISKAKGQPREPQVYTLP | |
| | PSQEEMTKNQVSLTCLVKGFYPSDIAVEWESNGQPENNYKTTPP | |
| | VLDSDGSFFLYSRLTVDKSRWQEGNVFSCSVMHEALHNHYTQKS | |
| | LSLSLGK | |
| 33 | IYIWAPLAGTCGVLLLSLVITLYC | CD8α |
| | | transmembrane |
| | | domain |
| 34 | FWVLVVVGGVLACYSLLVTVAFIIFWV | CD28 |
| | | transmembrane |
| | | domain |
| 35 | MFWVLVVVGGVLACYSLLVTVAFIIFWV | CD28 |
| | | transmembrane |
| | | domain |
| 36 | KRGRKKLLYIFKQPFMRPVQTTQEEDGCSCRFPEEEEGGCEL | 4-1BB |
| | | costimulatory |
| | | domain |
| 37 | RSKRSRLLHSDYMNMTPRRPGPTRKHYQPYAPPRDFAAYRS | CD28 costimulatory |
| | | domain |
| 38 | RVKFSRSADAPAYQQGQNQLYNELNLGRREEYDVLDKRRGRDP | CD3ζ signaling |
| | EMGGKPRRKNPQEGLYNELQKDKMAEAYSEIGMKGERRRGKGH | domain |
| | DGLYQGLSTATKDTYDALHMQALPPR | |
| 39 | RVKFSRSADAPAYKQGQNQLYNELNLGRREEYDVLDKRRGRDP | CD3ζ signaling |
| | EMGGKPRRKNPQEGLYNELQKDKMAEAYSEIGMKGERRRGKGH | domain (with Q to K |
| | DGLYQGLSTATKDTYDALHMQALPPR | mutation at position |
| | | 14) |
| 40 | DIQMTQTTSSLSASLGDRVTISCRASQDISKYLNWYQQKPDGTVK | Anti-CD19 FMC63 |
| | LLIYHTSRLHSGVPSRFSGSGSGTDYSLTISNLEQEDIATYFCQQG | scFv entire |
| | NTLPYTFGGGTKLEITGSTSGSGKPGSGEGSTKGEVKLQESGPGL | sequence, with |
| | VAPSQSLSVTCTVSGVSLPDYGVSWIRQPPRKGLEWLGVIWGSET | Whitlow linker |
| | TYYNSALKSRLTIIKDNSKSQVFLKMNSLQTDDTAIYYCAKHYYY | |
| | GGSYAMDYWGQGTSVTVSS | |
| 41 | DIQMTQTTSSLSASLGDRVTISCRASQDISKYLNWYQQKPDGTVK | Anti-CD19 FMC63 |
| | LLIYHTSRLHSGVPSRFSGSGSGTDYSLTISNLEQEDIATYFCQQG | scFv light chain |
| | NTLPYTFGGGTKLEIT | variable region |
| 42 | QDISKY | Anti-CD19 FMC63 |
| | | scFv light chain |
| | | CDR1 |
| 43 | HTS | Anti-CD19 FMC63 |
| | | scFv light chain |
| | | CDR2 |
| 44 | QQGNTLPYT | Anti-CD19 FMC63 |
| | | scFv light chain |
| | | CDR3 |
| 45 | GSTSGSGKPGSGEGSTKG | Whitlow linker |
| 46 | EVKLQESGPGLVAPSQSLSVTCTVSGVSLPDYGVSWIRQPPRKGL | Anti-CD19 FMC63 |
| | EWLGVIWGSETTYYNSALKSRLTIIKDNSKSQVFLKMNSLQTDDT | scFv heavy chain |
| | AIYYCAKHYYYGGSYAMDYWGQGTSVTVSS | variable region |
| 47 | GVSLPDYG | Anti-CD19 FMC63 |
| | | scFv heavy chain |
| | | CDR1 |
| 48 | IWGSETT | Anti-CD19 FMC63 |
| | | scFv heavy chain |
| | | CDR2 |
| 49 | AKHYYYGGSYAMDY | Anti-CD19 FMC63 |
| | | scFv heavy chain |
| | | CDR3 |
| 50 | DIQMTQTTSSLSASLGDRVTISCRASQDISKYLNWYQQKPDGTVK | Anti-CD19 FMC63 |
| | LLIYHTSRLHSGVPSRFSGSGSGTDYSLTISNLEQEDIATYFCQQG | scFv entire |
| | NTLPYTFGGGTKLEITGGGGSGGGGSGGGGSEVKLQESGPGLVA | sequence, with |
| | PSQSLSVTCTVSGVSLPDYGVSWIRQPPRKGLEWLGVIWGSETTY | 3xG₄S linker |
| | YNSALKSRLTIIKDNSKSQVFLKMNSLQTDDTAIYYCAKHYYYG | |
| | GSYAMDYWGQGTSVTVSS | |
| 51 | GGGGSGGGGSGGGGS | 3xG₄S linker |
| 52 | atggccttaccagtgaccgccttgctcctgccgctggccttgctgctccacgccgccaggccg | Exemplary CD19 |
| | gacatccagatgacacagactacatcctccctgtctgcctctctgggagacagagtcaccatc | CAR nucleotide |
| | agttgcagggcaagtcaggacattagtaaatatttaaattggtatcagcagaaaccagatgga | sequence |
| | actgttaaactcctgatctaccatacatcaagattacactcaggagtcccatcaaggttcagt | |
| | ggcagtgggtctggaacagattattctctcaccattagcaacctggagcaagaagatattgcc | |
| | acttacttttgccaacagggtaatacgcttccgtacacgttcggaggggggaccaagctggag | |
| | catcacaggctccacctctggatccggcaagcccggatctgggagggatccaccaagggcgag | |
| | gtgaaactgcaggagtcaggacctggcctggtggcgccctcacagagcctgtccgtcacatgc | |
| | actgtctcaggggtctcattacccgactatggtgtaagctggattcgccagcctccacgaaag | |
| | ggtctggagtggctgggagtaatatggggtagtgaaaccacatactataattcagctctcaaa | |
| | tccagactgaccatcatcaaggacaactccaagagccaagttttcttaaaaatgaacagtctg | |
| | caaactgatgacacagccatttactactgtgccaaacattattactacggtggtagctatgct | |
| | atggactactggggccaaggaacctcagtcaccgtctcctcaaccacgacgccagcgccgcga | |
| | ccaccaacaccggcgcccaccatcgcgtcgcagcccctgtccctgcgcccagaggcgtgccgg | |
| | ccagcggcggggggcgcagtgcacacgagggggctggacttcgcctgtgatatctacatctgg | |
| | gcgcccttggccgggacttgtggggtccttctcctgtcactggttatcaccctttactgcaaa | |
| | cggggcagaaagaaactcctgtatatattcaaacaaccatttatgagaccagtacaaactact | |
| | caagaggaagatggctgtagctgccgatttccagaagaagaagaaggaggatgtgaactgaga | |
| | gtgaagttcagcaggagcgcagacgcccccgcgtaccagcagggccagaaccagctctataac | |
| | gagctcaatctaggacgaagagaggagtacgatgttttggacaagagacgtggccgggaccct | |
| | gagatggggggaaagccgagaaggaagaaccctcaggaaggcctgtacaatgaactgcagaaa | |
| | gataagatggcggaggcctacagtgagattgggatgaaaggcgagcgccggaggggcaagggg | |
| | cacgatggcctttaccagggtctcagtacagccaccaaggacacctacgacgcccttcacatg | |
| | caggccctgccccctcgc | |
| 53 | MALPVTALLLPLALLLHAARPDIQMTQTTSSLSASLGDRVTISCR | Exemplary CD19 |
| | ASQDISKYLNWYQQKPDGTVKLLIYHTSRLHSGVPSRFSGSGSGT | CAR amino acid |
| | DYSLTISNLEQEDIATYFCQQGNTLPYTFGGGTKLEITGSTSGSGK | sequence |
| | PGSGEGSTKGEVKLQESGPGLVAPSQSLSVTCTVSGVSLPDYGVS | |
| | WIRQPPRKGLEWLGVIWGSETTYYNSALKSRLTIIKDNSKSQVFL | |
| | KMNSLQTDDTAIYYCAKHYYYGGSYAMDYWGQGTSVTVSSTTT | |
| | PAPRPPTPAPTIASQPLSLRPEACRPAAGGAVHTRGLDFACDIYIW | |
| | APLAGTCGVLLLSLVITLYCKRGRKKLLYIFKQPFMRPVQTTQEE | |
| | DGCSCRFPEEEEGGCELRVKFSRSADAPAYQQGQNQLYNELNLG | |
| | RREEYDVLDKRRGRDPEMGGKPRRKNPQEGLYNELQKDKMAEA | |
| | YSEIGMKGERRRGKGHDGLYQGLSTATKDTYDALHMQALPPR | |
| 54 | atggccttaccagtgaccgccttgctcctgccgctggccttgctgctccacgccgccaggccg | Tisagenlecleucel |
| | gacatccagatgacacagactacatcctccctgtctgcctctctgggagacagagtcaccatc | CD19 CAR |
| | agttgcagggcaagtcaggacattagtaaatatttaaattggtatcagcagaaaccagatgga | nucleotide sequence |
| | actgttaaactcctgatctaccatacatcaagattacactcaggagtcccatcaaggttcagt | |
| | ggcagtgggtctggaacagattattctctcaccattagcaacctggagcaagaagatattgc | |
| | cacttacttttgccaacagggtaatacgcttccgtacacgttcggaggggggaccaagctgga | |
| | gatcacaggtggcggtggctcgggcggtggtgggtcgggtggcggcggatctgaggtgaaact | |
| | gcaggagtcaggacctggcctggtggcgccctcacagagcctgtccgtcacatgcactgtctc | |
| | aggggtctcattacccgactatggtgtaagctggattcgccagcctccacgaaagggtctgga | |
| | gtggctgggagtaatatggggtagtgaaaccacatactataattcagctctcaaatccagact | |
| | gaccatcatcaaggacaactccaagagccaagttttcttaaaaatgaacagtctgcaaactga | |
| | tgacacagccatttactactgtgccaaacattattactacggtggtagctatgctatggacta | |
| | ctggggccaaggaacctcagtcaccgtctcctcaaccacgacgccagcgccgcgaccaccaac | |
| | accggcgcccaccatcgcgtcgcagcccctgtccctgcgcccagaggcgtgccggccagcggg | |
| | gggggcgcagtgcacacgagggggctggacttcgcctgtgatatctacatctgggcgcccttg | |
| | gccgggacttgtggggtccttctcctgtcactggttatcaccctttactgcaaacggggcaga | |
| | aagaaactcctgtatatattcaaacaaccatttatgagaccagtacaaactactcaagagga | |
| | agatggctgtagctgccgatttccagaagaagaagaaggaggatgtgaactgagagtgaagtt | |
| | cagcaggagcgcagacgcccccgcgtacaagcagggccagaaccagctctataacgagctca | |
| | catctaggacgaagagaggagtacgatgttttggacaagagacgtggccgggaccctgagatg | |
| | gggggaaagccgagaaggaagaaccctcaggaaggcctgtacaatgaactgagaaagataaga | |
| | tggcggaggcctacagtgagattgggatgaaaggcgagcgccggaggggcaaggggcacgatg | |
| | gcctttaccagggtctcagtacagccaccaaggacacctacgacgcccttcacatgcaggccc | |
| | tgccccctcgc | |
| 55 | MALPVTALLLPLALLLHAARPDIQMTQTTSSLSASLGDRVTISCR | Tisagenlecleucel |
| | ASQDISKYLNWYQQKPDGTVKLLIYHTSRLHSGVPSRFSGSGSGT | CD19 CAR amino |
| | DYSLTISNLEQEDIATYFCQQGNTLPYTFGGGTKLEITGGGGSGG | acid sequence |
| | GGSGGGGSEVKLQESGPGLVAPSQSLSVTCTVSGVSLPDYGVSWI | |
| | RQPPRKGLEWLGVIWGSETTYYNSALKSRLTIIKDNSKSQVFLKM | |
| | NSLQTDDTAIYYCAKHYYYGGSYAMDYWGQGTSVTVSSTTTPA | |
| | PRPPTPAPTIASQPLSLRPEACRPAAGGAVHTRGLDFACDIYIWAP | |
| | LAGTCGVLLLSLVITLYCKRGRKKLLYIFKQPFMRPVQTTQEEDG | |
| | CSCRFPEEEEGGCELRVKFSRSADAPAYKQGQNQLYNELNLGRR | |
| | EEYDVLDKRRGRDPEMGGKPRRKNPQEGLYNELQKDKMAEAYS | |
| | EIGMKGERRRGKGHDGLYQGLSTATKDTYDALHMQALPPR | |
| 56 | atgctgctgctggtgaccagcctgctgctgtgcgagctgccccaccccgcctttctgctgatc | Lisocabtagene |
| | cccgacatccagatgacccagaccacctccagcctgagcgccagcctgggcgaccgggtgacc | maraleucel CD19 |
| | atcagctgccgggccagccaggacatcagcaagtacctgaactggtatcagcagaagcccgac | CAR nucleotide |
| | ggcaccgtcaagctgctgatctaccacaccagccggctgcacagcggcgtgcccagccggtt | sequence |
| | tagcggcagcggctccggcaccgactacagcctgaccatctccaacctggaacaggaagatat | |
| | cgccacctacttttgccagcagggcaacacactgccctacacctttggcggcggaacaaagc | |
| | tggaaatcaccggcagcacctccggcagcggcaagcctggcagcggcgagggcagcaccaagg | |
| | gcgaggtgaagctgcaggaaagcggccctggcctggtggcccccagccagagcctgagcgtga | |
| | cctgcaccgtgagcggcgtgagcctgcccgactacggcgtgagctggatccggcagcccccca | |
| | ggaagggcctggaatggctgggcgtgatctggggcagcgagaccacctactacaacagcgccc | |
| | tgaagagccggctgaccatcatcaaggacaacagcaagagccaggtgttcctgaagatgaaca | |
| | gcctgcagaccgacgacaccgccatctactactgcgccaagcactactactacggcggcagct | |
| | acgccatggactactggggccagggcaccagcgtgaccgtgagcagcgaatctaagtacggac | |
| | cgccctgccccccttgccctatgttctgggtgctggtggtggtcggaggcgtgctggcctgc | |
| | tacagcctgctggtcaccgtggccttcatcatcttttgggtgaaacggggcagaaagaaactc | |
| | tctgtatatattcaaacaaccatttatgagaccagtacaaactactcaagaggaagatggctg | |
| | tagctgccgattccagaagaagaagaaggaggatgtgaactgcgggtgaagttcagcagaagc | |
| | gccgacgcccctgcctaccagcagggccagaatcagctgtacaacgagctgaacctgggcaga | |
| | agggaagagtacgacgtcctggataagcggagaggccgggaccctgagatgggcggcaagcct | |
| | cggcggaagaacccccaggaaggcctgtataacgaactgcagaaagacaagatggccgaggc | |
| | ctacagcgagatcggcatgaagggcgagcggaggcggggcaagggccacgacggcctgtatc | |
| | agggcctgtccaccgccaccaaggatacctacgacgccctgcacatgcaggccctgccccca | |
| | agg | |
| 57 | MLLLVTSLLLCELPHPAFLLIPDIQMTQTTSSLSASLGDRVTISCRA | Lisocabtagene |
| | SQDISKYLNWYQQKPDGTVKLLIYHTSRLHSGVPSRFSGSGSGTD | maraleucel CD19 |
| | YSLTISNLEQEDIATYFCQQGNTLPYTFGGGTKLEITGSTSGSGKP | CAR amino acid |
| | GSGEGSTKGEVKLQESGPGLVAPSQSLSVTCTVSGVSLPDYGVS | sequence |
| | WIRQPPRKGLEWLGVIWGSETTYYNSALKSRLTIIKDNSKSQVFL | |
| | KMNSLQTDDTAIYYCAKHYYYGGSYAMDYWGQGTSVTVSSESK | |
| | YGPPCPPCPMFWVLVVVGGVLACYSLLVTVAFIIFWVKRGRKKL | |
| | LYIFKQPFMRPVQTTQEEDGCSCRFPEEEEGGCELRVKFSRSADA | |
| | PAYQQGQNQLYNELNLGRREEYDVLDKRRGRDPEMGGKPRRKN | |
| | PQEGLYNELQKDKMAEAYSEIGMKGERRRGKGHDGLYQGLSTA | |
| | TKDTYDALHMQALPPR | |
| 58 | atgcttctcctggtgacaagccttctgctctgtgagttaccacacccagcattcctcctgatc | Axicabtagene |
| | ccagacatccagatgacacagactacatcctccctgtctgcctctctgggagacagagtcac | ciloleucel CD19 |
| | catcagttgcagggcaagtcaggacattagtaaatatttaaattggtatcagcagaaaccaga | CAR nucleotide |
| | tggaactgttaaactcctgatctaccatacatcaagattacactcaggagtcccatcaaggtt | sequence |
| | cagtggcagtgggtctggaacagattattctctcaccattagcaacctggagcaagaagatat | |
| | tgccacttacttttgccaacagggtaatacgcttccgtacacgttcggaggggggactaag | |
| | ttggaaataacaggctccacctctggatccggcaagcccggatctggcgagggatccaccaag | |
| | ggcgaggtgaaactgcaggagtcaggacctggcctggtggcgccctcacagagcctgtccgtc | |
| | acatgcactgtctcaggggtctcattacccgactatggtgtaagctggattcgccagcctcc | |
| | acgaaagggtctggagtggctgggagtaatatggggtagtgaaaccacatactataattcagc | |
| | tctcaaatccagactgaccatcatcaaggacaactccaagagccaagttttcttaaaaatgaa | |
| | cagtctgcaaactgatgacacagccatttactactgtgccaaacattattactacggtggta | |
| | gctatgctatggactactggggtcaaggaacctcagtcaccgtctcctcagcggccgcaattg | |
| | aagttatgtatcctcctccttacctagacaatgagaagagcaatggaaccattatccatgtga | |
| | aagggaaacacctttgtccaagtcccctatttcccggaccttctaagcccttttgggtgctg | |
| | gtggtggttgggggagtcctggcttgctatagcttgctagtaacagtggcctttattattttc | |
| | tgggtgaggagtaagaggagcaggctcctgcacagtgactacatgaacatgactccccgccgc | |
| | cccgggcccacccgcaagcattaccagccctatgccccaccacgcgacttcgcagcctatcgc | |
| | tccagagtgaagttcagcaggagcgcagacgcccccgcgtaccagcagggccagaaccagctc | |
| | tataacgagctcaatctaggacgaagagaggagtacgatgttttggacaagagacgtggccgg | |
| | gaccctgagatggggggaaagccgagaaggaagaaccctcaggaaggcctgtacaatgaactg | |
| | cagaaagataagatggcggaggcctacagtgagattgggatgaaaggcgagcgccggagggg | |
| | caaggggcacgatggcctttaccagggtctcagtacagccaccaaggacacctacgacgccc | |
| | ttcacatgcaggccctgccccctcgc | |
| 59 | MLLLVTSLLLCELPHPAFLLIPDIQMTQTTSSLSASLGDRVTISCRA | Axicabtagene |
| | SQDISKYLNWYQQKPDGTVKLLIYHTSRLHSGVPSRFSGSGSGTD | ciloleucel CD19 |
| | YSLTISNLEQEDIATYFCQQGNTLPYTFGGGTKLEITGSTSGSGKP | CAR amino acid |
| | GSGEGSTKGEVKLQESGPGLVAPSQSLSVTCTVSGVSLPDYGVS | sequence |
| | WIRQPPRKGLEWLGVIWGSETTYYNSALKSRLTIIKDNSKSQVFL | |
| | KMNSLQTDDTAIYYCAKHYYYGGSYAMDYWGQGTSVTVSSAA | |
| | AIEVMYPPPYLDNEKSNGTIIHVKGKHLCPSPLFPGPSKPFWVLVV | |
| | VGGVLACYSLLVTVAFIIFWVRSKRSRLLHSDYMNMTPRRPGPTR | |
| | KHYQPYAPPRDFAAYRSRVKFSRSADAPAYQQGQNQLYNELNL | |
| | GRREEYDVLDKRRGRDPEMGGKPRRKNPQEGLYNELQKDKMAE | |
| | AYSEIGMKGERRRGKGHDGLYQGLSTATKDTYDALHMQALPPR | |
| 60 | DIVLTQSPAILSASPGEKVTMTCRASSSVNYMDWYQKKPGSSPKP | Anti-CD20 Leu16 |
| | WIYATSNLASGVPARFSGSGSGTSYSLTISRVEAEDAATYYCQQW | scFv entire |
| | SFNPPTFGGGTKLEIKGSTSGSGKPGSGEGSTKGEVQLQQSGAEL | sequence, with |
| | VKPGASVKMSCKASGYTFTSYNMHWVKQTPGQGLEWIGAIYPG | Whitlow linker |
| | NGDTSYNQKFKGKATLTADKSSSTAYMQLSSLTSEDSADYYCAR | |
| | SNYYGSSYWFFDVWGAGTTVTVSS | |
| 61 | DIVLTQSPAILSASPGEKVTMTCRASSSVNYMDWYQKKPGSSPKP | Anti-CD20 Leu16 |
| | WIYATSNLASGVPARFSGSGSGTSYSLTISRVEAEDAATYYCQQW | scFv light chain |
| | SFNPPTFGGGTKLEIK | variable region |
| 62 | RASSSVNYMD | Anti-CD20 Leu16 |
| | | scFv light chain |
| | | CDR1 |
| 63 | ATSNLAS | Anti-CD20 Leu16 |
| | | scFv light chain |
| | | CDR2 |
| 64 | QQWSFNPPT | Anti-CD20 Leu16 |
| | | scFv light chain |
| | | CDR3 |
| 65 | EVQLQQSGAELVKPGASVKMSCKASGYTFTSYNMHWVKQTPGQ | Anti-CD20 Leu16 |
| | GLEWIGAIYPGNGDTSYNQKFKGKATLTADKSSSTAYMQLSSLTS | scFv heavy chain |
| | EDSADYYCARSNYYGSSYWFFDVWGAGTTVTVSS | |
| 66 | SYNMH | Anti-CD20 Leu16 |
| | | scFv heavy chain |
| | | CDR1 |
| 67 | AIYPGNGDTSYNQKFKG | Anti-CD20 Leu16 |
| | | scFv heavy chain |
| | | CDR2 |
| 68 | SNYYGSSYWFFDV | Anti-CD20 Leu16 |
| | | scFv heavy chain |
| | | CDR3 |
| 69 | QVQLQQSGPGLVKPSQTLSLTCAISGDSVSSNSAAWNWIRQSPSR | Anti-CD22 m971 |
| | GLEWLGRTYYRSKWYNDYAVSVKSRITINPDTSKNQFSLQLNSV | scFv entire |
| | TPEDTAVYYCAREVTGDLEDAFDIWGQGTMVTVSSGGGGSGGG | sequence, with |
| | GSGGGGSDIQMTQSPSSLSASVGDRVTITCRASQTIWSYLNWYQQ | 3xG4S linker |
| | RPGKAPNLLIYAASSLQSGVPSRFSGRGSGTDFTLTISSLQAEDFA | |
| | TYYCQQSYSIPQTFGQGTKLEIK | |
| 70 | QVQLQQSGPGLVKPSQTLSLTCAISGDSVSSNSAAWNWIRQSPSR | Anti-CD22 m971 |
| | GLEWLGRTYYRSKWYNDYAVSVKSRITINPDTSKNQFSLQLNSV | scFv heavy chain |
| | TPEDTAVYYCAREVTGDLEDAFDIWGQGTMVTVSS | variable region |
| 71 | GDSVSSNSAA | Anti-CD22 m971 |
| | | scFv heavy chain |
| | | CDR1 |
| 72 | TYYRSKWYN | Anti-CD22 m971 |
| | | scFv heavy chain |
| | | CDR2 |
| 73 | AREVTGDLEDAFDI | Anti-CD22 m971 |
| | | scFv heavy chain |
| | | CDR3 |
| 74 | DIQMTQSPSSLSASVGDRVTITCRASQTIWSYLNWYQQRPGKAPN | Anti-CD22 m971 |
| | LLIYAASSLQSGVPSRFSGRGSGTDFTLTISSLQAEDFATYYCQQS | scFv light chain |
| | YSIPQTFGQGTKLEIK | |
| 75 | QTIWSY | Anti-CD22 m971 |
| | | scFv light chain |
| | | CDR1 |
| 76 | AAS | Anti-CD22 m971 |
| | | scFv light chain |
| | | CDR2 |
| 77 | QQSYSIPQT | Anti-CD22 m971 |
| | | scFv light chain |
| | | CDR3 |
| 78 | QVQLQQSGPGMVKPSQTLSLTCAISGDSVSSNSVAWNWIRQSPSR | Anti-CD22 m971- |
| | GLEWLGRTYYRSTWYNDYAVSMKSRITINPDTNKNQFSLQLNSV | L7 scFv entire |
| | TPEDTAVYYCAREVTGDLEDAFDIWGQGTMVTVSSGGGGSGGG | sequence, with |
| | GSGGGGSDIQMIQSPSSLSASVGDRVTITCRASQTIWSYLNWYRQ | 3xG4S linker |
| | RPGEAPNLLIYAASSLQSGVPSRFSGRGSGTDFTLTISSLQAEDFAT | |
| | YYCQQSYSIPQTFGQGTKLEIK | |
| 79 | QVQLQQSGPGMVKPSQTLSLTCAISGDSVSSNSVAWNWIRQSPSR | Anti-CD22 m971- |
| | GLEWLGRTYYRSTWYNDYAVSMKSRITINPDTNKNQFSLQLNSV | L7 scFv heavy chain |
| | TPEDTAVYYCAREVTGDLEDAFDIWGQGTMVTVSS | variable region |
| 80 | GDSVSSNSVA | Anti-CD22 m971- |
| | | L7 scFv heavy chain |
| | | CDR1 |
| 81 | TYYRSTWYN | Anti-CD22 m971- |
| | | L7 scFv heavy chain |
| | | CDR2 |
| 82 | AREVTGDLEDAFDI | Anti-CD22 m971- |
| | | L7 scFv heavy chain |
| | | CDR3 |
| 83 | DIQMIQSPSSLSASVGDRVTITCRASQTIWSYLNWYRQRPGEAPN | Anti-CD22 m971- |
| | LLIYAASSLQSGVPSRFSGRGSGTDFTLTISSLQAEDFATYYCQQS | L7 scFv light chain |
| | YSIPQTFGQGTKLEIK | variable region |
| 84 | QTIWSY | Anti-CD22 m971- |
| | | L7 scFv light chain |
| | | CDR1 |
| 85 | AAS | Anti-CD22 m971- |
| | | L7 scFv light chain |
| | | CDR2 |
| 86 | QQSYSIPQT | Anti-CD22 m971- |
| | | L7 scFv light chain |
| | | CDR3 |
| 87 | DIVLTQSPASLAMSLGKRATISCRASESVSVIGAHLIHWYQQKPG | Anti-BCMA |
| | QPPKLLIYLASNLETGVPARFSGSGSGTDFTLTIDPVEEDDVAIYSC | C11D5.3 scFv entire |
| | LQSRIFPRTFGGGTKLEIKGSTSGSGKPGSGEGSTKGQIQLVQSGP | sequence, with |
| | ELKKPGETVKISCKASGYTFTDYSINWVKRAPGKGLKWMGWINT | Whitlow linker |
| | ETREPAYAYDFRGRFAFSLETSASTAYLQINNLKYEDTATYFCAL | |
| | DYSYAMDYWGQGTSVTVSS | |
| 88 | DIVLTQSPASLAMSLGKRATISCRASESVSVIGAHLIHWYQQKPG | Anti-BCMA |
| | QPPKLLIYLASNLETGVPARFSGSGSGTDFTLTIDPVEEDDVAIYSC | C11D5.3 scFv light |
| | LQSRIFPRTFGGGTKLEIK | chain variable |
| | | region |
| 89 | RASESVSVIGAHLIH | Anti-BCMA |
| | | C11D5.3 scFv light |
| | | chain CDR1 |
| 90 | LASNLET | Anti-BCMA |
| | | C11D5.3 scFv light |
| | | chain CDR2 |
| 91 | LQSRIFPRT | Anti-BCMA |
| | | C11D5.3 scFv light |
| | | chain CDR3 |
| 92 | QIQLVQSGPELKKPGETVKISCKASGYTFTDYSINWVKRAPGKGL | Anti-BCMA |
| | KWMGWINTETREPAYAYDFRGRFAFSLETSASTAYLQINNLKYE | C11D5.3 scFv |
| | DTATYFCALDYSYAMDYWGQGTSVTVSS | |
| | | heavy chain variable |
| | | region |
| 93 | DYSIN | Anti-BCMA |
| | | C11D5.3 scFv |
| | | heavy chain CDR1 |
| 94 | WINTETREPAYAYDFRG | Anti-BCMA |
| | | C11D5.3 scFv |
| | | heavy chain CDR2 |
| 95 | DYSYAMDY | Anti-BCMA |
| | | C11D5.3 scFv |
| | | heavy chain CDR3 |
| 96 | DIVLTQSPPSLAMSLGKRATISCRASESVTILGSHLIYWYQQKPGQ | Anti-BCMA |
| | PPTLLIQLASNVQTGVPARFSGSGSRTDFTLTIDPVEEDDVAVYYC | C12A3.2 scFv entire |
| | LQSRTIPRTFGGGTKLEIKGSTSGSGKPGSGEGSTKGQIQLVQSGP | sequence, with |
| | ELKKPGETVKISCKASGYTFRHYSMNWVKQAPGKGLKWMGRIN | Whitlow linker |
| | TESGVPIYADDFKGRFAFSVETSASTAYLVINNLKDEDTASYFCSN | |
| | DYLYSLDFWGQGTALTVSS | |
| 97 | DIVLTQSPPSLAMSLGKRATISCRASESVTILGSHLIYWYQQKPGQ | Anti-BCMA |
| | PPTLLIQLASNVQTGVPARFSGSGSRTDFTLTIDPVEEDDVAVYYC | C12A3.2 scFv light |
| | LQSRTIPRTFGGGTKLEIK | chain variable |
| | | region |
| 98 | RASESVTILGSHLIY | Anti-BCMA |
| | | C12A3.2 scFv light |
| | | chain CDR1 |
| 99 | LASNVQT | Anti-BCMA |
| | | C12A3.2 scFv light |
| | | chain CDR2 |
| 100 | LQSRTIPRT | Anti-BCMA |
| | | C12A3.2 scFv light |
| | | chain CDR3 |
| 101 | QIQLVQSGPELKKPGETVKISCKASGYTFRHYSMNWVKQAPGKG | Anti-BCMA |
| | LKWMGRINTESGVPIYADDFKGRFAFSVETSASTAYLVINNLKDE | C12A3.2 scFv |
| | DTASYFCSNDYLYSLDFWGQGTALTVSS | heavy chain variable |
| | | region |
| 102 | HYSMN | Anti-BCMA |
| | | C12A3.2 scFv |
| | | heavy chain CDR1 |
| 103 | RINTESGVPIYADDFKG | Anti-BCMA |
| | | C12A3.2 scFv |
| | | heavy chain CDR2 |
| 104 | DYLYSLDF | Anti-BCMA |
| | | C12A3.2 scFv |
| | | heavy chain CDR3 |
| 105 | EVQLLESGGGLVQPGGSLRLSCAASGFTFSSYAMSWVRQAPGKG | Anti-BCMA |
| | LEWVSSISGSGDYIYYADSVKGRFTISRDISKNTLYLQMNSLRAED | FHVH33 entire |
| | TAVYYCAKEGTGANSSLADYRGQGTLVTVSS | sequence |
| 106 | GFTFSSYA | Anti-BCMA |
| | | FHVH33 CDR1 |
| 107 | ISGSGDYI | Anti-BCMA |
| | | FHVH33 CDR2 |
| 108 | AKEGTGANSSLADY | Anti-BCMA |
| | | FHVH33 CDR3 |
| 109 | DIQMTQSPSSLSASVGDRVTITCRASQSISSYLNWYQQKPGKAPK | Anti-BCMA |
| | LLIYAASSLQSGVPSRFSGSGSGTDFTLTISSLQPEDFATYYCQQK | CT103A scFv entire |
| | YDLLTFGGGTKVEIKGSTSGSGKPGSGEGSTKGQLQLQESGPGLV | sequence, with |
| | KPSETLSLTCTVSGGSISSSSYYWGWIRQPPGKGLEWIGSISYSGST | Whitlow linker |
| | YYNPSLKSRVTISVDTSKNQFSLKLSSVTAADTAVYYCARDRGDT | |
| | ILDVWGQGTMVTVSS | |
| 110 | DIQMTQSPSSLSASVGDRVTITCRASQSISSYLNWYQQKPGKAPK | Anti-BCMA |
| | LLIYAASSLQSGVPSRFSGSGSGTDFTLTISSLQPEDFATYYCQQK | CT103A scFv light |
| | YDLLTFGGGTKVEIK | chain variable |
| | | region |
| 111 | QSISSY | Anti-BCMA |
| | | CT103A scFv light |
| | | chain CDR1 |
| 112 | AAS | Anti-BCMA |
| | | CT103A scFv light |
| | | chain CDR2 |
| 113 | QQKYDLLT | Anti-BCMA |
| | | CT103A scFv light |
| | | chain CDR3 |
| 114 | QLQLQESGPGLVKPSETLSLTCTVSGGSISSSSYYWGWIRQPPGKG | Anti-BCMA |
| | LEWIGSISYSGSTYYNPSLKSRVTISVDTSKNQFSLKLSSVTAADT | CT103A scFv heavy |
| | AVYYCARDRGDTILDVWGQGTMVTVSS | chain variable |
| | | region |
| 115 | GGSISSSSYY | Anti-BCMA |
| | | CT103A scFv heavy |
| | | chain CDR1 |
| 116 | ISYSGST | Anti-BCMA |
| | | CT103A scFv heavy |
| | | chain CDR2 |
| 117 | ARDRGDTILDV | Anti-BCMA |
| | | CT103A scFv heavy |
| | | chain CDR3 |
| 118 | atggccttaccagtgaccgccttgctcctgccgctggccttgctgctccacgccgccaggccg | Exemplary BCMA |
| | gacatccagatgacccagtctccatcctccctgtctgcatctgtaggagacagagtcaccatc | CAR nucleotide |
| | acttgccgggcaagtcagagcattagcagctatttaaattggtatcagcagaaaccagggaaa | sequence |
| | gcccctaagctcctgatctatgctgcatccagtttgcaaagtggggtcccatcaaggttcagt | |
| | ggcagtggatctgggacagatttcactctcaccatcagcagtctgcaacctgaagattttgca | |
| | acttactactgtcagcaaaaatacgacctcctcacttttggcggagggaccaaggttgagat | |
| | caaaggcagcaccagcggctccggcaagcctggctctggcgagggcagcacaaagggacagct | |
| | gcagctgcaggagtcgggcccaggactggtgaagccttcggagaccctgtccctcacctgcac | |
| | tgtctctggtggctccatcagcagtagtagttactactggggctggatccgccagcccccag | |
| | ggaaggggctggagtggattgggagtatctcctatagtgggagcacctactacaacccgtcc | |
| | ctcaagagtcgagtcaccatatccgtagacacgtccaagaaccagttctccctgaagctgagt | |
| | tctgtgaccgccgcagacacggcggtgtactactgcgccagagatcgtggagacaccatact | |
| | agacgtatggggtcagggtacaatggtcaccgtcagctcattcgtgcccgtgttcctgcccg | |
| | ccaaacctaccaccacccctgcccctagacctcccaccccagccccaacaatcgccagccagc | |
| | ctctgtctctgcggcccgaagcctgtagacctgctgccggcggagccgtgcacaccagaggcc | |
| | tggacttcgcctgcgacatctacatctgggcccctctggccggcacctgtggcgtgctgctg | |
| | ctgagcctggtgatcaccctgtactgcaaccaccggaacaaacggggcagaaagaaactcct | |
| | gtatatattcaaacaaccatttatgagaccagtacaaactactcaagaggaagatggctgta | |
| | gctgccgatttccagaagaagaagaaggaggatgtgaactgagagtgaagttcagcagatccg | |
| | ccgacgcccctgcctaccagcagggacagaaccagctgtacaacgagctgaacctgggcagac | |
| | gggaagagtacgacgtgctggacaagcggagaggccgggaccccgagatgggcggaaagccca | |
| | gacggaagaacccccaggaaggcctgtataacgaactgcagaaagacaagatggccgaggcct | |
| | acagcgagatcggcatgaagggcgagcggaggcgcggcaagggccacgatggcctgtaccagg | |
| | gcctgagcaccgccaccaaggacacctacgacgccctgcacatgcaggccctgccccccaga | |
| 119 | MALPVTALLLPLALLLHAARPDIQMTQSPSSLSASVGDRVTITCR | Exemplary BCMA |
| | ASQSISSYLNWYQQKPGKAPKLLIYAASSLQSGVPSRFSGSGSGT | CAR amino acid |
| | DFTLTISSLQPEDFATYYCQQKYDLLTFGGGTKVEIKGSTSGSGKP | sequence |
| | GSGEGSTKGQLQLQESGPGLVKPSETLSLTCTVSGGSISSSSYYWG | |
| | WIRQPPGKGLEWIGSISYSGSTYYNPSLKSRVTISVDTSKNQFSLK | |
| | LSSVTAADTAVYYCARDRGDTILDVWGQGTMVTVSSFVPVFLPA | |
| | KPTTTPAPRPPTPAPTIASQPLSLRPEACRPAAGGAVHTRGLDFAC | |
| | DIYIWAPLAGTCGVLLLSLVITLYCNHRNKRGRKKLLYIFKQPFM | |
| | RPVQTTQEEDGCSCRFPEEEEGGCELRVKFSRSADAPAYQQGQN | |
| | QLYNELNLGRREEYDVLDKRRGRDPEMGGKPRRKNPQEGLYNE | |
| | LQKDKMAEAYSEIGMKGERRRGKGHDGLYQGLSTATKDTYDAL | |
| | HMQALPPR | |
| 120 | QVQLVQSGAEVKKPGASVKVSCKASGGTFSSYAISWVRQAPGQG | CD8_1 VH |
| | LEWMGIIDPSDGNTNYAQNFQGRVTMTRDTSTSTVYMELSSLRS | |
| | EDTAVYYCAKERAAAGYYYYMDVWGQGTTVTVSS | |
| 121 | DIQMTQSPSSLSASVGDRVTITCRASQSISSYLNWYQQKPGKAPK | CD8_1 VL |
| | LLIYAASSLQSGVPSRFSGSGSGTDFTLTISSLQPEDFATYYCQQSY | |
| | STPLTFGGGTKVEIKR | |
| 122 | QVQLVQSGAEVKKPGASVKVSCKASGYTFTDYYIQWVRQAPGQ | CD8_2 VH |
| | GLEWMGWINPNSGGTSYAQKFQGRVTMTRDTSTSTVYMELSSL | |
| | RSEDTAVYYCAKEGDYYYGMDAWGQGTMVTVSS | |
| 123 | DIVMTQSPLSLPVTPGEPASISCRSSQSLLHSNGYNYLDWYLQKP | CD8_2 VL |
| | GQSPQLLIYLGSNRASGVPDRFSGSGSGTDFTLKISRVEAEDVGV | |
| | YYCMQGLQTPHTFGQGTKVEIKR | |
| 124 | QVQLVQSGAEVKKPGASVKVSCKASGYTFTSYYMHWVRQAPG | CD8_3 VH |
| | QGLEWMGGFDPEDGETIYAQKFQGRVTMTRDTSTSTVYMELSSL | |
| | RSEDTAVYYCARDQGWGMDVWGQGTTVTVSS | |
| 125 | DIQMTQSPSSLSASVGDRVTITCRASQSISSYLNWYQQKPGKAPK | CD8_3 VL |
| | LLIYAASSLQSGVPSRFSGSGSGTDFTLTISSLQPEDFATYYCQQTY | |
| | STPYTFGQGTKLEIKR | |
| 126 | QVQLVQSGAEVKKPGASVKVSCKASGYTFTNHYMHWVRQAPG | CD8_4 VH |
| | QGLEWMGWMNPNSGNTGYAQKFQGRVTMTRDTSTSTVYMELS | |
| | SLRSEDTAVYYCASSESGSDLDYWGQGTLVTVSS | |
| 127 | DIQMTQSPSSLSASVGDRVTITCRASQTIGNYVNWYQQKPGKAPK | CD8_4 VL |
| | LLIYGASNLHTGVPSRFSGSGSGTDFTLTISSLQPEDFATYYCQQT | |
| | YSAPLTFGGGTKVEIKR | |
| 128 | QVQLVESGGGLVQAGGSLRLSCAASGRTFSGYVMGWFRQAPGK | CD8_VHH |
| | QRKFVAAISRGGLSTSYADSVKGRFTISRDNAKNTVFLQMNTLKP | |
| | EDTAVYYCAADRSDLYEITAASNIDSWGQGTLVTVSS | |
| 129 | GFTFSGYW | CDR-H1 |
| 130 | ISPGGGST | CDR-H2 |
| 131 | ASSLTATHTYEYDY | CDR-H3 |
| 132 | EVQLVESGGGLVQSGGSLRLSCAASGFTFSGYWMYWVRQAPGK | VHH |
| | GLEWVSAISPGGGSTYYPDSVKGRFTISRDNAKNTLYLQMNSLEP | |
| | EDTALYYCASSLTATHTYEYDYWGQGTQVTVSS | |
| 133 | GYTFSNYW | CDR-H1 |
| 134 | ILPGSGST | CDR-H2 |
| 135 | ARRGYGYDEGFDY | CDR-H3 |
| 136 | QDINSY | CDR-L1 |
| 137 | RAN | CDR-L2 |
| 138 | LQYDEFPPT | CDR-L3 |
| 139 | QVQLQQSGAELMKPGASVKMSCKATGYTFSNYWIEWVKQRPGH | VH |
| | GLEWIGEILPGSGSTSYNEKFKGKATFTADTSSSTAYMQLSSLTSE | |
| | DSAVYYCARRGYGYDEGFDYWGQGSTLTVSS | |
| 140 | DIKMTQSPSSMYASLGERVTITCKASQDINSYLSWFQQKPGKSPK | VL |
| | TLIYRANRLVDGVPSRFSGSGSGQDYSLTISSLEYEDMGIYYCLQY | |
| | DEFPPTFGAGTKLELKR | |
| 141 | QVQLQQSGAELMKPGASVKMSCKATGYTFSNYWIEWVKQRPGH | scFv |
| | GLEWIGEILPGSGSTSYNEKFKGKATFTADTSSSTAYMQLSSLTSE | |
| | DSAVYYCARRGYGYDEGFDYWGQGSTLTVSSGGGGSGGGGSGG | |
| | GGSDIKMTQSPSSMYASLGERVTITCKASQDINSYLSWFQQKPGK | |
| | SPKTLIYRANRLVDGVPSRFSGSGSGQDYSLTISSLEYEDMGIYYC | |
| | LQYDEFPPTFGAGTKLELKR | |
| 142 | GYTFTDYV | CDR-H1 |
| 143 | IYPGSGSS | CDR-H2 |
| 144 | ARPGDLGFAY | CDR-H3 |
| 145 | QSVDYDGDSY | CDR-L1 |
| 146 | AAS | CDR-L2 |
| 147 | QQSNKDPFT | CDR-L3 |
| 148 | QVQLQQSGPELVKPGASVKMSCKASGYTFTDYVISWVRQAPGQ | VH |
| | GLEWIGEIYPGSGSSYYNEKFKGRATLTADKSSNTAYMQLSSLRS | |
| | EDSAVYFCARPGDLGFAYWGQGTLVTVSS | |
| 149 | DIVLTQSPSSLAVSLGQRATISCKASQSVDYDGDSYMNWYQQKP | VL |
| | GQPPKLLIYAASNLESGIPARFSGSGSGTDFTLTIHPVEEEDAATY | |
| | YCQQSNKDPFTFGGGTKLELKR | |
| 150 | QVQLQQSGPELVKPGASVKMSCKASGYTFTDYVISWVRQAPGQ | scFv |
| | GLEWIGEIYPGSGSSYYNEKFKGRATLTADKSSNTAYMQLSSLRS | |
| | EDSAVYFCARPGDLGFAYWGQGTLVTVSSGGGGSGGGGSGGGG | |
| | SDIVLTQSPSSLAVSLGQRATISCKASQSVDYDGDSYMNWYQQK | |
| | PGQPPKLLIYAASNLESGIPARFSGSGSGTDFTLTIHPVEEEDAATY | |
| | YCQQSNKDPFTFGGGTKLELKR | |
| 151 | GGTFSSYA | CDR-H1 |
| 152 | INPNSGGT | CDR-H2 |
| 153 | ARDGYSGSYSD | CDR-H3 |
| 154 | QSVLSSSYNKNY | CDR-L1 |
| 155 | WAS | CDR-L2 |
| 156 | QQYYSTPWT | CDR-L3 |
| 157 | QVQLVQSGAEVKKPGASVKVSCKASGGTFSSYAISWVRQAPGQG | VH |
| | LEWMGWINPNSGGTNYAQKFQGRVTMTRDTSTSTVYMELSSLR | |
| | SEDTAVYYCARDGYSGSYSDWGQGTLVTVSS | |
| 158 | DIVMTQSPDSLAVSLGERATINCKSSQSVLSSSYNKNYLAWYQQ | VL |
| | KPGQPPKLLIYWASTRESGVPDRFSGSGSGTDFTLTISSLQAEDVA | |
| | VYYCQQYYSTPWTFGQGTKVEIK | |
| 159 | QVQLVQSGAEVKKPGASVKVSCKASGGTFSSYAISWVRQAPGQG | scFv |
| | LEWMGWINPNSGGTNYAQKFQGRVTMTRDTSTSTVYMELSSLR | |
| | SEDTAVYYCARDGYSGSYSDWGQGTLVTVSSGGGGSGGGGSGG | |
| | GGSDIVMTQSPDSLAVSLGERATINCKSSQSVLSSSYNKNYLAWY | |
| | QQKPGQPPKLLIYWASTRESGVPDRFSGSGSGTDFTLTISSLQAED | |
| | VAVYYCQQYYSTPWTFGQGTKVEIK | |
| 160 | GYTFTSYD | CDR-H1 |
| 161 | IIPLSGAP | CDR-H2 |
| 162 | ARGALYNWNDGWFDP | CDR-H3 |
| 163 | QDIGDY | CDR-L1 |
| 164 | DAS | CDR-L2 |
| 165 | QQANSFPLT | CDR-L3 |
| 166 | QVQLVQSGAEVKKPGSSVKVSCKASGYTFTSYDINWVRQAPGQ | VH |
| | GLEWMGGIIPLSGAPNYAHKFQGRVTITADESTSTAYMELSSLRS | |
| | EDTAVYYCARGALYNWNDGWFDPWGQGTLVTVSS | |
| 167 | DIQMTQSPSSLSASVGDRVTITCRASQDIGDYLAWYQQKPGKAPK | VL |
| | LLIYDASSLQSGVPSRFSGSGSGTDFTLTISSLQPEDFATYYCQQA | |
| | NSFPLTFGGGTKVEIK | |
| 168 | QVQLVQSGAEVKKPGSSVKVSCKASGYTFTSYDINWVRQAPGQ | scFv |
| | GLEWMGGIIPLSGAPNYAHKFQGRVTITADESTSTAYMELSSLRS | |
| | EDTAVYYCARGALYNWNDGWFDPWGQGTLVTVSSGGGGSGGG | |
| | GSGGGGSDIQMTQSPSSLSASVGDRVTITCRASQDIGDYLAWYQQ | |
| | KPGKAPKLLIYDASSLQSGVPSRFSGSGSGTDFTLTISSLQPEDFAT | |
| | YYCQQANSFPLTFGGGTKVEIK | |
| 169 | GDTFSDHA | CDR-H1 |
| 170 | MNPKIGNT | CDR-H2 |
| 171 | VYDSSGYDAFDI | CDR-H3 |
| 172 | QSVLSTSYNRNF | CDR-L1 |
| 173 | QQYYSTPYT | CDR-L3 |
| 174 | QVQLVQSGAEVKKPGASVKVSCKASGDTFSDHAINWVRQAPGQ | VH |
| | GLEWMGWMNPKIGNTGYAQKFQGRVTMTRDTSTSTVYMELSSL | |
| | RSEDTAVYYCVYDSSGYDAFDIWGQGTTVTVSS | |
| 175 | DIVMTQSPDSLAVSLGERATINCKSSQSVLSTSYNRNFLAWYQQK | VL |
| | PGQPPKLLIYWASTRQSGVPDRFSGSGSGTDFTLTISSLQAEDVAV | |
| | YYCQQYYSTPYTFGQGTKLEIK | |
| 176 | QVQLVQSGAEVKKPGASVKVSCKASGDTFSDHAINWVRQAPGQ | scFv |
| | GLEWMGWMNPKIGNTGYAQKFQGRVTMTRDTSTSTVYMELSSL | |
| | RSEDTAVYYCVYDSSGYDAFDIWGQGTTVTVSSGGGGSGGGGS | |
| | GGGGSDIVMTQSPDSLAVSLGERATINCKSSQSVLSTSYNRNFLA | |
| | WYQQKPGQPPKLLIYWASTRQSGVPDRFSGSGSGTDFTLTISSLQ | |
| | AEDVAVYYCQQYYSTPYTFGQGTKLEIK | |
| 177 | GYSLITHW | CDR-H1 |
| 178 | INPSDGVT | CDR-H2 |
| 179 | AREYYGEGFDY | CDR-H3 |
| 180 | QGISNY | CDR-L1 |
| 181 | SAS | CDR-L2 |
| 182 | QQSYSTPLT | CDR-L3 |
| 183 | QVQLVQSGAEVKKPGASVKVSCKASGYSLITHWMHWVRQAPG | VH |
| | QGLEWMGMINPSDGVTYYAQTFQGRVTMTRDTSTSTVYMELSS | |
| | LRSEDTAVYYCAREYYGEGFDYWGQGTLVTVSS | |
| 184 | DIQMTQSPSSLSASVGDRVTITCRASQGISNYLAWYQQKPGKAPK | VL |
| | LLIYSASNLQSGVPSRFSGSGSGTDFTLTISSLQPEDFATYYCQQSY | |
| | STPLTFGGGTKVEIKR | |
| 185 | QVQLVQSGAEVKKPGASVKVSCKASGYSLITHWMHWVRQAPG | scFv |
| | QGLEWMGMINPSDGVTYYAQTFQGRVTMTRDTSTSTVYMELSS | |
| | LRSEDTAVYYCAREYYGEGFDYWGQGTLVTVSSGGGGSGGGGS | |
| | GGGGSDIQMTQSPSSLSASVGDRVTITCRASQGISNYLAWYQQKP | |
| | GKAPKLLIYSASNLQSGVPSRFSGSGSGTDFTLTISSLQPEDFATYY | |
| | CQQSYSTPLTFGGGTKVEIKR | |
| 186 | GYWMY | CDR-H1 |
| 187 | AISPGGGSTYYPDSVKG | CDR-H2 |
| 188 | SLTATHTYEYDY | CDR-H3 |
| 189 | GFTFSGY | CDR-H1 |
| 190 | SPGGGS | CDR-H2 |
| 191 | NYWIE | CDR-H1 |
| 192 | EILPGSGSTSYNEKFKG | CDR-H2 |
| 193 | RGYGYDEGFDY | CDR-H3 |
| 194 | KASQDINSYLS | CDR-L1 |
| 195 | RANRLVD | CDR-L2 |
| 196 | GYTFSNY | CDR-H1 |
| 197 | LPGSGS | CDR-H2 |
| 198 | DYVIS | CDR-H1 |
| 199 | EIYPGSGSSYYNEKFKG | CDR-H2 |
| 200 | PGDLGFAY | CDR-H3 |
| 201 | KASQSVDYDGDSYMN | CDR-L1 |
| 202 | AASNLES | CDR-L2 |
| 203 | GYTFTDY | CDR-H1 |
| 204 | YPGSGS | CDR-H2 |
| 205 | SYAIS | CDR-H1 |
| 206 | WINPNSGGTNYAQKFQG | CDR-H2 |
| 207 | DGYSGSYSD | CDR-H3 |
| 208 | KSSQSVLSSSYNKNYLA | CDR-L1 |
| 209 | WASTRES | CDR-L2 |
| 210 | GGTFSSY | CDR-H1 |
| 211 | NPNSGG | CDR-H2 |
| 212 | SYDIN | CDR-H1 |
| 213 | GIIPLSGAPNYAHKFQG | CDR-H2 |
| 214 | GALYNWNDGWFDP | CDR-H3 |
| 215 | RASQDIGDYLA | CDR-L1 |
| 216 | DASSLQS | CDR-L2 |
| 217 | GYTFTSY | CDR-H1 |
| 218 | IPLSGA | CDR-H2 |
| 219 | DHAIN | CDR-H1 |
| 220 | WMNPKIGNTGYAQKFQG | CDR-H2 |
| 221 | DSSGYDAFDI | CDR-H3 |
| 222 | KSSQSVLSTSYNRNFLA | CDR-L1 |
| 223 | WASTRQS | CDR-L2 |
| 224 | GDTFSDH | CDR-H1 |
| 225 | NPKIGN | CDR-H2 |
| 226 | THWMH | CDR-H1 |
| 227 | MINPSDGVTYYAQTFQG | CDR-H2 |
| 228 | EYYGEGFDY | CDR-H3 |
| 229 | RASQGISNYLA | CDR-L1 |
| 230 | SASNLQS | CDR-L2 |
| 231 | GYSLITH | CDR-H1 |
| 232 | NPSDGV | CDR-H2 |

What is claimed:

1. A method of transducing cells in subject, the method comprising:
(a) administering to a subject an inhibitor of the mammalian target of rapamycin (mTOR), and
(b) administering to the subject a viral vector comprising a viral fusogen embedded in the lipid bilayer.

2. The method of claim 1, wherein the viral vector comprises an exogenous agent.

3. A method of delivering an exogenous agent to a subject, the method comprising:
(a) administering to a subject an inhibitor of mTOR, and
(b) administering to the subject a viral vector comprising an exogenous agent, wherein the viral vector comprises a fusogen embedded in the lipid bilayer.

4. A method of transducing cells in a subject, the method comprising
(a) administering to a subject an inhibitor of the mammalian target of rapamycin (mTOR), and
(b) administering to the subject a lentiviral vector comprising one or more Nipah envelope proteins or functional variants thereof wherein the vector comprises a polynucleotide encoding a chimeric antigen receptor (CAR), and
(c) administering to the subject IL-7 or a functional variant thereof.

5. A method of transducing cells in a subject, the method comprising
(a) administering to a subject an inhibitor of the mammalian target of rapamycin (mTOR), and
(b) administering to the subject a lentiviral vector comprising one or more Nipah envelope proteins or functional variants thereof wherein the vector comprises
(i) a viral VPX protein, and
(ii) a polynucleotide encoding a chimeric antigen receptor (CAR).

6. The method of any of claims 1-5, wherein the inhibitor of mTOR and the viral vector are administered separately or in the same composition.

7. The method of any of claims 1-6, wherein the inhibitor of mTOR and the viral vector are administered separately.

8. The method of any of claims 1-5 and 7, wherein the inhibitor of mTOR is administered prior to, consecutively, or after administering the viral vector.

9. The method of any of claims 1-8, wherein the time period between the administration of the inhibitor of mTOR and viral vector is no more than three days.

10. The method of any of claims 1-9, wherein the time period between the administration of the inhibitor of mTOR and viral vector is no more than one day.

11. The method of any of claims 1-10, wherein the time period between the administration of the inhibitor of mTOR and viral vector is no more than 12, 6, or 3 hours.

12. The method of any of claims 1-11, wherein the inhibitor of mTOR and the viral vector are administered on the same day.

13. The method of any of claims 1-12, wherein the method further comprises administering to the subject an inhibitor of an antiviral restriction factor.

14. A method of transducing cells in subject, the method comprising:
(a) administering to a subject an inhibitor of an antiviral restriction factor, and
(b) administering to the subject a viral vector comprising a viral fusogen embedded in the lipid bilayer.

15. The method of claim 13 and 14, wherein the inhibitor of an antiviral restriction factor is an inhibitor of SAMHD1, an inhibitor of IFITM1, and/or an inhibitor of IFITM3.

16. The method of claim 13, 14, or 15, wherein the inhibitor of an antiviral restriction factor is an inhibitor of SAMHD1, optionally wherein the inhibitor increases phosphorylation and/or degradation of SAMHD1.

17. The method of claims 13, 14 or 15, wherein the inhibitor of an antiviral restriction factor is an inhibitor of IFITM1 and/or an inhibitor of IFITM3, optionally wherein the inhibitor reduces expression of IFITM1.

18. The method of claims 13-17, wherein the inhibitor of an antiviral restriction factor is an oligonucleotide, optionally wherein the inhibitor of an antiviral restriction factor is an anti-sense oligonucleotide complementary to an RNA encoding said cellular restriction factor.

19. The method of any of claims 13, 14, 15, or 17, wherein the inhibitor of an antiviral restriction factor is a resveratrol cyclotrimer, optionally caraphenol A, a-viniferin or resveratrol, or an analog compound thereof.

20. The method of any of claims 13-17, wherein the inhibitor of an antiviral restrictions factor is an antifungal agent.

21. The method of any claims 13-20, wherein the inhibitor of an antiviral restriction factor is a polyene antifungal agent, optionally nystatin, pimaricin, or amphotericin B.

22. The method of any of claim 13-21, wherein the inhibitor of an antiviral restriction factor is amphotericin B.

23. The method of any of claims 13-22, wherein the time period between the administration of the inhibitor of the antiviral restriction factor and viral vector is no more than one day.

24. The method of any of claims 13-23, wherein the time period between the administration of the inhibitor of the antiviral restriction factor and viral vector is no more than 12 hours.

25. The method of any of claims 13-24, wherein the time period between the administration of the inhibitor of the antiviral restriction factor and viral vector is no more than 1, 2, 3, 4, or 5 hours.

26. The method of any of claims 13-25, wherein the inhibitor of an antiviral restriction factor is administered at a dose of at or about 0.1-10 mg, 1-10 mg, 10-20 mg, 20-30 mg, 30-40 mg, or 40-50, or any value between the foregoing.

27. A method for administering a viral vector to a subject, the method comprising:
(a) contacting peripheral blood mononuclear cells (PBMCs) or a subset thereof from a subject with a composition comprising a viral vector and an inhibitor of mTOR to create a transduction mixture; and
(b) administering the transduction mixture to the subject, thereby administering the viral vector to the subject.

28. A method for administering a viral vector to a subject, the method comprising:
(a) contacting peripheral blood mononuclear cells (PBMCs) or a subset thereof from a subject with a composition comprising a viral vector and an inhibitor of mTOR that is temsirolimus to create a transduction mixture; and
(b) administering the transduction mixture to the subject, thereby administering the viral vector to the subject.

29. A method for administering a viral vector to a subject, the method comprising:
(a) contacting peripheral blood mononuclear cells (PBMCs) or a subset thereof from a subject with a composition comprising a viral vector and an inhibitor of an antiviral restriction factor to create a transduction mixture; and
(b) administering the transduction mixture to the subject, thereby administering the viral vector to the subject.

30. A method for administering a viral vector to a subject, the method comprising:
(a) contacting peripheral blood mononuclear cells (PBMCs) or a subset thereof from a subject with a composition comprising a viral vector and a polyene antifungal agent to create a transduction mixture; and
(b) administering the transduction mixture to the subject, thereby administering the viral vector to the subject.

31. The method of claims 23-30, wherein the contacting and administering is performed in a closed fluid circuit.

32. The method of claim 27, 28, or 31, wherein an inhibitor of mTOR is administered prior to contacting in step (A).

33. The method of claim 29, wherein the inhibitor of an antiviral restriction factor is administered prior to contacting in step (A).

34. The method of claim 30, wherein the polyene antifungal agent is administered prior to contacting in step (A).

35. The method of claim 30 or 34, wherein the polyene antifungal agent is selected from the group comprising nystatin, pimaricin, or amphotericin B.

36. A method for administering a viral vector to a subject, the method comprising:
(a) obtaining whole blood from a subject;
(b) collecting from the whole blood a fraction of blood containing PBMCs or a subset thereof;
(c) contacting the fraction of blood containing PBMCs or a subset thereof with a viral vector and an inhibitor of mTOR to create a transduction mixture; and
(d) reinfusing the transduction mixture to the subject, thereby administering the viral vector to the subject,
wherein steps (a)-(d) are performed in-line in a closed fluid circuit.

37. A method for administering a viral vector to a subject, the method comprising:
(a) obtaining whole blood from a subject;
(b) collecting from the whole blood a fraction of blood containing PBMCs or a subset thereof;
(c) contacting the fraction of blood containing PBMCs or a subset thereof with a viral vector and an inhibitor of an antiviral restriction factor to create a transduction mixture; and
(d) reinfusing the transduction mixture to the subject, thereby administering the viral vector to the subject,
wherein steps (a)-(d) are performed in-line in a closed fluid circuit.

38. A method for administering a viral vector to a subject, the method comprising:
(a) administering to a subject an inhibitor of mTOR;
(b) obtaining whole blood from a subject;
(c) collecting from the whole blood a fraction of blood containing PBMCs or a subset thereof;
(d) contacting the fraction of blood containing PBMCs or a subset thereof with a viral vector to create a transduction mixture; and
(e) reinfusing the transduction mixture to the subject, thereby administering the viral vector to the subject,
wherein steps (a)-(e) are performed in-line in a closed fluid circuit.

39. A method for administering a viral vector to a subject, the method comprising:
(a) administering to a subject an inhibitor of an antiviral restriction factor;
(b) obtaining whole blood from a subject;
(c) collecting from the whole blood a fraction of blood containing PBMCs or a subset thereof;
(d) contacting the fraction of blood containing PBMCs or a subset thereof with a viral vector to create a transduction mixture; and
(e) reinfusing the transduction mixture to the subject, thereby administering the viral vector to the subject,
wherein steps (a)-(e) are performed in-line in a closed fluid circuit.

40. The method of claim 38, wherein the method further comprises contacting the fraction of blood containing PBMCs or a subset thereof with a viral vector and an inhibitor of mTOR to create a transduction mixture in step (d).

41. The method of claim 39, wherein the method further comprises contacting the fraction of blood containing PBMCs or a subset thereof with a viral vector and an inhibitor of an antiviral restriction factor to create a transduction mixture in step (d).

42. The method of claim 27-41, wherein the viral vector comprises an exogenous agent.

43. A method for delivering an exogenous agent to a subject, the method comprising:
(a) contacting PBMCs or a subset thereof from a subject with a viral vector and an inhibitor of mTOR to create a transduction mixture, wherein the viral vector comprises an exogenous agent; and
(b) administering the transduction mixture to the subject, thereby administering the exogenous agent to the subject.

44. A method for delivering an exogenous agent to a subject, the method comprising:
(a) contacting PBMCs or a subset thereof from a subject with a viral vector and an inhibitor of mTOR that is temsirolimus to create a transduction mixture, wherein the viral vector comprises an exogenous agent; and
(b) administering the transduction mixture to the subject, thereby administering the exogenous agent to the subject.

45. A method for delivering an exogenous agent to a subject, the method comprising:
(a) contacting PBMCs or a subset thereof from a subject with a viral vector and an inhibitor of an antiviral restriction factor to create a transduction mixture, wherein the viral vector comprises an exogenous agent; and
(b) administering the transduction mixture to the subject, thereby administering the exogenous agent to the subject.

46. A method for delivering an exogenous agent to a subject, the method comprising:
(a) contacting PBMCs or a subset thereof from a subject with a viral vector and a polyene antifungal agent to create a transduction mixture, wherein the viral vector comprises an exogenous agent; and
(b) administering the transduction mixture to the subject, thereby administering the exogenous agent to the subject.

47. The method of claim 43-46, wherein the contacting and administering is performed in a closed fluid circuit.

48. The method of claim 43, 44, or 47, wherein an inhibitor of mTOR is administered prior to contacting in step (A)

49. The method of claim 45, wherein the inhibitor of an antiviral restriction factor is administered prior to contacting in step (A).

50. The method of claim 46, wherein the polyene antifungal agent is administered prior to contacting in step (A).

51. The method of claim 46 or 50, wherein the polyene antifungal agent is selected from the group comprising nystatin, pimaricin, or amphotericin B.

52. A method for delivering an exogenous agent to a subject, the method comprising:
(a) obtaining whole blood from a subject;
(b) collecting from the whole blood a fraction of blood containing peripheral blood mononuclear cells (PBMCs) or a subset thereof;
(c) contacting the fraction of blood containing peripheral blood mononuclear cells (PBMCs) or a subset thereof with a viral vector and an inhibitor of mTOR to create a transduction mixture, wherein the viral vector comprises an exogenous agent; and
(d) reinfusing the transduction mixture to the subject, thereby administering the exogenous agent to the subject,
wherein steps (a)-(d) are performed in-line in a closed fluid circuit.

53. A method for delivering an exogenous agent to a subject, the method comprising:
(a) obtaining whole blood from a subject;
(b) collecting from the whole blood a fraction of blood containing peripheral blood mononuclear cells (PBMCs) or a subset thereof;
(c) contacting the fraction of blood containing peripheral blood mononuclear cells (PBMCs) or a subset thereof with a viral vector and an inhibitor of an antiviral restriction factor to create a transduction mixture, wherein the viral vector comprises an exogenous agent; and
(d) reinfusing the transduction mixture to the subject, thereby administering the exogenous agent to the subject,
wherein steps (a)-(d) are performed in-line in a closed fluid circuit.

54. A method for delivering an exogenous agent to a subject, the method comprising:
(a) administering to the subject inhibitor of mTOR;
(b) obtaining whole blood from a subject;
(c) collecting from the whole blood a fraction of blood containing PBMCs or a subset thereof;
(d) contacting the fraction of blood containing PBMCs or a subset thereof with a viral vector to create a transduction mixture; and
(e) reinfusing the transduction mixture to the subject, thereby administering the viral vector to the subject,
wherein steps (a)-(e) are performed in-line in a closed fluid circuit.

55. A method for delivering an exogenous agent to a subject, the method comprising:
(a) administering to the subject inhibitor of an antiviral restriction factor;
(b) obtaining whole blood from a subject;
(c) collecting from the whole blood a fraction of blood containing PBMCs or a subset thereof;
(d) contacting the fraction of blood containing PBMCs or a subset thereof with a viral vector to create a transduction mixture; and
(e) reinfusing the transduction mixture to the subject, thereby administering the viral vector to the subject,
wherein steps (a)-(e) are performed in-line in a closed fluid circuit.

56. The method of claim 54, wherein the method further comprises contacting the fraction of blood containing PBMCs or a subset thereof with a viral vector and an inhibitor of mTOR to create a transduction mixture in step (d).

57. The method of claim 55, wherein the method further comprises contacting the fraction of blood containing PBMCs or a subset thereof with a viral vector and an inhibitor of an antiviral restriction factor to create a transduction mixture in step (d).

58. A method for administering a viral vector to a subject, the method comprising:
(a) contacting peripheral blood mononuclear cells (PBMCs) or a subset thereof from a subject with a composition comprising a lentiviral vector comprising one or more Nipah envelope proteins or functional variants thereof, an inhibitor of an antiviral restriction factor, and Il-7 or a functional variant thereof to create a transduction mixture; and
(b) administering the transduction mixture to the subject, thereby administering the viral vector to the subject, wherein the vector comprises a chimeric antigen receptor (CAR).

59. A method for administering a viral vector to a subject, the method comprising:
(a) contacting peripheral blood mononuclear cells (PBMCs) or a subset thereof from a subject with a composition comprising a lentiviral vector comprising one or more Nipah envelope proteins or functional variants thereof and an inhibitor of an antiviral restriction factor, to create a transduction mixture; and
(b) administering the transduction mixture to the subject, thereby administering the viral vector to the subject, wherein the vector comprises a viral VPX protein and a chimeric antigen receptor (CAR).

60. A method for transducing cells in a subject, the method comprising:
(a) contacting peripheral blood mononuclear cells (PBMCs) or a subset thereof from a subject with a composition comprising a lentiviral vector comprising one or more Nipah envelope proteins or functional variants thereof, and an inhibitor of an antiviral restriction factor to create a transduction mixture; and
(b) administering the transduction mixture to the subject, thereby administering the viral vector to the subject, wherein the vector comprises a viral VPX protein and a chimeric antigen receptor (CAR).

61. A method for transducing cells in a subject, the method comprising:
(a) contacting peripheral blood mononuclear cells (PBMCs) or a subset thereof from a subject with a composition comprising a lentiviral vector comprising one or more Nipah envelope proteins or functional variants thereof, an inhibitor of an antiviral restriction factor, and Il-7 or a functional variant thereof to create a transduction mixture; and
(b) administering the transduction mixture to the subject, thereby administering the viral vector to the subject, wherein the vector comprises a chimeric antigen receptor (CAR).

62. A method for administering a viral vector to a subject, the method comprising:
(a) contacting peripheral blood mononuclear cells (PBMCs) or a subset thereof from a subject with a composition comprising a lentiviral vector comprising one or more Nipah envelope proteins or functional variants thereof, an inhibitor of mTOR, and Il-7 or a functional variant thereof to create a transduction mixture; and
(b) administering the transduction mixture to the subject, thereby administering the viral vector to the subject, wherein the vector comprises a chimeric antigen receptor (CAR).

63. A method for administering a viral vector to a subject, the method comprising:
(a) contacting peripheral blood mononuclear cells (PBMCs) or a subset thereof from a subject with a composition comprising a lentiviral vector comprising one or more Nipah envelope proteins or functional variants thereof and an inhibitor of mTOR, to create a transduction mixture; and
(b) administering the transduction mixture to the subject, thereby administering the viral vector to the subject, wherein the vector comprises a viral VPX protein and a chimeric antigen receptor (CAR).

64. A method for transducing cells in a subject, the method comprising:
(a) contacting peripheral blood mononuclear cells (PBMCs) or a subset thereof from a subject with a composition comprising a lentiviral vector comprising one or more Nipah envelope proteins or functional variants thereof, and an inhibitor of mTOR to create a transduction mixture; and
(b) administering the transduction mixture to the subject, thereby administering the viral vector to the subject, wherein the vector comprises a viral VPX protein and a chimeric antigen receptor (CAR).

65. A method for transducing cells in a subject, the method comprising:
(a) contacting peripheral blood mononuclear cells (PBMCs) or a subset thereof from a subject with a composition comprising a lentiviral vector comprising one or more Nipah envelope proteins or functional variants thereof, an inhibitor of mTOR, and Il-7 or a functional variant thereof to create a transduction mixture; and
(b) administering the transduction mixture to the subject, thereby administering the viral vector to the subject, wherein the vector comprises a chimeric antigen receptor (CAR).

66. The method of any of claims 27-65, wherein the PBMCs or subset are further contacted with an inhibitor of an antiviral restriction factor.

67. The method of any of claims 27-65, wherein the transduction mixture further comprises an inhibitor of an antiviral restriction factor.

68. The method of claim 57-61, 66 or 67, wherein the inhibitor of an antiviral restriction factor is an inhibitor of SAMHD1, an inhibitor of IFITM1, and/or an inhibitor of IFITM3.

69. The method of claim 68, wherein the inhibitor of an antiviral restriction factor is an inhibitor of SAMHD1, optionally wherein the inhibitor increases phosphorylation and/or degradation of SAMHD1.

70. The method of claim 68, wherein the inhibitor of an antiviral restriction factor is an inhibitor of IFITM1, optionally wherein the inhibitor reduces expression of IFITM1.

71. The method of claims 57-61, 66-70, wherein the inhibitor of an antiviral restriction factor is an oligonucleotide, optionally wherein the inhibitor of an antiviral restriction factor is an anti-sense oligonucleotide complementary to an RNA encoding said cellular restriction factor.

72. The method of any of claims 57-61, 66-68, or 70, wherein the inhibitor of an antiviral restriction factor is a resveratrol cyclotrimer, optionally caraphenol A, a-viniferin or resveratrol, or an analog compound thereof.

73. The method of any of claims 57-61, 66-70, wherein the inhibitor of an antiviral restriction factor is an antifungal agent, optionally a polyene antifungal agent, further optionally amphotericin B.

74. The method of any of claims 57-61, 66-73, wherein the inhibitor of an antiviral restriction factor is contacted with PBMCs at a dose of at or about 0.1-10 μM, 1-10 μM, 10-20 μM, 20-M, 30-40 μM, 40-50 μM, 50-60 μM, 60-70 μM, 70-80 μM, 80-90 μM, or 90-100 μM, or any value between the foregoing.

75. The method of any of claims 27-74, wherein the method is carried out in a single in-line procedure to maintain a closed or functionally closed fluid circuit.

76. The method of any of claims 27-75, wherein the method is characterized by the whole blood, PBMCs or subset thereof, and transduction mixture having not been subjected to cryopreservation or freezing.

77. The method of any of claims 27-76, wherein the PBMCs or subset thereof, and transfection mixture are not formulated with a cryoprotectant (e.g., DMSO).

78. The method of any of claims 27-77, wherein the transduction mixture is directly reinfused to the subject, optionally without any further processing or washing steps.

79. The method of any of claims 31-42, or 47-56, or 75, wherein the closed fluid circuit comprises one or more of a blood processing set for obtaining the whole blood from the subject, a separation chamber for the separating the PBMCS or subset from the blood to collect the PBMCs or subset, a contacting container for the contacting the collected PBMCs or subset thereof with the composition comprising lipid particles (e.g. lentiviral vector), and a transfer container containing the contacted PBMCs or subset thereof and/or the transfection mixture for reinfusion to the subject.

80. The method of claim 79, wherein the closed fluid circuit further comprises a collection container operably connected to the separation chamber to collect the PBMCs or subset, optionally wherein the collection container is a bag, more optionally a sterile bag.

81. The method of any of claims 27-80, wherein during at least a portion of the contacting the method comprises mixing the transduction mixture comprising the PBMCs or subset and the composition comprising the viral vector.

82. The method of claim 81, wherein the mixing is by physical manipulation and/or centrifugation.

83. The method of any of claims 26-29, 33-55, wherein the collected fraction of blood contains PBMCs or subset thereof separated from other blood components.

84. The method of any of claims 36-42, 52-83, wherein collecting the fraction of blood is by apheresis.

85. The method of claim 84, wherein the apheresis device comprises membrane apheresis or centrifugal apheresis.

86. The method of any of claims 36-42, 52-85, wherein the collected fraction comprises leukocytes or precursors thereof.

87. The method of claim 86, wherein the precursors thereof comprise hematopoietic stem cells.

88. The method of any of claims 36-42, 52-87, wherein collecting the fraction of blood is by leukapheresis.

89. The method of claim 88, wherein the collected fraction of blood contains leukocytes.

90. The method of claim 13-26, 66-89, wherein the inhibitor of an antiviral restriction factor is a cytokine.

91. The method of claim 90, wherein the cytokine comprises IL-7, IL-15, or both IL-7 and IL-15.

92. The method of claims 13-26, 66-89, wherein the inhibitor of an antiviral restriction factor is an antifungal agent, optionally a polyene antifungal agent, further optionally amphotericin B.

93. The method of any of claims 1-92, wherein the inhibitor of mTOR is rapamycin or a rapamycin analogue.

94. The method of any of claims 1-93, wherein the inhibitor of mTOR is selected from the group comprising rapamycin, everolimus, temsirolimus, or ridaforolimus.

95. The method of any of claims 1-94, wherein the inhibitor of mTOR is rapamycin.

96. The method of any of claims 1-26 and 93-95, wherein the inhibitor of mTOR is administered at a dose of 1 mg to 1000 mg per day or 1 mg/m²/day to 500 mg/m²/day, or as a single dose of 1 mg to 1000 mg or 1 mg/m² to 500 mg/m²/dose.

97. The method of claim 96, wherein the inhibitor of mTOR is administered as a single dose of 2 mg to 50 mg.

98. The method of claim 96 or claim 97, wherein the inhibitor of mTOR is administered as a single dose of 25 mg.

99. The method of claim 96, wherein the inhibitor of mTOR is administered as a single dose of 100 mg/m² to 300 mg/m²/dose.

100. The method of claim 99, wherein the inhibitor of mTOR is administered at a dose of 220 mg/m²/dose.

101. The method of claim 96, wherein administration of the inhibitor of mTOR further comprising a loading dose.

102. The method of claim 101, wherein the loading dose is administered at a dose of 1 mg to 1000 mg per day or 1 mg/m²/day to 500 mg/m²/day, or as a single dose of 1 mg to 1000 mg or 1 mg/m2 to 500 mg/m²/dose.

103. The method of claim 101 or 102, wherein the loading dose is administered at a dose of 25 mg per day, 50 mg per day, or 500 mg per day.

104. The method of any of claims 1-26 and 93-103, wherein the inhibitor of mTOR is administered orally or intravenously, optionally wherein the inhibitor of mTOR is administered intravenously.

105. The method of any of claims 27-95, wherein the inhibitor of mTOR is contacted with the PBMCs or the subset thereof in an amount from 1 μM to 50 μM.

106. The method of any of claims 27-95, and 105, wherein the inhibitor of mTOR is contacted with the PBMCs or the subset thereof in an amount of at or about 5 μM, 10 μM, 15 μM, 20 μM, 25 μM, 30 μM, 35 μM, or 40 μM, or any value between any of the foregoing.

107. The method of any of claims 1-26, and 95-106 wherein the method further comprises administration of one or more recombinant cytokine to the subject.

108. The method of any of claims 25-65, wherein the PBMCs or subset are further contacted with one or more recombinant cytokine.

109. The method of claims 25-89, wherein the transduction mixture further comprises one or more recombinant cytokines.

110. The method of any of claims 107-109, wherein the one or more recombinant cytokines comprise recombinant IL-7, recombinant IL-15, or both recombinant IL-7 and recombinant IL-15.

111. The method of claim 109 or claim 110, wherein the one or more recombinant cytokine further comprises IL-2.

112. The method of claim 107, 108, 110, or 111, wherein the time period between the administration of the recombinant cytokine and viral vector is no more than one day.

113. The method of any of claims 107, 108, or 110-112, wherein the time period between the administration of the cytokine and viral vector is no more than 12 hours.

114. The method of any of claims 107, 108, or 110-113, wherein the time period between the administration of the cytokine and viral vector is no more than 1, 2, 3, 4, or 5 hours.

115. The method of any of claims 107-114, wherein the recombinant cytokine is administered at a dose of from at or about 0.001 mg/kg to at or about 0.1 mg/kg, at or about 0.001 mg/kg to at or about 0.05 mg/kg, at or about 0.001 mg/kg to at or about 0.01 mg/kg, at or about 0.01 mg/kg to at or about 0.1 mg/kg, at or about 0.01 mg/kg to at or about 0.05 mg/kg or at or about 0.05 mg/kg to at or about 0.1 mg/kg.

116. The method of claim 115, wherein the recombinant cytokine is administered at a dose of from or from about 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.02 mg/kg, 0.03 mg/kg, 0.04 mg/kg, or 0.05 mg/kg, or any value between any of the foregoing.

117. The method of any of claims 1-4, 6-62, 65-116, wherein the viral vector further comprises a viral accessory protein, optionally wherein the viral accessory protein is a viral kinase.

118. The method of claim 117, wherein the viral accessory protein is an inhibitor of an antiviral restriction factor, optionally wherein the viral accessory protein is an inhibitor of SAMHD1 activity.

119. The method of claim 117 or claim 118, wherein the viral accessory protein directly or indirectly phosphorylates SAMHD1.

120. The method of claims 117-119, wherein the viral accessory protein is selected from the group consisting of: BLG4, UL97, and U69.

121. The method of claims 117 or 118, wherein the viral accessory protein degrades SAMHD1.

122. The method of any of claims 117-121, wherein the viral accessory protein is a fusion protein, optionally a fusion protein with VPX and/or Vpr.

123. The method of any of claims 1-3, 6-61, 66-122, wherein the viral vector is a retroviral vector.

124. The method of any of claims 1-3, 6-61, 66-123, wherein the viral vector is a lentiviral vector.

125. The method of any of claims 1-3, 6-61, 66-124, wherein the viral vector is pseudotyped with the fusogen.

126. The method of any of claims 1-3, 6-61, 66-125, wherein the viral fusogen is selected from a Class I viral membrane fusion protein, a Class II viral membrane protein, a Class II viral membrane fusion protein, a viral membrane glycoprotein, or a viral envelope protein.

127. The method of any of claims 1-3, 6-61, 66-126, wherein the viral fusogen comprises a viral envelope protein or a functional variant thereof.

128. The method of any of claims 1-3, 6-61, 66-127, wherein the viral fusogen is a vesicular stomatitis virus envelope glycoprotein (VSV-G).

129. The method of 1-3, 6-61, 66-127, wherein the viral fusogen is a baboon endogenous virus (BaEV) envelope glycoprotein.

130. The method of 1-3, 6-61, 66-127, wherein the viral fusogen is a Cocal virus envelope glycoprotein.

131. The method of 1-3, 6-61, 66-127, wherein the viral fusogen is an Alphavirus class II fusion protein or a functional variant thereof, optionally wherein the Alphavirus is a Sindbis virus.

132. The method of 1-3, 6-61, 66-127, wherein the viral fusogen comprises a Paramyxovirus fusion (F) protein or a biologically active portion thereof, optionally wherein the Paramyxovirus is a Morbillivirus or a Henipavirus.

133. The method of any of claims 1-3, 6-61, 66-127, and 132, wherein the viral fusogen comprises a Morbillivirus fusion (F) protein.

134. The method of claim 133, wherein the Morbillivirus F protein is from a measles virus (MeV), canine distemper virus, Cetacean morbillivirus, Peste-des-petits-ruminants virus, Phocine distemper virus, Rinderpest virus or a biologically active portion or functional variant thereof of any of the foregoing.

135. The method of any of claims 1-3, 6-61, 66-127, and 132, wherein the viral fusogen comprises a Henipavirus F protein from a Nipah virus, Hendra virus, Cedar virus, Kumasi virus, Mojiang virus or a biologically active portion or functional variant thereof.

136. The method of any of claims 1-3, 6-61, 66-127, 132 and 135, wherein the viral fusogen comprises a Nipah virus F protein or a biologically active portion or functional variant thereof.

137. The method of any of claims 132-136, wherein the fusogen comprises a paramyxovirus G, paramyxovirus H, or paramyxovirus HN protein, or a biologically active portion or functional variant thereof.

138. The method of claim 137, wherein the paramyxovirus G, paramyxovirus H, or paramyxovirus HN protein further comprises a targeting moiety that binds to a molecule on a target cell.

139. The method of any of claims 1-3, 6-61, 66-127, and 132-138, wherein the viral fusogen comprises an F protein molecule or a biologically active portion thereof from a Paramyxovirus and a glycoprotein G (G protein) or a biologically active portion thereof from a Paramyxovirus.

140. The method of claim 139, wherein the Paramyxovirus is a henipavirus.

141. The method of claim 139 or claim 140, wherein the Paramyxovirus is Nipah virus.

142. The method of any of claims 1-127 and 132-141, wherein the viral fusogen and/or Nipah envelope protein comprises a Nipah virus F glycoprotein (NiV-F) or a biologically active portion or functional variant thereof and a Nipah virus G glycoprotein (NiV-G) or a biologically active portion or functional variant thereof.

143. The method of claim 139 or claim 140, wherein the Paramyxovirus is Hendra virus.

144. The method of any of claims 139-142, wherein the G protein or the biologically active portion thereof is a mutant NiV-G protein or biologically active portion thereof that exhibits reduced binding to Ephrin B2 or Ephrin B3.

145. The method of claim 144, wherein the mutant NiV-G protein comprises one or more amino acid substitutions corresponding to amino acid substitutions selected from the group consisting of E501A, W504A, Q530A and E533A with reference to numbering set forth in SEQ ID NO:14.

146. The method of any of claims 139-142, 144 and 145, wherein the G protein or biologically active portion is a biologically active portion of wild-type NiV-G that has a deletion of up to 40 amino acids at or near the N-terminus, optionally not including the initial methionine.

147. The method of any of claims 139-142, and 144-146, wherein the G protein is a biologically active portion that is a truncated NiV-G that has a deletion of amino acids 2-34 at or near the N-terminus of wild-type NiV-G set forth in SEQ ID NO:14.

148. The method of any of claims 139-142, and 144-147, wherein the G protein or the biologically active portion has the amino acid sequence set forth in SEQ ID NO: 19 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:19.

149. The method of any of claims 139-142, and 144-146, wherein the F protein or the biologically active portion thereof is a NiV-F protein or a biologically active portion thereof.

150. The method of claim 149, wherein the F protein or the biologically active portion is a truncated NiV-F that is truncated by at least or at 22 amino acids or at least or at 20 amino acids at or near the C-terminus of wild-type NiV-F set forth in SEQ ID NO:2, optionally not including the initial methionine.

151. The method of any of claims 139-142, and 144-150, wherein the F protein or the biologically active portion is a truncated NiV-F that lacks amino acids 525-546 of SEQ ID NO:2.

152. The method of any of claims 139-142, and 144-151, wherein the F protein or the biologically active portion has the amino acid sequence set forth in SEQ ID NO: 12 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:12.

153. The method of any of claims 139-142, and 144-151, wherein the NiV-G protein comprises the amino acid sequence set forth in SEQ ID NO: 19, and the NiV-F protein comprises the amino acid sequence set forth in SEQ ID NO:12.

154. The method of any of claims 138-153, wherein the targeted moiety is specific for a cell surface receptor on a target cell.

155. The method of any of claim 138-154, wherein the targeting domain is a Design ankyrin repeat proteins (DARPin), a single domain antibody (sdAb), a VHH fragment, a single chain variable fragment (scFv), or an antigen-binding fibronectin type III (Fn3) scaffold.

156. The method of any one of claims 138-155, wherein the fusogen and the targeting moiety are directly linked.

157. The method of any one of claims 138-156, wherein the fusogen and targeting moiety are indirectly linked via a linker.

158. The method of claim 157, wherein the linker is a peptide linker.

159. The method of claim 158, wherein the peptide linker is (GmS)n (SEQ ID NO: 11), wherein each of m and n is an integer between 1 to 4, inclusive.

160. The method of any of claims 2-3, 6-26, 42-56, 66-159, wherein the exogenous agent is a nucleic acid or a polypeptide.

161. The method of claim 160, wherein the exogenous agent is a nucleic acid encoding a payload gene, optionally wherein the nucleic acid encodes a chimeric antigen receptor.

162. The method of claim 154, wherein the target cell is one or more of a monocyte, macrophage, neutrophil, dendritic cell, eosinophil, mast cell, platelet, large granular lymphocyte, Langerhans' cell, natural killer (NK) cell, T lymphocyte (e.g., T cell), a Gamma delta T cell, B lymphocyte (e.g., B cell), CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, enuma Her2/Neu+ cancer cell, a GluA2+ neuron, a GluA4+ neuron, a NKG2D+ natural killer cell, a SLC1A3+ astrocyte, a SLC7A10+ adipocyte, a CD30+ lung epithelial cell, a liver sinusoidal endothelial cell or myocyte.

163. The method of claim 162, wherein the target cell is a T cell, optionally wherein the target cell is a resting T cell.

164. The method of claim 162 or 163, wherein the target cell is a T cell that has not been activated.

165. The method of any of claims 139-142 and 144-164, wherein the viral vector comprises
(i) a re-targeted Nipah virus G glycoprotein (NiV-G) that is a truncated NiV-G set forth in SEQ ID NO:19 linked to a targeting moiety that binds to a T cell, and
(ii) a truncated Nipah virus F glycoprotein (NiV-F) set forth in SEQ ID NO:12.

166. The method of claim 139-165, wherein the targeting moiety that binds to a T cell is a CD8 binding agent, CD4 binding agent or CD3 binding agent

167. The method of claim 165 or claim 166, wherein the targeting moiety is an sdAb or an ScFv.

168. A composition, comprising the transduction mixture of the method of any of claims 27-167.

169. A composition comprising a leukapheresis composition for delivering a viral vector to a subject, wherein the leukapheresis composition comprises peripheral blood mononuclear cells (PBMCs) or a subset thereof from the subject, a viral vector, and an inhibitor of mTOR.

170. A composition comprising a leukapheresis composition for delivering a viral vector to a subject, wherein the leukapheresis composition comprises peripheral blood mononuclear cells (PBMCs) or a subset thereof from the subject, a viral vector, and an inhibitor of an antiviral restriction factor.

171. The composition of claim 169 or claim 170, wherein the delivery to the subject is with an apheresis device.

172. A method of treating a disease or condition in a subject comprising administering a viral vector or exogenous agent by the method of any of claims 1-167 to a subject in need thereof.

173. A method of treating a disease or condition comprising infusing the composition of any of claims 168-170 into a subject in need thereof.

174. The method of claim 172 or claim 173, wherein the disease or disorder is treatable by administration of the viral vector or the exogenous agent.

175. The method of any of claims 172-174, wherein the disease or condition is a cancer.

176. The lipid particle therapy or method of any of claims 172-175, wherein the cancer is a solid tumor, a lymphoma or a leukemia.


SEQUENCE LISTING (232 sequences)

SEQ ID NO: 1 - protein, 546 aa, synthetic construct
  REGION 1..546: note = Hendra F
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 2 - protein, 546 aa, synthetic construct
  REGION 1..546: note = Nipah F0
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 3 - protein, 557 aa, synthetic construct
  REGION 1..557: note = CedV F
MSNKRTTVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 4 - protein, 545 aa, synthetic construct
  REGION 1..545: note = Mojiang F
MALNKNMFSSLFLGYLLVYATTVQSSIHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 5 - protein, 662 aa, synthetic construct
  REGION 1..662: note = Bat PV F
MKKKTDNPTISKRGHNHSRGIKSRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 6 - protein, 520 aa, synthetic construct
  REGION 1..520: note = Hendra virus F Protein, Without signal sequence
ILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 7 - protein, 520 aa, synthetic construct
  REGION 1..520: note = Nipah virus F Protein, without signal sequence
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 8 - protein, 551 aa, synthetic construct
  REGION 1..551: note = Cedar Virus F Protein, without signal sequence
TVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 9 - protein, 519 aa, synthetic construct
  REGION 1..519: note = Mojiang virus, Tongguan 1 F Protein, without signal sequence
IHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 10 - protein, 640 aa, synthetic construct
  REGION 1..640: note = Bat Paramyxovirus F Protein, without signal sequence
SRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 11 - protein, 524 aa, synthetic construct
  REGION 1..524: note = Truncated NiV fusion glycoprotein (FcDelta22) at cytoplasmic tail (with signal sequence)
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 12 - protein, 498 aa, synthetic construct
  REGION 1..498: note = Truncated mature NiV fusion glycoprotein (FcDelta22) at cytoplasmic tail
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 13 - protein, 604 aa, synthetic construct
  REGION 1..604: note = Hendra virus G protein Uniprot O89343
MMADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 14 - protein, 602 aa, synthetic construct
  REGION 1..602: note = Uniprot Q9IH62
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 15 - protein, 622 aa, synthetic construct
  REGION 1..622: note = gb:JQ001776:8170-10275
MLSQLQKNYLDNSNQQGDKMNNPDKKLSVNFNPLELDKGQKDLNKSYYVKNKNYNVSNLLNESLHDIKFCIYCIFSLLIIITIINIITISIVITRLKVHEENNGMESPNLQSIQDSLSSLTNMINTEITPRIGILVTATSVTLSSSINYVGTKTNQLVNELKDYITKSCGFKVPELKLHECNISCADPKISKSAMYSTNAYAELAGPPKIFCKSVSKDPDFRLKQIDYVIPVQQDRSICMNNPLLDISDGFFTYIHYEGINSCKKSDSFKVLLSHGEIVDRGDYRPSLYLLSSHYHPYSMQVINCVPVTCNQSSFVFCHISNNTKTLDNSDYSSDEYYITYFNGIDRPKTKKIPINNMTADNRYIHFTFSGGGGVCLGEEFIIPVTTVINTDVFTHDYCESFNCSVQTGKSLKEICSESLRSPTNSSRYNLNGIMIISQNNMTDFKIQLNGITYNKLSFGSPGRLSKTLGQVLYYQSSMSWDTYLKAGFVEKWKPFTPNWMNNTVISRPNQGNCPRYHKCPEICYGGTYNDIAPLDLGKDMYVSVILDSDQLAENPEITVFNSTTILYKERVSKDELNTRSTTTSCFLFLDEPWCISVLETNRFNGKSIRPEIYSYKIPKYC

SEQ ID NO: 16 - protein, 632 aa, synthetic construct
  REGION 1..632: note = gb:NC_025256:9117-11015|Organism:Bat Paramyxovirus Eid_hel/GH-M74a/GHA/2009|Strain Name:BatPV/Eid_hel/GH-M74a/GHA/2009|Protein Name:glycoprotein|Gene Symbol:G
MPQKTVEFINMNSPLERGVSTLSDKKTLNQSKITKQGYFGLGSHSERNWKKQKNQNDHYMTVSTMILEILVVLGIMFNLIVLTMVYYQNDNINQRMAELTSNITVLNLNLNQLTNKIQREIIPRITLIDTATTITIPSAITYILATLTTRISELLPSINQKCEFKTPTLVLNDCRINCTPPLNPSDGVKMSSLATNLVAHGPSPCRNFSSVPTIYYYRIPGLYNRTALDERCILNPRLTISSTKFAYVHSEYDKNCTRGFKYYELMTFGEILEGPEKEPRMFSRSFYSPTNAVNYHSCTPIVTVNEGYFLCLECTSSDPLYKANLSNSTFHLVILRHNKDEKIVSMPSFNLSTDQEYVQIIPAEGGGTAESGNLYFPCIGRLLHKRVTHPLCKKSNCSRTDDESCLKSYYNQGSPQHQVVNCLIRIRNAQRDNPTWDVITVDLTNTYPGSRSRIFGSFSKPMLYQSSVSWHTLLQVAEITDLDKYQLDWLDTPYISRPGGSECPFGNYCPTVCWEGTYNDVYSLTPNNDLFVTVYLKSEQVAENPYFAIFSRDQILKEFPLDAWISSARTTTISCFMFNNEIWCIAALEITRLNDDIIRPIYYSFWLPTDCRTPYPHTGKMTRVPLRSTYNY

SEQ ID NO: 17 - protein, 625 aa, synthetic construct
  REGION 1..625: note = gb:NC_025352:8716-11257|Organism:Mojiang virus|Strain Name:Tongguan1|Protein Name:attachment glycoprotein|Gene Symbol:G
MATNRDNTITSAEVSQEDKVKKYYGVETAEKVADSISGNKVFILMNTLLILTGAIITITLNITNLTAAKSQQNMLKIIQDDVNAKLEMFVNLDQLVKGEIKPKVSLINTAVSVSIPGQISNLQTKFLQKYVYLEESITKQCTCNPLSGIFPTSGPTYPPTDKPDDDTTDDDKVDTTIKPIEYPKPDGCNRTGDHFTMEPGANFYTVPNLGPASSNSDECYTNPSFSIGSSIYMFSQEIRKTDCTAGEILSIQIVLGRIVDKGQQGPQASPLLVWAVPNPKIINSCAVAAGDEMGWVLCSVTLTAASGEPIPHMFDGFWLYKLEPDTEVVSYRITGYAYLLDKQYDSVFIGKGGGIQKGNDLYFQMYGLSRNRQSFKALCEHGSCLGTGGGGYQVLCDRAVMSFGSEESLITNAYLKVNDLASGKPVIIGQTFPPSDSYKGSNGRMYTIGDKYGLYLAPSSWNRYLRFGITPDISVRSTTWLKSQDPIMKILSTCTNTDRDMCPEICNTRGYQDIFPLSEDSEYYTYIGITPNNGGTKNFVAVRDSDGHIASIDILQNYYSITSATISCFMYKDEIWCIAITEGKKQKDNPQRIYAHSYKIRQMCYNMKSATVTVGNAKNITIRRY

SEQ ID NO: 18 - protein, 622 aa, synthetic construct; identical to SEQ ID NO: 15
  REGION 1..622: note = gb:JQ001776:8170-10275|Organism:Cedar virus|Strain Name:CG1a|Protein Name:attachment glycoprotein|Gene Symbol:G

SEQ ID NO: 19 - protein, 568 aa, synthetic construct
  REGION 1..568: note = (E501 A, W504A, Q530A, E533A) NiV G protein (Gcdel34)
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQC

SEQ ID NO: 20 - protein, 5 aa, synthetic construct
  REGION 1..5: note = linker
GGGGS

SEQ ID NO: 21 - protein, 6 aa, synthetic construct
  REGION 1..6: note = Linker
GGGGGS

SEQ ID NO: 22 - protein, 5 aa, synthetic construct
  REGION 1..5: note = Linker
  REPEAT 1..5: note = Repeat any positive whole integer number of times
GGGGS

SEQ ID NO: 23 - protein, 6 aa, synthetic construct
  REGION 1..6: note = Linker
  REPEAT 1..6: note = Repeat any positive whole integer number of times
GGGGGS

SEQ ID NO: 24 - protein, 21 aa, synthetic construct
  REGION 1..21: note = CD8aplpha signal peptide
MALPVTALLLPLALLLHAARP

SEQ ID NO: 25 - protein, 20 aa, synthetic construct
  REGION 1..20: note = IgK signal peptide
METDTLLLWVLLLWVPGSTG

SEQ ID NO: 26 - protein, 22 aa, synthetic construct
  REGION 1..22: note = GMCSFR-aplha (CSF2RA) signal peptide
MLLLVTSLLLCELPHPAFLLIP

SEQ ID NO: 27 - protein, 45 aa, synthetic construct
  REGION 1..45: note = CD8aplha hinge domain
TTTPAPRPPTPAPTIASQPLSLRPEACRPAAGGAVHTRGLDFACD

SEQ ID NO: 28 - protein, 39 aa, synthetic construct
  REGION 1..39: note = CD28 hinge domain
IEVMYPPPYLDNEKSNGTIIHVKGKHLCPSPLFPGPSKP

SEQ ID NO: 29 - protein, 42 aa, synthetic construct
  REGION 1..42: note = CD28 hinge domain
AAAIEVMYPPPYLDNEKSNGTIIHVKGKHLCPSPLFPGPSKP

SEQ ID NO: 30 - protein, 12 aa, synthetic construct
  REGION 1..12: note = IgG4 hinge domain
ESKYGPPCPPCP

SEQ ID NO: 31 - protein, 12 aa, synthetic construct
  REGION 1..12: note = IgG4 hinge domain
ESKYGPPCPSCP

SEQ ID NO: 32 - protein, 229 aa, synthetic construct
  REGION 1..229: note = IgG4 hinge-CH2-CH3 domain
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 24 aa, synthetic construct
  REGION 1..24: note = CD8aplha transmembrane domain
IYIWAPLAGTCGVLLLSLVITLYC

SEQ ID NO: 34 - protein, 27 aa, synthetic construct
  REGION 1..27: note = CD28 transmembrane domain
FWVLVVVGGVLACYSLLVTVAFIIFWV

SEQ ID NO: 35 - protein, 28 aa, synthetic construct
  REGION 1..28: note = CD28 transmembrane domain
MFWVLVVVGGVLACYSLLVTVAFIIFWV

SEQ ID NO: 36 - protein, 42 aa, synthetic construct
  REGION 1..42: note = 4-1BB costimulatory domain
KRGRKKLLYIFKQPFMRPVQTTQEEDGCSCRFPEEEEGGCEL

SEQ ID NO: 37 - protein, 41 aa, synthetic construct
  REGION 1..41: note = CD28 costimulatory domain
RSKRSRLLHSDYMNMTPRRPGPTRKHYQPYAPPRDFAAYRS

SEQ ID NO: 38 - protein, 112 aa, synthetic construct
  NON_STD 1..112: note = CD3z signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 112 aa, synthetic construct
  REGION 1..112: note = CD3z signaling domain (with Q to K mutation at position 14)
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 245 aa, synthetic construct
  REGION 1..245: note = Anti-CD19 FMC63 scFv entire sequence, with Whitlow linker
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 41 - protein, 107 aa, synthetic construct
  REGION 1..107: note = Anti-CD19 FMC63 scFv light chain variable region
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 42 - protein, 6 aa, synthetic construct
  REGION 1..6: note = Anti-CD19 FMC63 scFv light chain CDR1
QDISKY

SEQ ID NO: 43 - skipped

SEQ ID NO: 44 - protein, 9 aa, synthetic construct
  REGION 1..9: note = Anti-CD19 FMC63 scFv light chain CDR3
QQGNTLPYT

SEQ ID NO: 45 - protein, 18 aa, synthetic construct
  REGION 1..18: note = Whitlow linker
GSTSGSGKPGSGEGSTKG

SEQ ID NO: 46 - protein, 120 aa, synthetic construct
  REGION 1..120: note = Anti-CD19 FMC63 scFv heavy chain variable region
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 47 - protein, 8 aa, synthetic construct
  REGION 1..8: note = Anti-CD19 FMC63 scFv heavy chain CDR1
GVSLPDYG

SEQ ID NO: 48 - protein, 7 aa, synthetic construct
  REGION 1..7: note = Anti-CD19 FMC63 scFv heavy chain CDR2
IWGSETT

SEQ ID NO: 49 - protein, 14 aa, synthetic construct
  REGION 1..14: note = Anti-CD19 FMC63 scFv heavy chain CDR3
AKHYYYGGSYAMDY

SEQ ID NO: 50 - protein, 242 aa, synthetic construct
  REGION 1..242: note = Anti-CD19 FMC63 scFv entire sequence, with 3xG4S linker
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 51 - protein, 15 aa, synthetic construct
  REGION 1..15: note = 3xG4S linker
GGGGSGGGGSGGGGS

SEQ ID NO: 52 - DNA, 1467 bp, synthetic construct
  source 1..1467: mol_type = other DNA
  misc_feature 1..1467: note = Exemplary CD19 CAR nucleotide sequence
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 53 - protein, 489 aa, synthetic construct
  REGION 1..489: note = Exemplary CD19 CAR amino acid sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - DNA, 1458 bp, synthetic construct
  source 1..1458: mol_type = other DNA
  misc_feature 1..1458: note = Tisagenlecleucel CD19 CAR nucleotide sequence
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 55 - protein, 486 aa, synthetic construct
  REGION 1..486: note = Tisagenlecleucel CD19 CAR amino acid sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - DNA, 1383 bp, synthetic construct
  source 1..1383: mol_type = other DNA
  misc_feature 1..1383: note = Lisocabtagene maraleucel CD19 CAR nucleotide sequence
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 57 - protein, 461 aa, synthetic construct
  REGION 1..461: note = Lisocabtagene maraleucel CD19 CAR amino acid sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - DNA, 1467 bp, synthetic construct
  source 1..1467: mol_type = other DNA
  misc_feature 1..1467: note = Axicabtagene ciloleucel CD19 CAR nucleotide sequence
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 59 - protein, 489 aa, synthetic construct
  REGION 1..489: note = Axicabtagene ciloleucel CD19 CAR amino acid sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 246 aa, synthetic construct
  REGION 1..246: note = Anti-CD20 Leu16 scFv entire sequence, with Whitlow linker
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGSGKPGSGEGSTKGEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 61 - protein, 106 aa, synthetic construct
  REGION 1..106: note = Anti-CD20 Leu16 scFv light chain variable region
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 62 - protein, 10 aa, synthetic construct
  REGION 1..10: note = Anti-CD20 Leu16 scFv light chain CDR1
RASSSVNYMD

SEQ ID NO: 63 - protein, 7 aa, synthetic construct
  REGION 1..7: note = Anti-CD20 Leu16 scFv light chain CDR2
ATSNLAS

SEQ ID NO: 64 - protein, 9 aa, synthetic construct
  REGION 1..9: note = Anti-CD20 Leu16 scFv light chain CDR3
QQWSFNPPT

SEQ ID NO: 65 - protein, 122 aa, synthetic construct
  REGION 1..122: note = Anti-CD20 Leu16 scFv heavy chain
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 66 - protein, 5 aa, synthetic construct
  REGION 1..5: note = Anti-CD20 Leu16 scFv heavy chain CDR1
SYNMH

SEQ ID NO: 67 - protein, 17 aa, synthetic construct
  REGION 1..17: note = Anti-CD20 Leu16 scFv heavy chain CDR2
AIYPGNGDTSYNQKFKG

SEQ ID NO: 68 - protein, 13 aa, synthetic construct
  REGION 1..13: note = Anti-CD20 Leu16 scFv heavy chain CDR3
SNYYGSSYWFFDV

SEQ ID NO: 69 - protein, 246 aa, synthetic construct
  REGION 1..246: note = Anti-CD22 m971 scFv entire sequence, with 3xG4S linker
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 70 - protein, 124 aa, synthetic construct
  REGION 1..124: note = Anti-CD22 m971 scFv heavy chain variable region
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 71 - protein, 10 aa, synthetic construct
  REGION 1..10: note = Anti-CD22 m971 scFv heavy chain CDR1
GDSVSSNSAA

SEQ ID NO: 72 - protein, 9 aa, synthetic construct
  REGION 1..9: note = Anti-CD22 m971 scFv heavy chain CDR2
TYYRSKWYN

SEQ ID NO: 73 - protein, 14 aa, synthetic construct
  REGION 1..14: note = Anti-CD22 m971 scFv heavy chain CDR3
AREVTGDLEDAFDI

SEQ ID NO: 74 - protein, 107 aa, synthetic construct
  REGION 1..107: note = Anti-CD22 m971 scFv light chain
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 75 - protein, 6 aa, synthetic construct
  REGION 1..6: note = Anti-CD22 m971 scFv light chain CDR1
QTIWSY

SEQ ID NO: 76 - skipped

SEQ ID NO: 77 - protein, 9 aa, synthetic construct
  REGION 1..9: note = Anti-CD22 m971 scFv light chain CDR3
QQSYSIPQT

SEQ ID NO: 78 - protein, 246 aa, synthetic construct
  REGION 1..246: note = Anti-CD22 m971-L7 scFv entire sequence, with 3xG4S linker
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 79 - protein, 124 aa, synthetic construct
  REGION 1..124: note = Anti-CD22 m971-L7 scFv heavy chain variable region
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSS

SEQ ID NO: 80 - protein, 10 aa, synthetic construct
  REGION 1..10: note = Anti-CD22 m971-L7 scFv heavy chain CDR1
GDSVSSNSVA

SEQ ID NO: 81 - protein, 9 aa, synthetic construct
  REGION 1..9: note = Anti-CD22 m971-L7 scFv heavy chain CDR2
TYYRSTWYN

SEQ ID NO: 82 - protein, 14 aa, synthetic construct
  REGION 1..14: note = Anti-CD22 m971-L7 scFv heavy chain CDR3
AREVTGDLEDAFDI

SEQ ID NO: 83 - protein, 107 aa, synthetic construct
  REGION 1..107: note = Anti-CD22 m971-L7 scFv light chain variable region
DIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 84 - protein, 6 aa, synthetic construct
  REGION 1..6: note = Anti-CD22 m971-L7 scFv light chain CDR1
QTIWSY

SEQ ID NO: 85 - skipped

SEQ ID NO: 86 - protein, 9 aa, synthetic construct
  REGION 1..9: note = Anti-CD22 m971-L7 scFv light chain CDR3
QQSYSIPQT

SEQ ID NO: 87 - protein, 246 aa, synthetic construct
  REGION 1..246: note = Anti-BCMA C11D5.3 scFv entire sequence, with Whitlow linker
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 88 - protein, 111 aa, synthetic construct
  REGION 1..111: note = Anti-BCMA C11D5.3 scFv light chain variable region
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 89 - protein, 15 aa, synthetic construct
  REGION 1..15: note = Anti-BCMA C11D5.3 scFv light chain CDR1
RASESVSVIGAHLIH

SEQ ID NO: 90 - protein, 7 aa, synthetic construct
  REGION 1..7: note = Anti-BCMA C11D5.3 scFv light chain CDR2
LASNLET

SEQ ID NO: 91 - protein, 9 aa, synthetic construct
  REGION 1..9: note = Anti-BCMA C11D5.3 scFv light chain CDR3
LQSRIFPRT

SEQ ID NO: 92 - protein, 117 aa, synthetic construct
  REGION 1..117: note = Anti-BCMA C11D5.3 scFv heavy chain variable region
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 93 - protein, 5 aa, synthetic construct
  REGION 1..5: note = Anti-BCMA C11D5.3 scFv heavy chain CDR1
DYSIN

SEQ ID NO: 94 - protein, 17 aa, synthetic construct
  REGION 1..17: note = Anti-BCMA C11D5.3 scFv heavy chain CDR2
WINTETREPAYAYDFRG

SEQ ID NO: 95 - protein, 8 aa, synthetic construct
  REGION 1..8: note = Anti-BCMA C11D5.3 scFv heavy chain CDR3
DYSYAMDY

SEQ ID NO: 96 - protein, 246 aa, synthetic construct
  REGION 1..246: note = Anti-BCMA C12A3.2 scFv entire sequence, with Whitlow linker
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 97 - protein, 111 aa, synthetic construct
  REGION 1..111: note = Anti-BCMA C12A3.2 scFv light chain variable region
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 98 - protein, 15 aa, synthetic construct
  REGION 1..15: note = Anti-BCMA C12A3.2 scFv light chain CDR1
RASESVTILGSHLIY

SEQ ID NO: 99 - protein, 7 aa, synthetic construct
  REGION 1..7: note = Anti-BCMA C12A3.2 scFv light chain CDR2
LASNVQT

SEQ ID NO: 100 - protein, 9 aa, synthetic construct
  REGION 1..9: note = Anti-BCMA C12A3.2 scFv light chain CDR3
LQSRTIPRT

SEQ ID NO: 101 - protein, 117 aa, synthetic construct
  REGION 1..117: note = Anti-BCMA C12A3.2 scFv heavy chain variable region
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 102 - protein, 5 aa, synthetic construct
  REGION 1..5: note = Anti-BCMA C12A3.2 scFv heavy chain CDR1
HYSMN

SEQ ID NO: 103 - protein, 17 aa, synthetic construct
  REGION 1..17: note = Anti-BCMA C12A3.2 scFv heavy chain CDR2
RINTESGVPIYADDFKG

SEQ ID NO: 104 - protein, 8 aa, synthetic construct
  REGION 1..8: note = Anti-BCMA C12A3.2 scFv heavy chain CDR3
DYLYSLDF

SEQ ID NO: 105 - protein, 121 aa, synthetic construct
  REGION 1..121: note = Anti-BCMA FHVH33 entire sequence
EVQLLESGGGLVQPGGSLRLSCAASGFTFSSYAMSWVRQAPGKGLEWVSSISGSGDYIYYADSVKGRFTISRDISKNTLYLQMNSLRAEDTAVYYCAKEGTGANSSLADYRGQGTLVTVSS

SEQ ID NO: 106 - protein, 8 aa, synthetic construct
  REGION 1..8: note = Anti-BCMA FHVH33 CDR1
GFTFSSYA

SEQ ID NO: 107 - protein, 8 aa, synthetic construct
  REGION 1..8: note = Anti-BCMA FHVH33 CDR2
ISGSGDYI

SEQ ID NO: 108 - protein, 14 aa, synthetic construct
  REGION 1..14: note = Anti-BCMA FHVH33 CDR3
AKEGTGANSSLADY

SEQ ID NO: 109 - protein, 243 aa, synthetic construct
  REGION 1..243: note = Anti-BCMA CT103A scFv entire sequence, with Whitlow linker
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 110 - protein, 106 aa, synthetic construct
  REGION 1..106: note = Anti-BCMA CT103A scFv light chain variable region
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIK

SEQ ID NO: 111 - protein, 6 aa, synthetic construct
  REGION 1..6: note = Anti-BCMA CT103A scFv light chain CDR1
QSISSY

SEQ ID NO: 112 - skipped

SEQ ID NO: 113 - protein, 8 aa, synthetic construct
  REGION 1..8: note = Anti-BCMA CT103A scFv light chain CDR3
QQKYDLLT

SEQ ID NO: 114 - protein, 119 aa, synthetic construct
  REGION 1..119: note = Anti-BCMA CT103A scFv heavy chain variable region
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 115 - protein, 10 aa, synthetic construct
  REGION 1..10: note = Anti-BCMA CT103A scFv heavy chain CDR1
GGSISSSSYY

SEQ ID NO: 116 - protein, 7 aa, synthetic construct
  REGION 1..7: note = Anti-BCMA CT103A scFv heavy chain CDR2
ISYSGST

SEQ ID NO: 117 - protein, 11 aa, synthetic construct
  REGION 1..11: note = Anti-BCMA CT103A scFv heavy chain CDR3
ARDRGDTILDV

SEQ ID NO: 118 - DNA, 1503 bp, synthetic construct
  source 1..1503: mol_type = other DNA
  misc_feature 1..1503: note = Exemplary BCMA CAR nucleotide sequence
atggccttaccagtgaccgccttgctcctgccgctggccttgctgctccacgccgccaggccggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaaaaatacgacctcctcacttttggcggagggaccaaggttgagatcaaaggcagcaccagcggctccggcaagcctggctctggcgagggcagcacaaagggacagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctcctatagtgggagcacctactacaacccgtccctcaagagtcgagtcaccatatccgtagacacgtccaagaaccagttctccctgaagctgagttctgtgaccgccgcagacacggcggtgtactactgcgccagagatcgtggagacaccatactagacgtatggggtcagggtacaatggtcaccgtcagctcattcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccaga

SEQ ID NO: 119 - protein, 501 aa, synthetic construct
  REGION 1..501: note = Exemplary BCMA CAR amino acid sequence
MALPVTALLLPLALLLHAARPDIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 120 - protein, 122 aa, synthetic construct
  REGION 1..122: note = CD8_1 VH
QVQLVQSGAEVKKPGASVKVSCKASGGTFSSYAISWVRQAPGQGLEWMGIIDPSDGNTNYAQNFQGRVTMTRDTSTSTVYMELSSLRSEDTAVYYCAKERAAAGYYYYMDVWGQGTTVTVSS

SEQ ID NO: 121 - protein, 108 aa, synthetic construct
  REGION 1..108: note = CD8_1 VL
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 122 - protein, 119 aa, synthetic construct
  REGION 1..119: note = CD8_2 VH
QVQLVQSGAEVKKPGASVKVSCKASGYTFTDYYIQWVRQAPGQGLEWMGWINPNSGGTSYAQKFQGRVTMTRDTSTSTVYMELSSLRSEDTAVYYCAKEGDYYYGMDAWGQGTMVTVSS

SEQ ID NO: 123 - protein, 113 aa, synthetic construct
  REGION 1..113: note = CD8_2 VL
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLQTPHTFGQGTKVEIKR

SEQ ID NO: 124 - protein, 117 aa, synthetic construct
  REGION 1..117: note = CD8_3 VH
QVQLVQSGAEVKKPGASVKVSCKASGYTFTSYYMHWVRQAPGQGLEWMGGFDPEDGETIYAQKFQGRVTMTRDTSTSTVYMELSSLRSEDTAVYYCARDQGWGMDVWGQGTTVTVSS

SEQ ID NO: 125 - protein, 108 aa, synthetic construct
  REGION 1..108: note = CD8_3 VL
DIQMTQSPSSLSASVGDRVTITCRASQSISSYLNWYQQKPGKAPKLLIYAASSLQSGVPSRFSGSGSGTDFTLTISSLQPEDFATYYCQQTYSTPYTFGQGTKLEIKR

SEQ ID NO: 126 - protein, 118 aa, synthetic construct
  REGION 1..118: note = CD8_4 VH
QVQLVQSGAEVKKPGASVKVSCKASGYTFTNHYMHWVRQAPGQGLEWMGWMNPNSGNTGYAQKFQGRVTMTRDTSTSTVYMELSSLRSEDTAVYYCASSESGSDLDYWGQGTLVTVSS

SEQ ID NO: 127 - protein, 108 aa, synthetic construct
  REGION 1..108: note = CD8_4 VL
DIQMTQSPSSLSASVGDRVTITCRASQTIGNYVNWYQQKPGKAPKLLIYGASNLHTGVPSRFSGSGSGTDFTLTISSLQPEDFATYYCQQTYSAPLTFGGGTKVEIKR

SEQ ID NO: 128 - protein, 125 aa, synthetic construct
  REGION 1..125: note = CD8_VHH
QVQLVESGGGLVQAGGSLRLSCAASGRTFSGYVMGWFRQAPGKQRKFVAAISRGGLSTSYADSVKGRFTISRDNAKNTVFLQMNTLKPEDTAVYYCAADRSDLYEITAASNIDSWGQGTLVTVSS

SEQ ID NO: 129 - protein, 8 aa, synthetic construct
  REGION 1..8: note = CDR-H1
GFTFSGYW

SEQ ID NO: 130 - protein, 8 aa, synthetic construct
  REGION 1..8: note = CDR-H2
ISPGGGST

SEQ ID NO: 131 - protein, 14 aa, synthetic construct
  REGION 1..14: note = CDR-H3
ASSLTATHTYEYDY

SEQ ID NO: 132 - protein, 121 aa, synthetic construct
  REGION 1..121: note = VHH
EVQLVESGGGLVQSGGSLRLSCAASGFTFSGYWMYWVRQAPGKGLEWVSAISPGGGSTYYPDSVKGRFTISRDNAKNTLYLQMNSLEPEDTALYYCASSLTATHTYEYDYWGQGTQVTVSS

SEQ ID NO: 133 - protein, 8 aa, synthetic construct
  REGION 1..8: note = CDR-H1
GYTFSNYW

SEQ ID NO: 134 - protein, 8 aa, synthetic construct
  REGION 1..8: note = CDR-H2
ILPGSGST

SEQ ID NO: 135 - protein, 13 aa, synthetic construct
  REGION 1..13: note = CDR-H3
ARRGYGYDEGFDY

SEQ ID NO: 136 - protein, 6 aa, synthetic construct
  REGION 1..6: note = CDR-L1
QDINSY

SEQ ID NO: 137 - skipped

SEQ ID NO: 138 - protein, 9 aa, synthetic construct
  REGION 1..9: note = CDR-L3
LQYDEFPPT

SEQ ID NO: 139 - protein, 120 aa, synthetic construct
  REGION 1..120: note = VH
QVQLQQSGAELMKPGASVKMSCKATGYTFSNYWIEWVKQRPGHGLEWIGEILPGSGSTSYNEKFKGKATFTADTSSSTAYMQLSSLTSEDSAVYYCARRGYGYDEGFDYWGQGSTLTVSS

SEQ ID NO: 140 - protein, 108 aa, synthetic construct
  REGION 1..108: note = VL
DIKMTQSPSSMYASLGERVTITCKASQDINSYLSWFQQKPGKSPKTLIYRANRLVDGVPSRFSGSGSGQDYSLTISSLEYEDMGIYYCLQYDEFPPTFGAGTKLELKR

SEQ ID NO: 141 - protein, 243 aa, synthetic construct
  REGION 1..243: note = scFv
QVQLQQSGAELMKPGASVKMSCKATGYTFSNYWIEWVKQRPGHGLEWIGEILPGSGSTSYNEKFKGKATFTADTSSSTAYMQLSSLTSEDSAVYYCARRGYGYDEGFDYWGQGSTLTVSSGGGGSGGGGSGGGGSDIKMTQSPSSMYASLGERVTITCKASQDINSYLSWFQQKPGKSPKTLIYRANRLVDGVPSRFSGSGSGQDYSLTISSLEYEDMGIYYCLQYDEFPPTFGAGTKLELKR

SEQ ID NO: 142 - protein, 8 aa, synthetic construct
  REGION 1..8: note = CDR-H1
GYTFTDYV

SEQ ID NO: 143 - protein, 8 aa, synthetic construct
  REGION 1..8: note = CDR-H2
IYPGSGSS

SEQ ID NO: 144 - protein, 10 aa, synthetic construct
  REGION 1..10: note = CDR-H3
ARPGDLGFAY

SEQ ID NO: 145 - protein, 10 aa, synthetic construct
  REGION 1..10: note = CDR-L1
QSVDYDGDSY

SEQ ID NO: 146 - skipped

SEQ ID NO: 147 - protein, 9 aa, synthetic construct
  REGION 1..9: note = CDR-L3
QQSNKDPFT

SEQ ID NO: 148 - protein, 117 aa, synthetic construct
  REGION 1..117: note = VH
QVQLQQSGPELVKPGASVKMSCKASGYTFTDYVISWVRQAPGQGLEWIGEIYPGSGSSYYNEKFKGRATLTADKSSNTAYMQLSSLRSEDSAVYFCARPGDLGFAYWGQGTLVTVSS

SEQ ID NO: 149 - protein, 112 aa, synthetic construct
  REGION 1..112: note = VL
DIVLTQSPSSLAVSLGQRATISCKASQSVDYDGDSYMNWYQQKPGQPPKLLIYAASNLESGIPARFSGSGSGTDFTLTIHPVEEEDAATYYCQQSNKDPFTFGGGTKLELKR

SEQ ID NO: 150 - protein, 244 aa, synthetic construct
  REGION 1..244: note = scFv
QVQLQQSGPELVKPGASVKMSCKASGYTFTDYVISWVRQAPGQGLEWIGEIYPGSGSSYYNEKFKGRATLTADKSSNTAYMQLSSLRSEDSAVYFCARPGDLGFAYWGQGTLVTVSSGGGGSGGGGSGGGGSDIVLTQSPSSLAVSLGQRATISCKASQSVDYDGDSYMNWYQQKPGQPPKLLIYAASNLESGIPARFSGSGSGTDFTLTIHPVEEEDAATYYCQQSNKDPFTFGGGTKLELKR

SEQ ID NO: 151 - protein, 8 aa, synthetic construct
  REGION 1..8: note = CDR-H1
GGTFSSYA

SEQ ID NO: 152 - protein, 8 aa, synthetic construct
  REGION 1..8: note = CDR-H2
INPNSGGT

SEQ ID NO: 153 - protein, 11 aa, synthetic construct
  REGION 1..11: note = CDR-H3
ARDGYSGSYSD

SEQ ID NO: 154 - protein, 12 aa, synthetic construct
  REGION 1..12: note = CDR-L1
QSVLSSSYNKNY

SEQ ID NO: 155 - skipped

SEQ ID NO: 156 - protein, 9 aa, synthetic construct
  REGION 1..9: note = CDR-L3
QQYYSTPWT

SEQ ID NO: 157 - protein, 118 aa, synthetic construct
  REGION 1..118: note = VH
QVQLVQSGAEVKKPGASVKVSCKASGGTFSSYAISWVRQAPGQGLEWMGWINPNSGGTNYAQKFQGRVTMTRDTSTSTVYMELSSLRSEDTAVYYCARDGYSGSYSDWGQGTLVTVSS

SEQ ID NO: 158 - protein, 113 aa, synthetic construct
  REGION 1..113: note = VL
DIVMTQSPDSLAVSLGERATINCKSSQSVLSSSYNKNYLAWYQQKPGQPPKLLIYWASTRESGVPDRFSGSGSGTDFTLTISSLQAEDVAVYYCQQYYSTPWTFGQGTKVEIK

SEQ ID NO: 159 - protein, 246 aa, synthetic construct
  REGION 1..246: note = scFv
QVQLVQSGAEVKKPGASVKVSCKASGGTFSSYAISWVRQAPGQGLEWMGWINPNSGGTNYAQKFQGRVTMTRDTSTSTVYMELSSLRSEDTAVYYCARDGYSGSYSDWGQGTLVTVSSGGGGSGGGGSGGGGSDIVMTQSPDSLAVSLGERATINCKSSQSVLSSSYNKNYLAWYQQKPGQPPKLLIYWASTRESGVPDRFSGSGSGTDFTLTISSLQAEDVAVYYCQQYYSTPWTFGQGTKVEIK

SEQ ID NO: 160 - protein, 8 aa, synthetic construct
  REGION 1..8: note = scFv
GYTFTSYD

SEQ ID NO: 161 - protein, 8 aa, synthetic construct
  REGION 1..8: note = CDR-H2
IIPLSGAP

SEQ ID NO: 162 - protein, 15 aa, synthetic construct
  REGION 1..15: note = CDR-H3
ARGALYNWNDGWFDP

SEQ ID NO: 163 - protein, 6 aa, synthetic construct
  REGION 1..6: note = CDR-L1
QDIGDY

SEQ ID NO: 164 - skipped

SEQ ID NO: 165 - protein, 9 aa, synthetic construct
  REGION 1..9: note = CDR-L3
QQANSFPLT

SEQ ID NO: 166 - protein, 122 aa, synthetic construct
  REGION 1..122: note = VH
QVQLVQSGAEVKKPGSSVKVSCKASGYTFTSYDINWVRQAPGQGLEWMGGIIPLSGAPNYAHKFQGRVTITADESTSTAYMELSSLRSEDTAVYYCARGALYNWNDGWFDPWGQGTLVTVSS

SEQ ID NO: 167 - protein, 107 aa, synthetic construct
  REGION 1..107: note = VL
DIQMTQSPSSLSASVGDRVTITCRASQDIGDYLAWYQQKPGKAPKLLIYDASSLQSGVPSRFSGSGSGTDFTLTISSLQPEDFATYYCQQANSFPLTFGGGTKVEIK

SEQ ID NO: 168 - protein, 244 aa, synthetic construct
  REGION 1..244: note = scFv
QVQLVQSGAEVKKPGSSVKVSCKASGYTFTSYDINWVRQAPGQGLEWMGGIIPLSGAPNYAHKFQGRVTITADESTSTAYMELSSLRSEDTAVYYCARGALYNWNDGWFDPWGQGTLVTVSSGGGGSGGGGSGGGGSDIQMTQSPSSLSASVGDRVTITCRASQDIGDYLAWYQQKPGKAPKLLIYDASSLQSGVPSRFSGSGSGTDFTLTISSLQPEDFATYYCQQANSFPLTFGGGTKVEIK

SEQ ID NO: 169 - protein, 8 aa, synthetic construct
  REGION 1..8: note = CDR-H1
GDTFSDHA

SEQ ID NO: 170 - protein, 8 aa, synthetic construct
  REGION 1..8: note = CDR-H2
MNPKIGNT

SEQ ID NO: 171 - protein, 12 aa, synthetic construct
  REGION 1..12: note = CDR-H3
VYDSSGYDAFDI

SEQ ID NO: 172 - protein, 12 aa, synthetic construct
  REGION 1..12: note = CDR-L1
QSVLSTSYNRNF

SEQ ID NO: 173 - protein, 9 aa, synthetic construct
  REGION 1..9: note = CDR-L3
QQYYSTPYT

SEQ ID NO: 174 - protein, 119 aa, synthetic construct
  REGION 1..119: note = VH
QVQLVQSGAEVKKPGASVKVSCKASGDTFSDHAINWVRQAPGQGLEWMGWMNPKIGNTGYAQKFQGRVTMTRDTSTSTVYMELSSLRSEDTAVYYCVYDSSGYDAFDIWGQGTTVTVSS

SEQ ID NO: 175 - protein, 113 aa, synthetic construct
  REGION 1..113: note = VL
DIVMTQSPDSLAVSLGERATINCKSSQSVLSTSYNRNFLAWYQQKPGQPPKLLIYWASTRQSGVPDRFSGSGSGTDFTLTISSLQAEDVAVYYCQQYYSTPYTFGQGTKLEIK

SEQ ID NO: 176 - protein, 247 aa, synthetic construct
  REGION 1..247: note = scFv
QVQLVQSGAEVKKPGASVKVSCKASGDTFSDHAINWVRQAPGQGLEWMGWMNPKIGNTGYAQKFQGRVTMTRDTSTSTVYMELSSLRSEDTAVYYCVYDSSGYDAFDIWGQGTTVTVSSGGGGSGGGGSGGGGSDIVMTQSPDSLAVSLGERATINCKSSQSVLSTSYNRNFLAWYQQKPGQPPKLLIYWASTRQSGVPDRFSGSGSGTDFTLTISSLQAEDVAVYYCQQYYSTPYTFGQGTKLEIK

SEQ ID NO: 177 - protein, 8 aa, synthetic construct
  REGION 1..8: note = CDR-H1
GYSLITHW

SEQ ID NO: 178 - protein, 8 aa, synthetic construct
  REGION 1..8: note = CDR-H2
INPSDGVT

SEQ ID NO: 179 - protein, 11 aa, synthetic construct
  REGION 1..11: note = CDR-H3
AREYYGEGFDY

SEQ ID NO: 180 - protein, 6 aa, synthetic construct
  REGION 1..6: note = CDR-L1
QGISNY

SEQ ID NO: 181 - skipped

SEQ ID NO: 182 - protein, 9 aa, synthetic construct
  REGION 1..9: note = CDR-L3
QQSYSTPLT

SEQ ID NO: 183 - protein, 118 aa, synthetic construct
  REGION 1..118: note = VH
QVQLVQSGAEVKKPGASVKVSCKASGYSLITHWMHWVRQAPGQGLEWMGMINPSDGVTYYAQTFQGRVTMTRDTSTSTVYMELSSLRSEDTAVYYCAREYYGEGFDYWGQGTLVTVSS

SEQ ID NO: 184 - protein, 108 aa, synthetic construct
  REGION 1..108: note = VL
DIQMTQSPSSLSASVGDRVTITCRASQGISNYLAWYQQKPGKAPKLLIYSASNLQSGVPSRFSGSGSGTDFTLTISSLQPEDFATYYCQQSYSTPLTFGGGTKVEIKR

SEQ ID NO: 185 - protein, 241 aa, synthetic construct
  REGION 1..241: note = scFv
QVQLVQSGAEVKKPGASVKVSCKASGYSLITHWMHWVRQAPGQGLEWMGMINPSDGVTYYAQTFQGRVTMTRDTSTSTVYMELSSLRSEDTAVYYCAREYYGEGFDYWGQGTLVTVSSGGGGSGGGGSGGGGSDIQMTQSPSSLSASVGDRVTITCRASQGISNYLAWYQQKPGKAPKLLIYSASNLQSGVPSRFSGSGSGTDFTLTISSLQPEDFATYYCQQSYSTPLTFGGGTKVEIKR

SEQ ID NO: 186 - protein, 5 aa, synthetic construct
  REGION 1..5: note = CDR-H1
GYWMY

SEQ ID NO: 187 - protein, 17 aa, synthetic construct
  REGION 1..17: note = CDR-H2
AISPGGGSTYYPDSVKG

SEQ ID NO: 188 - protein, 12 aa, synthetic construct
  REGION 1..12: note = CDR-H3
SLTATHTYEYDY

SEQ ID NO: 189 - protein, 7 aa, synthetic construct
  REGION 1..7: note = CDR-H1
GFTFSGY

SEQ ID NO: 190 - protein, 6 aa, synthetic construct
  REGION 1..6: note = CDR-H2
SPGGGS

SEQ ID NO: 191 - protein, 5 aa, synthetic construct
NYWIE

SEQ ID NO: 192 - protein, 17 aa, synthetic construct
  REGION 1..17: note = CDR-H2
EILPGSGSTSYNEKFKG

SEQ ID NO: 193 - protein, 11 aa, synthetic construct
  REGION 1..11: note = CDR-H3
RGYGYDEGFDY

SEQ ID NO: 194 - protein, 11 aa, synthetic construct
  REGION 1..11: note = CDR-L1
KASQDINSYLS

SEQ ID NO: 195 - protein, 7 aa, synthetic construct
  REGION 1..7: note = CDR-L2
RANRLVD

SEQ ID NO: 196 - protein, 7 aa, synthetic construct
  REGION 1..7: note = CDR-H1
GYTFSNY

SEQ ID NO: 197 - protein, 6 aa, synthetic construct
  REGION 1..6: note = CDR-H2
LPGSGS

SEQ ID NO: 198 - protein, 5 aa, synthetic construct
  REGION 1..5: note = CDR-H1
DYVIS

SEQ ID NO: 199 - protein, 17 aa, synthetic construct
  REGION 1..17: note = CDR-H2
EIYPGSGSSYYNEKFKG

SEQ ID NO: 200 - protein, 8 aa, synthetic construct
  REGION 1..8: note = CDR-H3
PGDLGFAY

SEQ ID NO: 201 - protein, 15 aa, synthetic construct
  REGION 1..15: note = CDR-L1
KASQSVDYDGDSYMN

SEQ ID NO: 202 - protein, 7 aa, synthetic construct
  REGION 1..7: note = CDR-L2
AASNLES

SEQ ID NO: 203 - protein, 7 aa, synthetic construct
  REGION 1..7: note = CDR-H1
GYTFTDY

SEQ ID NO: 204 - protein, 6 aa, synthetic construct
  REGION 1..6: note = CDR-H2
YPGSGS

SEQ ID NO: 205 - protein, 5 aa, synthetic construct
  REGION 1..5: note = CDR-H1
SYAIS

SEQ ID NO: 206 - protein, 17 aa, synthetic construct
  REGION 1..17: note = CDR-H2
WINPNSGGTNYAQKFQG

SEQ ID NO: 207 - protein, 9 aa, synthetic construct
  REGION 1..9: note = CDR-H3
DGYSGSYSD

SEQ ID NO: 208 - protein, 17 aa, synthetic construct
  REGION 1..17: note = CDR-L1
KSSQSVLSSSYNKNYLA

SEQ ID NO: 209 - protein, 7 aa, synthetic construct
  REGION 1..7: note = CDR-L2
WASTRES

SEQ ID NO: 210 - protein, 7 aa, synthetic construct
  REGION 1..7: note = CDR-H1
GGTFSSY

SEQ ID NO: 211 - protein, 6 aa, synthetic construct
  REGION 1..6: note = CDR-H2
NPNSGG

SEQ ID NO: 212 - protein, 5 aa, synthetic construct
  REGION 1..5: note = CDR-H1
SYDIN

SEQ ID NO: 213 - protein, 17 aa, synthetic construct
  REGION 1..17: note = CDR-H2
GIIPLSGAPNYAHKFQG

SEQ ID NO: 214 - protein, 13 aa, synthetic construct
  REGION 1..13: note = CDR-H3
GALYNWNDGWFDP

SEQ ID NO: 215 - protein, 11 aa, synthetic construct
  REGION 1..11: note = CDR-L1
RASQDIGDYLA

SEQ ID NO: 216 - protein, 7 aa, synthetic construct
  REGION 1..7: note = CDR-L2
DASSLQS

SEQ ID NO: 217 - protein, 7 aa, synthetic construct
  REGION 1..7: note = CDR-H1
GYTFTSY

SEQ ID NO: 218 - protein, 6 aa, synthetic construct
  REGION 1..6: note = CDR-H2
IPLSGA

SEQ ID NO: 219 - protein, 5 aa, synthetic construct
  REGION 1..5: note = CDR-H1
DHAIN

SEQ ID NO: 220 - protein, 17 aa, synthetic construct
  REGION 1..17: note = CDR-H2
WMNPKIGNTGYAQKFQG

SEQ ID NO: 221 - protein, 10 aa, synthetic construct
  REGION 1..10: note = CDR-H3
DSSGYDAFDI

SEQ ID NO: 222 - protein, 17 aa, synthetic construct
  REGION 1..17: note = CDR-L1
KSSQSVLSTSYNRNFLA

SEQ ID NO: 223 - protein, 7 aa, synthetic construct
  REGION 1..7: note = CDR-L2
WASTRQS

SEQ ID NO: 224 - protein, 7 aa, synthetic construct
  REGION 1..7: note = CDR-H1
GDTFSDH

SEQ ID NO: 225 - protein, 6 aa, synthetic construct
  REGION 1..6: note = CDR-H2
NPKIGN

SEQ ID NO: 226 - protein, 5 aa, synthetic construct
  REGION 1..5: note = CDR-H1
THWMH

SEQ ID NO: 227 - protein, 17 aa, synthetic construct
  REGION 1..17: note = CDR-H2
MINPSDGVTYYAQTFQG

SEQ ID NO: 228 - protein, 9 aa, synthetic construct
  REGION 1..9: note = CDR-H3
EYYGEGFDY

SEQ ID NO: 229 - protein, 11 aa, synthetic construct
  REGION 1..11: note = CDR-L1
RASQGISNYLA

SEQ ID NO: 230 - protein, 7 aa, synthetic construct
  REGION 1..7: note = CDR-L2
SASNLQS

SEQ ID NO: 231 - protein, 7 aa, synthetic construct
  REGION 1..7: note = CDR-H1
GYSLITH

SEQ ID NO: 232 - protein, 6 aa, synthetic construct
NPSDGV